(19) United States
(12) Patent Application Publication
(10) Pub. No.: US 2025/0241944 A1
(43) Pub. Date: Jul. 31, 2025

(54) TIMED AND/OR TARGETED CHLORATE ADMINISTRATION, AND RELATED MATRICES, COMPOSITIONS, IMPLANTS, METHODS AND SYSTEMS FOR PREVENTION AND/OR TREATMENT OF INFECTIONS

(71) Applicants: CALIFORNIA INSTITUTE OF TECHNOLOGY, Pasadena, CA (US); THE REGENTS OF THE UNIVERSITY OF CALIFORNIA, Oakland, CA (US)

(72) Inventors: Dianne K NEWMAN, Pasadena, CA (US); Melanie A SPERO, Eugene, OR (US); Manuela MARTINS-GREEN, Riverside, CA (US)

(21) Appl. No.: 18/805,379

(22) Filed: Aug. 14, 2024

Related U.S. Application Data
(60) Provisional application No. 63/519,537, filed on Aug. 14, 2023.
(60) Provisional application No. 63/670,084, filed on Jul. 11, 2024.

(51) Int. Cl. A61K 33/00; A01N 59/00; A01P 1/00; A61B 5/145; A61F 13/00; A61L 26/00; A61P 31/02

(52) U.S. Cl. CPC A61K 33/00; A01N 59/00; A01P 1/00; A61F 13/00063; A61L 26/0066; A61P 31/02; A61B 5/14542

(57) ABSTRACT

Methods and systems and related compositions, matrices and devices, for timed and/or targeted administration of chlorate for treatment and/or prevention of infections of a biological environment and related compositions, devices, matrices and implants. Chlorate administration can be performed alone or in combination with an antibiotic in a location and/or time targeted manner, the concentration and use of the chlorate and/or the antibiotic agents depending on the oxic/hypoxic/anoxic condition of the area being treated.

### CROSS-REFERENCE TO RELATED APPLICATION

The present application is claims priority to U.S. Provisional Application No. 63/519,537, entitled “Timed and/or targeted chlorate administration matrices, compositions, implants, methods and systems for prevention and/or treatment of infections” filed on Aug. 14, 2023, with docket number P2957-USP, the content of which is incorporated by reference in its entirety. The present application also claims priority to U.S. Provisional Application No. 63/670,084, entitled “Timed and/or targeted chlorate administration matrices, compositions, implants, methods and systems for prevention and/or treatment of infections” filed on Jul. 11, 2024, with docket number P2957-USP2, the content of which is incorporated by reference in its entirety.

The present application may further be related to U.S. Ser. No. 17,234,656, entitled ‘Wound Prevention and/or Treatment and Related Compounds, Matrices, Compositions, Methods and Systems” filed on Apr. 19, 2021 with docket number P2493-US, to U.S. Provisional Application No. 63/012,036 entitled “Wound Prevention and/or Treatment and Related Compounds, Matrices, Compositions, Methods and Systems” filed on Apr. 17, 2020 with docket number P2493-USP, the content of each of which is incorporated by reference in its entirety. The present application may also be related to U.S. provisional application No. 62/571,009, entitled “New Therapeutic Strategy to Combat Diverse Chronic Infections” filed on Oct. 11, 2017 with docket number CIT 7310-P3, to U.S. application Ser. No. 16/157,885 entitled “Methods and Systems to interfere with viability of bacteria and related Antimicrobials and Compositions” filed on Oct. 11, 2018, with docket number P2286-US, and to PCT application PCT/US2018/055416 entitled “Methods and Systems to interfere with viability of bacteria and related Antimicrobials and Compositions” filed on Oct. 11, 2018, with docket number P2286-PCT, the content of each of which is also incorporated by reference in its entirety.

### STATEMENT OF GOVERNMENT GRANT

This invention was made with government support under Grant No. AI146987 awarded by the National Institutes of Health. The government has certain rights in the invention.

### REFERENCE TO SEQUENCE LISTING

Further, the computer readable form of the sequence listing of the ASCII (XML) text file P2957-US-2025-04-11-Seq-ID-ST26.xml, created on Apr. 11, 2025, with a size of 113,331 bytes measured on Windows Server 2019, is incorporated herein by reference in its entirety.

### FIELD

The present disclosure relates to treatment and/or prevention of infections and related compounds, matrices, compositions methods and systems. In particular the present disclosure relates to timed and/or targeted chlorate administration, matrices compositions, implant methods and systems for treatment and/or prevention of bacterial infections of an individual.

### BACKGROUND

Bacterial viability has been the focus of research in the field of biological analysis, in particular, when aimed at medical applications such as therapeutic or diagnostic applications.

Whether for pathological examination or for fundamental biology studies, several methods are commonly used for the detection of and interference with the viability of bacteria.

Various methods, systems and compositions have been developed to interfere with, and in particular, to reduce bacterial viability to the extent of killing the bacteria. However, antibiotic resistance and additional defense mechanisms of the microorganism have made the development of methods, systems and compositions that are able to interfere with and in particular to inhibit bacterial viability particularly challenging.

### SUMMARY

Provided herein are methods, systems for timed and/or targeted administration of chlorate to treat a bacterial infection such as wound infection and related matrices, compounds, compositions and implants, which in several embodiments can be used to effectively inhibit bacteria biofilm formation and/or disrupt bacterial biofilm formed within an infected biological environment within an individual and/or to promote healing of the infection in the individual.

In particular, methods and systems herein described and related matrices, compounds, compositions and implants are based on an optimization of the timing of chlorate administration and/or in the optimization of the delivery of the chlorate to an infected biological environment to maximize the antibacterial effect of the chlorate against Nar-containing bacteria, both in case the chlorate is administered alone, and in combination with antibiotics, antimicrobials and/or wound healing agents as will be understood by a skilled person upon reading of the present disclosure.

According to a first aspect, a timed chlorate administration method and a system are described of treating an infected biological environment which are based on an optimization of the timing of chlorate administration. The method comprises contacting the biological environment with an effective amount of chlorate to treat and/or prevent infection of Nar-containing bacteria, the contacting performed at a chlorate administration time when the Nar-containing bacteria, if any is present, undergo anaerobic respiration within the biological environment.

In particular, in the timed chlorate administration method of the first aspect, the timing for chlorate administration can be identified by detecting in the biological environment at least one of oxygen level, redox potential and nitrate concentration, preferably oxygen level alone or in combination with nitrate concentration and optionally redox potential to detect an oxygenation status of the biological environment. The chlorate timed method further comprises selecting the timing of chlorate administration when the oxygen level is below a threshold detected oxygen level of for example 200 uM, the threshold being preferably of 150 uM or more, or preferably 100 uM, redox potential is below a threshold detected redox potential of for example 300 mV, or preferably of 200 mV, and/or nitrate concentration is above a threshold nitrate concentration, preferably of 500 uM. Preferably, the timing for chlorate administration is identified following quantitative detection of all those three markers when oxygen level is below the detected threshold, preferably 100 uM, redox potential is below the detected threshold, preferably 200 mV, and nitrate concentration is above the detected threshold, preferably 500 uM.

More preferably, in the chlorate timed administration method of the disclosure, the method further comprises administering chlorate alone at a chlorate administration time when the environment is under anoxic condition, with a detected oxygen level lower than 20 uM, and in combination with antibiotic a chlorate administration time when the environment is under hypoxic condition with a detected oxygen level from 200-20 μM.

Preferably chlorate can be administered alone without need of antibiotic administration or in combination with an antibiotic at a chlorate administration time when the detected oxygen level of the biological environment is lower than 20 uM, the detected nitrate concentration is above 100 uM more preferably above 500 uM, more preferably in combination with detected redox potential of the biological environment below 200 mV. More preferably chlorate can be administered in combination with an antibiotic at a chlorate administration time when the detected oxygen level of the biological environment is from 200-20 uM, the detected nitrate concentration is below 500 uM, more preferably from luM to 100 uM more preferably in combination with detected redox potential of the biological environment below 200 mV.

Preferably, chlorate can be administered in a chlorate effective amount under hypoxic conditions which ranges from 0.001 to 10 uM, more preferably or 0.01 to 10 mM and most preferably 0.1 mM to 10 mM.

Preferably, chlorate can be administered in a chlorate effective amount under anoxic conditions from 0.001 to 10 uM, more preferably or 0.01 to 10 mM and most preferably 0.1 mM to 10 mM.

In some preferred embodiments the detecting can be performed on a same biological environment at a plurality of times to detect an oxygenation status of the biological environment over time, the oxygenation status selected from oxic condition, hypoxic condition and anoxic condition. In those embodiments, the chlorate administration, the chlorate effective amount, can be performed based on the oxygenation status of the biological environment detected over time.

The timed chlorate administration system according the first aspect comprises one or more chlorates in hypoxic and/or anoxic effective concentrations and at least one of a look up table connecting amounts of chlorate, timing of administrations and stage of the biological environment, according to the timed chlorate administration method of the first aspect, at least one of an oxygen sensor, electrodes for detection of redox potentials, and electrodes and/or reagents for detection of nitrate with corresponding instructions in the look-up table in connection with detected values with respect to threshold value, optionally reagents to detect biomarker of anaerobic respiration and reagents to detect biomarker of aerobic respiration.

Additional optional components comprise nitrate in effective concentrations to be administered in accordance with the indications of the look-up table, at least one antibiotic in oxic, hypoxic and/or anoxic effective concentrations, reagents to detect biomarker of anaerobic respiration and reagents to detect biomarker of aerobic respiration optionally in connection with corresponding instructions in the look up table in connection with detected quantitative and/or qualitative values with respect to threshold values, at least one antimicrobial and at least one wound healing agent as would be understood by a skilled person upon reading of the present disclosure.

According to a second aspect, an antibiotic timed chlorate administration method and a system are described to treat an infected biological environment by chlorate administration. The method comprises contacting the biological environment with an antibiotic before and/or following the chlorate administration. In the method, contacting the antibiotic with the biological environment is performed at an antibiotic administration time when the Nar-containing bacteria, if any is present, undergo aerobic respiration.

In particular, in some embodiments of the antibiotic timed chlorate administration method according to the second aspect, the timing for antibiotic administration can be identified by detecting in the biological environment at least one of oxygen level, redox potential and nitrate concentration, preferably oxygen level alone or in combination with nitrate concentration and optionally redox potential, to detect an oxygenation status of the biological environment.

The antibiotic timed chlorate administration method further comprises selecting the timing of antibiotic administration of antibiotic when the detected oxygen level is above or below a threshold detected oxygen level of 200 uM, or preferably 150 uM or more, or preferably 100 uM, redox potential is above a threshold detected redox potential of 300 mV, or preferably of 200 mV, and/or nitrate concentration is below 500 uM.

Preferable timing for antibiotic administration is identified following quantitative detection of all those three markers when oxygen level is above the detected threshold, preferably 100 uM, redox potential is above the detected threshold, preferably 200 mV, and nitrate concentration is below the detected threshold, preferably 500 uM. In some most preferred embodiments antibiotic administration to the biological environment can occur in combination with the administration of chlorate, before, concurrently and/or subsequently to chlorate administration.

More preferably, in the antibiotic timed chlorate administration method of the disclosure the method further comprises administering one or more antibiotics alone at an antibiotic administration time when the biological environment is under oxic condition with detected oxygen level is higher than 200 uM, and in combination with chlorate at an antibiotic administration time when the biological environment is under hypoxic condition with detected oxygen levels are from 200-20 uM.

Preferably antibiotic can be administered alone at an antibiotic administration time when the detected oxygen level of the biological environment is higher than 200 uM, the detected nitrate concentration is below 100 uM, more preferably in combination with detected redox potential of the biological environment above 200 mV. More preferably antibiotic can be administered in combination with a chlorate at an antibiotic administration time when the detected oxygen level of the biological environment is from 200-20 uM, the detected nitrate concentration is below 500 uM, more preferably from 100 uM to 500 uM more preferably in combination with detected redox potential of the biological environment below 200 mV.

In some preferred embodiments of the antibiotic timed chlorate administration method according to the second aspect, no antibiotic is administered or antibiotic is administered at an anoxic effective concentration, when the detected oxygen level is lower than 20 uM, and the detected nitrate concentration is above 100 uM more preferably above 500 uM. In some of those embodiments, the detected redox potential is preferably below 200 mV. In most preferred embodiments, when the one or more antibiotic are administered the concentration of antibiotics administered under hypoxic conditions in combination with chlorate can be reduced to a fraction of the Minimum Inhibitory Concentration (MIC).

Preferably, in the antibiotic timed chlorate administration method the antibiotic can be administered in an antibiotic effective amount under oxic conditions are amounts resulting in delivery of the antibiotic MIC. Typically, an oxic antibiotic effective concentration comprise an amount ranging from 0.1 ug/mL to 500 ug/mL preferably from 1 to 500 ug/mL, from 1-30 ug/mL or 1-5 ug/mL in view of the bacteria susceptibility to the antibiotic, the target objective of the treatment (e.g. desired therapeutic effect) the antibiotic used and the biological environment treated (e.g. skin, blood, muscles, lungs, mucosa and others identifiable by a skilled person).

Preferably, in the antibiotic timed chlorate administration method, the antibiotic can be administered in an antibiotic effective amount under hypoxic conditions are amounts resulting in delivery of amounts of antibiotic which is lower than the MIC and/or MBC, a fraction of the MIC for the antibiotic, preferably half the MIC, more preferably one quarter of the MIC, or lower up to one tenth of the MIC thus resulting in amounts typically ranging from 0.001 to 500 ug/ml, with higher concentrations corresponding from 0.1-500 ug/ml preferably 1-30 ug/mL or 1-5 ug/mL to increase the efficacy of the treatment in view of the bacteria susceptibility to the antibiotic, the target objective of the treatment (e.g. desired therapeutic effect) the antibiotic used and the biological environment treated (e.g. skin, blood, muscles, lungs, mucosa and others identifiable by a skilled person).

In some preferred embodiments the detecting can be performed on a same biological environment at a plurality of times to detect an oxygenation status of the biological environment selected from oxic condition, hypoxic condition and anoxic condition. In those embodiments, the antibiotic administration, the antibiotic effective amount are selected base on the oxygenation status of the biological environment detected over time.

The antibiotic timed chlorate administration system of the second aspect comprises one or more chlorate in hypoxic and/or anoxic effective concentrations, one or more antibiotic in oxic, hypoxic and/or anoxic effective concentrations and at least one of a look up table connecting amounts and timing of administration of chlorate, amounts and timing of administration of antibiotic with respect to the administration of the chlorate in accordance with the antibiotic timed chlorate administration of the second aspect, and at least one of an oxygen sensor, electrodes for detection of redox potential, and electrodes and/or reagents for detection of nitrate concentrations with corresponding instructions in the look-up table in connection with detected values with respect to threshold value optionally reagents to detect biomarker of anaerobic respiration and reagents to detect biomarker of aerobic respiration with corresponding instructions in the look-up table in connection with detected qualitative and/or quantitative values with respect to threshold value. Additional optional components comprise, nitrate at effective concentrations to be administered in accordance with the indications of the look-up table, at least one antimicrobial and at least one wound healing agent as would be understood by a skilled person upon reading of the present disclosure.

According to a third aspect, a targeted chlorate administration method and a system are described of treating or preventing an infection in a biological environment. The method comprises contacting the chlorate with a hypoxic and/or anoxic region of a biological environment, comprising oxygen at a level enabling anaerobic respiration by a Nar containing bacteria if any is present. In the targeted chlorate administration method of the disclosure, the contacting of chlorate with the anoxic region of the biological environment can be performed in the absence of one or more antibiotics. In the targeted chlorate administration method of the disclosure, contacting of chlorate with the hypoxic portion of the infected biological environment is performed in combination with one or more antibiotics. In preferred embodiments, the method further comprises contacting an antibiotic, with an oxic region of the infected biological environment comprising oxygen at a level enabling aerobic respiration by a Nar containing bacteria if any is present.

In particular, in the targeted chlorate administration method of the third aspect, the hypoxic and/or anoxic regions of the biological environment targeted by chlorate administration can be identified by detecting in the biological environment at least one of oxygen level, redox potential and nitrate concentration preferably oxygen level alone or in combination with nitrate concentration and optionally redox potential to detect an oxygenation status of the target region. The targeted chlorate administration method further comprises selecting the target region for chlorate administration when the oxygen level is below a detected threshold of 200 uM, preferably 100 uM, a redox potential is below the detected threshold, preferably 200 mV, and/or nitrate concentration is above a detected threshold of 100 uM, preferably 500 uM.

In the targeted chlorate administration method of the disclosure, preferably one or more hypoxic regions of the biological environment targeted for chlorate administration can be identified following quantitative detection of all those three markers when oxygen level is below a detected threshold of 200 um, preferably 100 uM and above 20 uM, a redox potential is below the detected threshold, preferably 200 mV, and nitrate concentration is above a detected threshold of 100 uM, preferably 500 uM.

In the targeted chlorate administration method of the disclosure, preferably the method further comprises administering chlorate alone to a targeted anoxic region, having a detected oxygen level is lower than 20 uM, and in combination with antibiotic in a targeted hypoxic region having a detected oxygen levels are from 200-20 uM. Preferably chlorate can be administered alone to a targeted anoxic region having a oxygen level lower than 20 uM, a detected nitrate concentration above 100 uM more preferably above 500 uM, more preferably in combination with a detected redox potential below 200 mV.

Preferably, in targeted chlorate administration method of the disclosure, chlorate can be administered in a chlorate effective amount to hypoxic region of a biological environment which preferably ranges from 0.001 to 10 uM, more preferably or 0.01 to 10 mM and most preferably 0.1 mM to 10 mM.

Preferably, in targeted chlorate administration method of the disclosure, chlorate can be administered in a chlorate effective amount to an anoxic region of the biological environment which preferably ranges from 0.001 to 10 uM, more preferably or 0.01 to 10 mM and most preferably 0.1 mM to 10 mM.

In some preferred embodiments, the targeted chlorate administration method of the disclosure, further comprises detecting in at least one target region of the biological environment at least one of oxygen level, redox potential and nitrate concentration, preferably oxygen level alone or in combination with nitrate concentration and optionally redox potential, to detect an oxygenation status in the at least one target region. In those embodiments the selecting is performed by selecting the target region for chlorate administration and the chlorate effective amount based the detected oxygenation status of the target region.

In preferred embodiments, the detecting can be performed on a same biological environment at a plurality of target region to detect an oxygenation status of the plurality of target regions of the biological environment, the oxygenation status selected from oxic condition, hypoxic condition and anoxic condition, and the chlorate administration time and the chlorate effective amount are selected based the detected oxygenation status each target region of the plurality of target region.

The targeted chlorate administration system of the third aspect comprises at least one chlorate anoxic and/or hypoxic effective concentrations and a look up table connecting amounts of chlorate with anoxic target portions of at least one biological environment for targeted administration of the chlorate in at least one target region of a biological environment (e.g. target region of a wound or infected tissues) according to the targeted chlorate administration method of the third aspect. Additional optional components comprise at least one antibiotic in oxic, hypoxic and/or anoxic effective concentrations, at, least one of an oxygen sensor, electrodes for detection of redox potential, and electrodes and/or reagents for detection of nitrate concentrations with corresponding instructions in the look-up table in connection with detected values with respect to threshold value, nitrate at effective concentrations to be administered in accordance with the indications of the look-up table, at least one antimicrobial and at least one wound healing agent as well as reagents for detecting biomarker of anaerobic respiration and reagents for detecting biomarker of aerobic respiration with corresponding instructions in the look-up table in connection with detected qualitative and/or quantitative values with respect to threshold value as would be understood by a skilled person upon reading of the present disclosure.

According to a fourth aspect, a timed and targeted chlorate administration method and a system are described of treating an infected biological environment which are based on an optimization of the timing and targeting of chlorate and antibiotic administration. The method comprises contacting the biological environment with an effective amount of chlorate to treat and/or prevent infection of Nar-containing bacteria, the contacting performed with a hypoxic region and/or an anoxic region of targeted biological environment at a chlorate administration time when the Nar-containing bacteria, if any is present, undergo anaerobic respiration within the biological environment.

In particular, in the timed and targeted chlorate administration method of the disclosure the hypoxic region and/or anoxic region which is targeted by chlorate administration and the related timing of administration can be identified by detecting in at least one target region of the biological environment at least one of oxygen level, redox potential and nitrate concentration preferably oxygen level alone or in combination with nitrate concentration and optionally redox potential, the detecting performed at least one detection time, to detect an oxygenation status of at least one target region selected from oxic, hypoxic and anoxic.

The chlorate timed and targeted method for chlorate administration further comprises administering an effective amount of chlorate to a target region of the at least one target region when the target region is under hypoxic or anoxic condition with oxygen level below the detected threshold of 200 nM, preferably below 100 uM, a redox potential below the detected threshold, preferably 200 mV, and/or a nitrate concentration s above the detected threshold, preferably 500 uM. Preferably one or more hypoxic regions that can be targeted by chlorate administration and/or related timing of administration can be identified following quantitative detection of all those three markers when oxygen level is below the detected threshold of 200 uM, preferably below 100 uM and above 20 uM, a redox potential is below the detected threshold, preferably 200 mV, and a nitrate concentration is above the detected threshold, preferably 500 uM An anoxic region can be identified by a detected oxygen level is lower than 20 uM.

The timed and targeted chlorate administration method further comprises contacting an antibiotic to an oxic and/or hypoxic region of the at least one target region of the biological environment. In some embodiments the oxic and/or hypoxic region comprises one or more chlorate treated portions of the biological environment. In some embodiments the contacting of the antibiotic is performed at an antibiotic administration time following chlorate administration to one or more portions of the biological environment for example at a time when the chlorate has penetrated into a biofilm formed by the Nar-containing bacteria, if any is present, in the biological environment. In some embodiments the contacting of the antibiotic is performed at an antibiotic administration time preceding or concurrent to chlorate administration to one or more portions of the biological environment.

Accordingly in a timed and targeted chlorate administration method of the fourth aspect, chlorate is administered alone and without need of antibiotic to one or more anoxic target regions of the biological environment which can be identified by a detected oxygen level is lower than 20 uM, In the timed and target method of chlorate administration of the fourth aspect chlorate in combination with antibiotic in a targeted region under hypoxic condition having a detected oxygen levels are from 200-20 uM, while antibiotic is administered without need of chlorate to oxic region of biological environment.

Preferably, in a timed and targeted method of chlorate administration of the fourth aspect, chlorate can be administered in a chlorate effective amount to target region of a biological environment under hypoxic condition which preferably ranges from 0.001 to 10 uM, more preferably or 0.01 to 10 mM and most preferably 0.1 mM to 10 mM.

Preferably, in a timed and targeted method of chlorate administration of the fourth aspect, chlorate can be administered in a chlorate effective amount to a target region of the biological environment under anoxic condition which preferably ranges from 0.001 to 10 uM, more preferably or 0.01 to 10 mM and most preferably 0.1 mM to 10 mM.

Preferably, in a timed and targeted method of chlorate administration of the fourth aspect, antibiotic can be administered in an antibiotic effective amount under oxic conditions (herein oxic antibiotic effective concentrations) are amounts resulting in delivery of the antibiotic MIC or MBC. Typically an oxic antibiotic effective concentration comprise an amount ranging from 0.1 ug/mL to 500 ug/mL preferably from 1 to 500 ug/mL, from 1-30 ug/mL or 1-5 ug/mL in view of the bacteria susceptibility to the antibiotic, the target objective of the treatment (e.g. desired therapeutic effect) the antibiotic used and the biological environment treated (e.g. skin, blood, muscles, lungs, mucosa and others identifiable by a skilled person).

Preferably, in a timed and targeted method of chlorate administration of the fourth aspect, antibiotic can be administered in an antibiotic effective amount under hypoxic conditions are amounts resulting in delivery of amounts of antibiotic which is lower than the MIC for the antibiotic, preferably half the MIC, more preferably one quarter of the MIC, or lower up to one tenth of the MIC, thus resulting in amounts typically ranging from 0.001 to 500 ug/ml, with higher concentrations corresponding from 0.1-500 ug/ml preferably 1-30 ug/mL or 1-5 ug/mL to increase the efficacy of the treatment in view of the bacteria susceptibility to the antibiotic, the target objective of the treatment (e.g. desired therapeutic effect) the antibiotic used and the biological environment treated (e.g. skin, blood, muscles, lungs, mucosa and others identifiable by a skilled person).

In some preferred embodiments the detecting can be performed on the at least one target region of the biological environment at a plurality of times to monitor the change in oxygenation status of the at least one target region of the biological environment. In those embodiments, the chlorate administration, the chlorate effective amount, the antibiotic administration time and the antibiotic effective amount for a target region of the at least one target region are selected in time based on an oxygenation status to the target region detected over time.

The chlorate timed and targeted administration system comprises one or more chlorates, in hypoxic and/or anoxic effective concentrations, one or more antibiotics in oxic, hypoxic and/or anoxic effective concentrations and a look up table connecting amounts and timing of administration of chlorate, amounts and timing of administration of antibiotic with respect to the administration of the chlorate, stages of the infection and preferably also targeted portions of the biological environment where chlorate administration should be performed (e.g. regions of a wound, or other tissues such as a infected tissues such as lungs or muscles as will be understood by a skilled person) according to the chlorate timed and targeted administration of the fourth aspect. Additional optional components comprise at least one of an oxygen sensor, electrodes for detection of redox potentials, and electrodes and or reagents for detection of nitrate, with corresponding instructions in the look-up table in connection with detected values with respect to threshold value, nitrate in effective concentrations to be administered in accordance with the indications of the look-up table at least one antimicrobial and at least one wound healing agent as well as other reagents for detecting a biomarker of anaerobic respiration, a biomarker of aerobic respiration with corresponding instructions in the look-up table in connection with detected qualitative and/or quantitative values with respect to threshold value, as would be understood by a skilled person upon reading of the present disclosure.

According to a fifth aspect, a method for timed and/or targeted chlorate administration of a biological environment or a region thereof, are described the method comprising
administering an antibiotic to the biological environment or region thereof when the biological environment or region thereof is in an oxic condition, the administering performed at an oxic antibiotic effective amount to inhibit viability of Nar-containing bacteria in an oxic environment,
administering chlorate in combination with an antibiotic to the biological environment or region thereof, when the biological environment or region thereof in a hypoxic condition, the administering performed at a hypoxic chlorate effective amount and a hypoxic antibiotic effective amount to inhibit viability of Nar-containing bacteria in a hypoxic environment; and/or
administering chlorate to the biological environment or the region thereof, the biological environment or region thereof in an anoxic condition, the administering performed at an anoxic chlorate effective amount to inhibit viability of Nar-containing bacteria in an anoxic environment
the administering chlorate to the biological environment or region thereof in anaerobic conditions, performed preferably in combination with an antibiotic in an antibiotic effective among in anoxic environment.

Preferably the method also comprises detecting in at least one target region of the biological environment at least one of oxygen level, nitrate concentration and redox potential of a target region of the biological environment, preferably oxygen level alone or in combination with nitrate concentration and optionally redox potential to detect an oxygenation status of the target region selected from oxic status hypoxic status and anoxic status.

The chlorate timed and targeted administration system of the fifth aspect comprises one or more chlorates hypoxic and/or anoxic effective concentrations, one or more antibiotics in an oxic, hypoxic and/or anoxic effective concentrations and a look up table connecting amounts and timing and/or targeting of administration of chlorate, amounts and timing and/or targeting of administration of antibiotic with respect to the administration of the chlorate, stages of the infection and preferably also targeted portions of the biological environment where chlorate administration and antibiotic administration should be performed preferably in combination with detected values of oxygen level, redox potential and nitrate concentration according to the chlorate timed and targeted administration method of the fifth aspect. Additional components comprise at least one of an oxygen sensor, electrodes for detection of redox potentials, and electrodes and or reagents for detection of nitrate, with corresponding instructions in the look-up table in connection with detected values with respect to threshold value, nitrate in effective concentrations to be administered in combination with chlorate as indicated in the look up table, at least one antimicrobial and at least one wound healing agent as well as other reagents for detecting a biomarker of anaerobic respiration, a biomarker of aerobic respiration with corresponding instructions in the look-up table in connection with detected qualitative and/or quantitative values with respect to threshold value, as would be understood by a skilled person upon reading of the present disclosure.

According to a sixth aspect, a timed and targeted chlorate administration method and a system are described of treating a biological environment infected by a Nar-containing bacteria, which are based on an optimization of the timing and targeting of chlorate and antibiotic administration. The method comprises detecting in at least one target region of the biological environment at least one of oxygen level, nitrate concentration and redox potential of a target region of the biological environment, preferably oxygen level alone or in combination with nitrate concentration and optionally redox potential to detect an oxygenation status of the target region selected from oxic status hypoxic status and anoxic status. The timed and targeted chlorate administration method further comprise
contacting a target region of the least one target regions having a detected an oxic status with an antibiotic effective amount in an oxic environment.
contacting a target region of the least one target regions having a detected hypoxic status with a chlorate effective amount in combination with an antibiotic effective amount in an hypoxic environment and/or
contacting a target region of the least one target regions in an anoxic status with a chlorate effective amount in an anoxic environment.

In the timed and targeted method of chlorate administration of the sixth aspect, the contacting is performed for a time and under conditions to treat and/or prevent infection of Nar-containing bacteria.

Preferably, in chlorate timed and targeted method of the disclosure, chlorate can be administered alone in a targeted region under anoxic condition, having a detected oxygen level is lower than 20 uM, and in combination with antibiotic in a targeted region under hypoxic condition having a detected oxygen levels are from 200-20 uM. Preferably chlorate can be administered alone in a targeted region under anoxic condition having a oxygen level lower than 20 uM, a detected nitrate concentration above 100 uM more preferably above 500 uM, more preferably in combination with a detected redox potential below 200 mV.

Preferably, in the timed and targeted method of chlorate administration one or more antibiotics can be administered alone at an antibiotic administration time when the biological environment is under oxic condition with detected oxygen level is higher than 200 uM, and in combination with chlorate at an antibiotic administration time when the biological environment is under hypoxic condition with detected oxygen levels are from 200-20 uM.

In the timed and targeted method of chlorate administration preferred amounts of chlorate to be administered to hypoxic region of a biological environment which preferably ranges from 0.001 to 10 uM, more preferably or 0.01 to 10 mM and most preferably 0.1 mM to 10 mM.

In the timed and targeted method of chlorate administration preferred amounts of chlorate to be administered to an anoxic region of the biological environment which preferably ranges from 0.001 to 10 uM, more preferably or 0.01 to 10 mM and most preferably 0.1 mM to 10 mM.

In the timed and targeted method of chlorate administration preferred amounts of antibiotic to be administered under oxic conditions are amounts resulting in delivery of the antibiotic MIC typically amounts ranging from 0.1 ug/mL to 500 ug/mL preferably from 1 to 500 ug/mL, from 1-30 ug/mL or 1-5 ug/mL depending on the specific antibiotic used as will be understood by a skilled person upon reading of the present disclosure.

In the timed and targeted method of chlorate administration preferred amounts of antibiotic to be administered under hypoxic conditions are amounts resulting in delivery of the antibiotic typically amounts ranging from 0.1 ug/mL to 500 ug/mL preferably from 1 to 500 ug/mL, from 1-30 ug/mL or 1-5 ug/mL depending on the specific antibiotic used as will be understood by a skilled person upon reading of the present disclosure.

In the timed and targeted method of chlorate administration preferred amounts of antibiotic to be administered under hypoxic conditions are amounts resulting in delivery of amounts of antibiotic which is a fraction of the MIC for the antibiotic, preferably half the MIC, more preferably one quarter of the MIC, or lower up to one tenth of the MIC Typically, an oxic antibiotic effective concentration comprise an amount ranging from 0.1 ug/mL to 500 ug/mL preferably from 1 to 500 ug/mL, from 1-30 ug/mL or 1-5 ug/mL in view of the bacteria susceptibility to the antibiotic, the target objective of the treatment (e.g. desired therapeutic effect) the antibiotic used and the biological environment treated (e.g. skin, blood, muscles, lungs, mucosa and others identifiable by a skilled person).

In some preferred embodiments, of the timed and targeted method of chlorate administration, the detecting can be performed on the at least on target region of the biological environment at a plurality of times to monitor the change in oxygenation status of the at least one target region of the biological environment. In those embodiments, the chlorate administration, the chlorate effective amount, the antibiotic administration time and the antibiotic effective amount for a target region of the at least one target region are selected in time based on an oxygenation status to the target region detected over time.

According to a seventh aspect, a hypoxic timed and targeted chlorate administration method and system are described for a hypoxic biological environment, the method comprises contacting the hypoxic environment s with a chlorate effective amount in combination with an antibiotic effective amount to inhibit viability of a Nar-containing bacteria.

In the hypoxic timed and targeted chlorate administration method of the seventh aspect the chlorate effective amount is preferably selected from 0.001 to 10 mM, more preferably or 0.01 to 1 mM and most preferably 0.1 mM to 0.5 mM.

In the hypoxic timed and targeted chlorate administration method, the antibiotic effective amount being lower than the MIC and/or MBC e.g. a fraction of the MIC for the antibiotic, selected from half the MIC, more preferably one quarter of the MIC, or lower up to one tenth of the MIC Typically, an oxic antibiotic effective concentration comprise an amount ranging from 0.1 ug/mL to 500 ug/mL preferably from 1 to 500 ug/mL, from 1-30 ug/mL or 1-5 ug/mL in view of the bacteria susceptibility to the antibiotic, the target objective of the treatment (e.g. desired therapeutic effect) the antibiotic used and the biological environment treated (e.g. skin, blood, muscles, lungs, mucosa and others identifiable by a skilled person).

In some preferred embodiments of the hypoxic timed and targeted chlorate administration method, the detecting can be performed on the at least on target region of the biological environment at a plurality of times to monitor the change in oxygenation status of the biological environment or a target region thereof. In those embodiments, the chlorate effective amount, and the antibiotic effective amount are administered to the biological environment or the target region thereof when oxygenation status detected over time indicated that the biological environment or target region thereof. is under hypoxic conditions.

The hypoxic timed and targeted chlorate administration system of the seventh aspect comprises one or more chlorate in hypoxic effective concentrations, one or more antibiotic in hypoxic effective concentrations and at least one of a look up table connecting amounts and timing of administration of chlorate, amounts and timing of administration of antibiotic with respect to the administration of the chlorate in accordance with the hypoxic timed and targeted chlorate administration method of the seventh aspect.

Additional optional components comprise, at least one of an oxygen sensor, electrodes for detection of redox potential, and electrodes and/or reagents for detection of nitrate with corresponding instructions in the look-up table in connection with detected values with respect to threshold values, nitrate at effective concentrations to be administered in accordance with the indications of the look-up table, reagents to detect biomarker of anaerobic respiration and reagents to detect biomarker of aerobic respiration optionally in connection with corresponding instructions in the look up table in connection with detected quantitative and/or qualitative values with respect to threshold values, at least one antimicrobial and at least one wound healing agent as would be understood by a skilled person upon reading of the present disclosure.

According to an eighth aspect, a hypoxic timed and targeted chlorate administration method and a system are described of treating a biological. The method comprises detecting in at least one target region of the biological environment at least one of oxygen level, nitrate concentration and redox potential of a target region of the biological environment, preferably oxygen level alone or in combination with nitrate concentration and optionally redox potential to detect a hypoxic target region of the biological environment.

The hypoxic timed and targeted chlorate administration method further comprises
contacting the hypoxic target region of the least one target regions having a detected hypoxic status with a chlorate effective amount in combination with an antibiotic effective amount to inhibit viability of Nar-containing bacteria.

In the hypoxic timed and targeted chlorate administration method of the seventh aspect the chlorate effective amount preferably selected from 0.001 to 10 uM, more preferably or 0.01 to 1 mM and most preferably 0.1 mM to 0.5 mM.

In the hypoxic timed and targeted chlorate administration method of the seventh aspect the antibiotic effective amount being a a fraction of the MIC for the antibiotic, selected from half the MIC, more preferably one quarter of the MIC, or lower up to one tenth of the MIC, thus resulting in amounts typically ranging from 0.001 to 500 ug/ml, with higher concentrations corresponding from 0.1-500 ug/ml preferably 1-30 ug/mL or 1-5 ug/mL to increase the efficacy of the treatment in view of the bacteria susceptibility to the antibiotic, the target objective of the treatment (e.g. desired therapeutic effect) the antibiotic used and the biological environment treated (e.g. skin, blood, muscles, lungs, mucosa and others identifiable by a skilled person).

In the hypoxic timed and targeted method of chlorate administration of the eighth aspect, the contacting is performed for a time and under conditions to treat and/or prevent infection of Nar-containing bacteria.

In some preferred embodiments of the hypoxic timed and targeted chlorate administration method, the detecting can be performed on the at least on target region of the biological environment at a plurality of times to monitor the change in oxygenation status of the at least one target region of the biological environment. In those embodiments, the chlorate administration, the chlorate effective amount, the antibiotic administration time and the antibiotic effective amount for a target region of the at least one target region are selected in time based on an oxygenation status to the target region detected over time, when the detected oxygenation status indicates that the at least one target region is in hypoxic condition.

The hypoxic timed and targeted chlorate administration system of the seventh aspect comprises one or more chlorate in hypoxic effective concentrations, one or more antibiotic in hypoxic effective concentrations and at least one of a look up table connecting amounts and timing of administration of chlorate, amounts and timing of administration of antibiotic with respect to the administration of the chlorate in accordance with the hypoxic timed and targeted chlorate administration method of the eighth aspect.

Additional optional components comprise, at least one of an oxygen sensor, electrodes for detection of redox potential, and electrodes and/or reagents for detection of nitrate with corresponding instructions in the look-up table in connection with detected values with respect to threshold values, nitrate at effective concentrations to be administered in accordance with the indications of the look-up table, reagents to detect biomarker of anaerobic respiration and reagents to detect biomarker of aerobic respiration optionally in connection with corresponding instructions in the look up table in connection with detected quantitative and/or qualitative values with respect to threshold values, at least one antimicrobial and at least one wound healing agent as would be understood by a skilled person upon reading of the present disclosure.

According to an ninth aspect, a medical implant is described configured to release chlorate to one or more anoxic portions of a biological environment within an individual. In preferred embodiments, the medical implant is further configured to release at least one antibiotic to oxic portions of the biological environment within the individual and to hypoxic portions of the individual at a same or more preferably at a different amounts in accordance with any one of the chlorate administration methods herein described. Medical implants can comprise prosthetic devices, such as bone/cartilage replacement parts, shunts, cardiac assistance devices, internal pumps, and additional components identifiable by a skilled person. In some embodiments, the medical implant can further be configured to release at least one antimicrobial and/or one wound healing agent as will be understood by a skilled person upon reading of the disclosure.

According to a tenth aspect, a matrix is described for treatment of an infected biological environment according to timed and/or targeted chlorate administration of the disclosure. The matrix comprises an effective amount of a chlorate embedded in a delivery matrix, alone or in combination with one or more antibiotics and/or antimicrobials. In particular the matrix of the disclosure comprises one or more chlorate in an anoxic matrix region configured to release the one or more chlorate to one or more anoxic regions of a biological environment of an individual in a chlorate effective amount for an anoxic environment.

The matrix of the disclosure can further comprise one or more antibiotics in an oxic matrix region configured to release the one or more antibiotic to one or more oxic regions portions of the biological environment in an antibiotic effective amount for an oxic environment.

The matrix of the disclosure further comprises one or more chlorate and one or more antibiotics in a hypoxic matrix region configured to release the one or more chlorate and the one or more antibiotic to one or more hypoxic regions of a biological environment of an individual in a chlorate effective amount and an antibiotic effective amount for a hypoxic environment.

In some embodiments, the oxic matrix regions are configured to release the one or more antibiotic to oxic portions or hypoxic portion of the biological environment of the individual, which were or at the time of the detection are previously anoxic, the antibiotic release can precede be concurrent or follow release of the chlorate release, in accordance with the timed administration of antibiotic and/or chlorate herein described as understood by a skilled person upon reading of the present disclosure.

In preferred embodiments, matrix of the disclosure is biofilm treatment matrix comprising one or more biofilm treatment agents in an effective amount to disrupt bacterial biofilm in a biological environment. In some embodiments, the biofilm treatment agent does not include nitrate. In some embodiments the matrix of the disclosure is a wound treatment matrix configured for wound treatment, and in particular, chronic wound treatment according to timed and/or targeted methods of chlorate administration of the present disclosure.

In most preferred embodiments of methods and systems of the disclosure as well as related compositions matrices and implants, the contacting of chlorate is performed in absence of chlorite and/or hypochlorite and/or in absence of any chlorine oxyanion other than chlorate.

In some embodiments, any one of the methods of the disclosure can be delivered by way of a wearable medical device, such as a smart bandage or wrap, that monitors various biological statistics (oxygen levels, redox potential, nitrate levels, presence of bacteria, etc.) through sensors (e.g., functionalized electrodes) and deliver the required substances (antibiotics, chlorate, nitrate, etc.) automatically (e.g. by a processor) in accordance with the methods. Sensors used by these systems can be configured to measure levels at specific depths of tissue/wounds (such as with a needle probe electrode).

In some embodiments, systems/devices begin performing the method at the instruction of a user. In some embodiments, for pre-determined bacteria strains, electrodes can be functionalized to detect markers configured to tag those strains, thereby allowing the systems to automatically detect an infection and automatically begin the antibiotic methods described herein.

In embodiments of the methods and system the contacting is preferably performed for a time and under condition to inhibit formation of a bacteria biofilm and/or disrupt a bacteria biofilm in the wound thus preventing the wound from becoming a chronic wound.

In some embodiments the method can further comprise contacting the wound with an antibiotic before after and/or in combination with the chlorate. In some embodiments the method can further comprise applying to the wound an effective amount of a wound healing agent to promote re-epithelization and granulation tissue formation of the wound. The systems can further comprise wound healing agent as will be understood by a skilled person.

In preferred embodiments methods, systems and related compounds and composition herein described are used in connection with treatment of chronic wound to effectively promote the related healing.

In some embodiments, methods, systems and related compounds and compositions herein described can be used to treat and/or prevent systemic infections and/or chronic infections, such as pulmonary infections and/or infections associated with implanted medical devices.

The methods and systems for timed and/or targeted administration of chlorate to treat a bacterial infection and related matrices, compounds, compositions and implants herein described, allow in several embodiments to minimize waste and/or overuse of chlorate and antibiotic by optimizing the timing and/or location of the related administration. In particular in preferred embodiments, the methods and systems for timed and/or targeted administration of chlorate to treat a bacterial infection and related matrices, compounds, compositions and implants herein described allow use of reduced yet effective amounts of antibiotics which are below their related MIC as will be understood by a skilled person.

The methods and systems for timed and/or targeted administration of chlorate to treat a bacterial infection and related matrices, compounds, compositions and implants herein described, allow in several embodiments to maximize the synergic chlorate/antibiotic effectiveness by administering chlorate and antibiotic at an optimized administration time and/or to target region where the effectiveness is maximized.

The methods and systems for timed and/or targeted administration of chlorate to treat a bacterial infection and related matrices, compounds, compositions and implants herein described, in embodiments directed to treat hypoxic target region and/or hypoxic environment allow to broaden the range of antibiotic capable of use for treatment, by using the antibiotic in a combination with chlorate to stimulate antibiotic susceptibility.

The methods and systems for timed and/or targeted administration of chlorate to treat a bacterial infection and related matrices, compounds, compositions and implants herein described, allow in several embodiments to overall improve the effectiveness of the treatment for the patient because the infection does not become chronic or in general worsens and the patient experience a faster and better healing process.

The methods and systems for timed and/or targeted administration of chlorate to treat a bacterial infection and related matrices, compounds, compositions and implants herein described, are based on the finding that optimized timing and location of chlorate and/or antibiotic administration result in a treatment of Nar-containing bacteria at least as effective as existing methods and systems (see showing in Examples 13 to 15 as a proof of principle).

In particular the methods and systems for timed and/or targeted administration of chlorate to treat a bacterial infection and related matrices, compounds, compositions and implants herein described, can result in a better improvement of particularly difficult biological environments such as chronic wound by killing the bacteria in the wound, thereby disrupting biofilm develop and allowing surrounding tissues to heal more effectively either simultaneously or prior to applying the wound healing agents.

In particular the methods and systems for timed and/or targeted administration of chlorate to treat a bacterial infection and related matrices, compounds, compositions and implants herein described, can address multiple biological characteristics associated with wound healing such as prolonged inflammation, poor angiogenesis, thus promoting wound healing and closure.

The methods, systems and related compounds and compositions herein described can be used in connection with applications wherein treating of infection involving Nar-Containing bacteria is desired. For example, matrices, compounds, compositions, methods and systems herein described can be used to develop therapeutic approaches and tools to treat and/or prevent infections in individuals, in particular, in connection with use of medical implants and/or wound treatment, with more particular reference to treatment and/or prevention of chronic wound formation. Additional exemplary applications include uses of the methods, systems and related compounds and compositions herein described in several fields including basic biology research, applied biology, bio-engineering, biological analysis, aetiology, medical research, medical therapeutics, with particular reference to clinical applications and in additional fields identifiable by a skilled person upon reading of the present disclosure.

The details of one or more embodiments of the disclosure are set forth in the accompanying drawings, annexes and the description below. Other features, objects, and advantages will be apparent from the description and drawings, and from the claims.

### BRIEF DESCRIPTION OF THE DRAWINGS

The accompanying drawings, which are incorporated into and constitute a part of this specification, illustrate one or more embodiments of the present disclosure and, together with the detailed description and the examples, serve to explain the principles and implementations of the disclosure.

FIG. 1: Chlorate hijacks the hypoxically-induced Nar enzyme to kill anoxic, antibiotic-tolerant P. aeruginosa. A. Chlorate is a prodrug: while chlorate itself is relatively nontoxic, it is reduced to toxic chlorite by the hypoxically-induced nitrate reductase, Nar. B. The log₁₀(% survival) of WT P. aeruginosa cultures treated with tobramycin or chlorate for 24 hours incubated under oxic, hypoxic, or anoxic conditions. All data points on the x-axis are below the detection limit. Data show the means of 3 replicates and error bars show standard error of the mean. Statistical significance was determined by one-way ANOVA; ns=not significant, *=p <0.05, **=p<0.01, ***=p<0.001, ****=p<0.0001.

FIG. 2: P. aeruginosa exhibits antibiotic recalcitrance due to resistance or hypoxia-induced antibiotic tolerance. The log₁₀(% survival) of WT P. aeruginosa cultures treated with chlorate (Chlor), tobramycin (Tob), ciprofloxacin (Cip), colistin (Col), or ceftazidime (Ceft) for 24 hours incubated under hypoxic or oxic conditions. All data points on the x-axis are below the detection limit. Data show the means of 3 replicates and error bars show standard error of the mean. Statistical significance was determined by two-tailed t-tests; ns=not significant, *=p<0.05, **=p<0.01, ***=p<0.001, ****=p<0.0001.

FIG. 3: Chlorate synergizes with different classes of antibiotics to kill hypoxic P. aeruginosa populations. The log₁₀(% survival) of WT P. aeruginosa cultures treated with chlorate (Chlor), tobramycin (Tob), ciprofloxacin (Cip), colistin (Col), ceftazidime (Ceft), or each chlorate-antibiotic combination for 24 hours incubated under hypoxic conditions. All data points on the x-axis are below the detection limit. Data show the means of 3 replicates and error bars show standard error of the mean. Statistical significance was determined by two-tailed t-tests; ns=not significant, *=p<0.05, **=p<0.01, ***=p<0.001, ****=p<0.0001.

FIG. 4: Chlorate-antibiotic synergy is Nar-dependent. The log₁₀(% survival) of Anar P. aeruginosa cultures treated with chlorate (Chlor), tobramycin (Tob), ciprofloxacin (Cip), colistin (Col), ceftazidime (Ceft), or each chlorate-antibiotic combination for 24 hours incubated under hypoxic conditions. All data points on the x-axis are below the detection limit. Data show the means of 3 replicates and error bars show standard error of the mean. Statistical significance was determined by two-tailed t-tests; ns=not significant, *=p<0.05, **=p<0.01, ***=p<0.001, ****=p<0.0001.

FIG. 5: Chlorate substantially lowers the effective ceftazidime dose for killing hypoxic P. aeruginosa. The log₁₀(% survival) of WT P. aeruginosa cultures treated with chlorate (Chlor), a range of ceftazidime (Ceft) concentrations, or chlorate-ceftazidime combinations for 24 hours incubated under hypoxic conditions. All data points on the x-axis are below the detection limit. Data show the means of 3 replicates and error bars show standard error of the mean. Statistical significance was determined by two-tailed t-tests; ns=not significant, *=p<0.05, **=p<0.01, ***=p<0.001, ****=p<0.0001.

FIG. 6: Chlorate-antibiotic synergy is effective across a range of O₂ availabilities. The log₁₀(% survival) of WT P. aeruginosa cultures treated with chlorate (Chlor), tobramycin (Tob), ceftazidime (Ceft), or each chlorate-antibiotic combination for 24 hours incubated under A. oxic, B. hypoxic, or C. anoxic conditions. All data points on the x-axis are below the detection limit. Data show the means of 3 replicates and error bars show standard error of the mean. Statistical significance was determined by two-tailed t-tests; ns=not significant, *=p<0.05, **=p<0.01, =p<0.001, ****=p<0.0001.

FIG. 7: Most antibiotics do not exhibit synergistic interactions across different classes of antibiotics. The log₁₀(% survival) of WT P. aeruginosa cultures treated with colistin (Col), tobramycin (Tob), ciprofloxacin (Cip), ceftazidime (Ceft), or each antibiotic-antibiotic combination for 24 hours under hypoxic conditions. Compared to single-antibiotic treatments, only colistin displays synergistic interactions with other classes of antibiotics. All data points on the x-axis are below the detection limit. Data show the means of 3 replicates and error bars show standard error of the mean. Statistical significance was determined by one-way ANOVA; ns=not significant, *=p<0.05, **=p<0.01, ***=p<0.001, ****=p<0.0001 for each antibiotic-antibiotic combination compared to the single-antibiotic treatments that comprise the combination.

FIG. 8: Representative oxygen microprofiles. (FIG. 8 Panel A) STOX microprofile, with the sputum sample marked in tan. The green rectangle shows an expanded view of a portion of the anoxic zone in the sputum sample. The detection limit is 2 nM. (FIG. 8 Panel B) Oxygen microelectrode profiles of 5-mm-deep expectorated sputum samples from 5 different patients. At the oxic air-sputum interface, a steep oxycline begins through a hypoxic zone into an anoxic zone that persists for the remaining portion of the sputum. (FIG. 8 Panel C) Oxygen microelectrode profiles of 8-mm-deep expectorated sputum samples from 5 different patients that were larger in volume. The same trend in profile occurs, with a steep oxycline through hypoxia into anoxia.

FIG. 9: Representative examples of high and low sputum ORPs. The tan-shaded boxes indicate the extent of the sputum sample. (FIG. 9 Panel A) Of 23 of the redox-profiled samples, 11 displayed a positive redox potential (16 mV to 355 mV) indicative of an oxidizing microenvironment. (FIG. 9 Panel B) Of 23 of the profiled samples, 17 displayed a negative redox potential (−300 mV to −107 mV) indicative of a reducing microenvironment. Oxygen concentration measurements for these samples are shown on the left, for B in particular indicating how the redox potential decreases following the oxycline.

FIG. 10: Comparison of levels of oxygen diffusion into mucus based on different respiratory airway geometry constraints and bacterial densities. Data represent oxygen diffusion into different respiratory airways clogged with mucus at various bacterial densities. The described scenarios correspond to a modeled mucus thickness (x) of 500 μm and an airway diameter of 1.5 mm for scenario B (model scenarios A and C are not affected by the airway diameter). The upper blue shaded area indicates air, and the tan lower layer indicates mucus.

FIG. 11: The effect of considering different values for oxygen diffusivity in various materials on oxygen penetration depth. The three oxygen diffusivities considered are in water, biofilms, and rat colon mucus.

FIG. 12: mRNA probes are target specific. (FIG. 12 Panel A) Micrographs of single-cell deletion validation controls for mRNA probes. (FIG. 12 Panel B) Micrographs of aggregate deletion validation controls for mRNA probes. Images reflect the region 50 to 100 μm from the air-agar interface. (FIG. 12 Panel C) Quantification of mean mRNA probe intensities for single cells grown in liquid culture of wild-type and deletion mutants for target genes under upregulating growth conditions. Mean intensity was 10-fold higher in the wild type than in the deletion for all probe sets. Each boxplot summarizes approximately 10 images per replicate.

Three replicates were performed per condition, and each colored box represents a different biological replicate. Whiskers represent 1.5 times the interquartile range, while diamonds on the boxplots represent outliers. (FIG. 12 Panel D) Quantification of mean mRNA probe intensities for aggregates grown in agar blocks wild-type and deletion mutants for ackA under upregulating growth conditions. Mean intensity was 2-fold higher in the wild type, and each boxplot summarizes 3 to 5 images per replicate. Three replicates were performed per condition.

FIG. 13: Catabolic Genes Show Distinct Patterns Across Intra-Aggregate Gradients 3D Micrographs of Probe Signal in LB+40 mM Nitrate ABBAs. Each image represents a 50 micron slice of agar, compiled from 8 individual z-slices with an interslice distance of 6.24 microns, viewed from the top of the block, with each sequential image from the top of the figure representing the section directly below the slice above it. rRNA signal is colored cyan, while mRNA signal is colored magenta.

FIG. 14: ABBA images Replicate 2: 3D Micrographs of Probe Signal in LB+40 mM Nitrate ABBAs. Each image represents a 50 micron slice of agar, compiled from 8 individual z-slices with an interslice distance of 6.24 microns, viewed from the top of the block, with each sequential image from the top of the figure representing the section directly below the slice above it. rRNA signal is colored cyan, while mRNA signal is colored magenta. All replicates were performed concurrently.

FIG. 15: Metabolic genes show distinct patterns across three-dimensional oxygen gradients. (FIG. 15 Panel A) Mean oxygen levels of ABBA samples grown with LB plus 40 mM nitrate for 12 h. The dark line is the mean, while the shading represents the standard deviation of 8 biological replicates grown under identical conditions. (FIG. 15 Panel B) Three-dimensional micrographs of probe signal in LB plus 40-mM nitrate ABBAs. Each image represents a 50-um slice of agar, compiled from 8 individual z-slices with an interslice distance of 6.24 um, viewed from the top of the block, with each sequential image from the top of the figure representing the section directly below the slice above it. The rRNA signal is colored cyan, while the mRNA signal is colored magenta. (FIG. 15 Panel C) Mean mRNA channel intensity per aggregate (x axis) plotted by depth. Each plot represents four images of one replicate each of an experimental and control condition, and each point represents one aggregate. Filled points represent the experimental condition, while the open circles represent the background autofluorescence intensity of aggregates imaged in the mRNA channel in a control condition where only rRNA probes were used.

FIG. 16: RPA requires Nar for chlorate susceptibility. Survival of WT and Anar RPA after treatment with ciprofloxacin, chlorate, or both drugs under oxic and anoxic planktonic conditions and grown as biofilms. For each treatment, n≥3 replicates and bars show mean±standard error of the mean. RPA, Riverside Pseudomonas aeruginosa; Nar, nitrate respiration.

FIG. 17: Ciprofloxacin and chlorate treatment support healing of RPA-infected wounds. Wounds were infected with RPA (106 CFU) 24 h after injury and daily treatment began 10 days after infection. Wounds were treated daily with either vehicle (n=5), ciprofloxacin (cip, n=7), chlorate (chlor, n=8), or both ciprofloxacin and chlorate (cip/chlor, n=9). (FIG. 17 Panel A) Visualization of RPA biofilms using MiPACT-HCR on a section of chronic wound tissue collected at 10 days postwounding and before treatment (wound surface is on the top).

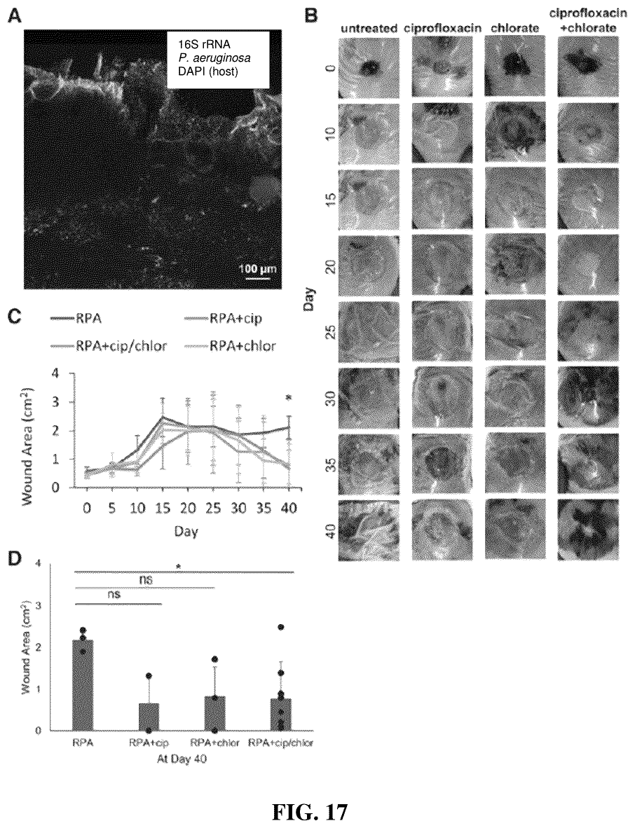

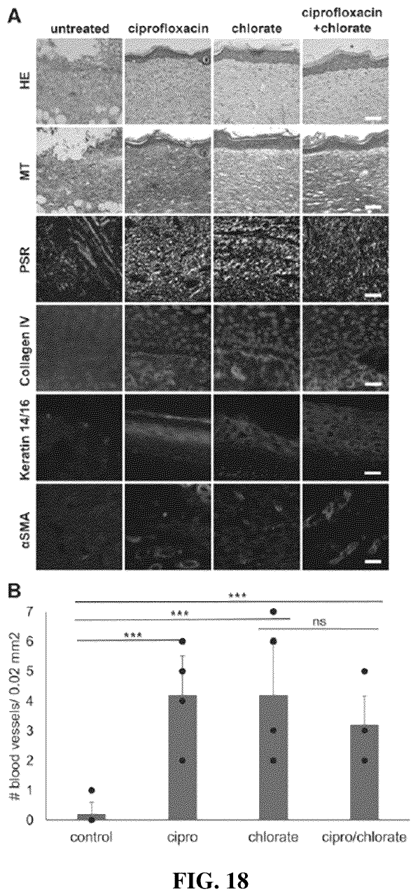

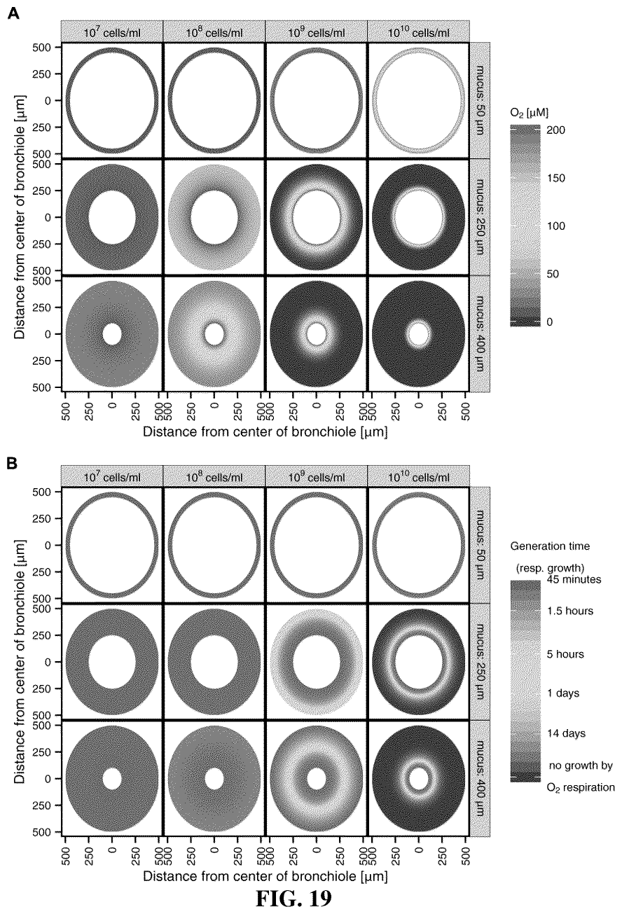

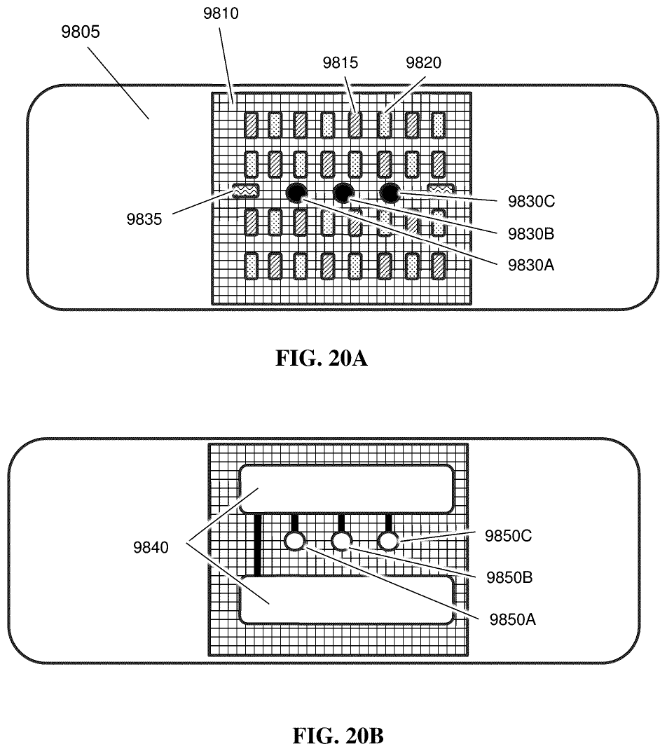

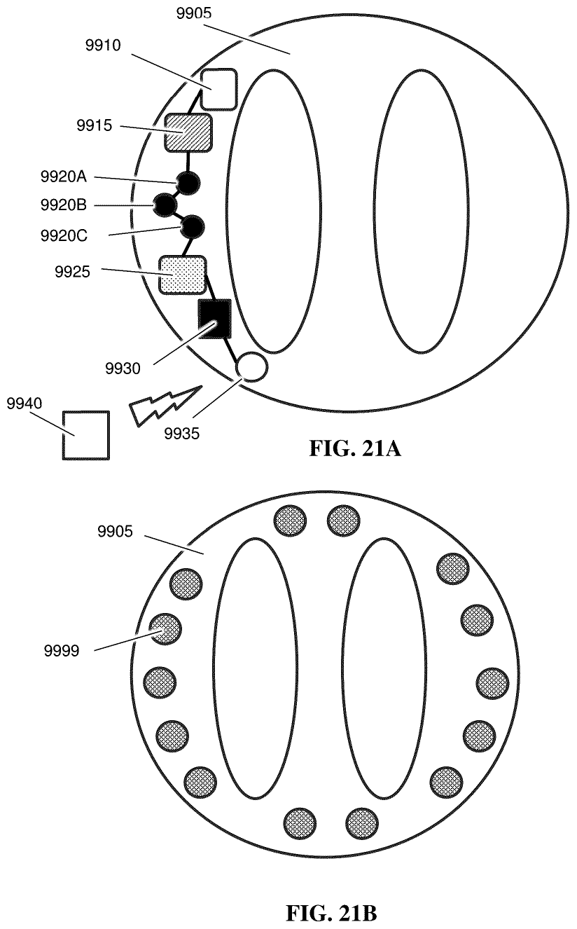

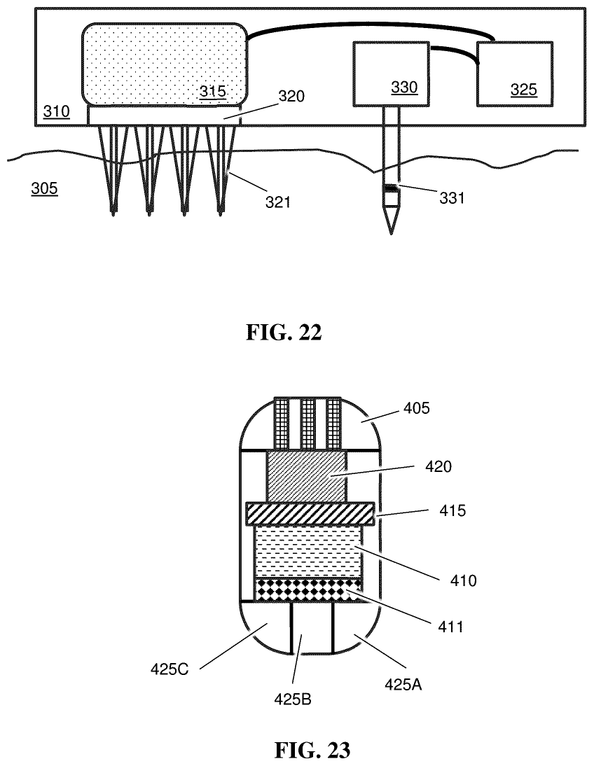

Yellow fluorescence shows RPA, as detected by 16S rRNA amplification, and DAPI (blue) was used to visualize the nuclei of the cells in the mouse tissue. (FIG. 17 Panel B) Representative images of untreated and treated RPA-infected wounds over 40 days. Untreated RPA-infected wounds did not undergo wound closure and have robust biofilm formation at day 40, whereas treated wounds had decreased amount of biofilm and were smaller in size. (FIG. 17 Panel C) Quantification of wound areas over time for untreated and treated RPA-infected wounds. The student t-test was used to determine significant differences between treatment groups compared with the untreated control. *p-value <0.05 is between RPA+cip/chlor and RPA. (FIG. 17 Panel D) Individual data points for control and treatments groups are shown for RPA-infected wounds at day 40. Student's t-test was used to determine significant differences between treatment groups compared with the untreated control. *p-value <0.05. CFU, colony-forming unit; MiPACT-HCR, Microbial identification after Passive Clarity Technique-Hybridization Chain Reaction; ns, non significant.

FIG. 18: Ciprofloxacin and chlorate treatment support wound healing. (FIG. 18 Panel A) Wound tissue was collected at day 40 for treated wounds that had healed and also for untreated wounds to perform histology and immunofluorescence staining. Cryosections of the skin were taken from wound tissue for HE, MT, and PSR. Scale bar=100 um. Collagen III is shown in green, collagen I is shown in red, and the colocalization of collagen III and collagen I is shown in yellow. Wound tissues were immunolabeled with collagen IV (red), aSMA (red), keratin 14/16 (red), and all samples were labeled with 4′,6-diamidino-2-phenylindole to stain the DNA in the nuclei of the mouse cells (blue). Scale bar=20 um. (FIG. 18 Panel B) The number of blood vessels were counted in five frames (area=0.02 mm²) of the granulation tissue. Blood vessel of RPA-infected wounds were compared using one-way analysis of variance: ***p-value <0.001.

FIG. 19: Predicted oxygen concentrations and generation times due to aerobic respiration in bronchioles clogged with mucus. (FIG. 19 Panel A) Differential oxygen concentrations affecting the thickness of the mucus and the bacterial density. (FIG. 19 Panel B) A two-dimensional visualization of the bronchiole with variation in the mucus thickness and bacterial density and the resulting predicted generation time of the pathogens in the mucus during growth only via aerobic respiration (resp.). This accounts only for aerobic respiration and neglects other catabolic pathways.

FIGS. 20A and 20B show an example of a smart bandage configured to enact embodiments of the methods described herein. FIG. 20A shows the skin-facing side of the bandage and FIG. 20B shows the exterior of the bandage.

FIGS. 21A and 21B show an example of a prosthetic (in this example, a portion of a knee replacement) configured to enact embodiments of the methods described herein. FIG. 21A shows an example of the components embedded in the prosthetic (not to scale) and FIG. 21B shows an example of multiple sets of the components embedded in the prosthetic (not necessarily to scale).

FIG. 22 shows an example of depth-based drug release for the methods described herein.

FIG. 23 shows an example of a smart pill configured for the methods described herein.

### DETAILED DESCRIPTION

Provided herein are methods, systems, and related compounds and composition suitable for treating and/or preventing an infection in a biological environment.

The term “environment” as used herein indicates a sum total of all the elements in a defined space of interest and subject to investigation. An environment can be a biological environment if it includes at least one biological element, elements of an environment comprise molecule and in particular biological molecule. Accordingly, environments can include different defined biological spaces of interest. For example, a biological environment can include, one or more tissues, organs and/or biofluids of an individual subjected to treatment in vitro, in vivo or ex vivo.

The term “individual” as used herein in the context of treatment and/or prevention includes a single biological organism. Exemplary individuals in the sense of the disclosure include plants, and animals, and in particular higher animals and in particular vertebrates such as mammals and in particular human beings.

Accordingly, the term “individual” or “host” as used herein indicates any multicellular organism that can comprise microorganisms, thus providing a biological environment for microbes and in in particular an environment for microbial communities, in any of their tissues, organs, and/or biofluids. Exemplary individual in the sense of the disclosure includes plants, algae, animals, and in particular, vertebrates, mammals more particularly humans.

Accordingly, biological environments in the sense of the disclosure comprise one or more organs of an individual such a heart, brain, lungs, liver, kidney, joints, skin, as well systems such as digestive system, skeletal systems and nervous systems of a mammal and in particular a human being.

Biological environments in the sense of the disclosure also comprise one or more tissues of an individual. Exemplary tissues that can be comprised in a biological environment in the sense of the disclosure comprise epithelial tissue: covering various surfaces and cavities of the body of an animal, both internal and external, such as simple squamous epithelium found in areas like the alveoli of the lungs and blood vessels, stratified squamous epithelium: providing protection to areas subjected to abrasion, like the skin and the lining of the oral cavity, cuboidal epithelium: cube-shaped cells that function in secretion and absorption, found in glands and kidney tubules, and columnar epithelium: involved in secretion and absorption, lining the digestive tract and respiratory tract of mammals.

Further exemplary tissues that can be comprised in a biological environment in the sense of the disclosure comprise connective tissues supporting and connecting different structures in the body of an animal individual, such as: loose connective tissue found throughout the body of animals. dense connective tissue found in structures like tendons (connect muscle to bone) and ligaments (connect bone to bone). cartilage: a firm and flexible tissue found in joints and other areas, providing support and reducing friction. bone: a hard tissue that provides structural support, protection, serves as a reservoir for minerals end encompasses bone marrow.

Additional exemplary tissues that can be comprised in a biological environment in the sense of the disclosure comprise muscle tissues supporting and connecting different structures in the body an animal individual, such as: skeletal muscle attached to bones smooth muscle found in the walls of organs and blood vessels, controlling involuntary movements, and cardiac muscle: exclusive to the heart.

Further exemplary tissues that can be comprised in a biological environment in the sense of the disclosure comprise nervous tissue: which consists of neurons and neuroglia, which support and protect neurons.

Biological environments in the sense of the disclosure comprise various combinations of organ and/or tissue forming target parts of the body. For example, a biological environment in the sense of the disclosure can comprise joints which are the points where two or more bones come together. Joints can be classified into different types based on their structure and function, such as hinge joints (like the knee) and ball-and-socket joints (like the hip). Other target biological environments can comprise combination of tissues and organs that can be subjected to infections such as the lungs, pancreas, and intestines affected by cystic fibrosis as will be understood by a skilled person.

Exemplary tissues organs and/or biofluids from an individual comprise the following: whole venous and arterial blood, capillary blood, blood plasma, blood serum, dried blood spots, cerebrospinal fluid, interstitial fluid, sweat, lumbar fluid, nasal tissues and fluids, sinus tissues and fluids, tears, corneal, saliva, sputum or expectorate, bronchoscopy secretions, transtracheal tissue and/or fluid, endotracheal tissue and/or fluid, bronchoalveolar tissue and/or fluid, gastric tissue and/or fluid, colon tissue and/or fluid, subcutaneous and mesenteric adipose tissue and/or fluid, bile, vaginal tissue and/or fluid such as secretions, endometrial tissues and/or fluids such as secretions, urethral fluids and secretions, mucosal secretions, synovial fluid, ascitic fluid, peritoneal tissue and/or fluid, tympanic membrane fluid, urine, including clean-catch midstream urine, catheterized urine, suprapubic tissue and fluids, kidney stones, prostatic secretions, feces, mucus, pus, wound, skin, hair, nail, cheek tissue, bones, bone marrow, muscular tissues solid organ, solid organ tissue such as lung tissues, breast milk, or tumor cells, among others identifiable by a skilled person.

The biological environment can be a medium in vivo as part of the individual or in vitro or ex vivo as part of sample taken from an individual will also be understood by a skilled person.

The term “infection” as used herein indicates the invasion of tissues by pathogens, their multiplication, and the reaction of host tissues to the infectious agent and the toxins they produce Infections can be caused by a wide range of pathogens, most prominently bacteria and viruses.

Hosts can fight infections using their immune systems. Individuals have defense mechanisms to infections. For example mammalian hosts react to infections with an innate response, often involving inflammation, followed by an adaptive response as will be understood by a skilled person upon reading of the disclosure. [1]

In embodiments, herein describe infections that can be treated and/or prevented are infections by and/or involving bacteria (e.g. bacterial infection and polymicrobial infection by various microorganisms including bacteria).

In embodiments herein described bacteria in the sense of the disclosure comprise Nar-containing bacteria. “Nar-containing bacteria” refer to the types of bacteria containing a gene set encoding cytoplasmic nitrate reductase (“Nar”), thus capable of conducting Nar-mediated nitrate respiration.

The term “Nar” “nitrate reductase” refers to a group of membrane-bound protein complexes that reduce nitrate to nitrite. Nar is bound to the inner membrane and its active site is located in the cytoplasm. In its reaction, Nar transfers electrons from a membrane-associated reduced quinone to nitrate, thus producing nitrite. This energetically favorable reaction is coupled to proton translocation to generate a proton motive force, which can ultimately be used to power the cell (e.g. ATP synthesis) [2] Nar is capable of using nitrate as an electron acceptor to reduce nitrate to nitrite during anaerobic respiration. as an alternative to using oxygen as a terminal electron acceptor. The membrane-bound Nar complex is composed of three subunits: a) a catalytic a subunit, encoded by narG, containing a molybdopterin cofactor; b) a soluble β subunit, encoded by narH, containing four [4Fe-4S] centers; and c) the γ subunit, encoded by narI, containing two b-type hemes. In some embodiments, formation of the Nar complex further requires a chaperone-like component required for the maturation of the αβ complex encoded by narJ gene.

Accordingly, in some embodiments, Nar in the sense of the current disclosure is encoded by a narGHJI operon possessed by the Nar-containing bacteria. narG, H, I encode the α, β, and γ subunit respectively, while narJ encodes the chaperone-like component required for the maturation of the αβ complex. The transcription of narGHJI is typically activated under hypoxic or anoxic conditions and further stimulated by the presence of nitrate.

The term “operon” is a functioning unit of DNA containing a cluster of genes under the control of a single promoter as will be understood by a person of ordinary skill in the art. The term “gene” as used herein indicates a polynucleotide encoding for a protein that in some instances can take the form of a unit of genomic DNA within a bacteria, plant or other organism.

The term “polynucleotide” as used herein indicates an organic polymer composed of two or more monomers including nucleotides, nucleosides or analogs thereof. The term “nucleotide” refers to any of several compounds that consist of a ribose or deoxyribose sugar joined to a purine or pyrimidine base and to a phosphate group and that are the basic structural units of nucleic acids. The term “nucleoside” refers to a compound (as guanosine or adenosine) that consists of a purine or pyrimidine base combined with deoxyribose or ribose and is found especially in nucleic acids. The term “nucleotide analog” or “nucleoside analog” refers respectively to a nucleotide or nucleoside in which one or more individual atoms have been replaced with a different atom or a with a different functional group. Accordingly, the term polynucleotide includes nucleic acids of any length, and in particular DNA RNA analogs and fragments thereof.

The term “protein” as used herein indicates a polypeptide with a particular secondary and tertiary structure that can interact with another molecule and in particular, with other biomolecules including other proteins, DNA, RNA, lipids, metabolites, hormones, chemokines, and/or small molecules. The term “polypeptide” as used herein indicates an organic linear, circular, or branched polymer composed of two or more amino acid monomers and/or analogs thereof. The term “polypeptide” includes amino acid polymers of any length including full-length proteins and peptides, as well as analogs and fragments thereof. A polypeptide of three or more amino acids is also called a protein oligomer, peptide, or oligopeptide. In particular, the terms “peptide” and “oligopeptide” usually indicate a polypeptide with less than 100 amino acid monomers. A protein “sequence” indicates the order of the amino acids that form the primary structure.

As used herein the term “amino acid”, “amino acid monomer”, or “amino acid residue” refers to organic compounds composed of amine and carboxylic acid functional groups, along with a side-chain specific to each amino acid. In particular, alpha- or αamino acid refers to organic compounds composed of amine (—NH2) and carboxylic acid (—COOH), and a side-chain specific to each amino acid connected to an alpha carbon. Different amino acids have different side chains and have distinctive characteristics, such as charge, polarity, aromaticity, reduction potential, hydrophobicity, and pKa. Amino acids can be covalently linked to form a polymer through peptide bonds by reactions between the amine group of a first amino acid and the carboxylic acid group of a second amino acid. Amino acid in the sense of the disclosure refers to any of the twenty naturally occurring amino acids, non-natural amino acids, and includes both D an L optical isomers.

Identification of a Nar-containing bacterium can be performed by various techniques. In some embodiments, Nar-containing bacteria can be identified by performing a database search using narG gene or amino acid sequence from a characterized Nar as a query sequence or reference sequence. Bacteria containing a gene or protein sequence having protein having at least 80% query coverage and at least 50% sequence similarity with respect to the reference sequence are identified as Nar-containing bacteria.

As used herein, “query coverage” refers to the percentage of the query sequence that overlaps the identified sequence. The term “sequence similarity” refers to a quantitative measurement of the similarity between sequences of a polypeptide or a polynucleotide. In particular, “sequence similarity” makes reference to the nucleotide bases or protein residues in the two sequences that are the same when aligned for maximum correspondence over a specified comparison window. When percentage of a sequence similarity is used in reference to proteins, it is recognized that residue position which are not identical often differ by conservative amino acids substitutions, where amino acid residues are substituted with a functionally equivalent residue of the amino acid residues with similar physiochemical properties and therefore do not change the functional properties of the molecule. Accordingly, similarity between two sequences can be expressed as percent sequence identity and/or percent positive substitutions. Widely used similarity searching programs, like BLAST, PSI-BLAST [3], SSEARCH [4] [5], FASTA [6] and the HMMER3 [7] programs produce accurate statistical estimates, ensuring protein sequences that share significant similarity also have similar structures.

A functionally equivalent residue of an amino acid used herein typically refers to other amino acid residues having physiochemical and stereochemical characteristics substantially similar to the original amino acid. The physiochemical characteristics include water solubility (hydrophobicity or hydrophilicity), dielectric and electrochemical properties, physiological pH, partial charge of side chains (positive, negative or neutral) and other properties identifiable to a person skilled in the art. The stereochemical characteristics include spatial and conformational arrangement of the amino acids and their chirality. For example, glutamic acid is considered to be a functionally equivalent residue to aspartic acid in the sense of the current disclosure. Tyrosine and tryptophan are considered as functionally equivalent residues to phenylalanine. Arginine and lysine are considered as functionally equivalent residues to histidine.

The similarity between sequences is typically measured by a process that comprises the steps of aligning the two polypeptide or polynucleotide sequences (a subject sequence and a reference sequence) to form aligned sequences, then detecting the number of matched characters in the subject sequence with respect to the reference sequence, i.e. characters similar or identical between the two aligned sequences, and calculating the total number of matched characters divided by the total number of aligned characters in each polypeptide or polynucleotide sequence, including gaps. The similarity result is expressed as a percentage of similarity.

As used herein, “reference sequence” is a defined sequence used as a basis for sequence comparison. A reference sequence may be a subset or the entirety of a specified sequence; for example, as a segment of a full-length protein or protein fragment. A reference sequence can comprise, for example, a sequence identifiable in a database such as GenBank and UniProt and others identifiable to those skilled in the art.

As understood by those skilled in the art, determination of percentage of similarity between any two sequences can be accomplished using a mathematical algorithm. Non-limiting examples of such mathematical algorithms are the algorithm of Myers and Miller [8], the local homology algorithm of Smith et al. [9]; the homology alignment algorithm of Needleman and Wunsch [10]; the search-for-similarity-method of Pearson and Lipman [11]; the algorithm of Karlin and Altschul [12], modified as in Karlin and Altschul [13]. Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA [11], and TFASTA in the Wisconsin Genetics Software Package, Version 8 (available from Genetics Computer Group (GCG), 575 Science Drive, Madison, Wis., USA). Alignments using these programs can be performed using the default parameters.

In some embodiments, identification of a Nar-containing bacterium can be performed by performing a database search using narG amino acid sequence from P. aeruginosa NarG having sequence:

(SEQ ID NO 1)
MSHLLDRLQFFKKKQGEFADGHGETSNESRAWEGAYRQRWQHDKIVR
STHGVNCTGSCSWKIYVKNGLITWETQQTDYPRTRPDLPNHEPRGCPRG
ASYSWYIYSANRLKYPKVRKPLLKLWREARAQHGDPVNAWASIVEDA
AKAKSYKSQRGLGGFVRSSWDEVTEIIAAANVYTAKTYGPDRVIGFSPI
PAMSMVSYAAGARYLSLIGGVCLSFYDWYCDLPPASPQIWGEQTDVPE
SADWYNSSYIIAWGSNVPQTRTPDAHFFTEVRYKGTKTVSITPDYSEVA
KLTDLWLNPKQGTDAALGMAFGHVILKEFHLDRPSAYFVDYCRQYTD
MPMLVLLEEHAGGAFKPTRYLRAADLADNLGQDNNPEWKTIAYDERS
GGLVSPTGAIGYRWGESGKWNIAELDGRSGDQTRLQLSLLDGPEHACE
VAFPYFAGQEHPHFKGVANDEVLLRRVPFREIVAADGKRLRVATVYDL
QMANYSIDRGLGGDNVATSYEDADTPYTPAWQERITGVPAARATQVA
REFADSADKTRGKAMVIIGAAMNHWYHMDMNYRAVINMLMMCGCIG
QSGGGWAHYVGQEKLRPQTGWAPLAFGLDWSRPPRQMNGTSFFYLHS
SQWRHEKLSMHEVLSPLADASRFAEHALDYNIQAERLGWLPSAPQLNR
NPLRIAAEAEAAGLPVADYVVRELKSGGLRFASESPDDPQNFPRNMFIW
RSNLLGSSGKGHEYMLKYLLGAKNGVMNDDLGKAGGPRPTEVDWVD
DGAEGKLDLVTTLDFRMSSTCMYSDIVLPTATWYEKDDLNTSDMHPFI
HPLSAATDPAWEAKSDWEIYKAIAKKFSAVAEGHLGVEQDLVTVPLLH
DTPTELAQPFGGDGHDWKKGECEPMPGRNLPTLHLVERDYPNVYRKFT
SLGPLLDKLGNGGKGIGWNTEKEVKLVGDLNHRVVESGVSQGRPRIDS
AIDAAEVVLALAPETNGQVAVKAWEALSKITGREHAHLALPKEDEKIRF
RDIQVQPRKIISSPTWSGLEDEHVSYNAGYTNVHELIPWRTITGRQQFY
QDHPWMQAFGEGFVSYRPPVNTRTTEKLLNRKPNGNPEITLNWITPHQK
WGIHSTYSDNLLMLTLSRGGPIIWLSEHDAAKAGIVDNDWVEVFNANG
AATCRAVVSQRVKDGMVMMYHAQERIVNVPGSETTGTRGGHHNSVT
RVVLKPTHMIGGYAQQAWGFNYYGTVGCNRDEFVVVRKMSKVDWLD
EPRHGGLGGDALPQPLPQDI

as a reference sequence to search for homologs in public databases such as GenBank, UniProt, EMBL, and others identifiable to a person skilled in the art, using tools such as BLASTp and additional tools identifiable by a skilled person. In those embodiments, Nar-containing bacteria can be identified as those containing a protein having at least 80% query coverage and at least 50% sequence similarity compared to SEQ ID NO:1.

In particular, bacteria containing a gene or protein sequence having protein having at least 80% query coverage and at least 50% sequence similarity with respect to the SEQ ID NO: 1 of P. aeruginosa.

In some embodiments, identification of a Nar-containing bacterium can be performed by isolating cell membrane fractions and performing membrane fraction assay for nitrate reduction by detecting nitrite concentration. In addition, identification of a Nar-containing bacterium can also be performed by constructing a bacterial culture supplemented with chlorate and detecting chlorite concentration after incubation. The procedure can further comprise testing whether the chlorate reduction is inhibited by other compounds such as azide, cyanide, and thiocyanate. [14]

In some embodiments, identification of a Nar-containing bacterium can be performed by culture-independent techniques, such as performing whole genome sequencing and BLAST annotated protein sequences to a P. aeruginosa Nar as described herein. In particular, whole genome sequencing can be performed using culture-dependent methods (e.g. isolate bacterium, culture, extract DNA, sequence) or through culture-independent methods (e.g. single-cell sequencing. Another culture independent technique that can be performed to detect Nar-containing bacteria is sequencing a community's metagenome from an environment, with or without culturing. Metagenomic will provide an indication of whether Nar exists within a community, or with enough depth/coverage allow one to assemble genomes of individuals from the community.

In some embodiments, identifying nar-containing bacterium can be performed by detecting genes encoding Nar. For example, detecting genes encoding Nar can be performed by detecting sequences of one or more of the narG, narH, narJ and narI in the genome, transcriptome, or proteome of one or more candidate bacteria as described above. Exemplary techniques that can be used to detecting sequences of one or more genes (e.g. where the genome is known), comprises computer-based tools for comparing gene sequences, transcript sequences, or protein sequences, such as those using the Basic Local Alignment Search Tool (BLAST) or any other similar methods known to those of ordinary skill in the art.

In some embodiments, detecting genes encoding Nar in the one or more candidate Nar-containing bacteria can be performed by detecting the genes and/or related transcript in the one or more candidate bacteria. Exemplary techniques comprise wet bench approaches such as DNA sequencing, PCR, Southern blotting, DNA microarrays, or other methods of hybridization of DNA or RNA probes to DNA, wherein probes are attached to a label capable of emitting a signal such as radiolabeling, fluorescence, luminescence, mass spectroscopy or colorimetric methods.

Exemplary probes that can be used comprise primers from known narG, narH, narJ and narI and/or related transcript as will be understood by a skilled person.

In some embodiments, detecting genes encoding Nar in the one or more candidate bacteria strains can be performed by detecting transcripts of narG, narH, narJ and narI.

Exemplary techniques comprise RNA sequencing, PCR, quantitative PCR, Northern blotting, in situ hybridization, RNA microarrays, or other methods of hybridization of DNA or RNA probes to RNA.

In some embodiments, detecting genes encoding Nar in the one or more candidate bacteria strains can be performed by detecting proteins encoded by narG, narH, narJ and narI.

Exemplary techniques comprise proteomics, antibody-based methods including immunohistochemistry, immunofluorescence, western blotting, or any other method of protein detection.

In embodiments herein described the conditions and parameters to use probes/primers to detect narG, narH, narJ and narI can be varied to permit lower or higher threshold or stringency of detection, to ensure hybridization within at least 80% sequence identity at gene level in view of the specific primers/probes selected. For example, use of oligonucleotides comprising one or more degenerated nucleotide bases or using an antibody that binds to more highly conserved protein regions, can require modification of the detection conditions as will be understood by a skilled person.

In an exemplary embodiment, the detection can be done, for example, by isolating genomic DNA from a candidate strain and performing PCR using primer sequences designed to amplify narG gene from known Nar-containing bacteria, including the primers listed in the Example section. Alternatively, RNA samples can be isolated from the candidate and these transcripts can be sequenced, and expression of the narG gene can be detected by identification of this gene using homology-based computational identification (e.g. BLAST).

Other methods for identifying a bacterium capable of nitrate respiration would be identifiable to a skilled person upon reading of the present disclosure.

In some embodiments, Nar containing bacteria that can be targeted with method and systems of the disclosure and related devices and composition, comprise proteobacteria and in particular some Alphaproteobacteria which possess nitrate reductase. Gammaproteobacteria; and in particular the family of Enterobacteriaceae which includes Nar containing bacteria such as Klebsiella pneumoniae, and Enterobacter species. As well as other gammaproteobacteria such as Pseudomonas aeruginosa and other members of this family are known for their ability to reduce nitrate to nitrite, all of which are known to contain Nar, and Firmicutes which possess Nar.

In some embodiments, exemplary Nar-containing bacteria include Pseudomonas aeruginosa, Staphylococcus aureus, Proteus spp. Escherichia coli, Propionibacterium acnes, Mycobacterium tuberculosis. Exemplary bacteria in the sense of the disclosure can also include Pseudomonas, Actinomyces israelii, Actinomyces gerencseriae, Brevibacterium, Brevibacterium linens, Coryneform Bacteria, Corynebacterium diphtheria, Nocardia, Bacillus anthracis, Bacillus cereus, Brucella melitensis, Brucella suis, Brucella abortus, Burkholderia cenocepacia, Burkholderia pseudomallei, Pantoea agglomerans, Pectobacterium atrosepticum, Propionibacterium propionicus, Pseudomonas fluorescens, Salmonella enterica, Shigella species, Staphylococcus epidermidis, Streptomyces anulatus, and related species that contains Nar to facilitate various physiological functions identifiable to a skilled person upon reading of the present disclosure.

In particular in some embodiments, Nar containing bacteria that can be targeted by methods and systems and related devise and compositions of the present disclosure comprise ESKAPE bacteria Klebsiella pneumoniae: Pseudomonas aeruginosa: Enterobacter species. In some embodiments, Nar containing bacteria that can be targeted by methods and systems and related devise and compositions of the present disclosure.

In some embodiments, the Nar-containing bacteria comprise P. aeruginosa, S. aureus, E. coli, wherein the Nar operon is expressed under hypoxic/anoxic conditions. In particular, in P. aeruginosa, the presence of nitrate is known to further increase transcription of narGHJI.

In some embodiments where P. aeruginosa is the microorganism, sequences for the genes of the Nar operon comprises

P. aeruginosa narG (SEQ ID NO: 2):
ATGAGTCACCTGCTCGACCGCCTGCAGTTCTTCAAGAAGAAGCAGGGCGAATTCG
CCGATGGCCACGGCGAGACCAGCAACGAGAGCCGCGCCTGGGAAGGTGCCTACCG
GCAGCGCTGGCAGCACGACAAGATCGTGCGCTCCACCCACGGGGTGAACTGCACC
GGCTCCTGCTCCTGGAAGATCTACGTGAAGAACGGCCTGATCACCTGGGAAACCC
AGCAGACCGACTACCCGCGCACCCGTCCGGACCTGCCCAACCACGAGCCGCGCGG
CTGCCCGCGCGGGGCCAGCTATTCCTGGTACATCTACAGCGCCAACCGCCTGAAGT
ACCCGAAGGTGCGCAAGCCGTTGCTCAAGCTCTGGCGCGAGGCGCGGGCGCAGCA
CGGCGACCCGGTGAACGCCTGGGCCAGCATCGTCGAGGACGCCGCCAAGGCGAAG
AGCTACAAGAGCCAGCGCGGCCTGGGCGGCTTCGTCCGTTCCAGCTGGGACGAGG
TCACCGAGATCATCGCCGCGGCCAACGTCTACACCGCCAAGACCTACGGTCCGGA
CCGGGTGATCGGCTTCTCGCCGATCCCGGCCATGTCGATGGTCAGCTACGCCGCCG
GCGCCCGCTACCTGTCGCTGATCGGCGGGGTCTGCCTGAGCTTCTACGACTGGTAC
TGCGACCTGCCGCCGGCCAGCCCGCAGATCTGGGGCGAGCAGACCGACGTGCCGG
AGTCGGCCGACTGGTACAACTCCAGCTACATCATCGCCTGGGGCTCCAACGTGCCG
CAGACGCGGACCCCGGACGCGCACTTCTTCACCGAGGTGCGCTACAAGGGCACCA
AGACCGTCTCCATCACCCCGGACTATTCCGAGGTGGCCAAGCTCACCGACCTCTGG
CTCAACCCCAAGCAGGGCACCGACGCCGCGCTGGGCATGGCCTTCGGTCACGTGA
TCCTGAAGGAATTCCACCTCGACCGGCCGAGCGCCTACTTCGTCGACTACTGCCGC
CAGTACACCGACATGCCGATGCTGGTGTTGCTGGAAGAACACGCCGGCGGCGCGT
TCAAGCCGACCCGCTACCTGCGCGCCGCCGACCTGGCGGACAACCTCGGCCAGGA
CAACAACCCCGAGTGGAAGACCATCGCCTACGACGAGCGCAGCGGCGGGCTGGTC
TCGCCCACCGGCGCCATCGGCTATCGCTGGGGCGAGTCAGGCAAGTGGAACATCG
CCGAGCTGGACGGCAGGAGCGGTGACCAGACGCGCCTGCAACTGTCGCTGCTCGA
TGGCCCGGAACATGCCTGCGAGGTGGCCTTCCCGTATTTCGCCGGGCAGGAGCAC
CCGCACTTCAAGGGCGTCGCCAACGACGAGGTACTGCTGCGCCGGGTGCCGTTCC
GCGAGATCGTCGCGGCGGACGGCAAGCGCCTGCGGGTGGCCACCGTCTACGACCT
GCAGATGGCCAACTACAGCATCGACCGCGGCCTGGGCGGCGACAACGTGGCGACC
TCCTACGAGGACGCCGACACGCCCTATACCCCGGCCTGGCAGGAGCGCATCACCG
GCGTTCCGGCGGCGCGCGCGACGCAGGTCGCCCGCGAGTTCGCCGACAGCGCCGA
CAAGACCCGCGGCAAGGCGATGGTGATCATCGGCGCGGCGATGAACCACTGGTAC
CACATGGACATGAACTACCGCGCGGTCATCAACATGCTGATGATGTGCGGCTGCA
TCGGCCAGAGCGGCGGCGGCTGGGCGCACTATGTCGGCCAGGAGAAGCTGCGCCC
GCAGACCGGCTGGGCGCCGCTGGCCTTCGGCCTGGACTGGAGCCGGCCGCCGCGG
CAGATGAACGGCACCAGCTTCTTCTACCTGCACAGCTCGCAATGGCGCCACGAGA
AGCTGTCGATGCACGAGGTGCTGTCGCCGCTGGCCGACGCCAGCCGCTTCGCCGA
ACACGCCCTGGACTACAACATCCAGGCCGAACGCCTCGGCTGGCTGCCGTCGGCG
CCGCAACTGAACCGCAACCCGCTGCGCATCGCCGCCGAGGCCGAGGCCGCCGGCC
TGCCGGTCGCCGACTACGTGGTGCGCGAACTGAAGAGCGGCGGCCTGCGCTTCGC
CAGCGAATCGCCGGACGATCCGCAGAACTTCCCGCGCAACATGTTCATCTGGCGCT
CCAACCTGCTGGGCTCCTCCGGCAAGGGCCACGAGTACATGCTCAAGTACCTGCTC
GGGGCGAAGAACGGGGTGATGAACGATGACCTCGGCAAGGCCGGCGGTCCGCGT
CCCACCGAGGTCGACTGGGTTGACGACGGTGCCGAGGGCAAGCTCGACCTGGTCA
CCACCCTGGACTTCCGCATGTCCTCCACCTGCATGTACTCGGACATCGTCCTGCCG
ACCGCTACCTGGTACGAGAAGGACGACCTCAACACCTCCGACATGCACCCCTTCAT
CCATCCGCTGTCGGCGGCCACCGATCCGGCCTGGGAAGCCAAGAGCGACTGGGAG
ATCTACAAGGCCATCGCCAAGAAGTTCTCCGCCGTCGCCGAAGGCCACCTCGGCG
TGGAGCAGGACCTGGTCACGGTGCCGCTGCTGCACGACACCCCCACCGAGCTGGC
GCAGCCGTTCGGCGGCGACGGCCATGACTGGAAGAAGGGCGAGTGCGAGCCGAT
GCCGGGACGCAACCTGCCGACGCTGCACCTGGTCGAGCGCGACTACCCGAACGTC
TACCGCAAGTTCACCTCGCTCGGTCCGCTGCTGGACAAGCTGGGCAACGGCGGCA
AGGGCATCGGCTGGAACACCGAGAAGGAAGTGAAGCTGGTCGGCGACCTCAACC
ATCGCGTCGTCGAGAGCGGCGTCAGCCAGGGCCGCCCGCGCATCGACAGCGCCAT
CGACGCCGCTGAGGTGGTCCTCGCCCTGGCTCCGGAAACCAACGGCCAGGTCGCG
GTCAAGGCCTGGGAAGCGCTGTCGAAGATCACCGGCCGCGAGCATGCCCACCTGG
CGCTGCCCAAGGAAGACGAGAAGATCCGCTTCCGCGACATCCAGGTGCAGCCGCG
CAAGATCATCTCCAGCCCGACCTGGTCCGGCCTCGAGGACGAGCACGTCAGCTAC
AACGCCGGCTACACCAACGTCCACGAGCTGATCCCGTGGCGCACCATCACCGGTC
GCCAGCAGTTCTACCAGGACCACCCGTGGATGCAGGCGTTCGGCGAAGGCTTCGT
CAGCTACCGGCCGCCGGTCAACACCCGGACCACCGAGAAACTGTTGAACAGGAAG
CCCAACGGCAACCCGGAGATCACCCTGAACTGGATCACCCCGCACCAGAAATGGG
GCATCCACTCCACCTACAGCGACAACCTGCTGATGCTCACCCTGTCGCGCGGCGGT
CCGATCATCTGGCTCAGCGAGCACGACGCGGCCAAGGCCGGGATCGTCGATAACG
ACTGGGTCGAGGTGTTCAACGCCAACGGCGCGGCGACCTGCCGCGCGGTGGTCAG
CCAGCGGGTCAAGGACGGCATGGTGATGATGTACCACGCCCAGGAACGCATCGTG
AACGTACCCGGCAGCGAGACCACCGGCACCCGTGGCGGCCACCACAACTCGGTGA
CCCGCGTGGTGCTCAAGCCGACCCACATGATCGGCGGCTACGCCCAGCAGGCCTG
GGGCTTCAACTACTACGGCACGGTCGGCTGCAACCGCGACGAGTTCGTCGTGGTG
CGCAAGATGAGCAAGGTCGACTGGCTGGACGAACCCCGCCACGGCGGACTCGGCG
GCGACGCCCTGCCGCAACCGCTGCCCCAGGACATTTGA
P. aeruginosa narH (SEQ ID NO: 3):
ATGAAAATTCGTTCGCAAGTCGGCATGGTGCTGAACCTCGACAAGTGCATTGGTTG
CCACACCTGCTCGATCACCTGCAAGAACGTCTGGACCAGCCGCGAAGGCATGGAG
TACGCCTGGTTCAACAACGTCGAGACCAAGCCCGGGATCGGCTACCCGAAGGAAT
GGGAAAACCAGGAGAAGTGGAAGGGCGGCTGGGTGCGCGCGGCGGACGGTTCGA
TCCGCCCGCGCATCGGCGGCAAGTTCCGCGTGCTGGCGAACATCTTCGCCAACCCG
GACCTGCCCGAGATCGACGACTACTACGAACCGTTCGACTTCGATTACCAGCACCT
GCATACCGCGCCCAAGGCCGAGCACCAGCCGGTGGCGCGCCCGCGCTCGCTGGTC
TCCGGGCAGCGCATGGAGAAGATCGAGTGGGGCCCGAACTGGGAGGAGATCCTCG
GCACCGAGTTCGCCAAGCGGCGCAAGGACAAGAACTTCGACCAGGTCCAGGCGGA
CATCTACGGTGAGTACGAGAACACCTTCATGATGTACCTGCCGCGCCTCTGCGAGC
ACTGCCTGAACCCGGCGTGCGTGGCGTCCTGCCCGAGCGGGGCGATCTACAAGCG
CGAGGAGGACGGCATCGTCCTGATCGACCAGGACAAGTGCCGCGGCTGGCGGATG
TGCATCTCCGGCTGCCCGTACAAGAAGATCTACTTCAACTGGAAGAGCGGCAAGT
CCGAGAAGTGCATCTTCTGCTACCCGCGCATCGAGGCCGGCCAGCCCACTGTCTGC
TCGGAGACCTGCGTCGGGCGCATCCGCTACCTCGGCGTGCTGCTCTACGACGCCGA
CCGCATCCACGAAGTGGCCAGTTGCGAGAACGAGCGCGAGCTGTACGAGAAGCAA
CTGGAGATCTTCCTCGATCCGTTCGACCCGGCGGTGATCGCCCAGGCGCGCAAGG
ACGGGGTGGCCGACAGCGTCATCGAGGCGGCGCAGAAGTCGCCGGTGTACAAGCT
GGCGATGGACTGGAAGCTGGCCCTGCCGCTGCACCCGGAATACCGCACGCTGCCG
ATGGTCTGGTACGTGCCACCGCTGTCGCCGATCCAGAACGCCGCCGCCGAGGGGC
ACATCGGCAGCGACGGGGTGATCCCGGACGTGGAGTCGCTGCGCATCCCCGTGCA
GTACCTGGCCAACCTGCTCACCGCCGGCGACACCGCGCCGGTGCTGCTGGCGCTCA
AGCGCCTGCTGGCGATGCGCGCCTACAAGCGCGCCGAGCACGTCGAAGGCCGCCA
GGACCTGGAGGTGCTGGCCAAGGTCGGGTTGAGCGTGGAGCAGGTGGAGGAGAT
GTACCGCTACCTGGCCATCGCCAACTACGAGGATCGCTTCGTGATCCCCAGCGCGC
ACCGCGAGGAAGCGCTTTCCGATGCCTTCGCCGAGCGTTCCGGCTGCGGCTTCAGC
TTCGGCAACGGCTGTTCCGGCGGCAGCAACTCCGCCGTCAACCTGTTCGGCGGCAA
GCCGACCAACCGCCGCGACGTGATCCAGGTCGTGCAGATCCAGGAGTGA
P. aeruginosa narJ (SEQ ID NO: 4):
ATGAACGATCACAGCCAACTGTTCCGCCTGCTCGCCCTGCTGCTCGACTATCCACG
CGCCGAGCTGCGCGAGGAGAGCCTCGGCCTGCATGCGCTGATCCGCACCTGCGAA
CTGCCGGAAGCGCTGCGCGACGGCCTCGCGGCGCTGCTCAACGAGCTCTGCCAGG
GCGACCTGCTGGACGTCCAGGCGCGCTACGACGGTCTCTTCGAGCGCGGCCGCTC
GGTCTCGCTGCTGCTCTTCGAGCACGTCCACGGCGAGAGCCGCGACCGTGGCCAG
GCGATGGTCGACCTGCTCGACCGCTATACCGGGGCCGGCCTGCAGATCGACGTAC
CGGAGCTGC-
CGGACTACCTGCCGCTGTACCTCGAATACCTGTCGCTGCTGCCGTTCGCGGCGGCC
AGCGAAGGGCTCGCCGAAGTGGCGCACATCCTCGGCCTGCTGGCGCTGCGCCTGG
AGGAACGCGGCAGCGCCTACGCGGCGATTTTCGAGGCGTTGCTGGAACTCGGCGG
CGAGCGCCCGGACCTCGGCGCGTTGCGTCGCGACCAGGCCCAGGAACAGCGCGAC
GACAGCCTGGAGGCCATCGACCGGGCCTGGGAGGAAACCCCGGTGAGCTTCACCG
ACCCTGCCGGCGGTTGCCCGTCGAGCAGCGGCCGCCGTCCGACGGCGTCCACCGA
ACAACCATTGCAATGGGTCGCCCAGCCGGTACCGCAGATGCAGTACCGCGCGGCC
CGCGAAGGAGTCTGA
P. aeruginosa narI (SEQ ID NO: 5):
ATGTCGACCAATCTTCTGTTCTTCGGGATCTATCCCTATGTCGCGCTGCTGATCTGC
CTGGTCGGCAGCTGGGCGCGCTTCGACCTCTCGCAGTACACCTGGAAGGCCGGTTC
CAGCCAGATGCTCAGCAAGAAGGGCATGCGGGTATACAGCAACCTGTTCCACGTC
GGCGTGCTGTTCATCCTCGCCGGCCACTTCGTCGGCCTGCTGACCCCGGCCTCGGT
CTACCACCACCTGATCAGCACCGAGAACAAGCAACTGCTGGCGATGGTCTCCGGC
GGCTTCTTCGGCGTGCTCTGCTTCATCGGCCTGAGCGGACTGATCCTGCGCCGCCT
GACCGACGCGCGGGTGCGCGCCACCGGCAACGCCTCTGACCTGATGATCCTGCTG
GTGCTCTACGCCCAGCTGATCCTCGGCCTCTCCACCATCGTCGCCTCGACCCATCA
CATGGATGGCTCGGTGATGGTGATGCTCGCCGACTGGGCCCAGGCCATCGTCACCC
TGCGTCCGCTGGCGGCGGCCGAAGCCATCGCGCCGGTGGGCCTGGTCTACAAGCT
GCACGTCGGCCTGGGCCTGACCCTGTTCGTGCTGTTCCCCTTCACCCGCCTGGTGC
ACATCGTCAGCGCGCCGGTGTGGTACCTGGGCCGGCGCTACCAGATCGTGCGTCA
GAAACGTCCTGCCTGA

Other NarG sequences similar to P. aeruginosa NarG include NarG from gamma proteobacteria such as E. coli having 98% query coverage and 83% sequence similarity with respect to SEQ ID NO:1, NarG sequence from the gram-positive bacterium S. aureus having 97% query coverage and 67% sequence similarity with respect to SEQ ID NO:1, and the NarG sequence from the delta proteobacterium Anaeromyxobacter sp. Fwl09-5 having 95% query coverage and 61% sequence similarity with respect to SEQ ID NO:1.

In some embodiments, the Nar-containing bacteria can comprise additional genetic features, such as mutations and or other changes which typically affect the rate of nitrate respiration and that in some instances can occur over the course of the bacteria's infection.

For example, in some embodiments, the Nar-containing bacteria herein described can comprise a lasR mutation in which lasR function is defective or lost. lasR is a gene encoding a quorum-sensing regulator, so the loss of this gene has pleiotropic effects [15]. One phenotypic trait of lasR mutants is their decreased rates of oxygen respiration and increased rates of Nar-dependent nitrate respiration [16]. lasR mutants have been isolated from human infections such as bacteremia, pneumonia, chronic wounds, and CF [17] and more resistant to some antibiotics.

The prominence of lasR mutants has been documented in CF studies, where they are among the most frequently isolated mutants from CF patients [17] and their presence is associated with worse lung function [18]. lasR mutants are also more resistant to antibiotics commonly used to treat P. aeruginosa infections [15, 16].

In particular, in some embodiments, Nar-containing bacteria comprising a lasR mutation show increased rates of nitrate respiration and chlorate consumption and reduce chlorate more rapidly than the wild type bacteria does.

Accordingly, in some of the embodiments herein described, the methods, systems, compounds, and composition herein described are directed to interfere with viability of Nar-containing bacteria comprising a lasR mutation.

Detailed description on Nar and Nar-containing organisms can be found in copending application U.S. Ser. No. 16/157,885 filed on Oct. 11, 2018, published as US 2019/0142864, incorporated by reference in its entirety.

Exemplary Nar-containing bacteria that can be found in wounds and/or infections are reported in Table 1

TABLE 1

| Nar-containing bacterium | Disease |
|---|---|
| Actinomyces israelii | Actinomycosis |
| Actinomyces gerencseriae | |
| Propionibacterium propionicus | |
| Bacillus anthracis | Anthrax |
| Bacillus cereus | infection |
| Brucella melitensis | Brucellosis |
| B. suis | |
| B. abortus | |
| Corynebacterium diphtheriae | Diphtheria |
| Escherichia coli and other species | various infections |
| Burkholderia pseudomallei | Melioidosis |
| Streptococcus pneumoniae | Pneumococcal infection |
| Salmonella species | Salmonellosis |
| Shigella species | Shigellosis |
| Mycobacterium tuberculosis | Tuberculosis |
| Some Staphylococcus species, | various infections |
| including S. aureus and | including chronic |
| S. epidermidis | wounds |
| Pseudomonas aeruginosa | chronic wounds, |
| | ventilator-associated |
| | pneumonia, and Cystic |
| | fibrosis |

Additional exemplary Nar-containing bacteria comprise Burkholderia cepacia complex, Achromobacter xylosoxidans, Stenotrophomonas maltophilia and additional species.

Nar-containing bacteria in can form a bioflm as understood by a skilled person. As used herein the term “biofilm” indicates an aggregate of microorganisms in which cells adhere to each other on a surface. These adherent cells are frequently embedded within a self-produced matrix of extracellular polymeric substance (EPS). Accordingly, a biofilm, comprises a multicellular aggregate, attached to a surface or embedded within mucus. Biofilms can form on living or non-living surfaces and can be prevalent in natural, industrial and hospital settings. The microbial cells growing in a biofilm are physiologically distinct from planktonic cells of the same organism, which, by contrast, are single cells that can float or swim in a liquid medium. Formation of a biofilm begins with the attachment of free-floating microorganisms to a surface. These first colonists adhere to the surface initially through weak, reversible adhesion via van der Waals forces. If the colonists are not immediately separated from the surface, they can anchor themselves more permanently using cell adhesion structures such as pili. When the biofilm growth is balanced with that of biofilm dispersion, the biofilm is considered “mature.” Methods to quantify and measure biofilms will be known to a skilled person and can include, for example, the COMSTAT method of [19].

The development of a biofilm can allow for an aggregate cell colony (or colonies) to be increasingly tolerant [20] or resistant to antibiotics. Cell-cell communication or quorum sensing has been shown to be involved in the formation of biofilm in several bacterial species. [21] [22]

In embodiments herein described, infections of a target biological environment can be treated by timed and/or targeted administration of chlorate which can be contacted with an infected environment at a chlorate administration time selected to administer chlorate at a time when the Nar-containing bacteria are undergoing or have undergone anaerobic respiration.

The term “chlorate” refers to chemical compounds containing chlorate oxyanion having the formula ClO₃⁻.

As used herein, “chlorine oxyanion” refers to an anion consisting of one or more oxygen atoms covalently bonded to a chlorine atom. Exemplary chlorine oxyanions include hypochlorite ion ClO⁻, chlorite ion ClO₂⁻, chlorate ion ClO₃⁻, and ClO₄⁻. Chlorine oxyanions are typically comprised within a salt. In particular, a salt of chlorine oxyanion as used herein contains the oxyanion together with a cation as a counterion.

The cation can be a metal cation and in particular the metal ion can have a charge of +1, +2, +3 or +4. Exemplary +1 cation includes Li¹⁺, Na¹⁺, K¹⁺, Cs¹⁺, and Ag¹⁺. Exemplary +2 cation includes Mg²⁺, Ca²⁺, Sr²⁺, Ba²⁺, Ni²⁺, Cu²⁺, Pb²⁺, Fe²⁺ and Zn²⁺. Exemplary +3 cation includes A13+, and Fe³⁺. Exemplary +4 cation includes Ti⁴⁺, Zr⁴⁺.

The cation can be an oxycation which, as used herein, refers to a cation consisting of one or more oxygen atoms covalently bonded to another atom. Exemplary oxycation includes nitronium ion, NO₂¹⁺, and vanadyl ion, VO²⁺.

Exemplary chlorates include potassium chlorate, sodium chlorate, magnesium chlorate, silver chlorate, or in solution as chloric acid. Chlorate can be produced commercially or in laboratory settings. For example, metal chlorates can be prepared by adding chlorine to hot metal hydroxide such as potassium hydroxide or sodium hydroxide as will be understood by a person skilled in the art. The industrial scale synthesis can start from aqueous chloride solution instead of chlorine gas. Chlorate can also be isolated and purified from natural sources as will be understood by a person skilled in the art.

In embodiments herein described, an effective amount of chlorate is an amount effective on Nar-containing bacteria in target environments at the time when the Nar-containing bacteria are hypoxic or anoxic and/or in a targeted manner in environments where nitrate is present at micromolar concentrations, as will be understood by a skilled person upon reading of the present disclosure.

In particular, in some embodiments of the disclosure chlorate is administered to a target biological environment at a time when (and/or at a location where) Nar-containing bacteria if any are present in the environment, become hypoxic/anoxic and thus express Nar. Accordingly, in those embodiments of the disclosure, chlorate therapy is to administer chlorate at chlorate administration time and administration site within the biological environment selected to deliver chlorate to a biological environment at a time when and/or at a location where Nar is expressed.

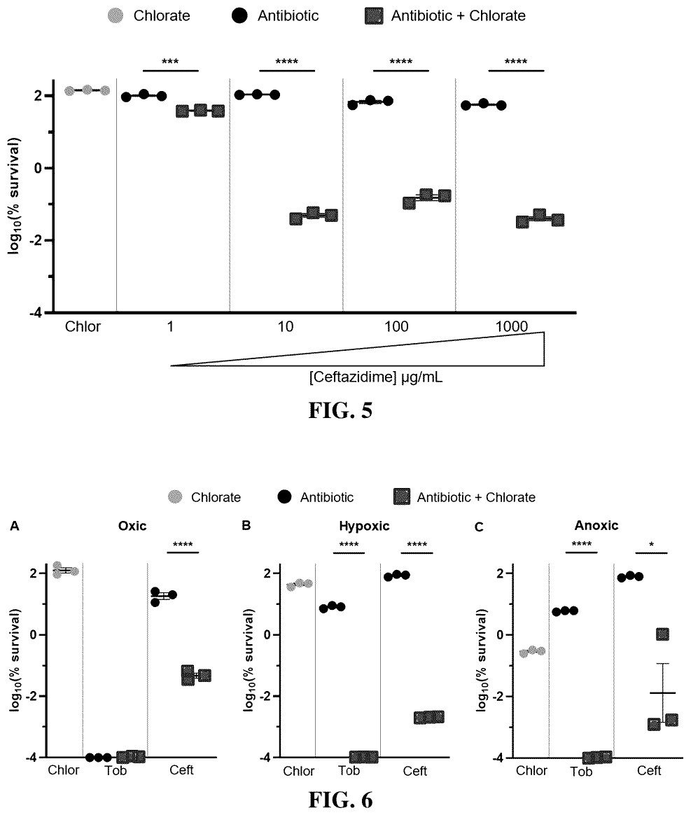

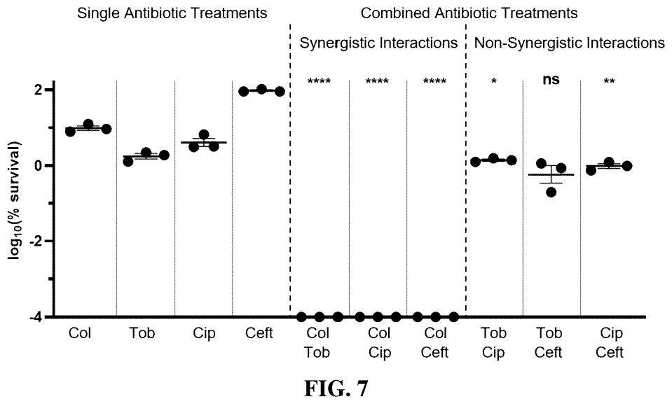

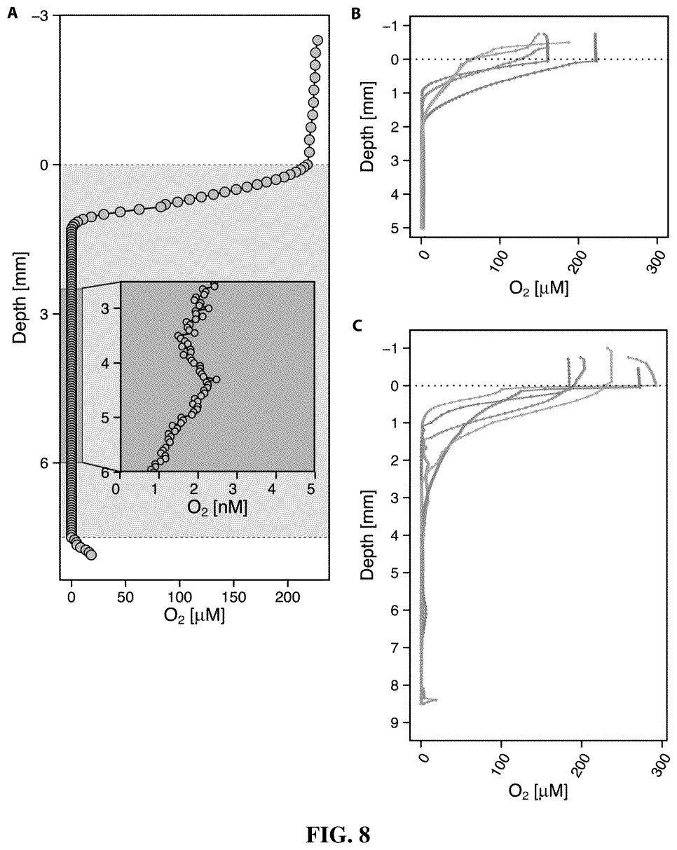

Accordingly, in embodiments of the disclosure the administration time of chlorate delivery based on the known or estimated time of onset of the infection, can be selected based on estimation performed through modeling and calculations such as the diffusion/consumption calculations, of the type done in the Examples 7 to 10 in particular reference to the portions related to FIG. 8 Panel A, 8 Panel B and 19. Generation of a model such as the ones discussed in Examples 7 to 10 can be performed based on first principles, such as those reported in Examples 7 to 10 and in Annex A of U.S. Provisional Application 63/519,537 and Annex A of U.S. Provisional Application Ser. 63/670,084 the content of each which is incorporated by reference in its entirety with particular reference to the passages discussed in FIGS. 6 and 7 of the Annex A. Those examples and passages shows the type of theoretical predictions one can make about what oxygen will look like (at steady state) in an infected human environment. A skilled person can perform this type of analysis for different geometries and have the output be how long it would take for the system to reach hypoxia or anoxia.

In some of those embodiments the timing for chlorate administration can be identified by detecting in the biological environment at least one of oxygen level, redox potential and nitrate concentration and selecting the timing of administration when the oxygen level is below 200 uM, preferably below 150 uM, and more preferably below 100 uM, the redox potential is below 300 mV, preferably below 200 mV, and/or nitrate concentration is above 500 uM. Preferable timing for chlorate administration is identified following quantitative detection of all those three markers when oxygen level is below a selected threshold level preferably below 100 uM, redox potential is below a selected threshold potential preferably below 200 mV, and nitrate concentration is above a selected threshold concentration preferably below 500 uM.

The terms “detect” or “detection” as used herein indicates the determination of the existence, presence, or fact of a target in a limited portion of space, including but not limited to a sample, a reaction mixture, a molecular complex and a substrate. The “detect” or “detection” as used herein can comprise determination of chemical and/or biological properties of the target, including but not limited to ability to interact, and in particular bind, other compounds, ability to activate another compound and additional properties identifiable by a skilled person upon reading of the present disclosure. The detection can be quantitative or qualitative. A detection or measurement is “quantitative” when it refers, relates to, or involves the measurement of quantity or amount of the target or signal (also referred to as quantitation), which includes but is not limited to any analysis designed to determine the amounts or proportions of the target or signal.

A detection is “qualitative” when it refers, relates to, or involves identification of a quality or kind of the target or signal in terms of relative abundance to another target or signal, which is not quantified.

In embodiments herein described detection of oxygen levels, redox potential and/or nitrate concentration is performed quantitively by measuring the amount or concentration of the reference item in the biological environment.

The term “oxygen level” as used herein indicates the concentration of O₂ present in an environment, for example in a liquid, a solid or a gel. Exemplary biological environments which can include oxygen comprise blood (liquid) and mucus (gel).

Oxygen level can be detected by electrochemical methods such as polarographic, pulsed polarographic and galvanic sensors, or by optical methods such as laser oximetry.

The term “redox potential” as used herein indicates a thermodynamic property of a system that describes the stability of the system under a set of conditions towards redox reactions involving the transfer of electrons to or from the system.

Redox potential can be detected by electrochemical methods in solution such as cyclic voltammetry, differential pulse voltammetry, square wave voltammetry, direct current polarography and redox titration. In particular, the redox potential of a material can be measured by using a working electrode made of platinum, gold, glassy carbon, or other suitable inert conductive substrate in combination with a suitable reference electrode (e.g. Ag/AgCl (sat. KCl) or the saturated calomel electrode, SCE) and reported with respect to a common reference value (often the standard hydrogen electrode, SHE, with value 0 mV).

Herein, “working electrode” indicates the electrode used to contact the solution for measurement. “Reference electrode” refers to an electrode used by the system to provide a reference voltage potential for the working electrode.

The term “nitrate” as used herein indicates the nitrogen (V) oxoanion NO₃⁻. This can be present in a system as the anion when dissolved in solution, or associated with a suitable cation to form a nitrate salt in the solid phase.

Nitrate concentration can be detected by electrochemical methods such as by using a nitrate ion selective electrode (ISE) or by optical methods such as infrared spectroscopy, Raman spectroscopy, UV-visible absorption spectroscopy and luminescence/emission spectroscopy.

In embodiments herein described detection of oxygen level, redox potential and nitrate concentration define the oxygenation status of a biological environment of a biological environments a region thereof or a sample thereof, the oxygenation status selected from oxic status, hypoxic status and anoxic status.

A “oxic status” or “oxic condition” as used herein indicates a set of condition of an environment or region thereof in which oxygen is present in quantities that can sustain aerobic biological processes of a Nar-containing bacteria and are incompatible with anaerobic biological process of Nar-containing bacteria. Accordingly, an oxic environment and an oxic region can be identified by an oxygen level above 100 uM, preferably above 150 uM, and more preferably above 200 uM. An oxic status in the sense of the disclosure can accompanied by a redox potential above 300 mV and nitrate in no detectable concentration or in a detectable concentration of nitrate lower than 100 uM.

An “hypoxic status” or “hypoxic condition” as used herein indicates a set of condition of an environment or region thereof in which oxygen is present in a quantities that impair normal function of aerobic biological processes of a Nar-containing bacteria while enabling anaerobic biological processes of a Nar-containing bacteria. Accordingly, a hypoxic environment and a hypoxic region can be identified by an oxygen level above 20 uM, and below 200 uM preferably below 150 uM, and more preferably below 100 uM. A hypoxic oxic status in the sense of the disclosure can accompanied by a redox potential below 300 mV and a detectable concentration of nitrate above 100 uM, possibly up to 500 uM or higher depending on the level of hypoxia.

An “anoxic status” used herein indicates a set of condition of an environment or region thereof in which oxygen is present in a quantities that sustain the normal function of anaerobic biological processes of a Nar-containing bacteria and are incompatible with aerobic biological processes of a Nar-containing bacteria. Accordingly, an anoxic environment and an anoxic region can be identified by an oxygen level below 20 uM. An anoxic status in the sense of the disclosure can accompanied by a redox potential below 300 mV and a detectable concentration of nitrate above than 500 uM, as will be understood by a skilled person upon reading of the present disclosure.

The oxygen level and oxygenation status of a biological environment or of a region thereof are influenced by a variety of factors that affect both the delivery and utilization of oxygen in the environment as will be understood by a skilled person. For example the key factors that determine oxygenation in tissues of an individual is the amount of blood delivered to the tissue, hemoglobin level in the blood delivered as well as the related oxygen saturation (percentage of hemoglobin binding sites occupied by oxygen) as well as oxygen metabolic demand due to a tissue activity, and pathological conditions such as infection or hypermetabolic states increase oxygen demand, potentially leading to tissue hypoxia if supply cannot meet demand. Additional factors which can impact oxygen level and oxygenation status of a biological environment or of a region thereof, comprise cellular process which can alter the efficiency of mitochondria in using oxygen for ATP production. Mitochondrial dysfunction in cells of a tissue of an induvial can impair this process, leading to cellular hypoxia despite adequate oxygen delivery as will be understood by a skilled person.

Accordingly, oxygen level and oxygenation status of a biological environment or of a region thereof, can vary in time and different regions of a same biological environment can have different oxygen levels (see e.g. region at different depth, with different vascularization and/or inflammation status).

In some embodiment herein described, the methods and systems comprise detecting at one time or over time the oxygen level, redox potential and/or nitrate concentration as marker of the oxygenation status of a biological environment or a target portion thereof over time.

In some embodiment herein described, the methods and systems comprise detecting the oxygen level, redox potential and/or nitrate concentration as marker of the oxygenation status of at least one target region and preferably a plurality of target regions of a biological environment to identify anoxic region, hypoxic region and/or oxic regions, if any is present, within the biological environment.

In embodiments herein described measurement of oxygen level, redox potential and nitrate concentration can be performed in a biological environments or portion thereof depending on whether the detection is performed in vivo, in vitro or ex-vivo. For example, analyses can be performed ex situ by taking a sample from a biological environment, measuring the oxygen level using an electrochemical sensor, measuring the redox potential with a redox electrode coupled with a suitable reference electrode, and measuring nitrate concentration with a nitrate ISE. Similarly, an ex-situ analysis can be performed by taking a sample from a biological environment, measuring the oxygen level by laser oximetry, measuring the redox potential with a redox electrode coupled with a suitable reference electrode, and measuring the nitrate concentration by Raman spectroscopy, or any combination of suitable methods for oxygen level, redox potential and nitrate concentration determination. Samples removed from a biological environment can be solid (like a biopsy or a wound or skin swab or a tissue sample), or liquid (like a blood sample or lymph sample or other fluid sample), or gel (like a mucus or sputum sample) or combinations of these.

Alternatively, measurement of oxygen level, redox potential and nitrate concentration can be performed in situ in a biological environment. For example, a device comprising a microelectrode array can be configured to have the capability to measure oxygen level, redox potential and nitrate concentration through side-by-side electrochemical measurement, and then the device can be contacted with the area of interest in the biological environment to perform these electrochemical measurements while interfaced externally to power and/or recording and analysis instrumentation. For example, such electrochemical microelectrode array devices can be installed inside bandages and prostheses for direct contact with a wound or other chronic injury, or inserted inside the body as part of a surgical procedure to measure oxygen level, redox potential and nitrate concentration while in contact with internal surfaces and structures. Similarly, devices for in situ measurement can be configured using any combination of suitable methods for oxygen level, redox potential and nitrate concentration determination (e.g., optical and/or electrochemical methods).

In some embodiments, the selected administering of antibiotic alone or in combination with chlorate is performed based upon detection in the biological environment of threshold level of the markers oxygen level, redox potential and/or nitrate concentration, which indicate if the conditions of the biological environment tested c can be considered “oxic” “hypoxic” or “anoxic” and certain treatments are preferred. Depending on the threshold level the biological environment can be considered “oxic” “hypoxic” or “anoxic” and certain other treatments are preferred.

Embodiments of timed and/or targeted chlorate administration methods herein described, comprise
administering an antibiotic to a biological environment or region thereof which is in oxic condition the antibiotic administered in an antibiotic effective amount to inhibit viability of Nar-containing bacteria in an oxic environment,
administering chlorate in combination with an antibiotic to a biological environment or region thereof which is in hypoxic condition the chlorate and antibiotic administered in a chlorate effective amount and an antibiotic effective amount to inhibit viability of Nar-containing bacteria in a hypoxic environment; and/or
administering chlorate to a biological environment or region thereof which is in anoxic condition the chlorate administered in a chlorate effective amount to inhibit viability of Nar-containing bacteria in an anoxic environment the administering performed optionally in combination with antibiotic in an antibiotic effective among in anoxic environment.

In some embodiments of the present disclosure, the related methods and system are based on the surprising finding that Nar-containing bacteria can exhibit an antibiotic recalcitrance under hypoxic condition which includes genetic resistance (see Example 2) and that administration of chlorate under hypoxic conditions can overcome this recalcitrance also reducing the amount of antibiotic which is effective in inhibit viability of Nar-containing bacteria (see Example 3) and/or broadening the spectrum of suitable antibiotics, through inclusion of antibiotics to which the Nar-containing bacteria would be resistant to in absence of chlorate (see Example 6).

Accordingly in preferred embodiments the antibiotic effective amount in a hypoxic condition can effectively be lower than the antibiotic effective amount under oxic condition. In particular in some of those embodiments the antibiotic effective amount under hypoxic conditions can be lower, e.g. a fraction of the minimum inhibitory concentration of antibiotic (MIC) as will be understood by a skilled person, in those embodiments the hypoxic antibiotic effective amount typically ranges from 0.001 to 500 ug/ml, with higher concentrations corresponding from 0.1-500 ug/ml preferably 1-30 ug/mL or 1-5 ug/mL to increase the efficacy of the treatment in view of the bacteria susceptibility to the antibiotic, the target objective of the treatment (e.g. desired therapeutic effect) the antibiotic used and the biological environment treated (e.g. skin, blood, muscles, lungs, mucosa and others identifiable by a skilled person), as will be understood by a skilled person upon reading of the present disclosure.

In embodiments of timed and/or targeted chlorate administration methods herein described chlorate and/or administration and the related effective amounts are selected based on oxic anoxic or hypoxic status of the biological environment or region thereof, and can be selected in particular, following detection of one or more of oxygen level, redox potential and nitrate concentration as makers of the related oxygenation status.

Detection of oxygen level can be performed with oxygen sensors configured o measure the concentration of oxygen in various settings, such as tissues, organs, or experimental setups. Exemplary sensors comprise Clark-type Electrodes, Optical Oxygen Sensors (Micro-optodes), Planar Oxygen Sensors Wireless Implantable Sensors, Tissue-Integrating Oxygen Sensors, Phosphorescent Oxygen Probes and additional sensors identifiable by a skilled person.

Detection of redox potential is made and reported relative to a standard reference electrode. This reference electrode can be chosen for a particular experiment, and is a standard electrode with known potential such as the Ag/AgCl (sat. KCl) reference (standard potential +0.197 V vs SHE at 25° C.) or the saturated calomel reference (+0.244 V vs SHE at 25° C.). In certain embodiments the measurement of redox potential for a biological sample was made using an Ag/AgCl reference electrode and reported relative to the standard hydrogen electrode (SHE) as described in Annex A of U.S. Provisional Application 63/519,537 and Annex A of U.S. Provisional Application Ser. 63/670,084 the content of each which is incorporated by reference in its entirety, and in Example 8 as will be understood by the skilled person.

Detection of nitrate concentration can be performed with sensors configured to detect nitrate concentrations in biological environments such as tools for monitoring and studying nitrate levels in various biological systems. Exemplary nitrate sensors comprise Electrochemical Sensors, Optical Sensors, Fluorescent Biosensors and additional sensors identifiable by a skilled person.

In some embodiments the oxygen sensor is an electrochemical sensor comprising an oxygen-sensitive electrode, as will be appreciated by the skilled person. The working electrode can be an amperometric, Clark-type electrode or a switchable trace oxygen sensor as described in Example 7 and Example 30, or other suitable electrode configuration capable of detecting the concentration of oxygen by electrochemical methods. The electrode can be a macroelectrode or a microelectrode, and can have tip diameter between 1-10 mm or between 10-50 um. Measurements can be made in situ directly in the infected biological sample or can be made ex situ on an excised section, tissue sample or fluid sample of the infected biological sample, or any combination of these scenarios. The electrode sensor can be connected to a suitable control device such as a potentiostat either externally through suitable wires or by incorporation of the control function into the device such as in a prosthetic device or bandage. In certain embodiments the signal from the electrochemical oxygen sensor electrode can be amplified, as will be understood by the skilled person, so the electrode probe can be connected to a high-sensitivity amplifier in a multimeter as part of the device control setup. The probe can be calibrated, as will be understood by the skilled person, and suitable methods such as a two-point or three-point calibration using standard solutions of known oxygen concentration can be carried out.

In some embodiments the redox potential sensor is an electrochemical sensor comprising a working electrode and a reference electrode, as described in Example 8 and Example 31. In certain embodiments, the working electrode can comprise glassy carbon or other carbon materials such as graphene or fullerenes or carbon nanotubes, or gold, platinum or other suitable metal as will be understood by the skilled person. The reference electrode can be a standard reference electrode with known potential such as the Ag/AgCl (sat. KCl) reference (standard potential +0.197 V vs SHE at 25° C.) or the saturated calomel reference (+0.244 V vs SHE at 25° C.). Either electrode can be a macroelectrode or a microelectrode, and can have tip diameter between 1-10 mm or between 10-50 um. Measurements can be made in situ directly in the infected biological sample or can be made ex situ on an excised section, tissue sample or fluid sample of the infected biological sample, or any combination of these scenarios. The electrode sensor can be connected to a suitable control device such as a potentiostat either externally through suitable wires or by incorporation of the control function into the device such as in a prosthetic device or bandage. In certain embodiments the signal from the redox potential electrode can be amplified, as will be understood by the skilled person, and the electrode probe can be connected to a high-sensitivity amplifier in a multimeter as part of the device control setup. The probe can be calibrated, as will be understood by the skilled person, and suitable methods such as a two-point or three-point calibration using standard solutions of known potential such as buffered quinones can be carried out.

In some embodiments the nitrate sensor is an electrochemical sensor comprising a nitrate ion sensitive electrode, as described in Example 32. This electrode can be a macroelectrode or a microelectrode, and have tip diameter between 1-10 mm or between 10-50 um respectively. Measurements can be made in situ directly in the infected biological sample or can be made ex situ on an excised section, tissue sample or fluid sample of the infected biological sample, or any combination of these scenarios. The electrode sensor can be connected to a suitable control device such as a potentiostat either externally through suitable wires or by incorporation of the control function into the device such as in a prosthetic device or bandage. In certain embodiments the signal from the nitrate ion sensitive electrode can be amplified, as will be understood by the skilled person, and the electrode probe can be connected to a high-sensitivity amplifier in a multimeter as part of the device control setup. The probe can be calibrated, as will be understood by the skilled person, and suitable methods such as a two-point or three-point calibration using standard solutions of known nitrate concentration can be carried out.

In some embodiments, the selected administering of antibiotic alone or in combination with chlorate is performed based upon detection of a threshold oxygen level of 200 uM with antibiotic administered if the measured oxygen level is 200 uM or more and antibiotic and/or chlorate administered if the oxygen level detected is less than 200 uM More preferably antibiotic and chlorate are administered when the oxygen level detected is less than 200 uM and higher than 20 uM and chlorate is administered when oxygen level detected is less than 20 uM.

In some preferred embodiments the selected administering of antibiotic alone or in combination with chlorate is performed based upon detection of a threshold oxygen level of 150 uM with antibiotic administered if the measured oxygen level is 150 uM or more and antibiotic and/or chlorate administered if the oxygen level detected is less than 150 uM More preferably antibiotic and chlorate are administered when the oxygen level detected is less than 200 uM and higher than 20 uM and chlorate is administered when oxygen level detected is less than 20 uM.

In some more preferred embodiments, the selected administering of antibiotic alone or in combination with chlorate is performed based upon detection of oxygen level of 100 uM with antibiotic administered if the measured oxygen level is 100 uM or more and antibiotic and/or chlorate administered if the oxygen level detected is less than 100 uM. More preferably antibiotic and chlorate are administered when the oxygen level detected is less than 200 uM and higher than 20 uM and chlorate is administered when oxygen level detected is less than 20 uM.

In some embodiments the selected administering of antibiotic alone or in combination with chlorate is performed based upon detection a threshold redox potential of +300 mV vs SHE, with antibiotic administered if the measured redox potential is +300 mV vs SHE, or more and antibiotic or chlorate administered if the redox potential is below +300 mV vs SHE.

In some preferred embodiments the selected administering of antibiotic alone or in combination with chlorate is performed based upon detection of a threshold redox potential of +250 mV vs SHE, with antibiotic administered if the measured redox potential is +250 mV vs SHE, or more and antibiotic or chlorate administered if the redox potential is below +250 mV vs SHE.

In some ore preferred embodiments the selected administering of antibiotic alone or in combination with chlorate is performed based upon detection of a threshold redox potential of +200 mV vs SHE, with antibiotic administered if the measured redox potential is +200 mV vs SHE, or more and antibiotic or chlorate administered if the redox potential is below +200 mV vs SHE.

In some embodiments the selected administering of antibiotic alone or in combination with chlorate is performed based upon detection of nitrate at a threshold amount of 500 uM, with antibiotic administered if the measured nitrate concentration is less than 500 uM and antibiotic or chlorate administered if the nitrate concentration detected is more than 500 uM.

In some embodiments the selected administering of antibiotic alone or in combination with chlorate is performed based upon detection of nitrate at a threshold amount of less than 500 uM e.g. 450 uM or less, additional nitrate administration is performed, before proceeding with administration antibiotic or chlorate when the nitrate concentration reaches 500 uM or more.

In more preferred embodiments, the selected administering of antibiotic alone or in combination with chlorate is performed based upon combined detection of at least two and preferably all three markers of oxic/hypoxic/anoxic conditions of the biological environment.

In some embodiments the measured oxygen level is less than 100 uM, the measured redox potential is below +200 mV vs SHE and the measured nitrate concentration is 500 uM or more indicating an anaerobic biological environment and the selected administering is administering an effective amount of chlorate and antibiotics.

In some embodiments the measured oxygen level is less than 100 uM, the measured redox potential is below +200 mV vs SHE and the measured nitrate concentration is less than 500 uM indicating an anaerobic biological environment with low level of nar and the selected administering is administering an effective amount of chlorate and antibiotics more preferably preceded by administration of nitrate in effective amount to increase the concentration of nitrate in the biological environment to 500 uM or more.

In some embodiments the measured oxygen level is greater than 100 uM, the measured redox potential is above +200 mV vs SHE and measured nitrate concentration below the detection limit indicating an aerobic biological environment and the selected administering is administering an effective amount of antibiotic.

In some embodiments the measured oxygen level is less than 150 uM, the measured redox potential is below +200 mV vs SHE and the measured nitrate concentration is 500 uM or more indicating an anaerobic biological environment and the selected administering is administering an effective amount of chlorate and antibiotics.

In some embodiments the measured oxygen level is less than 200 uM, the measured redox potential is below +200 mV vs SHE and the measured nitrate concentration is 500 uM indicating an anaerobic biological environment and the selected administering is administering an effective amount of chlorate and antibiotics.

In some embodiments the measured oxygen level is less than 200 uM, the measured redox potential is below +250 mV vs SHE and the measured nitrate concentration is 500 uM or more indicating an anaerobic biological environment and the selected administering is administering an effective amount of chlorate and antibiotics.

In some embodiments the measured oxygen level is less than 200 uM, the measured redox potential is below +300 mV vs SHE and the measured nitrate concentration is 500 uM or more indicating an anaerobic biological environment and the selected administering is administering an effective amount of chlorate and antibiotics.

In some embodiments the measured oxygen level is less than 200 uM, the measured redox potential is below +300 mV vs SHE and the measured nitrate concentration is 450 uM or more with low level of nar and the selected administering is administering an effective amount of chlorate and antibiotics more preferably preceded by administration of nitrate in effective amount to increase the concentration of nitrate in the biological environment to 500 uM or more.

In some embodiments the detection can be performed in sample of the biological environment.

The term “sample” as used herein indicates a limited quantity of something that is indicative of a larger quantity of that something, including but not limited to fluids from a specimen such as biological environment, cultures, tissues, commercial recombinant proteins, synthetic compounds or portions thereof.

In particular, a “biological sample” in the sense of the disclosure indicates a sample comprising cells and/or other biological compartments wherein the term “cell” in the sense of the disclosure indicates the basic structural and functional units of life, as will be understood by a skilled person. Accordingly, a biological sample can comprise one or more cells of any biological lineage such as cells of an individual, microbial cells and in particular prokaryotic cells as well as virus, as being representative of the total population of similar cells or virus in the biological environment which can be a sampled individual or a portion thereof such as a tissue or an organ. Biological sample can comprise a host sample combined with a reagent, a buffer, a dilutant as will be understood by a skilled person.

Exemplary biological samples from an individual comprise the following: whole venous and arterial blood, capillary blood, blood plasma, blood serum, dried blood spots, cerebrospinal fluid, interstitial fluid, sweat, lumbar punctures, nasal secretions, sinus washings, tears, corneal scrapings, saliva, sputum or expectorate, bronchoscopy secretions, transtracheal aspirate, endotracheal aspirations, bronchoalveolar lavage, vomit, endoscopic biopsies, colonoscopic biopsies, subcutaneous and mesenteric adipose tissue biopsies, bile, vaginal fluids and secretions, endometrial fluids and secretions, urethral fluids and secretions, mucosal secretions, synovial fluid, ascitic fluid, peritoneal washes, tympanic membrane aspirate, urine, clean-catch midstream urine, catheterized urine, suprapubic aspirate, kidney stones, prostatic secretions, feces, mucus, pus, wound draining, skin scrapings, skin snips and skin biopsies, hair, nail clippings, cheek tissue, bone marrow biopsy, solid organ biopsies, surgical specimens, solid organ tissue, cadavers, breast milk, or tumor cells, among others identifiable by a skilled person. Biological samples can be obtained using sterile techniques or non-sterile techniques, as appropriate for the sample type, as identifiable by persons skilled in the art. Depending on the type of biological sample and the intended analysis, biological samples can be used freshly for sample preparation and analysis, or can be fixed using fixative.

In methods and systems and related devices and composition following detection of oxygen level, redox potential and/or nitrate in the biological environment and/or sample thereof, chlorate, antibiotics and/or nitrate can be administered at a respective administration time and an administration target site estimated and selected according to the methods herein described.

In some of those embodiments, the estimated chlorate administration time can be determined based on the loading of the infection (how many bacterial cells a are present in the biological environment) as well as based on presence of additional cell (e.g. cells of an individual) also present in the biological environment and engaging in aerobic respiration thus contributing to oxygen consumption.

In some of those embodiments, chlorate administration time can be performed from minutes, to hours, to days from the known or estimated onset of the infection in the biological environment entirely depending on the rate of oxygen consumption vs. the rate of oxygen delivery in the biological environment, which in turn are influenced by the geometry of the infected environment and the cellular composition of that environment as understood by a skilled person. A skilled person understand that biological environment can include bacterial and non-bacteria cells, such as to host cells and cells of microbial communities formed by non-pathogenic microorganism possibly present in a biological environment of an individual. Additionally, different biological environments, such as different areas of the body of an individual, have different capacities to oxygenate and different microbiome composition as will also be understood by a skilled person.

Accordingly, a skilled person will understand the specific chlorate administration time for specific biological environment will depend on the particular details of the infection. Earlier on, cells are less abundant and there's more oxygen. As they proliferate, oxygen gets consumed. This is when infections often become chronic with biofilms formation and formation of hypoxic zone which can start pretty quickly (in wounds it can occur in about 24 hrs).

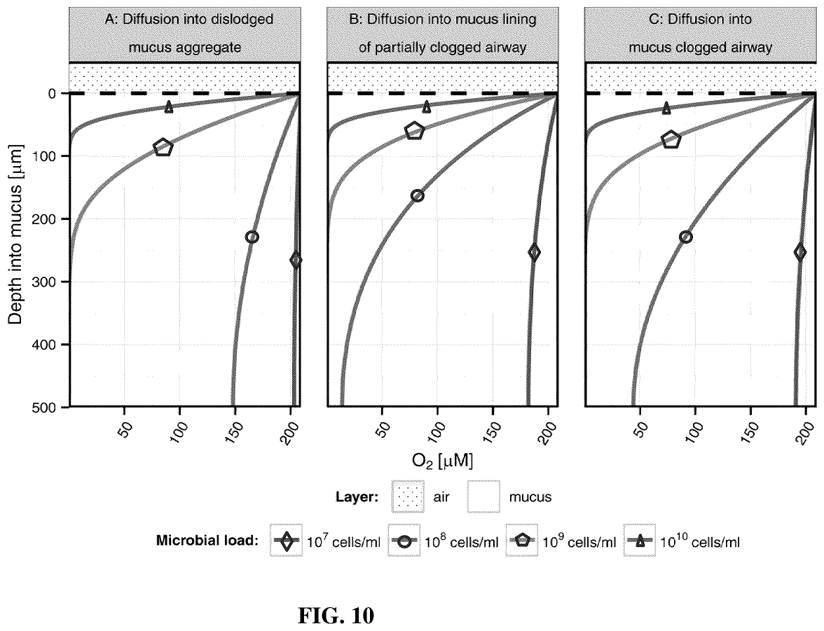

Accordingly, in some embodiments of the disclosure the administration time of chlorate delivery based on the known or estimated time of onset of the infection, can be selected based on estimation performed through modeling and calculations such as the diffusion/consumption calculations, of the type done in Examples 7 to 10 with particular reference to FIGS. 10, 11 and 19. Generation of a model such as the one included in FIGS. 10, 11 and 19 can be performed based on first principles, such as those reported in Examples 7 to 10 which shows the type of theoretical predictions one can make about what oxygen will look like (at steady state) in an infected human environment. A skilled person can perform this type of analysis for different geometries and have the output be how long it would take for the system to reach hypoxia or anoxia.

In addition or in alternative to using modeling a chlorate administration timing can be determined for example by making measurements across multiple infections using oxygen sensors to identify oxic and anoxic portions of an environment, to determine when an oxic portion of environment become anoxic, or when an anoxic portion of environment become oxic, Exemplary techniques comprise electrochemical probes, microdialysis, MRI, EPR, as well as tissue integrating sensors and additional sensors identifiable by a skilled person [23].

In some of embodiments the timing for chlorate administration can be identified by detecting in the biological environment at least one of oxygen level, redox potential and nitrate concentration and selecting the timing of administration when the one or more of these is below a certain threshold.

Threshold values for oxygen level can be determined by constructing an oxygen microprofile of the biological environment using oxygen sensor electrodes. A typical method is described in Examples 7 to 10. FIG. 8 shows a representative oxygen microprofile for sputum from a cystic fibrosis infection, and a skilled person will understand that other bacterial infections will have their own microprofiles that can be determined in the same manner. In addition, FIG. 8 shows that use of two different oxygen sensor electrode gives similar results, and a skilled person will appreciate that a variety of appropriately-configured and calibrated oxygen sensors can be used to obtain the same result.

FIG. 8 shows a typical oxygen microprofile, with three distinct regions: near the air-sputum interface (˜0-1 mm deep into the biological environment) there is a region where oxygen concentration is high (>200 uM O₂) that is defined here as the “oxic zone”. Following this, deeper into the biological environment a steep oxycline begins that traverses through a region defined here as the “hypoxic zone” (200-20 uM O₂, ˜1-1.5 mm deep) into the final region defined here as the “anoxic zone” (<20 uM O₂) that persists for the remaining portion of the biological environment. Hence by assessment of the oxygen microprofile the following thresholds can be obtained as will be understood by the skilled person.

The skilled person will appreciate that the specific thresholds defining oxic conditions hypoxic conditions and anoxic conditions is dependent on the specific biological environment and can vary depending on the specific biological target regions thereof or sample thereof. For example, gene expression of alginate by Pseudomonas aeruginosa has been observed in a hypoxic zone bounded by between ˜40 to 200 uM O₂ [24]), and hypoxic conditions for growth of a number of bacterial cultures for further study have been described as 7-9% O₂ (˜60-90 uM O₂; [25]). However, the oxygen level thresholds provided here are intended to give guidance to the skilled person who will know that careful modeling and/or measurement of oxygen concentration to generate a microprofile for a specific biological environment of interest can be carried out in addition if required.

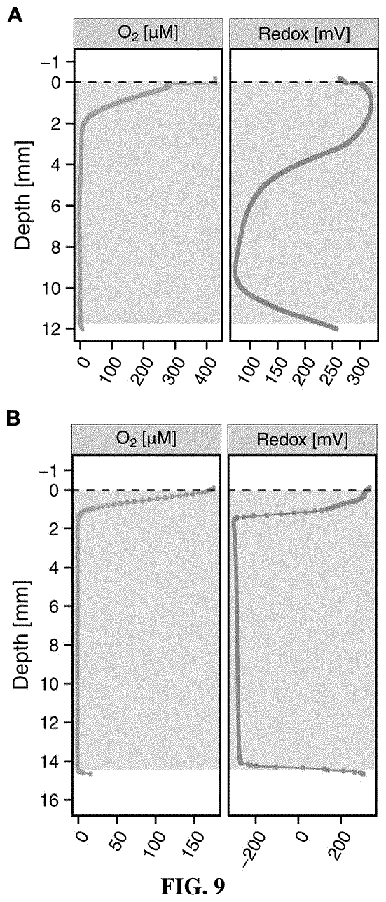

Threshold values for redox potential can be determined by profiling a biological sample of interest with a redox microelectrode in tandem with an oxygen sensor. A typical method is described in Examples 7 to 10, and FIG. 9 shows representative examples from cystic fibrosis sputum samples. As will be evident to the skilled person, the oxygen microprofile measured follows the general outline described above for all samples whereas the redox potential trend can vary considerably between samples as a function of depth into the sample (FIG. 9 Panel A compared to FIG. 9 Panel B). However, in all cases comparison of the oxygen and redox profiles indicates that a redox potential of <200 mV is measured only in the regions corresponding to hypoxic or anoxic oxygen concentrations. Therefore, a skilled person will appreciate that a measured redox potential of <200 mV is sufficient to characterize the biological sample environment as either hypoxic or anoxic.

Threshold values for nitrate concentration can be determined by profiling a biological sample of interest with a nitrate sensitive electrode in tandem with an oxygen sensor. Nitrate production by nitrate-respiring bacteria is characteristic of their anaerobic respiration, so any non-zero value for nitrate concentration measured in such a way for a sample region can be considered an indication that the sample region has a hypoxic or anoxic oxygen concentration, as a skilled person will appreciate. The following nitrate concentration ranges are provided as guidance to the skilled person: Oxic: no nitrate concentration detected Hypoxic: 1-100 uM nitrate Anoxic: >100 uM nitrate.

In addition or in alternative to using modeling and/or oxygen sensors, chlorate administration timing can be determined for example by detecting biomarkers of anaerobic and/or aerobic respiration of Nar-containing bacteria.

Exemplary biomarkers that can be detected for this purpose comprise the exemplary measurements described in Examples 11 and 12 and in Annex B of U.S. Provisional Application 63/519,537 and Annex B of U.S. Provisional Application Ser. 63/670,084 herein incorporated by reference in their entirety, for biofilm aggregates grown in the lab as a proof of principle.

Accordingly, exemplary biomarkers comprise dissimilatory nitrate reductase (narG), terminal oxidase (ccoN1), nitrite reductase (nirS), nitrous oxide reductase (nosZ), and acetate kinase (ackA), whose express can be detected to determine gene expression levels across heterogeneous populations such as biofilms. For example, using probes directed to the detection of one or more of these biomarkers allows one of skill to quantify gene expression across oxygen gradients in aggregate populations grown using the assays such as agar block biofilm assay (ABBA) (see e.g. tests performed in Examples 11 and 12 and in Annex B of U.S. Provisional Application 63/519,537 and Annex A of U.S. Provisional Application Ser. 63/670,084).

In particular, ccoN1 is a biomarker of aerobic respiration which peak expression under oxic conditions while detected ackA is a biomarker of aerobic respiration which peak expression under anoxic condition as discussed and exemplified in Examples 11 and 12 and Annex B of U.S. Provisional Application 63/519,537 and Annex B of U.S. Provisional Application Ser. 63/670,084. Similar considerations apply to denitrification genes narG, nirS, and nosZ biomarkers of anaerobic respiration which peak expression in hypoxic and anoxic regions, although nirS expression remained at peak levels deeper into anoxic environments than other denitrification genes, as also discussed and exemplified in Examples 11 and 12 and in Annex B of U.S. Provisional Application 63/519,537 and Annex B of U.S. Provisional Application Ser. 63/670,084.

Additional biomarkers comprise metabolites only generated under aerobic or anaerobic conditions. For example, detection of sulfide and nitrous oxide which are produced under anaerobic conditions only can be performed in time to determine when the related concentration peaks or drops as a marker of anoxic or oxic conditions respectively as will be understood by a skilled person.

A list of biomarkers that can be used in connection with the present disclosure is reported in Table 2 below.

TABLE 2
Target sequences for HCR probes

| | | SEQ |
|---|---|---|
| | | ID |
| Target | Sequence | NO |
| nosZ 1 | GAGCGACGACACGAAAAGCCCCCACGAAGAAACCCACGGCCTGA | 6 |
| | ACCGCC | |
| nosZ 2 | GCGCAGCAAGGCCGAGGTCGCCCCCGGCGAACTGGATGAGTACT | 7 |
| | ACGGGT | |
| nosZ 3 | GGAGCGGCGGACATTCCGGCGAAGTACGCGTGCTCGGCGTGCCG | 8 |
| | TCGATG | |
| nosZ 4 | GAGCTGATGCGCATACCGGTGTTCAACGTCGACTCGGCCACCGGC | 9 |
| | TGGGG | |
| nosZ 5 | GACCAACGAGAGCAAGCGGGTCCTCGGCGACAGCGCGCGCTTCC | 10 |
| | TCAACG | |
| nosZ 6 | ACTGCCACCATCCGCACATCTCGATGACCGACGGCAAGTACGACG | 11 |
| | GCAAG | |
| nosZ 7 | CATGAAATGCGACCGCATCGTCACCATTCCCAACGTCCAGGCGAT | 12 |
| | CCACG | |
| nosZ 8 | TGCGCCTGCAAAAGGTGCCGCATACCCGCTACGTGTTCTGCAACG | 13 |
| | CCGAG | |
| nosZ 9 | ATCATCCCCCATCCCAACGACGGCTCGACCTTCGACCTGTCCGGC | 14 |
| | GACAA | |
| nosZ | CTTCACCCTGTACAACGCCATCGACGCCGAGACCATGGAAGTGGC | 15 |
| 10 | CTGGC | |
| nosZ | TGATCGTCGACGGCAACCTCGACAACACCGACATGGACTACAGC | 16 |
| 11 | GGCAGG | |
| nosZ | GCCGCCTCCACCTGCTACAACTCGGAGAAGGCCGTCGACCTCGGC | 17 |
| 12 | GGCAT | |
| nosZ | AGATCAAGGCGAAGCGCTTCGTCACCCTCGGCGACTCGAAGGTG | 18 |
| 13 | CCGGTG | |
| nosZ | GACGGCCGGCGCAAGGACGGCAAGGACAGCCCGGTGACCCGCTA | 19 |
| 14 | CATCCC | |
| nosZ | ACCGAAGAACCCCCACGGGCTGAACACCTCGCCGGACGGCAAGT | 20 |
| 15 | ACTTCA | |
| nosZ | CCAACGGCAAGCTCTCGCCGACCTGCACCATGATCGCCATCGAGC | 21 |
| 16 | GCCTC | |
| nosZ | GACCTGTTCGCCGGCAAGCTGGCCGACCCGCGCGACGTGGTGGTG | 22 |
| 17 | GGCGA | |
| nosZ | GGAACTGGGCCTCGGCCCGCTGCACACCACTTTCGATGGCCGAGG | 23 |
| 18 | CAACG | |
| nosZ | ATACCACGCTGTTCATCGACAGCCAGTTGGTGAAGTGGAACCTGG | 24 |
| 19 | CCGAC | |
| nosZ | TGCCACGCCCTGCACATGGAAATGTGCGGGCGGATGCTGGTGGA | 25 |
| 20 | AAAGGC | |
| ccoN1 | TCGCCGTTATGACGGTGGTCTGGGGGGTCATTGGAATGGGTCTCG | 26 |
| 1 | GTGTC | |
| ccoN1 | TCGGTGGGTGCGCCCTCTTCGCCACCTCGTACTACGTGGTGCAAC | 27 |
| 2 | GTACC | |
| ccoN1 | CTGATTTCAGACACGCTGGCGGCCTTCACCTTCTGGGGTTGGCAG | 28 |
| 3 | GCCGT | |
| ccoN1 | TCGTGGGCGCCGTGCTGACCCTGCCGCAGGGTTTCACCACCTCCA | 29 |
| 4 | AGGAA | |
| ccoN1 | GCCGAACTGGAATGGCCGCTGGCCATCCTCCTGGCGATCGTCTGG | 30 |
| 5 | ATCAC | |
| ccoN1 | GCCTTCATCCTGGTGACGGCGATGCTGCACATCGTCAACCACATG | 31 |
| 6 | TCGCT | |
| ccoN1 | ACTGGTCGCAAGCCACCCGGGCTTCATCGTGCGCATGATCGGCGG | 32 |
| 7 | TGGTT | |
| ackA 1 | GCCCTCACGCAACATACTGGTGATCAACTGCGGCAGTTCGTCGAT | 33 |
| | CAAGT | |
| ackA 2 | CCCTGGTCAACGAGGCCCACTCCCTGTTTCCCCTGCACGGCCTCG | 34 |
| | CCGAG | |
| ackA 3 | CTGGGCAGCCGCGATGCGGTGCTGCGCTGGAAGCGCGGCGGCGA | 35 |
| | CAGCGA | |
| ackA 4 | CCTGATGATTCCCAACGCCGACCACCGCGCCGCCCTCGCCCAGTT | 36 |
| | GCTGC | |
| ackA 5 | TGGTGCAGAACGCCGCGGGCGGCAAGCTCCACGGCATCGGCCAC | 37 |
| | CGGGTG | |
| ackA 6 | CATGGCGGCGAGCTGTTCACCCATGCCACGCGCATCGACGACCGG | 38 |
| | GTGGT | |
| ackA 7 | GGCGATCCGGGCCACCGCGCCGCTGGCGCCGCTGCACAACCCGG | 39 |
| | CCAACC | |
| ackA 8 | AAGGCATCGAGGCAGCGATGACGCTGTTTCCCAAGCTGCCCCACG | 40 |
| | TCGCC | |
| ackA 9 | TTCGACACCGCCTTCCACCAGAGCCTGCCGGAGCACGCCTACCGC | 41 |
| | TACGC | |
| ackA | AGCATGGCGTGCGCCGCTACGGCTTCCACGGCACCAGCCACCGCT | 42 |
| 10 | ACGTC | |
| ackA | GGCGACAGCAGTTGGCTCAGCGCCCACCTCGGCAACGGCAGCTC | 43 |
| 11 | GACCTG | |
| ackA | CATCGTCAACGGCCAGAGCCTCGACACCAGCATGGGCCTGACCCC | 44 |
| 12 | GCTGG | |
| ackA | GCCTGGTAATGGGCACCCGCAGCGGCGACGTCGACCCCAACCTG | 45 |
| 13 | CACAGC | |
| ackA | CTGGCGCGGACCCTGGGCTGGAGCCTGGAGCGCATCGACTCGAT | 46 |
| 14 | GCTGAA | |
| ackA | CGAAAGCGGCCTGCTCGGCCTCTCCGACCTGTCCAACGACATGCG | 47 |
| 15 | CACCC | |
| ackA | AGCAGGAGCGCGAGCAGGGCCACCCCGGCGCGGCCCTGGCGATC | 48 |
| 16 | GAGGTG | |
| ackA | TGCTACCGCCTGGCCAAGTCCCTGGCGGCGATGAGCTGCGCCCTG | 49 |
| 17 | CCGCA | |
| ackA | GGACGGGGTGATCTTCACCGGTGGCATCGGCGAGAACTCGCCGCT | 50 |
| 18 | GGTGC | |
| ackA | CCAAGACCGCCGCCCACCTGCGGCTGTTCGACCTGCGCCTCGACC | 51 |
| 19 | AGGAG | |
| ackA | AACGCCCGCTGCGTGCGCGGCGTCGCCGGGCCGATCCAGGCCGC | 52 |
| 20 | GGGACA | |
| ackA | GCGGGTACTGGTGATCCCGACCAACGAAGAGCGGCAGATCGCCC | 53 |
| 21 | TCGACA | |
| nirS 1 | GCCATTTGGCAAGCCACTGGTGGGCACCTTGCTCGCCTCGCTGAC | 54 |
| | GCTGC | |
| nirS 2 | GCCTGGCCACCGCTCACGCCAAGGACGACATGAAAGCCGCCGAG | 55 |
| | CAATAC | |
| nirS 3 | GGTGCCGCTTCCGCCGTCGATCCCGCTCACGTGGTGCGCACCAAC | 56 |
| | GGTGC | |
| nirS 4 | CGACATGAGTGAAAGCGAGTTCAACGAGGCCAAGCAGATCTACT | 57 |
| | TCCAAC | |
| nirS 5 | CCGGACATCACCCAGCAACGCGGCCAGCAATACCTGGAAGCGCT | 58 |
| | GATCAC | |
| nirS 6 | CGGCACCCCGCTGGGCATGCCGAACTGGGGCAGCTCCGGCGAGC | 59 |
| | TGAGCA | |
| nirS 7 | AACAGATCACCCTGATGGCCAAGTACATCCAGCACACCCCGCCGC | 60 |
| | AACCG | |
| nirS 8 | GAGTGGGGCATGCCGGAGATGCGCGAATCGTGGAAGGTGCTGGT | 61 |
| | GAAGCC | |
| nirS 9 | GGACCGGCCGAAGAAACAGCTCAACGACCTCGACCTGCCCAACC | 62 |
| | TGTTCT | |
| nirS | TGACCCTGCGCGACGCCGGGCAGATCGCCCTGGTCGACGGCGAC | 63 |
| 10 | AGCAAG | |
| nirS | ATCGTCAAGGTCATCGATACCGGCTATGCCGTGCATATCTCGCGG | 64 |
| 11 | ATGTC | |
| nirS | TTCCGGCCGCTACCTGCTGGTGATCGGCCGCGACGCGCGGATCGA | 65 |
| 12 | CATGA | |
| nirS | ACCTGTGGGCCAAGGAGCCGACCAAGGTCGCCGAGATCAAGATC | 66 |
| 13 | GGCATC | |
| nirS | GCGCGCTCGGTGGAAAGCTCCAAGTTCAAGGGCTACGAGGACCG | 67 |
| 14 | CTACAC | |
| nirS | CGCCGGCGCCTACTGGCCGCCGCAGTTCGCGATCATGGACGGCGA | 68 |
| 15 | GACCC | |
| nirS | AACCGAAGCAGATCGTCTCCACCCGCGGCATGACCGTAGACACC | 69 |
| 16 | CAGACC | |
| nirS | CACCCGGAACCGCGCGTGGCGGCGATCATCGCCTCCCACGAGCA | 70 |
| 17 | CCCCGA | |
| nirS | CATCGTCAACGTGAAGGAGACCGGCAAGGTCCTGCTGGTCAACT | 71 |
| 18 | ACAAGG | |
| nirS | TCGACAACCTCACCGTCACCAGCATCGGTGCGGCGCCGTTCCTCC | 72 |
| 19 | ACGAC | |
| nirS | GGCTGGGACAGCAGCCACCGCTACTTCATGACCGCCGCCAACAA | 73 |
| 20 | CTCCAA | |
| nirS | GGTTGCCGTGATCGACTCCAAGGACCGTCGCCTGTCGGCCCTGGT | 74 |
| 21 | CGACG | |
| nirS | GCAAGACCCCGCACCCGGGGCGTGGCGCCAACTTCGTGCATCCCA | 75 |
| 22 | AGTAC | |
| narG 1 | ATGAGTCACCTGCTCGACCGCCTGCAGTTCTTCAAGAAGAAGCAG | 76 |
| | GGCGAAT | |
| narG 2 | CGGCAGCGCTGGCAGCACGACAAGATCGTGCGCTCCACCCACGG | 77 |
| | GGTGAACT | |
| narG 3 | ACCGGCTCCTGCTCCTGGAAGATCTACGTGAAGAACGGCCTGATC | 78 |
| | ACCTGGG | |
| narG 4 | CTGAAGTACCCGAAGGTGCGCAAGCCGTTGCTCAAGCTCTGGCGC | 79 |
| | GAGGCGC | |
| narG 5 | TGGGACGAGGTCACCGAGATCATCGCCGCGGCCAACGTCTACAC | 80 |
| | CGCCAAGA | |
| narG 6 | GTGCGCTACAAGGGCACCAAGACCGTCTCCATCACCCCGGACTAT | 81 |
| | TCCGAGG | |
| narG 7 | GGCATGGCCTTCGGTCACGTGATCCTGAAGGAATTCCACCTCGAC | 82 |
| | CGGCCGA | |
| narG 8 | GCCTACTTCGTCGACTACTGCCGCCAGTACACCGACATGCCGATG | 83 |
| | CTGGTGT | |
| narG 9 | CAGACGCGCCTGCAACTGTCGCTGCTCGATGGCCCGGAACATGCC | 84 |
| | TGCGAGG | |
| narG | GCCTTCCCGTATTTCGCCGGGCAGGAGCACCCGCACTTCAAGGGC | 85 |
| 10 | GTCGCCA | |
| narG | ATGGTGATCATCGGCGCGGCGATGAACCACTGGTACCACATGGA | 86 |
| 11 | CATGAACT | |
| narG | AGCTTCTTCTACCTGCACAGCTCGCAATGGCGCCACGAGAAGCTG | 87 |
| 12 | TCGATGC | |
| narG | CCGGACGATCCGCAGAACTTCCCGCGCAACATGTTCATCTGGCGC | 88 |
| 13 | TCCAACC | |
| narG | GAGGTCGACTGGGTTGACGACGGTGCCGAGGGCAAGCTCGACCT | 89 |
| 14 | GGTCACCA | |
| narG | CTGGACTTCCGCATGTCCTCCACCTGCATGTACTCGGACATCGTCC | 90 |
| 15 | TGCCGA | |
| narG | GCTACCTGGTACGAGAAGGACGACCTCAACACCTCCGACATGCA | 91 |
| 16 | CCCCTTCA | |
| narG | GTCTACCGCAAGTTCACCTCGCTCGGTCCGCTGCTGGACAAGCTG | 92 |
| 17 | GGCAACG | |
| narG | GGCAAGGGCATCGGCTGGAACACCGAGAAGGAAGTGAAGCTGGT | 93 |
| 18 | CGGCGACC | |
| narG | GTCGCGGTCAAGGCCTGGGAAGCGCTGTCGAAGATCACCGGCCG | 94 |
| 19 | CGAGCATG | |
| narG | CACCTGGCGCTGCCCAAGGAAGACGAGAAGATCCGCTTCCGCGA | 95 |
| 20 | CATCCAGG | |

In some embodiments, detection of biomarkers of the aerobic, hypoxic and anaerobic conditions of the biological environment as well as detection of biomarker of the presence of the Nar-containing bacteria can comprise detection of one or more biomarkers in the biological environment and/or a sample thereof, performed by electrochemical methods and techniques as will be understood by a skilled person.

In particular in embodiments where specific detection of RNA and/or protein is performed single-stranded DNA or RNA molecules that can bind specifically to target molecules and be immobilized on the electrode surface through thiol-gold interactions or using linkers like streptavidin/biotin. Also, proteins that specifically bind to antigens (target proteins) can be immobilized using various chemistries, such as glutaraldehyde cross-linking, EDC/NHS coupling, or direct adsorption onto nanomaterial-modified electrodes as will be understood by a skilled person (see e.g. Oberhaus, Franziska V., Dieter Frense, and Dieter Beckmann. “Immobilization techniques for aptamers on gold electrodes for the electrochemical detection of proteins: a review.” Biosensors 10.5 (2020): 45.).

In some embodiments signal amplification for detection of RNA or protein can also be performed with any technique identifiable by a skilled person.

Enzymes like horseradish peroxidase (HRP) can catalyze reactions that produce detectable electrochemical signals (see.g. (see. e.g Li, Haiping, et al. “Signal Amplification-Based Biosensors and Application in RNA Tumor Markers.” Sensors 23.9 (2023): 4237) nanomaterials such as gold nanoparticles (see e.g. Guo, Lanpeng, et al. “Electrochemical protein biosensors for disease marker detection: progress and opportunities.” Microsystems & Nanoengineering 10.1 (2024): 65.) and hybridization chain reaction (HCR) and rolling circle amplification (RCA) (see e.g. Li, Xinran, et al. “PCR Independent Strategy-Based Biosensors for RNA Detection.” Biosensors 14.4 (2024): 200) and additional techniques identifiable by a skilled person.

In some embodiments, one or more biomarkers can be detected with suitable techniques identifiable by a skilled person. For examples in embodiments where the biological environment is a wound it can be performed via “smart bandages” including biomarker detector sensors.

In some embodiments, where detected oxygen level redox potential and nitrate concentration indicate different oxygenation status of a biological environment, target region thereof or sample thereof (for example, oxygen concentration indicates the biological sample region has hypoxic characteristics, but no nitrate concentration is detected) after one or a plurality of detection, methods and systems of the disclosure and related matrices devices can comprise detecting one or more genetic markers expressed by bacteria during nitrate respiration as a further confirmatory method. For example in some embodiments biological samples can be removed for analysis by suitable techniques (such as fluorescence methods or electrochemical methods as described in Examples 11 and 12 and Example 34 and 35) and if characteristic markers for nar, nir and/or nos genes (for example) are detected the skilled person will understand that nitrate respiration is occurring and chlorate treatment is appropriate.

In some embodiments where detected oxygen level, redox potential, nitrate concentration and/or biomarker indicate different oxygenation status of a biological environment, target region thereof or sample thereof after one or a plurality of measurement, the method and systems of the disclosures comprise administering an antibiotic effective amount and a chlorate effective amount that are effective under hypoxic conditions, optionally followed by repeating the detecting oxygen level redox potential, nitrate concentration and/or biomarker and administering the antibiotic and/or chlorate depending on the detected oxygenation status.

In some embodiments, the chlorate administration time ranges from 1 day to 30 days possibly from 10 days to 12 days or 15 days from the formation of the infection, up several months, possibly performed by multiple applications within the time range.

The specific timing of chlorate administration will depend on the timing when the infected microenvironment becomes hypoxic or preferably anoxic. which can be determined by a skilled person based on the pathogen load (the higher the load, the more quickly the infection will go anoxic) and the depth of infection within the environment.

In some embodiments, detection of anoxic portions of a target environment can be performed by detection of biofilm formation as will also be understood by a skilled person.

In some embodiments, the chlorate can be administered to a target administration site of the treated biological environment known or detected to be under anoxic conditions, for example using techniques indicated in the presence disclosure and/or additional techniques identifiable by a skilled person.

In particular, in some embodiments, the hypoxic and/or anoxic portion of the infected biological environment targeted by chlorate administration can be identified by detecting in the biological environment at least one of oxygen level, redox potential and nitrate concentration and selecting the timing of administration when the oxygen level is below 100 uM, redox potential is below 200 mV, and/or nitrate concentration is above 500 uM. Preferable one or more targeted infected portions of the biological environment for chlorate administration can be identified following quantitative detection of all those three markers when oxygen level is below 100 uM, redox potential is below 200 mV, and nitrate concentration is above 500 uM.

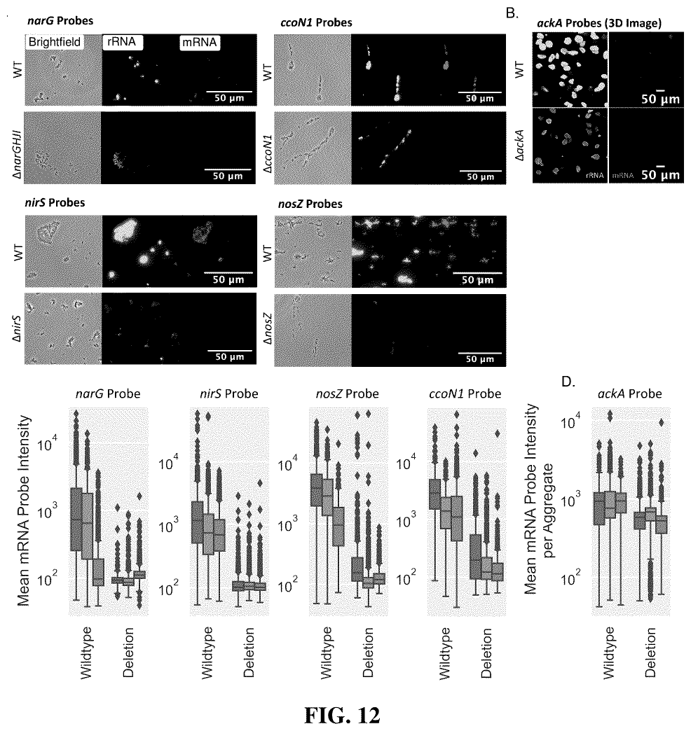

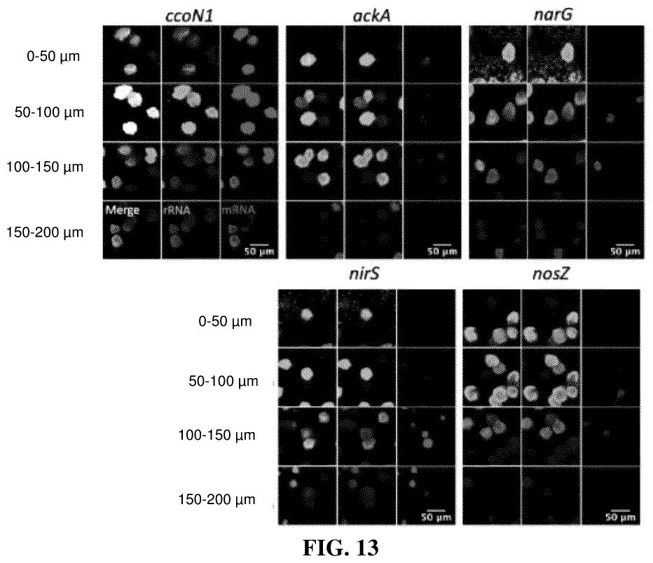

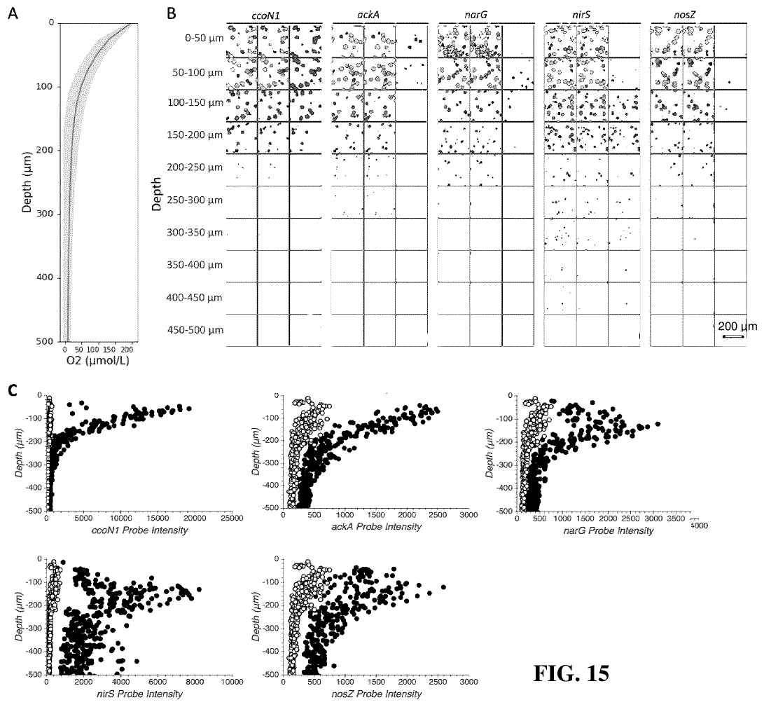

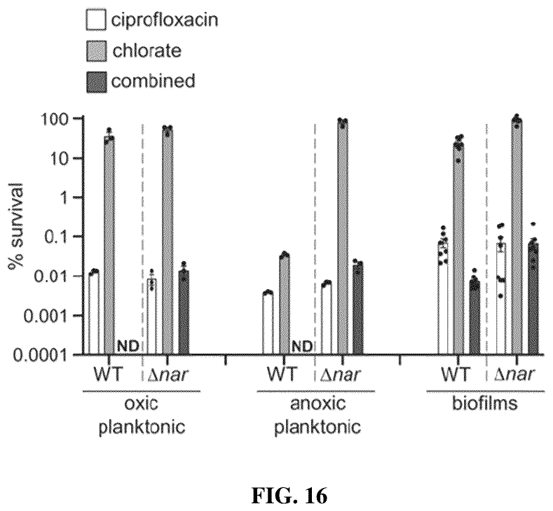

In some embodiments, the chlorate administration site of a target environment can be located a depth of 50-100 um below the surface of an infected environment, e.g. within individual biofilm aggregates a skilled user can detect. Reference is made in this connection to the exemplary detection of mRNA (in purple) on the right panel for narG biomarkers in FIGS. 12 and 15 discussed in Examples 11 and 12 of the present disclosure and the supplementary figures of Annex B of U.S. Provisional Application 63/519,537 and Annex A of U.S. Provisional Application Ser. 63/670,084. When this signal is overlaid with a marker for all the cells (rRNA), a skilled user can see that the Nar-expressing cells are in the core).

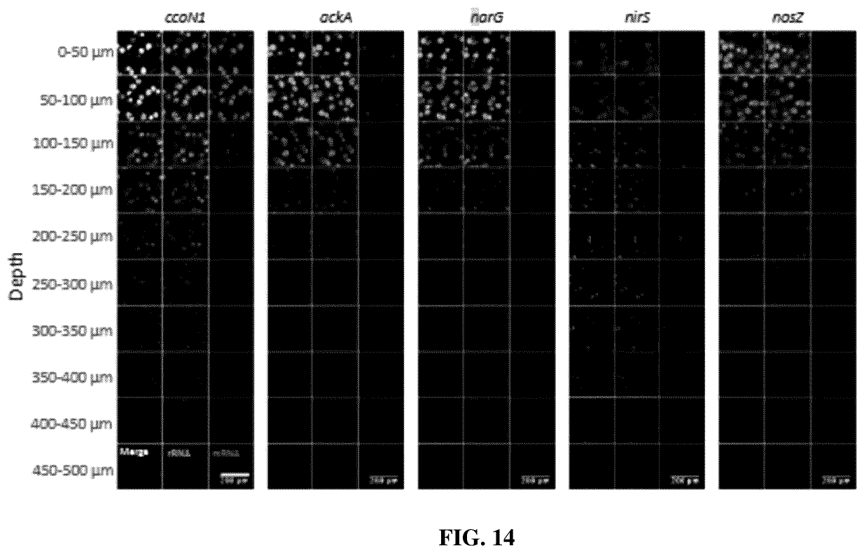

In some embodiments the chlorate administration site can be identified based on cell density (e.g. biofilms of a diameter >20 um, that can be found for example at a depth greater than −10-20 um into an infected tissue, or within −5 μm from the surface as shown FIGS. 13 and 14 discussed in Example 12 of the present disclosure in Figure S1 and S2 of Annex A of U.S. Provisional Application 63/519,537 and Annex A of U.S. Provisional Application Ser. 63/670,084 for biofilm aggregates grown in lab through detection of narG expression. In those exemplary figures an anoxic portion of the biological environment can be seen at a depth of 50-100 um below the surface of the infected sample, within individual biofilm aggregates (see in particular the mRNA on the right panel for narG which start to be visible).

Chlorate can be applied at concentrations ranging from hundreds of nanomolar to millimolar, with the precise concentration depending on the amount of nitrate expected to be present in the infected environment. In preferred embodiments, chlorate concentrations are equal or exceed those of the local nitrate concentration.

In particular in some embodiments herein described, chlorate can be provided in any one of the amounts from 0.001 mM to 200 mM.

In some of these embodiments, chlorate can be administered in an amount from 0.001 mM to 10 mM, 20 mM 30, mM, 50 mM, 100 mM, or to 150 mM.

In some of these embodiments, chlorate can be administered in an amount from 0.01 mM to 10 mM, 20 mM 30, mM, 50 mM, 100 mM, 150 mM, or to 200 mM.

In some of these embodiments, chlorate can be administered in an amount from 0.1 mM to 10 mM, 20 mM 30, mM, 50 mM, 100 mM, 150 mM, or to 200 mM.

In some of these embodiments, chlorate can be administered in an amount from 1 mM to 10 mM, 20 mM 30, mM, 50 mM, 100 mM, 150 mM, or to 200 mM.

In some of these embodiments, chlorate can be administered in an amount from 0.001 mM to 30 mM.

In some of these embodiments, chlorate can be administered in an amount from 0.001 mM to 10 mM.

In some of these embodiments, chlorate can be administered in an amount from 0.001 mM to 1 mM.

In some of these embodiments, chlorate can be administered in an amount from 0.001 mM to 1 mM possibly 0.001 to 0.01 mM, or 0.01 to 1 mM.

In some of these embodiments, chlorate can be administered in an amount from 0.1 mM to 10 mM, or in an amount from 1 mM to 20 mM.

In particular, in embodiments herein described an hypoxic chlorate effective concentration—can range from 0.001 to 200 mM, from 0.001 to 50 mM; from 0.01 to 50 mM; from 0.01 to 20 mM; 0.001 to 10 mM, from 0.1 to 50 mM; from 0.2 to 25 mM or from 0.1-10 mM as will be understood by a skilled person upon reading of the present disclosure. [00281] in embodiments herein described an anoxic chlorate effective concentration—can range from 0.001 to 200 mM, from 0.001 to 50 mM; from 0.01 to 50 mM; from 0.01 to 20 mM; 0.001 to 10 mM, from 0.1 to 50 mM; from 0.2 to 25 mM or from 0.1-10 mM as will be understood by a skilled person upon reading of the present disclosure.

A skilled person will be able to identify a concentration for a particular application in view of the specific medium and specific manner of administration upon review of the present disclosure.

In most preferred embodiments, matrix, compositions, methods and systems based on a chlorate is performed in absence of chlorite.

The term “chlorite” refers to chemical compounds containing chlorite oxyanion having the formula ClO₂⁻. Chlorite refers to a salt of chlorous acid (HClO₂).

Exemplary chlorites include potassium chlorite, sodium chlorite, magnesium chlorite.

Chlorite is the strongest oxidizer of the chlorine oxyanions on the basis of standard half-cell potentials under acid conditions.

However, in some embodiments, matrix, compositions, methods and systems based on a chlorate can further comprise administering to the wound an effective amount of chlorite, typically administered before administration of chlorate alone or in combination with one or more antibiotics, additional antimicrobial and/or wound healing agents.

In some of those embodiments, sodium chlorite is dissolved in water to make a sodium chlorite aqueous solution having a concentration of 0.1% to 25% by weight based on the total weight of the solution. Preferably the concentration of sodium chlorite in water is 0.5 to 5%.

More preferably concentration of sodium chlorite in water is 1 to 3%.

In one embodiment, a composition of sodium chlorite solution in water having a concentration of 0.1% to 25% by weight based on the total weight of the solution is topically applied to a wound in a subject wherein the wound is covered by the composition. In some embodiment, the composition is applied topically to a wound every 8 hours, or every 24 hours, or until the wound is healed.

In some embodiments, a composition of sodium chlorite solution in water is administered orally to a subject in need of the medication, in an amount of 0.1 to 180 mg per day, preferably 0.5 to 50 mg per day, more preferably 1 to 10 mg per day.

In some embodiments, the sodium chlorite can be formulated as an ointment composition comprising sodium chlorite and paraffin, wool fat, beeswax, macrogols, emulsifying wax, cetrimide or vegetable oil (olive oil, arachis oil, coconut oil) or a combination there of in an amount of 0.1% to 25% by weight based on the total weight of the ointment composition, preferably 0.5 to 5% by weight based on the total weight of the ointment composition, more preferably 1 to 3% by weight based on the total weight of the ointment composition. In some embodiment, the ointment composition is applied topically to a wound every 8 hours, or every 24 hours, or until the wound is healed.

In some embodiments, the chlorite can administered systemically in an amount between 3 mg/kg b.w. to 32 mg/kg per day [26] [cited in WHO, 2008] [27] [cited in WHO, 2008] [28][cited in WHO, 2008]. [29] [cited in WHO, 2008]. [30] [cited in WHO, 2008].

In preferred embodiments, of methods and systems of the disclosure and related compounds compositions, matrices and implants of the disclosure a chlorate is administered combination with one or more antibiotics.

The term “antibiotics” as used herein refers to a type of antimicrobial used in the treatment and prevention of bacterial infection. Some antibiotics can either kill or inhibit the growth of bacteria. Others can be effective against fungi and protozoans. The term “antibiotics” can be used to refer to any substance used against microbes. Antibiotics are commonly classified based on their mechanism of action, chemical structure, or spectrum of activity. Most antibiotics target bacterial functions or growth processes. Antibiotics having bactericidal activities target the bacterial cell wall, such as penicillins and cephalosporins, or target the cell membrane, such as polymyxins, or interfere with essential bacterial enzymes, such as rifamycins, lipiarmycins, quinolones and sulfonamides. Antibiotics having bacteriostatic properties target protein synthesis, such as macrolides, lincosamides and tetracyclines. Antibiotics can be further categorized based on their target specificity. “Narrow-spectrum” antibacterial antibiotics target specific types of bacteria, such as Gram-negative or Gram-positive bacteria. “Broad-spectrum” antibiotics affect a wide range of bacteria.

In some embodiments, the antibiotics can be administered in combination with the chlorate in one or more oxic and/or anoxic antibiotic administration sites of the biological environment in parallel with the chlorate administration, or after chlorate administration possibly on administration sites of the biological environment which were anoxic prior to chlorate administration as will be understood by a skilled person.

In particular, in some embodiments the sites and/or timing for antibiotic administration can be identified by detecting in the biological environment at least one of oxygen level, redox potential and nitrate concentration and selecting the timing of administration when the oxygen level is above a selected threshold level of 200 uM, preferably 100 uM, redox potential is above a selected threshold potential preferably 200 mV, and/or nitrate concentration is below a selected threshold concentration preferably 500 uM. Preferable timing for antibiotic administration is identified following quantitative detection of all those three markers when oxygen level is above a selected threshold level preferably 100 uM, redox potential is above a selected threshold potential preferably 200 mV, and nitrate concentration is below a selected threshold level preferably 500 uM.

In some most preferred embodiments antibiotic administration to the biological environment can occur in combination with the administration of chlorate, before, concurrently and/or subsequently to chlorate administration In particular administration of an antibiotic can be performed on a same biological environment or region thereof where chlorate is administered and which is in hypoxic condition, in particular when oxygen level is from 20 uM to 200 uM.

In particular in some embodiments, a method of treating an infected biological environment, comprises contacting the environment with an antibiotic before, concurrently and/or following chlorate administration to the wound to treat and/or prevent an infection of Nar-containing bacteria of in the biological environment. In the method, contacting the antibiotic with the biological environment is performed at time and/or antibiotic administration site when the Nar-containing bacteria, if any is present, undergo aerobic respiration In most preferred embodiments contacting the antibiotic with the biological environment can also be performed at time and/or antibiotic administration site when the Nar-containing bacteria, if any is present, undergo hypoxic and/or anaerobic respiration also in combination with chlorate administration.

In some embodiments, the antibiotic can be administered on a same chlorate administration site, at an antibiotic administration time, selected to time antibiotic delivery after the administered chlorate has penetrated a biofilm possibly present in the biological environment. In some preferred embodiments antibiotic can be administered on a same chlorate administration site, at an antibiotic administration time, selected to time antibiotic delivery before, concurrently and/or after the administered chlorate has penetrated a biofilm possibly present in the biological environment.

In some embodiments, the antibiotic is contacted with a biological environment such as a wound at an antibiotic administration time ranging from 1 h to several days from the chlorate administration to the biological environment.

In some embodiments, the method comprises contacting the wound with an effective chlorate at a time ranging from 1 day to 30 days up to several months following formation of the wound and contacting the wound with an antibiotic is after a time interval ranging from 1 h to 1 day from contacting the wound with the chlorate., wherein the contacting of chlorate is performed preferably by contacting the chlorate and most preferably the antibiotic with a targeted tissues layer within the wound.

In matrices, agents, compositions, methods and systems of the present disclosure antibiotics are comprised in a therapeutically effective amounts that can be identified by a skilled person based on the specific agent, wound and route of administration as will be understood by a skilled person.

In matrices, agents, compositions, methods and systems of the present disclosure the combined administration of a chlorate with one or more antibiotics is known and expected to result in a synergic antibacterial effect resulting from the combined administration as shown in Example 3 of U.S. Ser. No. 17,234,656, incorporated herein by reference in its entirety.

In embodiments herein described suitable antibiotics that can be used in combination with chlorate include ampicillin, kanamycin, ofloxacin, Aminoglycosides, Carbapenems, Ceftazidime, Cefepime, Ceftobiprole, Fluoroquinolones, Piperacillin, Ticarcillin, tobramycin, aztreonam, coliston, tazobactam, and others (or combinations of these antibiotics) that can be readily recognized by a person skilled in the art.

Additional antibiotics that can be used in combination with one or more chlorates herein described include Amoxicillin and clavulanic acid (Augmentin®), Methicillin, oxacillin, nafcillin, cloxacillin, dicloxacillin, cabenicillin, ticarcillin, piperacillin, mezlocillin, azlocillin, ticarcillin and clavulanic acid (Timentin®), piperacillin and tazobactam (Zosyn®), cephalexin, cefdinir, cefprozil, cefaclor, cefuroxime, sulfisoxazole, erythromycin/sulfisoxazole, tobramycin, amikacin, gentamicin, erythromycin, clarithromycin, azithromycin, tetracycline, doxycycline, minocycline, tigecycline, ciprofloxacin, levofloxacin, vancomycin, linezolid, imipenem, meripenem, and aztreonam.

As a person of ordinary skill in the art would understand, the antibiotics herein listed can be selected for treating infections and/or reducing inflammation caused by bacteria including Staphylococcus (S. aureus and S. epidermidis), Pseudomonas (P. aeruginosa), Burkholderia cepacia, Escherichia coli, Enterococcus spp., Corynebacterium spp., and some mycobacteria. In some embodiments, antibiotics can be selected to treat infections and/or reduce inflammation caused by the bacteria listed in Table 3 below.

TABLE 3
Bacteria found in chronic wounds

| Bacteria | References |
|---|---|
| Acinetobacter sp. | (Gjødsbøl et al., 2006, 2012; Dowd et al., 2008a; James et al., |
| | 2008; Gontcharova, 2010; Wolcott et al., 2016) |
| Anaerococcus sp. | (Gardner et al., 2013; Smith et al., 2016; Wolcott et al., 2016) |
| Bacillus sp. | (Gjødsbøl et al., 2006; Dowd et al., 2008b, 2008a; |
| | Gontcharova, 2010; Wolcott et al., 2016) |
| Corynebacterium sp. | (Gontcharova, 2010; Gjødsbøl et al., 2012; Gardner et al., |
| | 2013; Scales and Huffnagle, 2013; Smith et al., 2016; Wolcott |
| | et al., 2016) |
| Enterobacter sp. | (Gjødsbøl et al., 2006; Dowd et al., 2008a; James et al., 2008; |
| | Smith et al., 2016; Wolcott et al., 2016) |
| Enterobacter cloacae | (Gjødsbøl et al., 2006, 2012) |
| Enterococcus sp. | (Dowd et al., 2008a; James et al., 2008; Scales and Huffnagle, |
| | 2013; Smith et al., 2016; Wolcott et al., 2016) |
| Enterococcus faecalis | (Gjødsbøl et al., 2006, 2012; Wolcott et al., 2016) |
| Escherichia sp. | (Dowd et al., 2008a; James et al., 2008; Gontcharova, 2010; |
| | Gjødsbøl et al., 2012; Scales and Huffnagle, 2013) |
| Escherichia coli | (Gjødsbøl et al., 2006; Dowd et al., 2008a) |
| Finegoldia sp. | (Gontcharova, 2010; Gardner et al., 2013; Wolcott et al., |
| | 2016) |
| Finegoldia magna | (Smith et al., 2016; Wolcott et al., 2016) |
| Paenibacillus sp. | (Dowd et al., 2008a) |
| Peptoniphilus sp. | (Dowd et al., 2008a; Gardner et al., 2013; Smith et al., 2016; |
| | Wolcott et al., 2016) |
| Porphyromonas sp. | (Gardner et al., 2013) |
| Prevotella sp. | (Gontcharova, 2010; Gardner et al., 2013; Scales and |
| | Huffnagle, 2013; Smith et al., 2016; Wolcott et al., 2016) |
| Propionibacterium sp. | (Gontcharova, 2010; Wolcott et al., 2016) |
| Propionibacterium acnes | (Wolcott et al., 2016a) |
| Pseudomonas sp. | (Gjødsbøl et al., 2006; Dowd et al., 2008a; James et al., 2008; |
| | Gontcharova, 2010; Scales and Huffnagle, 2013; Smith et al., |
| | 2016; Wolcott et al., 2016) |
| Pseudomonas aeruginosa | (Gjødsbøl et al., 2006, 2012; Scales and Huffnagle, 2013; |
| | Wolcott et al., 2016) |
| Staphylococcus sp. | (Dowd et al., 2008a; James et al., 2008; Gontcharova, 2010; |
| | Gardner et al., 2013; Smith et al., 2016; Wolcott et al., 2016) |
| Staphylococcus epidermidis | (Scales and Huffnagle, 2013; Wolcott et al., 2016) |
| Streptococcus sp. | (Dowd et al., 2008a; James et al., 2008; Gontcharova, 2010; |
| | Scales and Huffnagle, 2013; Smith et al., 2016; Wolcott et al., |
| | 2016) |
| Turicibacter sp. | Wolcott et al., 2016b |

In some embodiments, suitable antibiotics comprise antibiotics effective against Pseudomonas aeruginosa such as Aminoglycosides, Carbapenems, Ceftazidime, Cefepime, Ceftobiprole, Fluoroquinolones, Piperacillin, Ticarcillin, tobramycin, aztreonam, coliston, and others (alone or in combination) that can be recognized by a skilled person.

Exemplary antibiotics that can be used in combination with chlorate for treating chronic wounds include tobramycin, amoxicillin, clavulanic acid, clindamycin, aminoglycosides, ciprofloxacin, cefalosporines, metronidazole and others identifiable to a person skilled in the art.

In some embodiments, suitable antibiotics comprise antibiotics effective against pathogen Pseudomonas aeruginosa such as Aminoglycosides, Carbapenems, Ceftazidime, Cefepime, Ceftobiprole, Fluoroquinolones, Piperacillin, Ticarcillin, tobramycin, aztreonam, coliston, and others (alone or in combination) that can be recognized by a skilled person.

Exemplary antibiotics that can be used in combination with chlorate administration comprise Amoxicillin and clavulanic acid (Augmentin®), Methicillin, oxacillin, nafcillin, cloxacillin, dicloxacillin, cabenicillin, ticarcillin, piperacillin, mezlocillin, azlocillin, ticarcillin and clavulanic acid (Timentin®), piperacillin and tazobactam (Zosyn®), cephalexin, cefdinir, cefprozil, cefaclor, cefuroxime, sulfisoxazole, erythromycin/sulfisoxazole, tobramycin, amikacin, gentamicin, erythromycin, clarithromycin, azithromycin, tetracycline, doxycycline, minocycline, tigecycline, ciprofloxacin, levofloxacin, vancomycin, linezolid, imipenem, meripenem, and aztreonam. As a person of ordinary skill in the art would understand, the antibiotics herein listed can be selected for treating infections or reducing inflammation caused by bacteria including Staphylococcus aureus, Pseudomona (P. aeruginosa).

Additional antibiotics suitable in particular for treatment of cystic fibrosis include Amoxicillin and clavulanic acid (Augmentin®), Methicillin, oxacillin, nafcillin, cloxacillin, dicloxacillin, cabenicillin, ticarcillin, piperacillin, mezlocillin, azlocillin, ticarcillin and clavulanic acid (Timentin®), piperacillin and tazobactam (Zosyn®), cephalexin, cefdinir, cefprozil, cefaclor, cefuroxime, sulfisoxazole, erythromycin/sulfisoxazole, tobramycin, amikacin, gentamicin, erythromycin, clarithromycin, azithromycin, tetracycline, doxycycline, minocycline, tigecycline, ciprofloxacin, levofloxacin, vancomycin, linezolid, imipenem, meripenem, and aztreonam. A person skilled in the art would be able to select appropriate antibiotics for treating cystic fibrosis caused by particular pathogen. An exemplary indication of antibiotic, is shown in Table 4 below which is a modified version of a table From Orenstein, D. Cystic Fibrosis: A Guide for Patient and Family, 4th ed. LWW; 2011. [31]

TABLE 4
An exemplary list of antibiotics

| | | |
|---|---|---|
| Type and kinds | Bacteria Treated | How Taken |

Penicillins

| | | |
|---|---|---|
| Amoxicillin and clavulanic acid (Augmentin ®) | Staphylococcus aureus (Staph) | |
| Methicillin, oxacillin and nafcillin | Pseudomonas (P. aeruginosa) | Intravenous, |
| | | intramuscular |
| Cloxacillin and dicloxacillin | Staph | Oral |
| Cabenicillin, ticarcillin, piperacillin, mezlocillin | P. aeruginosa | Intravenous |
| and azlocillin | | |
| Ticarcillin and clavulanic acid (Timentin ®) | Staph, P. aeruginosa | Intravenous |
| Piperacillin and tazobactam (Zosyn ®) | P. aeruginosa | Intravenous |

Cephalosporins

| | | |
|---|---|---|
| Cephalexin, cefdinir, cefprozil and cefaclor | Staph, P. aeruginosa | Oral |
| Cefuroxime | Staph | Oral |

Sulfa

| | | |
|---|---|---|
| Sulfisoxazole | P. aeruginosa | Oral |
| Erythromycin/sulfisoxazole | Staph | Oral |

Aminoglycosides

| | | |
|---|---|---|
| Tobramycin, amikacin, gentamicin | P. aeruginosa (in combination | Intravenous, inhaled |
| | with gentamicin, tobramycin, | |
| | and amikacin; also work well | |
| | with anti-Pseudomonas | |
| | penicillin drug) | |

Macrolides

| | | |
|---|---|---|
| Erythromycin, clarithromycin and azithromycin | Staph and may help reduce | Oral, intravenous |
| | inflammation from P. aeruginosa | |

Tetracyclines

| | | |
|---|---|---|
| Tetracycline, doxycycline, minocycline, and | Formerly P. aeruginosa, nd Staph | Oral, intravenous, |
| tigecycline | | intramuscular |

Quinolones

| | | |
|---|---|---|
| Ciprofloxacin, levofloxacin | Pseudomonas | Oral, intravenous |

Vancomycin

| | | |
|---|---|---|
| Vancomycin | Staph and methicillin-resistant | Intravenous |
| | Staphylococcus aureus (MRSA) | |

Linezolid

| | | |
|---|---|---|
| Linezolid | MRSA | Oral, intravenous |

Imipenem & Meripenem

| | | |
|---|---|---|
| Imipenem & Meripenem | P. aeruginosa, Staph | Intravenous |

Aztreonam (Cayston ®)

| | | |
|---|---|---|
| Aztreonam (Cayston ®) | P. aeruginosa | Intravenous, inhaled |

In preferred embodiments, antibiotics that can be used in combination with chlorate for treating chronic wounds include Ciprofloxacin, Piperacillin, Ceftazidime, Aztreonam, and Tobramycin. In some embodiments, one or more of Ciprofloxacin: 5 ug/mL, Piperacillin: 320 ug/mL, Ceftazidime: 40 ug/mL, Aztreonam: 160 ug/mL, and Tobramycin: 40 ug/mL can be administered alone or in combination.

In some embodiments, the effective amount of one or more antibiotics is a therapeutically effective amount which can be obtained according to drug description, FDA guidance, or recommendations by Centers for Disease Control and Prevention (CDC), Infectious Diseases Society of America (IDSA) or other health protection agencies as will be understood by a person skilled in the art.

Accordingly in embodiments herein described concentration of suitable antibiotics that can be used in the antimicrobial against phenazine producing bacteria can identified based on the respective breakpoint Minimum Inhibitory Concentration (MIC), or Minimum Bactericidal Concentration (MBC) in particular when the antibiotic is administered to a biological environment or a region thereof which is in an oxic condition.

The wording breakpoint minimum inhibitory concentration (MIC) indicates the concentration that inhibits visible bacterial growth at 24 hours of growth in specific media, at a specific temperature, and at a specific carbon dioxide concentration. Methods that can be used to measure the MIC of a microorganism comprise broth dilution, agar dilution and gradient diffusion (the ‘E test’), where twofold serial dilutions of antibiotic are incorporated into tubes of broth, agar plates or on a paper strip, respectively, as will be understood by a person skilled in the art. The disk diffusion method defines an organism as susceptible or resistant based on the extent of its growth around an antibiotic-containing disk. MIC values are influenced by several laboratory factors.

Laboratories follow standard for parameters such as incubation temperature, incubation environment, growth media, as well as inoculum and quality control parameters. In the U.S. Standards for determining breakpoint MIC values for various bacteria can be found in Clinical & Laboratory Standards Institute (CLSI) publications, with an example also provided as Appendix A of U.S. Provisional Application No. 62/722,124 incorporated herein by reference in its entirety, as will be understood by the skilled person. In Europe, standards for determining breakpoint MIC values for bacteria can be found in European Committee on Antimicrobial Susceptibility Testing (EUCAST) see www.eucast.org/clinical_breakpoints/dated March 2023 and at the time of filing of the instant disclosure) as will be understood by the skilled person.

The Minimum Bactericidal Concentration (MBC) is a critical parameter in microbiology and pharmacology, used to determine the efficacy of antibacterial agents. It is defined as the lowest concentration of an antibacterial agent required to kill a particular bacterium. This concentration is identified by determining the lowest level of the agent that reduces the viability of the initial bacterial inoculum by at least 90%.

While the MIC measures the lowest concentration of an antimicrobial agent that inhibits visible growth of bacteria, the MBC measures the lowest concentration that results in bacterial death the MBC is typically determined following an MIC test. After establishing the MIC, samples are subcultured onto agar plates without the antibacterial agent. The MBC is the lowest concentration at which no bacterial colonies grow on these plates, indicating that the bacteria have been killed rather than just inhibited as will be understood by a skilled person. The MBC can be used in clinical settings to distinguish between bacteriostatic and bactericidal agents, especially when treating severe infections where bacterial eradication is sought as will be understood by a skilled person. The MBC of an antibiotic can be higher than the MIC and can be one or two order of magnitudes higher than the MIC.

In preferred embodiments of methods and systems of the disclosure and related matrices compositions and devices, of the disclosure when the antibiotic is administered to a biological environment or region thereof which is in hypoxic condition, the antibiotic can be administered in an antibiotic effective amount which is lower of the MIC and/or MBC for the antibiotic.

In general in embodiments herein described an effective antibiotic concentration under oxic conditions typically ranges from 0.0005 to 0.500 ug/mL preferably usually from 1-500 ug/mL, 1-30 ug/mL or 1-5 ug/mL depending on the bacteria susceptibility to the antibiotic, the target objective of the treatment (e.g. desired therapeutic effect) the antibiotic used related MIC and MBC and the biological environment treated (e.g. skin, blood, muscles, lungs, mucosa and others identifiable by a skilled person).

Under hypoxic or anoxic conditions, the concentrations can go up one or two orders of magnitudes and can range from 0.0005 to 0.2100 ug/mL considering both MIC and MBC, depending on various factors such as the specific antibiotic and oxygenation status of the biological environment, as will be understood by a skilled person.

In some embodiments, in methods and systems herein described and related compositions one or more antibiotics can be administered in concentration of at least 0.00005 ug mL, preferably at least 0.002 ug mL, at least 0.01 ug mL, at least 0.025 ug mL, or at least 0.08 ug mL, or at least 0.1 ug mL, and in additional concentrations identifiable by a skilled person upon reading of the present disclosure. The specific concentration of each antibiotic can be determined based on the related MIC as will be understood by a skilled person.

In most preferred embodiments of methods and systems of the present disclosure, one or more antibiotics can be administered at a concentration of at least 2.0 ug mL, at least 10.0 ug mL, at least 25.0 ug mL, at least 50.0 ug mL, and at least 100.0 ug mL-1, in a concentration associated with a resulting synergic inhibition of bacteria viability herein described.

For example, in some embodiments, the antibiotic can comprise amikacin at concentration from 2 to 64 μg/ml, and in particular 8 μg/ml, 16 g/ml and 64 μg/ml, in particular to target Clinically significant aerobic gram-negative bacilli.

In some embodiments, the antibiotic can comprise ampicillin at a concentration of from 2 to 32 μg/ml, and in particular 4 μg/ml, 6 g/ml and 32 μg/ml, in particular to target Clinically significant aerobic gram-negative bacilli.

In some embodiments, the antibiotic can comprise ampicillin/sulbactam from 2 to 32/16 μg/ml, and in particular 4/2 μg/ml, 16/8, g/ml and 32/168 g/ml, in particular to target Clinically significant aerobic gram-negative bacilli.

In some embodiments, the antibiotic can comprise cefazolin at a concentration of from 4 to 64 μg/ml, and in particular 4 μg/ml, 16 g/ml and 64 g/ml, in particular to target Clinically significant aerobic gram-negative bacilli.

In some embodiments, the antibiotic can comprise cefepime at a concentration of from 1 to 64 μg/ml, and in particular 2 μg/ml, 8 μg/ml, 16 g/ml and 32 μg/ml, in particular to target Clinically significant aerobic gram-negative bacilli.

In some embodiments, the antibiotic can comprise cefoxitin at a concentration of from 4 to 64 μg/ml, and in particular 8 μg/ml, 16 g/ml and 32 μg/ml, in particular to target Clinically significant aerobic gram-negative bacilli.

In some embodiments, the antibiotic can comprise ceftazidime at a concentration of from 1 to 64 μg/ml, and in particular at 1 μg/ml, 2 μg/ml, 8 g/ml and 32 μg/ml, in particular to target Clinically significant aerobic gram-negative bacilli.

In some embodiments, the antibiotic can comprise ceftriaxone at a concentration of from 1 to 64 μg/ml, and in particular at 1 μg/ml, 2 μg/ml, 8 g/ml and 32 μg/ml, in particular to target Clinically significant aerobic gram-negative bacilli.

In some embodiments, the antibiotic can comprise Ciprofloxacin at a concentration of from 0.25 to 4 μg/ml, and in particular at 0.5 μg/ml, 2 μg/ml, and 4 μg/ml, in particular to target Clinically significant aerobic gram-negative bacilli.

In some embodiments, the antibiotic can comprise Gentamicin at a concentration of from 1 to 16 μg/ml, and in particular at 4 μg/ml, 16 μg/ml, and 32 μg/ml, in particular to target Clinically significant aerobic gram-negative bacilli.

In some embodiments, the antibiotic can comprise Levofloxacin at a concentration of from 0.12 to 8 μg/ml, and in particular at 0.25 μg/ml, 0.5 μg/ml, and 2.8 μg/ml, in particular to target E. cloacae, E. coli, K. pneumoniae, P. mirabilis, P. aeruginosa, S. marcescens, A. baumannii, A. lwoffii, C. koseri, C. freundii, E. aerogenes, E. sakazakii, K. oxytoca, M. morganii, P. agglomerans, P. vulgaris, Pv. rettgeri, Pv. stuartii, P. fluorescens, C. sakazakii.

In some embodiments, the antibiotic can comprise Meropenem at a concentration of from 0.25 to 16 μg/ml, and in particular at 0.5 μg/ml, 2 μg/ml, 6 g/ml and 12 μg/ml, in particular to target E. coli, K. pneumoniae, P. aeruginosa, P. mirabilis, Acinetobacter spp., C. freundii, E. cloacae, K. oxytoca, M. morganii, P. vulgaris, S. marcescens, A. hydrophila, C. diversus, H. alvei, P. multocida, Salmonella spp., Shigella spp.

In some embodiments, the antibiotic can comprise Nitrofurantoin at a concentration of from 16 to 512 μg/ml, and in particular at 16 μg/ml, 32 μg/ml, and 64 μg/ml, in particular to target Clinically significant aerobic gram-negative bacilli.

In some embodiments, the antibiotic can comprise Piperacillin/Tazobactam at a concentration of from 4/4 to 128/4 μg/ml, and in particular at 2/4 μg/ml, 8/4 μg/ml, 24/4 μg/ml, 32/4, μg/ml, 32/8 μg/ml, and 48/8 g/ml in particular to target Clinically significant aerobic gram-negative bacilli A. baumannii, E. coli, K. pneumoniae, P. aeruginosa, C. koseri, M. morganii, P. mirabilis, P. vulgaris, Pv. rettgeri, Pv. stuartii, S. enterica.

In some embodiments, the antibiotic can comprise Tobramycin at a concentration of from 1 to 16 μg/ml, and in particular at 8 μg/ml, 16 μg/ml, and 64 μg/ml, in particular to target Clinically significant aerobic gram-negative bacilli.

In some embodiments, the antibiotic can comprise Trimethoprim/Sulfamethoxazole at a concentration of from 1 to 16 μg/ml, and in particular at 1/19 g/ml, 4/76 μg/ml, and 16/304 μg/ml, in particular to target Klebsiella spp., Enterobacter spp., M. morganii, P. vulgaris, P. mirabilis, S. sonnei, S. flexneri, Eco(+ETEC)**, C. sakazakii.

In a further exemplary embodiment, of the biofilm treatment matrix, compositions, methods and systems herein described the antibiotic is Ciprofloxacin at 20 μg/100 μl and the chlorate is 100 μl of a 10 mM solution.

Additional therapeutic concentrations of antibiotics can be identified by a skilled person. according to drug description, FDA guidance, or recommendations by Centers for Disease Control and Prevention (CDC), Infectious Diseases Society of America (IDSA) or other health protection agencies as will be understood by a person skilled in the art. In some embodiments, the chlorate can be contacted with a target portion of the infected biological environment wherein oxygen levels are at a level enabling anaerobic respiration by a Nar containing bacteria if any is present and then the same chlorate administration site is contacted with one or more antibiotics. Accordingly, in some embodiments, following chlorate administration, when oxygen is able to penetrate more deeply into the infection (several hours), a standard antibiotic (e.g. tobramycin, ciprofloxacin, etc.) can be applied. Such a round of treatment can be repeated until the infection is fully cleared.

In embodiments herein described where the administration is performed under hypoxic condition or under anoxic conditions in combination with chlorate, the antibiotic can be administered in an antibiotic effective amount resulting in delivery of amounts of antibiotic which is lower than the MIC and/or MBC, a fraction of the MIC and/or MBC for the antibiotic, preferably half the MIC, more preferably one quarter of the MIC, or lower up to one tenth of the MIC thus resulting in amounts typically ranging from 0.001 to 500 ug/ml, with higher concentrations corresponding from 0.1-500 ug/ml preferably 1-30 ug/mL or 1-5 ug/mL to increase the efficacy of the treatment in view of the bacteria susceptibility to the antibiotic, the target objective of the treatment (e.g. desired therapeutic effect) the antibiotic used and the biological environment treated (e.g. skin, blood, muscles, lungs, mucosa and others identifiable by a skilled person).

In most preferred embodiments, methods to treat an infected biological environment comprise the combined administration of chlorate and at least one antibiotic following detection of oxygen level, redox potential and/or nitrate concentration of the biological environment.

In some embodiments, herein described, methods and systems and related compositions matrices and devices can comprise i) detecting at least one of the oxygen level redox potential and nitrate and ii) based on the related indication (above or below thresholds) provide the effective concentrations of chlorate and antibiotics, in some of those embodiments, when nitrate is detected and is detected below 500 uM give nitrate first to drive nar respiration.

In some embodiments, herein described, methods and systems and related compositions matrices and devices can comprise detecting an oxygen level in a biological environment or a region thereof, to obtain a detected oxygen level of the biological environment or a region thereof. The method further comprises
a) in embodiments in which the detected oxygen level is above 200 uM, administering antibiotic at an oxic antibiotic effective concentration,
b) in embodiments in which the detected oxygen level is from 200 uM to 250 uM, the method preferably further comprises in addition to the administering, detecting a redox potential in the biological environment or region thereof to obtain a detected redox potential of the biological environment or a region thereof; in those embodiments
   a. If the detected redox potential is above 200 mV the method further comprises administering antibiotic at an oxic antibiotic effective concentration
   b. If the detected redox potential is below 200 mV the method further comprises
      i. administering a hypoxic chlorate effective concentration in combination with a hypoxic antibiotic effective concentration or preferably
      ii. detecting a nitrate concentration in the biological environment or region thereof to obtain a detected nitrate concentration of the biological environment or a region thereof; in those embodiments
         a) if the detected nitrate concentration is lower than 500 uM the method further comprises
         a. administering an effective nitrate concentration in combination with
         b. administering a hypoxic chlorate effective concentration+hypoxic antibiotic effective concentration in combination no nitrate a chlorate nitrate effective concentration
         b) if the detected nitrate concentration is lower than 500 uM the method further comprises
         a. administering a hypoxic chlorate effective concentration in combination with an hypoxic antibiotic effective concentration no nitrate.

In embodiments in which
c) the detected oxygen level is below 150 uM and preferably below 100 uM and above 20 uM, the method further comprises administering a hypoxic chlorate effective concentration in combination with a hypoxic antibiotic effective concentration or optionally or preferably depending on the detected oxygen level,
   i. detecting a nitrate concentration in the biological environment or region thereof to obtain a detected nitrate concentration of the biological environment or a region thereof; in those embodiments
      a) if the detected nitrate concentration is lower than 500 uM the method further comprises
         a. administering an effective nitrate concentration in combination with
         b. administering a hypoxic chlorate effective concentration+hypoxic antibiotic effective concentration in combination no nitrate a chlorate nitrate effective concentration
      b) if the detected nitrate concentration is lower than 500 uM the method further comprises
         a. administering a hypoxic chlorate effective concentration in combination with an hypoxic antibiotic effective concentration or with no nitrate.

In embodiments in which
d) the detected oxygen level is below 200 uM and above 150 uM, the method further comprises administering a hypoxic chlorate effective concentration in combination with a hypoxic antibiotic effective concentration or most preferably also
   i. detecting a nitrate concentration in the biological environment or region thereof to obtain a detected nitrate concentration of the biological environment or a region thereof; in those embodiments
      a) if the detected nitrate concentration is lower than 500 uM the method further comprises
         a. administering an effective nitrate concentration in combination with
         b. administering a hypoxic chlorate effective concentration+hypoxic antibiotic effective concentration in combination no nitrate a chlorate nitrate effective concentration
      b) if the detected nitrate concentration is lower than 500 uM the method further comprises
         a. administering a hypoxic chlorate effective concentration in combination with an hypoxic antibiotic effective concentration or with no nitrate.

In embodiments in which
e) the detected oxygen level is below 10 uM the method further comprises administering a anoxic chlorate effective concentration alone or in combination with a anoxic antibiotic effective concentration.

In embodiments in which
f) the detected oxygen level is below 20 uM and above 15 uM, the method further comprises administering a anoxic chlorate effective concentration alone or in combination with a anoxic antibiotic effective concentration, possibly also
   i. detecting a nitrate concentration in the biological environment or region thereof to obtain a detected nitrate concentration of the biological environment or a region thereof; in those embodiments
      a) if the detected nitrate concentration is lower than 500 uM the method further comprises
         a. administering an effective nitrate concentration in combination with
         b. administering a hypoxic chlorate effective concentration+hypoxic antibiotic effective concentration in combination no nitrate a chlorate nitrate effective concentration
      b) if the detected nitrate concentration is lower than 500 uM the method further comprises
         a. administering a hypoxic chlorate effective concentration in combination with an hypoxic antibiotic effective concentration or with no nitrate.

In some preferred embodiment where the detected oxygen level, redox potential and/or nitrate concentration indicate different and/or for serious conditions (e.g. cystic fibrosis), the method can further comprise detecting one or more biomarkers. (e.g. through HCR fluorescence and/or other detection methods possibly performed on one or more samples of the biological environment and/or of a region thereof as will be understood by a skilled person.

In the method and in other methods and systems herein described, in the hypoxic chlorate effective concentration—can range from 0.001 to 200 mM, from 0.001 to 50 mM; from 0.01 to 50 mM; from 0.01 to 20 mM; 0.001 to 10 mM, from 0.1 to 50 mM; from 0.2 to 25 mM or from 0.1-10 mM as will be understood by a skilled person upon reading of the present disclosure.

In the method and in other methods and systems herein described an effective antibiotic concentration under oxic conditions ranges from 0.0005 to 0.500 ug/mL preferably usually from 1-500 ug/mL, 1-30 ug/mL or 1-5 ug/mL depending on the bacteria susceptibility to the antibiotic, the target objective of the treatment (e.g. desired therapeutic effect) the antibiotic used related MIC and MBC and the biological environment treated (e.g. skin, blood, muscles, lungs, mucosa and others identifiable by a skilled person).

In the method and in other methods and systems herein described and related compositions matrices and devices, the hypoxic and anoxic antibiotic effective amount can range from 0.001 to 500 ug/ml, with higher concentrations corresponding from 0.1-500 ug/ml preferably 1-30 ug/mL or 1-5 ug/mL to increase the efficacy of the treatment in view of the bacteria susceptibility to the antibiotic, the target objective of the treatment (e.g. desired therapeutic effect) the antibiotic used and the biological environment treated (e.g. skin, blood, muscles, lungs, mucosa and others identifiable by a skilled person).

In the method and in other methods and systems herein described and related compositions matrices and devices, the nitrated effective concentration can range from 0.1 mM and 50 mM. in embodiments herein described where nitrate is administered in combination with chlorate, the nitrate effective concentration is selected so that the chlorate amount and the nitrate amount in a ratio from 4:1 to 10:1.

In some preferred embodiments, treatment or prevention of an infected biological environment, the biological environment infected by a NAR containing bacteria, can be performed by a method comprising
(a) detecting at least one of i) an oxygen level, ii) a redox potential and iii) a nitrate concentration of the infected biological environment or of at least one target region thereof,
(b) administering to the infected biological environment or the at least one target region thereof, an antibiotic in an antibiotic amount effective to inhibit viability of the bacteria, when at least one of an oxygen level above the detected threshold of 200 uM, preferably 100 uM, a redox potential above the detected threshold, preferably 200 mV, and no nitrate concentration is detected, and
(c1) administering a chlorate and an antibiotic to the infected biological environment or the at least one target region thereof, the chlorate and the antibiotic administered in a chlorate effective amount and an antibiotic effective amount effective to inhibit viability of the bacteria, when at least one of an oxygen level below the detected threshold of 200 uM, preferably below 100 uM, a redox potential below the detected threshold of 300 uM, more preferably 200 mV and/or a nitrate concentration above the detected threshold, preferably 500 uM is detected.

In preferred embodiments, step c1 can be combined with or replaced by
(c2a) administering a chlorate and an antibiotic to the infected biological environment or the at least one target region thereof, the chlorate and the antibiotic administered in a hypoxic chlorate effective amount and a hypoxic antibiotic effective amount effective to inhibit viability of the bacteria, when an oxygen level detected in the biological environment or region thereof is below the detected threshold of 200 uM, preferably below 100 uM and above 20 uM, preferably the administering is performed when a redox potential is detected in the biological environment or region thereof below the detected threshold of 300 uM, more preferably 200 mV and/or a nitrate concentration above the detected threshold, preferably 500 uM is detected, and
(c2b) administering a chlorate, to the infected biological environment the chlorate in an anoxic chlorate effective amount to inhibit viability of the bacteria, when at least one of an oxygen level is below the detected threshold of 20 uM, preferably when a redox potential below the detected threshold of 300 uM, more preferably 200 mV and/or a nitrate concentration above the detected threshold, preferably 500 uM is detected, the administering can optionally be performed in combination with administering an anoxic antibiotic effective amount as will be understood by a skilled person.

In preferred embodiments, the method can further comprise
(d1) administering nitrate to the infected biological environment or a region thereof for a nitrate contacting time and in a nitrate amount effective to increase expression of a Nar gene in the Nar-containing bacteria, and
(d2) after the nitrate contacting time, administering to the biological environment or a region thereof, chlorate in combination with antibiotics in a chlorate amount and an antibiotic amount effective to inhibit viability of the Nar containing bacteria, the chlorate amount and the nitrate amount in a ratio from 4:1 to 10:1
   wherein steps (d1) and (d2) are performed at times and to target sites of the biological environment wherein at least one of an oxygen level below the detected threshold level of 200 uM, preferably 100 uM, a redox potential below the detected threshold potential, preferably 200 mV, and a nitrate concentration below the detected threshold concentration, preferably 500 uM is detected.

In some embodiments, the chlorate administration time is selected from 1 day to 4 months from onset of the infection in the biological environment.

In some embodiments, the chlorate administration time is selected from than 1 day to 30 days from onset of the infection in the biological environment.

In some embodiments, the chlorate administration time is selected from 10 days to 15 days from the onset of the infection.

In some embodiments, the chlorate administration time is 14 days from the onset of the infection.

In some embodiments, chlorate and antibiotic administration can be performed in combination in a hypoxic timed and targeted chlorate administration method for chlorate treatment of a biological environment, the method comprises contacting the hypoxic environment s with a chlorate effective amount combination with an antibiotic effective amount to inhibit viability of a Nar-containing bacteria in the hypoxic environment;

In some embodiments, the hypoxic or anoxic chlorate effective amount is selected from 0.001 to 10 uM, more preferably or 0.01 to 1 mM and most preferably 0.1 mM to 0.5 mM as will be understood by a skilled person upon reading of the present disclosure.

In some embodiments the hypoxic or anoxic antibiotic effective amount being a a fraction of the MIC for the antibiotic, selected from half the MIC and/or MBC, more preferably one quarter of the MIC, or lower up to one tenth of the MIC and/or MBC as will be understood aby a skilled person upon reading of the disclosure,

In some preferred embodiments the detecting can be performed on the at least on target region of the biological environment at a plurality of times to monitor the change in oxygenation status of the biological environment or a target region thereof. In those embodiments, the chlorate effective amount, and the antibiotic effective amount are administered to the biological environment or the target region thereof when oxygenation status detected over time indicated that the biological environment or target region thereof. is under hypoxic conditions.

Hypoxic environments in the body of an individual can be exploited by Nar-containing bacteria, which utilize nitrate respiration to survive in low-oxygen conditions. These environments are often characterized by reduced oxygen availability due to various physiological or pathological factors as will be understood by a skilled person.

Exemplary hypoxic environment in the human body comprise chronic wounds, such as diabetic ulcers or pressure sores, often have hypoxic regions due to impaired blood flow and tissue damage. The low oxygen levels can support the growth of bacteria like Pseudomonas aeruginosa*, which can exploit these conditions for survival and proliferation [32] In particular the presence of biofilms in these wounds further complicates treatment, as biofilms create anoxic microenvironments that protect bacteria from antibiotics and immune responses.

Exemplary hypoxic environment in the human body further comprise lung infections Conditions like cystic fibrosis and chronic obstructive pulmonary disease (COPD) can create hypoxic environments in the lungs. Thick mucus and damaged airways reduce oxygen diffusion, creating niches for bacteria such as *Pseudomonas aeruginosa* to thrive [32] The hypoxic sputum in cystic fibrosis patients, for example, supports the growth of nitrate-respiring bacteria, contributing to persistent infections despite aggressive treatment as will be understood by a skilled person.

Exemplary hypoxic environment in the human body further comprise tumor Microenvironments Tumors often have hypoxic cores due to rapid cell proliferation outpacing angiogenesis, leading to areas with low oxygen supply. This can create conditions favorable for certain bacteria that can exploit the hypoxic environment for growth [33]. Hypoxia in tumors is associated with resistance to therapy and can complicate treatment outcomes.

Exemplary hypoxic environment in the human body further comprise Ischemic Tissues where blood supply is restricted, which can become hypoxic. This can occur in areas affected by cardiovascular diseases, such as in the heart during myocardial infarction or in peripheral tissues in peripheral artery disease [34]. The hypoxic conditions can impair immune function and promote bacterial colonization and infection as will be understood by a skilled person.

Exemplary hypoxic environment in the human body further comprise Gastrointestinal Tract certain sections of the gastrointestinal tract, particularly the colon, can have hypoxic microenvironments due to dense microbial populations and high metabolic activity [33]. Bacteria capable of nitrate respiration can exploit these conditions, potentially leading to infections or dysbiosis.

Hypoxic environments not only provide niches for Nar-containing bacteria but also pose challenges for treatment due to the bacteria's ability to resist conventional therapies and exploit the host's compromised immune responses. Understanding these environments can help in developing targeted therapeutic strategies to manage infections effectively.

In some embodiments, these hypoxic environments can be treated with a hypoxic timed and targeted chlorate administration method which comprises detecting in at least one target region of the biological environment at least one of oxygen level, nitrate concentration and redox potential of a target region of the biological environment, preferably oxygen level alone or in combination with nitrate concentration and optionally redox potential to detect a hypoxic target region of the biological environment. The hypoxic timed and targeted chlorate administration method further comprise
contacting the hypoxic target region of the least one target regions having a detected hypoxic status with a chlorate effective amount in combination with an antibiotic effective amount to inhibit viability of Nar-containing bacteria.

In the timed and targeted method of chlorate administration of the seventh aspect, the contacting is performed for a time and under conditions to treat and/or prevent infection of Nar-containing bacteria.

In some preferred embodiments the detecting can be performed on the at least on target region of the biological environment at a plurality of times to monitor the change in oxygenation status of the at least one target region of the biological environment. In those embodiments, the chlorate administration, the chlorate effective amount, the antibiotic administration time and the antibiotic effective amount for a target region of the at least one target region are selected in time based on an oxygenation status to the target region detected over time.

In preferred embodiments the hypoxic timed and targeted methods herein described the method can further comprise
(d1) administering nitrate to the infected biological environment or a region thereof for a nitrate contacting time and in a nitrate amount effective to increase expression of a Nar gene in the Nar-containing bacteria, and
(d2) after the nitrate contacting time, administering to the biological environment or a region thereof, chlorate in combination with antibiotics in a chlorate amount and an antibiotic amount effective to inhibit viability of the Nar containing bacteria, the chlorate amount and the nitrate amount in a ratio from 4:1 to 10:1.

In preferred embodiments steps (d1) and (d2) are performed following contacting the hypoxic target region of the least one target regions having a detected hypoxic status with a chlorate effective amount in combination with an antibiotic effective amount, to drive the nitrate respiration and increase the efficacy of the chlorate administration.

In all embodiments herein described, where nitrate is administered, nitrated is supplemented at a concentration selected to stimulate the expression of Nar. In these embodiments, nitrate can be supplied to the medium in an amount from 0.1 mM and 50 mM, most preferably prior to administration of the chlorate and in some embodiments, also concurrently with the chlorate administration at concentration selected to stimulate expression of Nar without preventing processing of chlorate.

Preferably, in embodiments where nitrate is administered the related timing and concentration is selected to stimulate expression of Nar without preventing processing of chlorate. Preferably the nitrate administration precedes the chlorate administration and is in amount to minimizing nitrated interference with chlorate interaction with the nitrate reductase as will be understood by a skilled person upon reading of the present disclosure.

In some embodiments nitrate can be administered in a nitrate effective amount from 0.1 mM and 50 mM selected in view of preferred concentration ratios of chlorate: nitrate. In some embodiments, chlorate and nitration are provided at a concentration ratio of at least 10:1, possibly, 8:1, 6:1 or 5:1. In some embodiments, chlorate and nitration are provided at a concentration ratio of at least 4:1.

In some embodiments of methods herein described comprising administering nitrate, the administering can be performed by administering nitrate at an amount from 0.1 mM to 50 mM, the amount selected to stimulate expression of Nar without preventing processing of chlorate.

In some embodiments of methods herein described comprising administering nitrate, the administering can be performed by administering nitrate at an amount from 0.1 mM to 50 mM selected to have a chlorate:nitrate concentration ratio of at least 10:1 in the biological environment.

In some embodiments of methods herein described comprising administering nitrate, the administering can be performed by administering nitrate at an amount from 0.1 mM to 50 mM selected to have a chlorate:nitrate concentration ratio of at least 8:1.

In some embodiments of methods herein described comprising administering nitrate, the administering can be performed by administering nitrate at an amount from 0.1 mM to 50 mM selected to have a chlorate:nitrate concentration ratio of at least 6:1.

In some embodiments of methods herein described comprising administering nitrate, the administering can be performed by administering nitrate at an amount from 0.1 mM to 50 mM selected to have a chlorate:nitrate concentration ratio of at least 5:1.

In some embodiments of methods herein described comprising administering nitrate, the administering can be performed by administering nitrate at an amount from 0.1 mM to 50 mM selected to have a chlorate:nitrate concentration ratio of at least 4:1.

In some most preferred embodiments, treatment prevention of chronicity of an infection in an infected biological environment, the infected biological environment infected by Nar containing bacteria, can be obtained by a method comprising
(a) detecting nitrate in the infected biological environment
(b) administering to the infected biological environment a chlorate amount in combination with an antibiotic amount, the chlorate amount and the antibiotic amount effective to inhibit viability of the bacteria, when a nitrate concentration above the detected threshold, preferably 500 uM is detected and
(c1) administering to the infected biological environment, nitrate for a nitrate contacting time and in a nitrate amount effective to increase expression of a Nar gene in the Nar-containing bacteria, and
(d2) after the nitrate contacting time, administering to the biological environment chlorate in combination with antibiotics in a chlorate amount and an antibiotic amount effective to inhibit viability of the Nar containing bacteria, the chlorate amount and the nitrate amount in a ratio from 4:1 to 10:1
   wherein the steps (d1) and (d2) are performed in combination at a time and to target sites of the biological environment when a nitrate concentration higher than zero and below 500 uM is detected.

In some most preferred embodiments treatment of a biological environment, t infected by a NAR containing bacteria, can be performed by a method comprising
(a) repeatedly monitoring in one or more target areas of the infected biological environment at least one of i) an oxygen level, ii) a redox potential and iii) a nitrate concentration of the infected biological environment,
(b) upon detection in a target area of an oxygen level above the detected threshold, preferably 100 uM, and a redox potential above the detected threshold, preferably 200 mV and preferably no nitrate concentration,
administering to the target area an antibiotic in an antibiotic amount effective to inhibit viability of the bacteria
(c) upon detection in a target area of at least one of at least one of an oxygen level below the detected threshold, preferably 100 uM, a redox potential below the detected threshold, preferably 200 mV and a nitrate concentration above the detected threshold, preferably 500 uM
administering to the target a chlorate amount in combination with an antibiotic amount, the chlorate amount and the antibiotic amount effective to inhibit viability of the bacteria
(d) upon detection in a target area of a nitrate concentration below 500 uM,
(i) administering to the infected biological environment, nitrate for a nitrate contacting time and in a nitrate amount effective to increase expression of a Nar gene in the Nar-containing bacteria, and
(ii) after the nitrate contacting time, administering to the biological environment chlorate in combination with antibiotics in a chlorate amount and an antibiotic amount effective to inhibit viability of the Nar containing bacteria, the chlorate amount and the nitrate amount in a ratio from 4:1 to 10:1
   the administering of steps (b) (c) and (d) performed until completion of the treatment of the infected biological environment.

In embodiments of the chlorate administration methods herein described, an antibiotic effective amount for an oxic environment is an amount resulting antibiotic at concentrations equal to or higher than the MIC of the antibiotic.

In embodiments of the chlorate administration methods herein described, an antibiotic effective amount for a hypoxic environment is a concentration lower than the MIC and/or MBC of the antibiotic, possibly from 1/4 to 1/100 of the MIC and/or MBC of the antibiotic.

In some embodiments of chlorate administration methods herein described, an antibiotic can thus be administered to a biological environment and/or one or more target region thereof and one or more antibiotics effective amounts ranging from in at least 1/100 MIC and up the MIC or higher as will be understood by a skilled person.

In embodiments of chlorate administration methods herein described, a chlorate effective amount for a hypoxic environment chlorate in concentrations can range from 0.1-20 mM preferably from 10 to 200 mM.

In embodiments of chlorate administration methods herein described, a chlorate effective amount for a anoxic environment chlorate in concentrations from can range from 0.1-20 mM preferably from 10 to 200 mM.

In embodiments of chlorate administration methods herein described, the specific antibiotic effective amount and chlorate effective amount depend on the specific biological environment or target region thereof. For example, a small wound such as a skin lesion biofilm with indication of hypoxic bacteria can require application of suitable antibiotic at a concentration conforming to 1/10 of its typical MIC in combination with chlorate in a concentration of 10 mM. In contrast, a larger infection such as cystic fibrosis in lung tissue with indication of hypoxic bacteria can require application of suitable antibiotic at a concentration conforming to ¼ of its typical MIC in combination with chlorate in a concentration of 20 mM.

Similarly, a skilled person will appreciate that, for example, a small biological environment such as a skin lesion biofilm with indication of anoxic bacteria can require application of suitable antibiotic at a concentration conforming to its typical MIC in combination with chlorate in a concentration of 50 mM. In contrast, a larger infection such as cystic fibrosis in lung tissue with indication of anoxic bacteria can require application of suitable antibiotic at a concentration conforming to its typical MIC in combination with chlorate in a concentration of 200 mM.

A skilled person will appreciate that, for example, a small wound such as a skin lesion biofilm with indication of anoxic bacteria although with low concentration of nitrate can require application of nitrate at a concentration of 50 mM for a nitrate application time before further treatment. In contrast, a larger infection such as cystic fibrosis in lung tissue with indication of anoxic bacteria although with low concentration of nitrate can require application of nitrate at a concentration of 500 mM for a nitrate application time before further treatment.

In embodiments of chlorate administration methods of the disclosure further comprising administering nitrate the concentration of nitrate will be selected to minimize toxicity for cells other than the Nar-containing bacteria. The skilled person will appreciate that common salts of both chlorate and nitrate are toxic to humans at high concentrations (˜1 g/kg and −3 g/kg respectively), and therefore therapeutic application of chlorate and nitrate is constrained to those upper limits. Generally in embodiments of chlorate administration methods of the disclosure further comprising administering nitrate the nitrate can be administered in concentrations from 50 to 500 mM.

In some embodiments, any one of the methods herein described can further comprise detecting expression of one or more of any one of anr, narG or nirS to detect timing and/or target site of the biological environment where the Nar-containing bacteria undergoes anaerobic respiration, and chlorate is administered.

In some embodiments, any one of the methods herein described can further comprise detecting presence of one or more Nar-containing bacteria in the biological environment by detecting expression of at least marker of the presence of the Nar-containing bacteria. Exemplary marker specific for Nar-containing bacteria comprise product of any one of the narGHJI genes and/or compounds specific for the Nar containing bacteria, such as redox active compounds produced by Nar containing bacteria such as Pseudomonas aeruginosa. E coli and Klebsiella pneumoniae which are specific for the bacteria.

In some embodiments, any one of the methods herein described can be performed as a part of a method of treating an infected biological environment.

In some embodiments, the method can comprise contacting a chlorate with a chlorate administration site within a hypoxic and/or anoxic portion of the infected biological environment, the hypoxic and/or anoxic region comprising oxygen at a level enabling anaerobic respiration by a Nar containing bacteria if any are present.

Preferably at least the contacting under hypoxic condition is performed in combination with antibiotic an antibiotic effective amount which can be lower than the MIC and/or MBC as will be understood by a skilled person upon reading of the present disclosure.

In preferred embodiments the method can further comprise contacting an antibiotic to an oxic portion of the biological environment.

In some embodiments, the hypoxic region and/or anoxic region of the infected biological environment is located at a depth of 50-100 um from the surface of the biological environment.

In some embodiments, the hypoxic region and/or anoxic region of the infected biological environment is located at depth greater than −10-20 um from the surface of the biological environment.

In some embodiments, the hypoxic region and/or anoxic region of the infected biological environment is located at depth within −5 μm from the surface of the biological environment.

In some embodiments, the hypoxic region and/or anoxic region of the infected biological environment comprises biofilms of a diameter >20 um.

In some embodiments the method to treat an infected biological environment through chlorate timed and/or targeted administration further comprise contacting an antibiotic with an antibiotic administration site within an oxic region of the infected biological environment, the oxic portion comprising oxygen at a level enabling aerobic respiration by a Nar containing bacteria if any is present.

In some of those embodiments, contacting an antibiotic is performed following contacting the chlorate and the antibiotic administration site is within a chlorate treated region of the biological environment.

In some of those embodiments, the oxic region comprises one or more chlorate treated region of the biological environment.

In some of those embodiments, the contacting of the antibiotic is performed at an antibiotic administration time following chlorate administration to one or more portions of the biological environment for example at a time when the chlorate has penetrated into a biofilm formed by the Nar-containing bacteria, if any is present, in the biological environment.

In some embodiments, a method of treatment of a biological environment infected by a NAR containing bacteria is described that can be performed by a method comprising
(a) repeatedly monitoring in one or more target areas of the infected biological environment at least one of i) an oxygen level, ii) a redox potential and iii) a nitrate concentration of the infected biological environment,
(b) upon detection in a target area of an oxygen level above 100 uM, and a redox potential above 200 mV and no nitrate concentration,
administering to the target area an antibiotic in an antibiotic amount effective to inhibit viability of the bacteria
(c) upon detection in a target area of at least one of an oxygen level below a selected threshold level preferably 100 uM, a redox potential below a selected threshold potential preferably 200 mV and a nitrate concentration above a selected threshold concentration preferably 500 uM
administering to the target a chlorate amount in combination with an antibiotic amount, the chlorate amount and the antibiotic amount effective to inhibit viability of the bacteria
(d) upon detection in a target area of a nitrate concentration below a selected threshold concentration preferably 500 uM,
(i) administering to the infected biological environment, nitrate for a nitrate contacting time and in a nitrate amount effective to increase expression of a Nar gene in the Nar-containing bacteria, and
(ii) after the nitrate contacting time, administering to the biological environment chlorate in combination with antibiotics in a chlorate amount and an antibiotic amount effective to inhibit viability of the Nar containing bacteria, the chlorate amount and the nitrate amount in a ratio from 4:1 to 10:1
   the administering of steps (b), (c) and (d) performed until completion of the treatment of the infected biological environment.

In some embodiments, the chlorate administration time is selected from 1 day to 4 months from onset of the infection in the biological environment.

In some embodiments, the chlorate administration time is selected from than 1 day to 30 days from onset of the infection in the biological environment.

In some embodiments, the chlorate administration time is selected from 10 days to 15 days from the onset of the infection.

In some embodiments of any one of the methods and systems herein described and related devices and compositions the administration of chlorate and/or antibiotic can further be performed in combination with the administration of one or more antimicrobial.

The term “antimicrobial” as used herein indicates a substance that kills or inhibits the growth of microorganisms such as bacteria, fungi, or protozoans. Antimicrobial either kills microbes (microbiocidal) or prevent the growth of microbes (microbiostatic).

Exemplary antimicrobial that can be used in combination with chlorate for treating chronic wounds include sterile saline or hydrogel, povidone-iodine solutions, cadexomer iodine, hypochlorous acid, collagenase and others identifiable to a person skilled in the art.

In some embodiments, methods, systems for timed and/or targeted administration of chlorate to treat a bacterial infection and related matrices, compounds, compositions and implants are directed to treatment and/or prevention of infections in medical implants.

The term “medical implants” as used herein indicates devices that are placed inside the body to replace a missing biological structure, support a damaged one and/or or enhance bodily functions. Medical implants in the sense of the disclosure, comprise i) dental implants which comprise a metal (usually titanium) post that is surgically placed in the jawbone and supports a prosthetic tooth or crown; ii) cardiovascular implants such as pacemakers, implantable cardioverter-defibrillators (ICDs), stents (used to open narrowed arteries), and artificial heart valves; iii) neurological implants, such as deep brain stimulation (DBS) for treating conditions like Parkinson's disease and epilepsy, spinal cord stimulators for pain management, and cochlear implants to restore hearing; iv) breast implants for reconstructive or cosmetic purposes.; which are made of silicone or saline-filled shells and are placed either under the breast tissue or chest muscles; v) intraocular lenses (IOLs) used in cataract surgery to replace the natural lens of the eye. IOLs restore clear vision and may correct refractive errors; vi) implantable Drug Delivery Systems used to deliver medications directly to specific areas of the body, for example to treat conditions like chronic pain, cancer, or hormone imbalances; and vii) orthopedic implants: used to replace or support bones and joints, such as hip and knee implants, spinal implants (e.g. rods and screws), and plates used for fracture fixation.

Medical implants in the sense of the disclosure comprise various type of materials which interface with tissues and organs of the body of an individual and provide potential sites of infection especially in the immediate post-surgical period. Materials used in medical implants comprise i) metals such as titanium, stainless steel, and cobalt-chromium alloys used for example in orthopedic and cardiovascular implants due to their strength and compatibility with the body; ii) ceramics used for example in dental implants and some joint replacements due to their biocompatibility and resistance to wear, and iii) polymers: used in implants, such as temporary implants or drug-delivery systems due to their lightweight and flexible nature, and/or to coat medical devices to improve biocompatibility, reduce friction, or provide specific functionalities such as drug delivery, redirection of fluids, enablement of fluid passage, restriction of tissue ingrowth, prevention of leaks, and/or formation non-porous and impermeable covers.

In some embodiments medical devices that can be used in connection with the timed and/or targeted chlorate delivery according to methods systems and composition of the disclosure comprise osteoimplants.

The wording “osteoimplants” “medical bone implants”, or “orthopedic implants” as used herein indicates medical devices used to restore, support, or enhance the function of the musculoskeletal system, particularly bones and joints. These implants are commonly employed in orthopedic surgery to treat various conditions, including fractures, joint degeneration, and trauma, as will be understood by a skilled person.

Exemplary osteoimplants comprise i) joint replacement implants used to replace damaged or diseased joints, typically in the knees, hips, and shoulders, and in particular, ii) hip implants typically used to treat severe hip arthritis or fractures, and configured according to a ball-and-socket design, with the ball component attached to the femur (thigh bone) and the socket component inserted into the pelvis; iii) knee implants typically used to treat severe knee arthritis or joint damage and configured to involve the placement of metal components on the femur, tibia (shin bone), and sometimes the patella (kneecap) to recreate the natural joint structure; iv) shoulder implants typically are used to treat conditions like osteoarthritis or rotator cuff injuries, and configured to replace the humeral head (ball of the upper arm bone) and/or the glenoid (socket of the shoulder blade); and v) trauma implants used to treat traumatic injuries, such as fractures, dislocations, and complex injuries caused by accidents or falls.

Additional exemplary osteoimplants comprise fracture fixation implants, typically used to stabilize fractured bones, allowing them to heal properly. Exemplary fracture fixation implants comprise i) metal plates and screws used to hold fractured bone segments together, providing stability during the healing process; ii) intramedullary rods (also indicated as nails) typically inserted into the bone's medullary canal to stabilize long bone fractures, such as those in the femur or tibia; and iii) external fixators: Devices placed outside the body, connected to pins inserted into the bone. These are used for complex fractures or when internal fixation is not suitable.

Further exemplary osteoimplants comprise spinal implants: typically used to stabilize the spine, correct deformities, or relieve spinal compression. Exemplary spinal implants comprise: i) pedicle screws and rods used in spinal fusion surgeries to stabilize the spine after the removal of damaged discs; ii) interbody fusion cages: placed between vertebrae in spinal fusion procedures to promote bone growth and stabilize the spine and iii) artificial discs: Used as alternatives to traditional spinal fusion, these devices aim to preserve motion in the spine while treating disc-related issues.

Orthopedic implants are typically made from biocompatible materials, such as titanium, stainless steel, or cobalt-chromium alloys, to minimize the risk of adverse reactions within the body. The choice of implant type depends on the specific condition being treated, the patient's overall health, and the surgeon's judgment. Proper surgical technique and post-operative care are essential for successful outcomes with orthopedic implants.

In particular, in medical implants chlorate alone or in combination with antibiotic can be comprise in drug release comportments and/or in polymeric coatings as will be understood by a skilled person.

In some embodiments, prosthetic implants according with the present disclosure are configured for implantation in a body and comprise:
(a) i) a sensor configured to measure an oxygen level, ii) a sensor configured to measure a redox potential and iii) a sensor configured to measure a nitrate concentration; the sensors configured in the system to measure an environment of the prosthesis when in use;
(b) a processor in communication with each of the sensors from (a);
(c) i) a first compartment containing an antibiotic payload, ii) a second compartment containing a chlorate payload, and iii) a third compartment containing a nitrate payload.

In those embodiments the compartments of (c) can be actuated to release their payload selectively by the processor and the prosthesis can further comprise
(e) a communication module connected to the processor and configured to communicate with a system outside the body;
   and the processor is configured to, when instructed to begin antibiotic treatment by the system outside the body:
i) administer to the environment an antibiotic in an antibiotic amount effective to inhibit viability of the bacteria by actuating the first compartment when an oxygen level above 100 uM, a redox potential above 200 mV, and no nitrate concentration is measured,
ii) administer a chlorate and an antibiotic to the environment the chlorate and the antibiotic administered in a chlorate amount and an antibiotic amount effective to inhibit viability of the bacteria by actuating the first compartment and the second compartment, when at an oxygen level below the detected threshold, preferably 100 uM, a redox potential below the detected threshold, preferably 200 mV and a nitrate concentration above the detected threshold, preferably 500 uM is detected, and
iii) administer nitrate to the environment by actuating the third compartment for a nitrate contacting time and in a nitrate amount effective to increase expression of a Nar gene in the Nar-containing bacteria, and after the nitrate contacting time, administering to the environment chlorate in combination with antibiotics in a chlorate amount and an antibiotic amount effective to inhibit viability of the Nar containing bacteria by actuating the first compartment and the second compartment, the chlorate amount and the nitrate amount in a ratio from 4:1 to 10:1, the administer performed when an oxygen level below the detected threshold, preferably 100 uM, a redox potential below the detected threshold, preferably 200 mV, and a nitrate concentration below the detected threshold, preferably 500 uM is detected.

In some embodiments herein described methods, systems, and related compounds and composition of the disclosure are directed to treating and/or preventing infections of wounds and particularly chronic wounds.

In some embodiments, herein described, treating and/or preventing of a wound can be performed by inhibiting bacteria biofilm formation and/or disrupting bacterial biofilm in the wound with a biofilm treatment matrix, compositions, methods and systems based on a chlorate used alone or preferably in combination with one or more antibiotics and/or antimicrobials and possibly further in combination with a wound healing agent.

The term “wound” as used herein indicates the result of a disruption of normal anatomic structure and function of an individual [35] [36]. Accordingly, wounds in the sense of the disclosure encompass a wide range of a defects or breaks in a tissue and/or organs of an individual, resulting from physical, chemical and/or thermal damage, and/or as a result of the presence of an underlying medical or physiological condition” as will be understood by a skilled person [37].

Exemplary wounds comprise abrasions and tears of a tissue of an organ of an individual (e.g. skin) which can be caused by blunt and/or frictional contact with hard surfaces, such as when the an organ is torn, cut, or punctured (an open wound), when the organ is contused (a closed wound), as well as when the organ lesioned and comprise a region in an organ or tissue having abnormal structural change, e.g. following damage through injury or disease. [37]

Exemplary wounds comprise ulcers, like decubitis ulcers (bedsores or pressure sores) and leg ulcers (venous, ischaemic or of traumatic origin) [38] [39] [40], abscesses such as lesions caused by foreign bodies at the time of an injury, or by infections and tumors [37].

In particular wounds comprise abnormal structures in the body of an individual caused by mechanical forces (such as knives and guns but also surgical treatment), thermal sources, chemical agents, radiation, electricity and/or other sources identifiable by a skilled person [37][41]. Wounds also comprise abnormal anatomic structure and function of organs and/or tissues in an individual resulting from conditions such as autoimmune diseases or disorders, infections such as viral infections, cancer, as well as chronic diseases such as diabetes.

Exemplary wounds comprise superficial wounds (affecting only a surface epithelium of the organ, e.g. epidermal skin), partial thickness wounds (also affecting a connective tissues, of the organ such as skin's deep dermal layers) and full thickness wound (further affecting deeper tissues of the organ such as subcutaneous fat in addition to the epidermis and dermal layers) [37][42] [43].

Exemplary wounds also comprise lesions in eyes, ears, stomach intestine and additional portions of the gastrointestinal tract, and in additional tissue organ or body part., including lesions occurring in pulmonary infections such as cystic fibrosis and additional conditions, and in general to chronic infections such as the ones associated with implanted medical devices in lungs and additional tissues and organs of an individual.

Wounds in the sense of the present disclosure can be categorized based on the related characteristics in connection with the wound healing process in the individual.

The term “wound healing” as used herein indicates a biological process directed to growth and tissue regeneration in the individual [37]. In particular, during the wound healing process cellular and extracellular components of the injured tissue or organ interact to restore the integrity of the organ or tissue in interdependent and overlapping stages will be understood by a skilled person [44] [45] [46] [47] [48] and [49].

In particular, a wound heling process in the sense of the disclosure comprises hemostasis, inflammation, migration, proliferation and maturation phases [47] [50].

The term “hemostasis” in the sense of the disclosure indicates a stage of wound healing characterized by the presence of by exudate (blood without cells and platelets), exudate components such as clotting factors, coagulation of the exudate, formation of a fibrin network, and production of a clot in the wound causing bleeding to stop [37] [51].

The term “inflammation” in the sense of the disclosure indicates a stage of wound healing process characterized by release of protein-rich exudate, vasodilation through release of histamine and serotonin, presence of phagocytes and engulf dead cells forming necrotic tissue in the wound, sloughy (yellowish colored mass), and platelets aggregate as will be understood by a skilled person [37]. The inflammatory phase occurs almost simultaneously with hemostasis, sometimes from within a few minutes of injury to 24 h and lasts for about 3 days as also understood by a skilled person. [37]

The term “migration” in the sense of the disclosure indicates a stage of wound healing process characterized by movement of epithelial cells and fibroblasts to the injured area, regeneration and growth of fibroblast and epithelial cells accompanied by epithelial thickening. [37]

The term “proliferation” in the sense of the disclosure indicates a stage of wound healing process characterized by formation of granulation tissue, collagen synthesis and in-growth of capillaries and lymphatic vessels into the wound, formation of blood vessels, fibroblast proliferation and collagen thickening blood vessels decrease and oedema recedes., as will be understood by a skilled person. [37]. The proliferative phase occurs almost simultaneously or just after the migration phase (Day 3 onwards) and basal cell proliferation, which lasts for between 2 and 3 days, and continues for up to 2 weeks by which time blood vessels decrease and oedema recedes.as will also be understood by a skilled person. [37]

The term “maturation” or “remodeling” in the sense of the disclosure indicates a stage of wound healing process characterized by formation of cellular connective tissue and strengthening of the new epithelium which determines the nature of the final scar. [37] Cellular granular tissue is changed to an acellular mass from several months up to about 2 years.

A description of appearance of wound in connection with the wound heling process can be found in Table 1 of [37] enclosed, as Appendix III in U.S. provisional 63/012,036 incorporated herein by reference in its entirety.

Wounds in the sense of the disclosure can be categorized in connection with the related progression and repairs in the healing process, in acute wounds and chronic wounds.

“Acute wounds” in the sense of the disclosure are “tissue injuries that heal completely, with minimal scarring, within the expected time frame, usually 8-12 weeks” [37](see also [52]).

Conversely, a “chronic wound” or a “complex wound” in the sense of the disclosure indicates wounds that fail to proceed through the normal phases of wound healing in an orderly and timely manner and often stall in the inflammation phase of healing. In particular, the wording “chronic wound” refers a wound subjected to a disruption of the orderly sequence of events during the wound healing process which slows down or prevent healing of the wound [53] [54].

Typically, a chronic wound is wound not healed in 4 weeks and in some cases over 4 weeks, beyond, 12 weeks or later [53] typically following repeated tissue insults, underlying physiological conditions, pathological conditions (e.g. persistent infections) treatment of the individual and/or other patient related factors [37]. If healed, a chronic wound can often reoccur. [54]

Typically a chronic wound is a characterized by a high level of oxidative stress compared with non-chronic wounds and with tissue and organs with no lesions, Oxidative stress (OS) is present in tissues and cells when there is an imbalance between the levels of reactive oxygen species (ROS) and the ability of antioxidants in the tissues and cells to remove these species and repair the damage they cause, as will be understood by a skilled person (see [55] enclosed as Appendix VI in U.S. provisional 63/012,036 incorporated herein by reference in its entirety).

Oxidative stress can be detected by detecting expression levels of enzymes that produce ROS, e.g. XCT or Slc7a11, which can have up to an 8.6 times fold increase, Nox4 which can have up to a 2.1- or 3-fold increase and Hmox1 which can have up to 4.5-fold increase, in chronic wounds determined by Nanostring analysis during the first 48 hrs of chronicity initiation.

Additional methods to detect oxidative stress comprise measuring the levels of DNA/RNA damage, lipid peroxidation, and protein oxidation/nitration, directed to measure reactive oxygen species indirectly, as well as additional methods identifiable by a skilled person. Typically, a chronic would is also characterized by hypoxic or anoxic conditions. In particular, in a chronic wound the pO2 is typically halved compared to a non-chronic wound.

For example, chronic wound surfaces on skin have been identified to be hypoxic at ˜37 mmHg, with a mean pHe of ˜6.8 even in absence of an epidermal barrier absent in most areas. Additionally, it has been shown that one day after wounding pHe is above 8 and pO2 is ˜60 mmHg, and that both parameters decrease during epidermal barrier restoration in physiological healing [56].

Exemplary chronic wounds in the sense of the disclosure comprise wounds presenting an extensive loss of the integument (skin, hair, and associated glands), wounds presenting tissue death and/or signs of circulation impairment and, as well as wounds resulting from a pathology [37] [57].

Exemplary chronic wounds further comprise wounds presenting an excess exudate which typically is more corrosive as it includes a relatively higher levels of tissue destructive proteinase enzymes [37] [58] [40]. Accordingly, chronic wounds comprise oedema caused by inflammation, reduced mobility and venous or lymphatic insufficiency and additional wounds presenting an excess exudate as will be understood by a skilled person [37] [39].

Exemplary chronic wounds also comprise wounds including foreign bodies and possibly presenting granuloma or abscess formation, and wounds presenting keloid (raised) scars resulting from excess collagen production in the latter part of the wound healing process. [37][51].

Exemplary chronic wounds also comprise wounds presenting a persistent infection (e.g. Fournier's gangrene), and in particular infection of one of more pathogenic bacteria such as Staphylococcus aureus, Pseudomonas aeruginosa, Streptococcus pyrogenes and some Proteus, Clostridium and a Coliform. Typically, chronic wounds presenting persistent infections are infected with P. aeruginosa and/or S. aureus which significantly reduce skin graft healing [37][38].

Exemplary chronic wounds also comprise wounds of individuals in poor nutritional status (e.g. protein, vitamin (e.g. vitamin C) and mineral deficiencies) and/or of old age [59][60].

Exemplary chronic wounds further comprise wounds of individuals with underlying conditions such as diabetes and anaemia [37] [61]. and/or under treatment of drugs such as glucocorticoids or other steroids capable of suppressing the body's inflammatory responses and thereby impede the inflammatory stage of wound healing [62] [63] [64].

Exemplary chronic wounds include diabetic foot ulcers, venous leg ulcers, pressure ulcers, decubitus ulcers (bedsores or pressure sores) and leg ulcers (venous, ischaemic or of traumatic origin) and others identifiable to a person skilled in the art.

In some embodiments, compositions, methods and systems herein described can further comprise at least one wound healing agent.

The wording “wound healing agents” refer to agents that can stimulate and/or accelerate any one of the stages during the wound healing process, including inflammation, proliferation and remodeling as will be understood by a skilled person. In matrices, agents, compositions, methods and systems of the present disclosure wound healing agents are comprised in a therapeutically effective amounts that can be identified by a skilled person based on the specific agent, wound and route of administration as will be understood by a skilled person.

In some embodiments, wound healing agents comprise growth factors, which are substance capable of stimulating cell division, migration, differentiation, protein expression and enzyme production and/or cell proliferation, in an organ or tissue of the individual “Growth factor” at Dorland's Medical Dictionary 2011 [65] [66]. In particular, the wound healing properties of growth factors are typically mediated through stimulation of angiogenesis and cellular proliferation, which affects both the production and the degradation of the extracellular matrix and also plays a role in cell inflammation and fibroblast activity [67] and affect the inflammatory, proliferation and migratory phases of wound healing. [68]

Growth factors typically comprise secreted proteins or steroid hormones, signaling molecules between cells. Examples are cytokines and hormones that bind to specific receptors on the surface of their target cells and promote cell differentiation and maturation. Exemplary target cells re keratinocytes and fibroblasts which are involved in re epithelialization and collagen deposition, respectively [66] [69].

Exemplary growth factors possibly comprised in wound healing compositions, and related biomimetic matrix, methods and systems comprise epidermal growth factor (EGF), platelet derived growth factor (PDGF), fibroblast growth factor (FGF), transforming growth factor (TGF-b1), insulin-like growth factor (IGF-1), human growth hormone and granulocyte-macro-phage colony-stimulating factor (GM-CSF) [37] [70] [71].

Preferred growth factors comprise GM-CSF with particular reference to in full thickness wounds [72]. epidermal growth factor (EGF), [66] with particular reference to combined treatment with silver sulphadiazine [73], PDGF with particular reference to treatment where granulation tissue and re-epithelialization is desired (such as) in human patients with diabetic foot ulcers [66] [74] [75] [76] [77] [78] [79] [80], fibroblast growth factor (FGF), [66][77] [81] [82] [83] [84], and vascular endothelial growth factor (VEGF). [66] [85] [86] [87] [88][89].

A summary of growth factor modified materials and their corresponding strategies for growth factor encapsulation and delivery is reported in Table 1 of [66] enclosed as Appendix I in U.S. provisional 63/012,036 incorporated herein by reference in its entirety.

Therapeutically effective amount of growth factors can be identified by a skilled person in view of the specific factor and related formulation and route of administration as will be understood by a skilled person. For example. rhPDGF can be administered in an effective amount of 0.001% composition in the FDA approved Regranex. Additional, amounts can be identified by a skilled person.

In some embodiments, wound healing agents comprise supplements such as vitamins and mineral supplements [37] [90] including vitamins A, C, E as well as zinc and copper [37] comprised in an effective amount identifiable by a skilled person in view of the specific supplement as well as timing formulation and route of administration.

In some embodiments, supplements administered with timed and/or targeted chlorate administration methods and systems of the disclosure, and related matrices, implants and compositions herein described, comprise Vitamin A, in particular in embodiments where treatment is directed to promote epithelial cell differentiation, [37] [91] collagen synthesis and bone tissue development [37, 92], normal physiological wound healing as well as reversing the corticosteroid induced inhibition of cutaneous wound healing and post-operative immune depression [37, 93].

In some embodiments, supplements administered with timed and/or targeted chlorate administration methods and systems of the disclosure and related matrices implants and compositions herein described, comprise Vitamin C in particular in embodiments where treatment is directed to promote synthesis of collagen and other organic components of the intracellular matrix of tissues such as bones, skin and other connective tissues; [37] [91] normal responses to physiological stressors such as in accident and surgical trauma and the need for ascorbic acid increases during times of injury [37](Pugliese PT. 1998); immune function particularly during infection. [37] [94]. (Martins-Green and Saeed, 2020) [55] [95], [96], [97].

In some embodiments, supplements administered with timed and/or targeted chlorate administration methods and systems of the disclosure, and related matrices, implants and compositions herein described comprise Vitamin E in particular in embodiments where treatment is directed to promote wound healing; [98] preservation of important morphological and functional features of biological membranes; [67] [99] antioxidant and anti-inflammatory activity [100] as well as promoting angiogenesis and reduces scarring [101]. (Martins-Green and Saeed 2020) [55] [102],

In some embodiments, supplements included in administered with timed and/or targeted chlorate administration methods and systems of the disclosure, and related matrices implants, and compositions herein described, comprise Zinc in particular in embodiments where treatment is directed to promote healing of leg ulcers through enhancement of reepithelialization [103] upregulation of metallothioneins[104], rapid healing of wounds retarded by corticosteroid treatment [105], treatment of deep second-degree burn wounds, preferably in combination with FGF and EGF [106] decrease of Staphylococcus load in the wound [107].

In some embodiments the wound healing agent is an antioxidant agent, which, as used herein indicates a compound that inhibits oxidation. In particular, in a biological environment, antioxidants inactivate reactive oxygen species (herein also ROS) by donating their electrons to these species and preventing them from capturing electrons from other important molecules such as DNA, proteins and lipids, thus protecting the environment against excessive oxidative stress (herein also OS) as will be understood by a skilled person. (Martins-Green and Saeed, 2020) [55].

In preferred embodiments, of the wound healing combination compositions biomimetic matrix and related compositions, methods and systems applied to chronic wounds, comprise at least one antioxidant. In some embodiments, the at least one antioxidant comprises an antioxidant operating through enzymatic and/or an antioxidant operating through non-enzymatic reactions that can occur intracellularly in the cytosol and/or in organelles such as the mitochondria or in the extracellular environment, (Martins-Green and Saeed, 2020) [55], Antioxidants are comprised in wound healing combination compositions biomimetic matrix and related compositions, methods and systems in a therapeutically effective amounts identifiable by a skilled person based on the specific antioxidant as well as formulation, method and route of administration.

Exemplary types of antioxidants, those that perform enzymatic reactions and those that are non-enzymatic in their effects are shown in Table I of (Martins-Green and Saeed 2002) [55] enclosed Appendix VI in U.S. provisional 63/012,036 incorporated herein by reference in its entirety, inclusive these antioxidants targeting reactions occurring in the extracellular microenvironment, others occur intracellularly in the cytosol and/or in organelles such as the mitochondria [108].

In some embodiments, antioxidants included in wound healing combinations, compositions, biomimetic matrix, and related compositions methods and systems herein described, comprise one or more of superoxide dismutase (SOD), glutathione S-transferases (GSTs), glutathione peroxidases (GPx), NADPI-H), catalase, heme-oxygenase 1 (HO-1), peroxiredoxins (Prdx), thioredoxin-1 (Trx-1) and -2 (Trx-2). (Martins-Green and Saeed, 2020 [55].

In particular, in some embodiments, antioxidants included in wound healing combinations, compositions, biomimetic matrix, and related compositions methods and systems herein described, comprise Hemet-oxygenase 1 (HO-1) in particular in embodiments where treatment is directed to promote degradation of heme into CO and/or iron in the presence of O₂ and NADPH giving rise to biliverdin that is converted into bilirubin [109] [110], wound closure and angiogenesis resulting in increased wound healing. [109] [110], (Martins-Green and Saeed, 2020) [55].

In particular, in some embodiments, antioxidants included in wound healing combinations, compositions, biomimetic matrix, and related compositions methods and systems herein described, comprise peroxiredoxins and thioredoxins in particular in embodiments where treatment is directed to promote reduction of oxidative stress [111] [112] reduction of 1-202 as well as a broad range of peroxides [113] [114] detoxification of tissues and cells from peroxynitrite [115] [116] rapid wound [116] [117], reduction of other proteins by cysteine thiol-disulfide exchange and reduction of inflammation [118]. (Martins-Green and Saeed, 2020).

In particular, in some embodiments, antioxidants included in wound healing combinations, compositions, biomimetic matrix, and related compositions methods and systems herein described, comprise non-enzymatic antioxidants such as vitamin C (ascorbic), vitamin E (α-tocopherol), Vitamin D, glutathione, N acetyl cysteine (NAC), alpha lipoic acid (αLA), carotenoids (e.g. lycopenes), bilirubin and uric acid, [119] [120] [121] [116]

In some embodiments, antioxidants included in wound healing combinations, compositions, biomimetic matrix, and related compositions methods and systems herein described, comprise glutathione in particular in embodiments where treatment is directed to promote strength of the wound tissue. [122] healing of wounds in diabetic individual [123]. Preferably in combination with Vit E (α-tocopherol) [124] [125] [126] [127]. (Martins-Green and Saeed, 2020) [55].

In some embodiments, antioxidants included in wound healing combinations, compositions, biomimetic matrix, and related compositions methods and systems herein described, comprise Vitamin D in particular in embodiments where treatment is directed to promote cancer prevention and inhibition of inflammation [128], proliferation and migration of endothelial cells [129].

In some embodiments, antioxidants included in wound healing combinations, compositions, biomimetic matrix, and related compositions methods and systems herein described, comprise Alpha-Lipoic Acid (α-LA) in particular in embodiments where treatment is directed to promote chelation of toxic heavy metal ions including Fe²⁺ and Cu²⁺, Fe²⁺ can react with H₂O₂ to produce Fe³⁺+OH⁻+OH⁻ (Fenton reaction) which can cause protein modification, lipid peroxidation and DNA damage, scavenging of OS [130] regeneration of Vit E, Vit C, coenzyme Q10 and glutathione. (Martins-Green and Saeed, 2020).

In some embodiments, antioxidants included in wound healing combinations, compositions, biomimetic matrix, and related compositions methods and systems herein described, comprise N-acetyl-cysteine (NAC): in particular in embodiments where treatment is directed to promote antimicrobial activity in connection with biofilm formation and/or disruption, in particular in wounds infected by Pseudomonas aeruginosa, Escherichia coli, Staphylococcus epidermidis, Strepnococcus pneumoniae, Staphylococcus aureus and Klebsiella pneunoniae [131], [132] [133](Mohsen et al 2015) [134]. N-acetyl-cysteine (NAC): can also be comprised in in particular in embodiments where treatment is directed to promote modulation of granulocyte function, increase IL-12 secretion, activation NF-κB pathway, decrease of mnetalloproteinase-9, IL-8, IL-6, and/or inflammatory cytokines and oxidative stress at normal levels [135] [136][137] [138] [139] [140], burn wound healing [141]. Healing of incisional wound of diabetic and non-diabetic individual [142] faster healing [143] [125] [126] [127](Martins-Green and Saeed 2020).

In some embodiments, antioxidants included in wound healing combinations, compositions, biomimetic matrix, and related compositions methods and systems herein described, comprise other small molecules such as carotenoids (in particular lycopenes), bilirubin, and/or uric acid. (Martins-Green and Saeed 2020).

In some embodiments, antioxidants included in wound healing combinations, compositions, biomimetic matrix, and related compositions methods and systems herein described, comprise bilirubin in particular in embodiments where treatment is directed to promote healing, increased neovascularization and improved collagen deposition of diabetic wound [144] and reduction of oxidative stress in wound tissues [145](Martins-Green and Saeed 2020.

In some embodiments, antioxidants included in wound healing combinations, compositions, biomimetic matrix, and related compositions methods and systems herein described, comprise 6,8 dithio-uric acid in particular in embodiments where treatment is directed to promote wound healing protection of cells and in particular neural cells endothelial cells keratinocyte and fibroblasts from oxidative damage [146] [147] [97]. (Martins-Green and Saeed 2020).

In some embodiments, antioxidants included in administered in connection with timed and/or targeted chlorate administration methods and systems of the disclosure, and related matrices, implants and compositions herein described, comprise herbal extracts such as curcumin and honey. (Martins-Green and Saeed 2020).

In some embodiments, antioxidants administered in connection with timed and/or targeted methods and systems of the disclosure, and related matrices, implants and compositions herein described, comprise curcumin in particular in embodiments where treatment is directed to promote increase in collagen content and wound contraction [148] and/or in treatment of excision wounds. [149] [150] [151] [152]. (Martins-Green and Saeed 2020).

In some embodiments, antioxidants administered in connection with timed and/or targeted chlorate administration methods and systems of the disclosure, and related matrices implants and compositions herein described, comprise honey in particular in embodiments where treatment is directed to promote, antimicrobial treatment [153], anti-inflammatory treatment [154], early improvement in wound healing process [155] [156], imnunomodulatory treatment [157] [158]. (Martins-Green and Saeed 2020).

In some embodiments, antioxidants administered in connection with timed and/or targeted chlorate administration methods and systems of the disclosure, and related matrices implants and compositions herein described, comprise Factor-E2-related factor (Nrf2) in particular in embodiments where treatment is directed to improve healing under oxidative stress conditions in impaired wounds [159] in particular in diabetic wounds [160] [161] [159] [162][163]. (Martins-Green and Saeed 2020).

In some embodiments the wound healing agent is an anti-oxidant agent, such as N-acetyl cysteine, coenzyme Q (ubiquinol), vitamin A, vitamin C, vitamin E, glutathione, lipoic acid, carotenes, flavenoids, phenolics, and ergothioneine, melatonin, ellagic acid, punicic acid, luteolin, catalase, superoxide dismutase, peroxiredoxins, cysteine, or a physiological salt thereof, or a combination thereof. In some embodiments, the wound healing agent can be a free radical scavenger, a lipid peroxidation inhibitor, or a combination thereof.

Exemplary effective amounts of antioxidant agents comprise 0.1-3.0% NAC, 0.3% bilirubin ointment as well as 10 mg/kg of curcumin to increase collagen and 40 mg/kg for excision wounds (daily application).

Additional antioxidant agents, related concentration and applications are described in (Martins-Green and Saeed 2020) [55] enclosed as Appendix VI in U.S. provisional 63/012,036 incorporated herein by reference in its entirety.

In some embodiments, timed and/or targeted chlorate administration methods and systems of the disclosure, and related matrices, implants and compositions herein described, comprise in addition to chlorate, at least one antioxidant, small molecules such as alpha-tocopherol (Vitamin E), n-acetyl cysteine (NAC), proteins such as cytokines, growth factors (e.g. EGF, VEGF, TGF beta, PDGF), and/or other bioactive molecules identifiable to a skilled person. In preferred embodiments at least one antibiotic is further comprised.

Additional wound healing agents and various approaches to apply for targeted wound therapy can be found in [66] enclosed, as Appendix I in U.S. provisional 63/012,036 incorporated herein by reference in its entirety.

In some embodiments of the biofilm treatment matrix, compositions, methods and systems, herein described, chlorate can be administered together with one or more antibiotics either sequentially (such as the chlorate first and then the antibiotics) or in a single administration for a time period until the biofilm is disrupted. The wound can then be treated with wound healing compounds.

In embodiments of timed and/or targeted chlorate administration methods and systems of the disclosure, and related matrices, implants and compositions herein described applied in connection of treating and/or preventing chronic wound in an individual comprises contacting the chronic wound of the individual with the composition herein described comprising an effective amount of chlorate alone or in combination with an effective amount of antibiotics and/or antimicrobial. The contacting of the composition is performed for a time and under conditions to reduce antibiotic resistance and/or bacterial survivability, by producing chlorite which is toxic for the cell within the cytoplasm of the cell via Nar-mediated reduction of the chlorate into toxic chlorite. Accordingly, in embodiments herein described the contacting results in inhibition of viability of the Nar-containing bacteria via cytoplasmic chlorite production while minimizing the interference with the viability of possible neighboring cells lacking Nar.

In timed and/or targeted chlorate administration methods and systems of the disclosure and related matrices, compositions and implants, timing and dosages of administration of chlorate alone or in combination with one or more antibiotics and/or antimicrobials to treat and/or prevent bacterial infection herein described can vary depending on the individual treated, the effect to be achieved (treatment and/or prevention) and the severity of the infection as will be understood by a skilled person.

Suitable dosages can be used which provide the individual with a therapeutically effective amount or a prophylactically effective amount in accordance with the related embodiments of the disclosure. In particular, the term “effective amount” of one or more active ingredients refers to a nontoxic but sufficient amount of one or more drugs to provide the desired effect. For example, an “effective amount” of chlorate associated with the treating and/or preventing (herein also “therapeutically effective amount” or “pharmaceutically effective amount”) a condition in the individual in which bacterial infections are present, refers to a non-toxic but sufficient amount of the chlorate to provide the treatment and/or prevention of such condition in the individual. As another example, an “effective amount” of at least one antibiotic and/or antimicrobial associated with the treating and/or preventing bacterial infection in the individual refers to a non-toxic but sufficient amount of the at least one antibiotic and/or at least one antimicrobial to provide the treatment and/or prevention of the bacterial infection in the individual. A non-toxic amount for chlorate can be identified by a person skilled in the art based on the guidelines and health reference levels provided by health organizations such as WHO and environmental protection agencies such EPA.

In certain embodiments in timed and/or targeted chlorate administration methods and systems of the disclosure and related matrices, compositions and implants, administering the chlorate, antibiotics, antimicrobial and/or would healing agents of the disclosure can be performed by systemic administration. In some of those embodiments the systemic administration is performed by parenteral administration and more particularly intravenous, intradermic, and intramuscular administration. In some of those embodiments, systemic administration is performed by non-parenteral administration and more particularly intranasal, intratracheal, vaginal, oral, and sublingual administration.

Exemplary compositions for parenteral administration comprise sterile aqueous solutions, injectable solutions or suspensions including chlorate alone, antibiotics alone, antimicrobial alone, or wound healing agents lone, or a combination of chlorate with antibiotics, antimicrobials and additional wound healing agents s will be understood by a skilled person.

In certain embodiments, administering the with a chlorate, an antibiotic, an antimicrobial and/or a wound healing agent of the disclosure can be performed by topical administration [164] [165]. Topical administration includes, but is not limited to, epicutaneous administration, inhalational administration (e.g. in asthma medications), enema, eye drops (E.G. onto the conjunctiva), ear drops, intranasal route (e.g. decongestant nasal sprays), and vaginal administration.

In some embodiments, a chlorate, an antibiotic, an antimicrobial and/or a wound healing agent of the disclosure can be administered transdermally using tools such as micro/nanocarriers that can pass through the skin barrier and stratum corneum or microneedles that can poke through the barrier and deliver the composition to the viable tissue underneath.

In some embodiments in which the timed and or targeted methods and systems of the disclosure are performed to treat and/or prevent systemic infections and/or chronic infections (e.g. pulmonary infections, and/or infections associated with the use of implanted medical devices) administration through intravenously, intramuscularly, or inhaled as an aerosol or via a nebulizer allows an effective delivery of the agents and compositions of the instant disclosure.

Accordingly, in some embodiments, a timed and/or targeted composition for treating and/or preventing an infection of a biological environment is described. The composition comprises one or more chlorate alone, one or more antibiotics alone, one or more antimicrobial alone, one or more wound healing agent alone or in various combinations identifiable by a skilled person upon reading of the disclosure.

In some embodiments, a composition for treating and/or preventing an infection according to timed and/or targeted methods and systems of the disclosure can be formulated as liquid (solutions, suspensions and emulsions) and semi-solid (ointments and creams) In particular, solutions such as are most effective in the initial stages of wound healing for reducing bacterial load and as debriding and desloughing agents to prevent maceration of healthy tissue by the removal of necrotic tissue from the fresh wound. Antimicrobial agents such as silver, povidone-iodine.(Misra and Nanchahal 2003) [166] and polyhexamethylene biguanide [167] are sometimes incorporated into dressings to control or prevent infection. Physiological saline solution is used for wound cleansing to remove dead tissue and also washing away dissolved polymer dressings remaining in a wound. [168] [169]. Saline solution is also used to irrigate dry wounds during dressing change to aid removal with little or no pain. The major problem with liquid dosage forms, however, is short residence times on the wound site, especially where there is a measurable degree of suppuration (exuding) of wound fluid.

The composition herein described can further comprise one or more vehicles as would be identified by a skilled person.

The term “vehicle” as used herein indicates any of various media acting usually as solvents, carriers, binders or diluents for chlorate alone or in combination with antibiotics and/or additional wound healing agents, comprised in the composition as an active ingredient.

In some embodiments, where the composition is to be administered to an individual the composition can be a pharmaceutical wound healing composition and comprises chlorate alone or in combination with antibiotics and/or additional wound healing agents and a pharmaceutically acceptable vehicle.

In some embodiments, chlorate alone or in combination with antibiotics and/or additional wound healing agents can be included in pharmaceutical compositions together with an excipient or diluent. In particular, in some embodiments, pharmaceutical compositions are disclosed which contain chlorate alone or in combination with antibiotics and/or additional wound healing agents, in combination with one or more compatible and pharmaceutically acceptable vehicle, and in particular with pharmaceutically acceptable diluents or excipients.

The term “excipient” as used herein indicates an inactive substance used as a carrier for the active ingredients of a medication. Suitable excipients for the pharmaceutical compositions herein disclosed include any substance that enhances the ability of the body of an individual to absorb chlorate alone or in combination with antibiotics and/or additional wound healing agents. Suitable excipients also include any substance that can be used to bulk up formulations with chlorate alone or in combination with antibiotics and/or additional wound healing agents to allow for convenient and accurate dosage. In addition to their use in the single-dosage quantity, excipients can be used in the manufacturing process to aid in the handling of chlorate alone or in combination with antibiotics and/or additional wound healing agents. Depending on the route of administration, and form of medication, different excipients can be used. Exemplary excipients include but are not limited to antiadherents, binders, coatings disintegrants, fillers, flavors (such as sweeteners) and colors, glidants, lubricants, preservatives, sorbents.

The term “diluent” as used herein indicates a diluting agent which is issued to dilute or carry an active ingredient of a composition. Suitable diluents include any substance that can decrease the viscosity of a medicinal preparation.

In some embodiments, the composition can be in a form of a solution, patch, lotion, hydrogel, cream or embedded in a delivery matrix as will be understood by a skilled person.

In some embodiments of the compositions methods and systems herein described chlorate alone or in combination with one or more antibiotics and the wound healing agents are administered to the wound in a single formulation which can be re-applied regularly.

In some embodiments, chlorate alone or in combination with one or more antibiotics and the wound healing agents can be comprised in a topical liquid or semi-solid formulations such as silver sulpha diazine cream [170] and silver nitrate ointment [171].

In some embodiments, the wound healing combination and/or composition herein described can be in the form of a lotion, hydrogel, solution (in water or PBS) or cream and can thus be delivered topically, e.g. directly into the wounds of an individual and in particular a patient. Alternatively, the composition herein described can be provided to an individual intravenously, intramuscularly, or inhaled as an aerosol or via a nebulizer.

In preferred embodiments, the wound healing combination and/or composition can be administered in a wound dressing configured to deliver the chlorate to the wound site. preferably further configured to cover the wound area and maintains a suitable condition supporting the healing process.

In preferred embodiments, the wound healing combination and/or composition is administered within dressings such hydrocolloid, alginate, collage [172] ointment, film, foam, gel [173] [174] in particular in primary or island dressings [175] which can be used as debridement, antibacterial, occlusive, absorbent or adherence dressings [176].

As a person skilled in the art will understand, the wound dressing used herein in treating a wound are configured to cover the wound, preserve the body water content, be oxygen permeable to allow oxygen access to growing tissue, and prevent the growth of environmental pathogens without interfering with the wound healing. The utilized materials are configured to be immunocompatible, non-degradable, and should not support cell ingrowth and cellular adhesion so to avoid complications during their removal. The wound dressings used herein can preserve the activity of the composition components and should be able to release the components at the desired rate.

In some embodiments, wound dressings used herein are effective in removing wound exudates without dehydrating the tissues. The optimal material should guarantee gas and fluid permeability in order to absorb odors, maintain moist conditions and avoid dehydration and exudates accumulation which can result in the formation of necrotic tissue. Materials for wound dressings vary in terms of the origin of materials, physical forms, architecture, and properties.

Exemplary wound dressings are in the form of gauze, thin film, foam, hydrogels, hydrocolloids, membranes and other identifiable to a person skilled in the wound treatment. Detailed information about various materials used for wound dressing can be found in published literatures such as [177] enclosed as Appendix II in U.S. provisional 63/012,036 incorporated herein by reference in its entirety.

In preferred embodiments, a timed and or targeted chlorate antibiotic combination and/or composition can be administered on a scaffolding material configured to deliver active agents to the wound site and preferably further hosting the endogenous cells and facilitate their growth and wound closure.

The term “scaffold” or “scaffolding material: as used herein indicates a structure comprised of a polymeric central component which is configured to deliver cells, drugs, and genes into the body.

A scaffolding material used herein encompass material configured to facilitate the tissue regeneration, restore the tissue function, and promote a rapid healing process preventing chronic wounds. Preferably, scaffolding material herein described are configured to have a degradation rate that matches the rate of tissue growth. Scaffolding material in the sense of the disclosure are configured to minimize immunogenicity and toxicity of the material and related byproducts of the degradation process.

Exemplary scaffolding materials include bioactive materials such as collagen, hyaluronic acid, chitosan or electrospun nanofibers that mimic the natural collagen fibers in ECM, synthetic polymers including polyurethanes and polyesters, hydrogel scaffolds, foams and spongy biomaterials, composite scaffolds, bi-layered scaffolds and others identifiable to a person skilled in the art. Detailed information about various materials used for scaffolding materials can be found in published literatures such as [177] enclosed as Appendix II in U.S. provisional 63/012,036 incorporated herein by reference in its entirety.

In preferred embodiments, a scaffolding material used in wound healing combination and/or biomimetic matrix as well as in related compositions, methods and systems of the disclosure is configured to adhere properly to the surrounding tissues and to have mechanical properties matching the mechanical properties of the native tissue or organ where the wound is located, to avoid the detachment and breakage over the course of healing. The scaffolding material used in wound healing combination and/or biomimetic matrix as well as in related compositions, methods and systems in the sense of the disclosure are preferably configured to control and in particular maintain its water content or are used in connection with administering approach devised to prevent material dehydration. [177] [178] and [179].

In some embodiments, scaffolding material in the sense of the disclosure are configured to have a limited swelling capacity and maintain their shape over time. In these embodiments scaffolding materials can also be used as a depot of growth factors and the drug that are directly being delivered to the healing tissue. In this frame, engineered skin substitutes have been explored in order to create a 3-dimensional (3D) architecture that can mimic the ECM and reproduce the natural cell microenvironment. [177] [178] and [179].

A most preferred scaffolding material for wound healing combination and/or biomimetic matrix as well as in related compositions, methods and systems of the present disclosure, should guarantee gas and fluid permeability in order to absorb odors, maintain moist conditions and avoid dehydration and exudates accumulation. [177] [178] and [179].

In some embodiments, the chlorate and wound healing agents alone or together with one or more antibiotics and/or one or more antimicrobials can be delivered in a delivery matrix. The delivery matrix can be designed to incorporate the components with high loading efficiency, controlled release while maintaining the bioactivity. [177] [178] and [179].

In some embodiments, the composition herein described is embedded in a delivery matrix such as collagen (a natural component of tissues), hyaluronan (a natural component of tissues), hydrogels made of Poly(vinyl alcohol) (PVA), collagen-chitosan hydrogels, alginate matrices, carbopol gels, hydrocolloidal dressing, foam dressings, matrix enabling slow releases and/or differential releases, and others identifiable to a person skilled in the art. [177] [178] and [179].

Additional scaffolding materials and related features for use as delivery matrices in wound healing combination and/or biomimetic matrix as well as in related compositions, methods and systems of the present disclosure can be found for example in [177], [37] [178] and [179] enclosed as Appendix V, enclosed as Appendix II, Appendix III, Appendix IV, and Appendix V respectively in U.S. provisional 63/012,036 are herein incorporated herein by reference in their entirety.

Accordingly, in some embodiments, a biofilm treatment matrix for treating and/or preventing chronic wound is described, wherein a biofilm treatment agent comprising one or more chlorates alone or in combination with one or more antibiotics and/or antimicrobials is embedded in a delivery matrix.

The systems, compositions, and biofilm treatment matrices herein described can be applied to an individual in various stages of severity of biofilm development. In some embodiments, the systems, compositions, and biofilm treatment matrices herein described can be given to an individual in the early stages of wound healing (i.e. before a wound is defined as chronic) to inhibit the growth of Nar-containing bacteria in the wound or given to an individual after a wound is determined to be chronic to reduce viability of Nar-containing organisms.

In some embodiments, the systems, compositions, and biofilm treatment matrices can be administered shortly after stimulation of wound chronicity. This can be applied to bed sores and pressure sores which can be detected in very early stages.

In some embodiments, the composition for treating and/or preventing a chronic wound in an individual comprises chlorate in an effective amount between 0.001 mM and 200 mM and one or more antibiotics. In some embodiments, the chlorate is in an amount between 1 mM to 200 mM.

In some embodiments, the one or more antibiotics comprise tobramycin in an effective amount between 1 mg/kg/day and 10 mg/kg/day.

In some embodiments, the composition can comprise chlorate 5 mM 20 mM in 50-100 μl/application/day, antibiotic 5 μg/ml-20 μg/ml in 50-100 μl/application, and Wound Agents can comprise 100-500 mg/kg of NAC and 20 mg/kg-100 mg/kg of alpha-tocopherol.

In some embodiments, the composition can be administered once a day, twice a day, three times a day four times a day, or more often as necessary.

In some embodiments, the chlorate alone or in combination with one or more antibiotics and/or antimicrobials in the composition can be administered concurrently, combined in a single dosage form. For example, chlorate alone or in combination with one or more antibiotics and/or antimicrobials can be in a single vehicle dissolved in water or PBS.

In some embodiments, the chlorate alone or in combination with one or more antibiotics and/or antimicrobials can be administered at the same or at different times in separate dosage forms wherein antibiotic or antimicrobial can be administered before or after chlorate.

In some embodiments, methods herein described chlorate is administered in combination with an antibiotic to individuals in which the antibiotic treatment failed when isolate show in vitro sensitivity to the administered antibiotic. In those embodiments, the chlorate targets oxidant-starved pathogen populations, such as those found in chronic wound which are not reached by the antibiotic thus resulting in antibiotic tolerance and treatment failure.

In some embodiments, to stimulate wound healing once the biofilm of the wound is destroyed, a wound healing agent can be further applied to stimulate the healing of the wound.

Accordingly, the method can further comprise, following contacting the chronic wound of the individual with the composition herein describe, applying a wound healing agent in an effective amount to the chronic wound to stimulate the healing.

Exemplary wound healing agent comprise small molecules such as alpha-tocopherol (Vitamin E) (e.g. and 50 mg vitamin E per kg mouse or corresponding dosages in other individuals), n-acetyl cysteine (NAC) (e.g. 200 mg NAC per kg mouse or corresponding dosages in other individuals), proteins such as cytokines, growth factors (e.g. EGF, VEGF, TGF beta, PDGF), and other bioactive molecules identifiable to a skilled person. Additional wound healing agents and various approaches to apply for targeted wound therapy can be found in [66] and [180] enclosed as Appendix I, and Appendix VII, respectively in U.S. provisional 63/012,036 and which are incorporated herein by reference in their entirety.

The wound healing agents can be delivered in various delivery ways such as in protein itself, the cDNA of the proteins, cytokine and growth factor plasma rich fraction alone or embedded in a matrix.

In some embodiments of the methods herein described, the methods are provided to prevent wound chronicity and/or early stage of biofilm development. In these embodiments, contacting the chronic wound with an effective amount of the composition herein described can be performed shortly after wound development within hours or days. Examples of chronic wounds include bed sores and pressure sores that can be detected in very early stages, or diabetic foot ulcers as soon as a wounding event is recognized.

In some embodiments of the methods herein described, the methods are provided to treat chronic wounds and/or biofilm infections. In these embodiments, contacting the chronic wound with an effective amount of the composition herein described can be performed for wounds that are well-developed and have become infection. The composition can be embedded in matrices that permit slow releases such as over days or months or in matrices that allow for differential release.

The methods and composition herein described can allow for re-epithelialization, granulation tissue formation including angiogenesis and then remodeling of the tissue be stimulated.

As described herein, chlorate, nitrate, antibiotics, bacteria, antimicrobial agents and/or compositions herein described can be provided as a part of systems to perform any methods, including any of the assays described herein. The systems can be provided in the form of arrays or kits of parts.

Systems in the sense of the disclosure typically comprise at least one chlorate and a look up table connecting amounts of chlorate a timing of administration of the chlorate and/or with tissue layers of different types of wounds for targeted administration of the chlorate. In some embodiments the system can comprise one or more antibiotics, one or more antimicrobials and/or one or more wound healing agents. In those embodiments, the look-up table can also connect the amount and time of administration of the one or more antibiotic, one or more antimicrobials and/or one or more wound healing agents with times of administration of the chlorate and/or with tissue layers of different types of wounds for targeted administration of the chlorate.

A look-up table as used herein is an N-dimensional array of data indexed by one or more input parameters, such that providing the input parameters provides the system with the data required for the solution (either the final solution, or an intermediate value used to derive the solution). Look-up tables can be stored in firmware or software. Look-up tables can be stored in memory locally, or they can be stored in a remote server where a request is sent to the remote server with the input parameters and the remote server returns the data accessed in the table. The look-up table can be populated by pre-calculating equations using the methods described herein.

In some embodiments, a systems according to the present disclosure is a system of treating an infected biological environment, the system, comprising at least one chlorate and at least one of a look up table connecting amounts of chlorate, timing of administrations and stage of the biological environment, an oxygen sensor, reagents to detect biomarker of anaerobic respiration and reagents to detect biomarker of aerobic respiration. In some of those embodiments, the system of treating an infected biological environment further comprises at least one antibiotic, at least one antimicrobial and at least one wound healing agent.

In some embodiments, a systems according to the present disclosure is a system of treating an infected biological environment, comprising one or more chlorate, one or more antibiotic and at least one of a look up table connecting amounts and timing of administration of chlorate, amounts and timing of administration of antibiotic with respect to the administration of the chlorate, an oxygen sensor, reagents to detect biomarker of anaerobic respiration and reagents to detect biomarker of aerobic respiration. In some of those embodiments, the system of treating an infected biological environment further comprises at least one antimicrobial and at least one wound healing agent as would be understood by a skilled person upon reading of the present disclosure.

In some embodiments, a systems according to the present disclosure is a system of treating an infected biological environment, comprising at least one chlorate and a look up table connecting amounts of chlorate with anoxic target portions of at least a portion of the infected biological environment for targeted administration of the chlorate. In some of those embodiments, the system of treating an infected biological environment further comprises at least one antibiotic, at least one antimicrobial and at least one wound healing agent as well as reagents for detecting biomarker of anaerobic respiration and reagents for detecting biomarker of aerobic respiration.

In some embodiments, a systems according to the present disclosure is a system of treating an infected biological environment comprising at least chlorate, an antibiotic and a look up table connecting amounts and timing of administration of chlorate, amounts and timing of administration of antibiotic with respect to the administration of the chlorate, stages of the infection and preferably also chlorate administration times and/or administration and antibiotic administration times and/or administration site. In some of those embodiments, the system of treating an infected biological environment further comprises at least one antibiotic, at least one antimicrobial and at least one wound healing agent, reagents for detecting biomarker of anaerobic respiration of the Nar containing bacteria, and reagents for detecting biomarker of anaerobic respiration of the Nar containing bacteria.

In some embodiments a system to treat an infected biological environment, comprises: (a) i) a sensor configured to measure an oxygen level, ii) a sensor configured to measure a redox potential and iii) a sensor configured to measure a nitrate concentration; the sensors configured in the system to measure the infected biological environment when in use;
(b) a processor in communication with each of the sensors from (a);
(c) i) a first compartment containing an antibiotic payload, ii) a second compartment containing a chlorate payload, and iii) a third compartment containing a nitrate payload; (d) wherein the compartments of (c) can be actuated to release their payload selectively by the processor;
(e) wherein the processor is configured to:
i) administer to the infected biological environment an antibiotic in an antibiotic amount effective to inhibit viability of the bacteria by actuating the first compartment,
when an oxygen level above the detected threshold, preferably 100 uM, a redox potential above the detected threshold, preferably 200 mV, and preferably nitrate concentration lower than 500 uM or more preferably no nitrate concentration is measured,
ii) administer a chlorate and an antibiotic to the infected biological environment the chlorate and the antibiotic administered in a chlorate amount and an antibiotic amount effective to inhibit viability of the bacteria by actuating the first compartment and the second compartment
when at an oxygen level below the detected threshold, preferably 100 uM, a redox potential below the detected threshold, preferably 200 mV, and a nitrate concentration above the detected threshold, preferably 500 uM, is detected, and
iii) administer nitrate to the infected biological environment by actuating the third compartment for a nitrate contacting time and in a nitrate amount effective to increase expression of a Nar gene in the Nar-containing bacteria, and
after the nitrate contacting time, administering to the biological environment chlorate in combination with antibiotics in a chlorate amount and an antibiotic amount effective to inhibit viability of the Nar containing bacteria by actuating the first compartment and the second compartment, the chlorate amount and the nitrate amount in a ratio from 4:1 to 10:1
when an oxygen level below the detected threshold, preferably 100 uM, a redox potential below the detected threshold, preferably 200 mV, and a nitrate concentration below the detected threshold, preferably 500 uM, is detected.

In some embodiments, a system to prevent chronicity of an infection can comprise environment, the infected biological environment infected by Nar containing bacteria, the system comprising
(a) a sensor configured to measure a nitrate concentration of the infected biological environment when in use;
(b) a processor in communication with the sensor;
(c) i) a first compartment containing an antibiotic payload, ii) a second compartment containing a chlorate payload, and iii) a third compartment containing a nitrate payload;
(d) wherein the compartments of (c) can be actuated to release their payload selectively by the processor;
(e) wherein the processor is configured to:
   i) determine a nitrate concentration from the sensor;
   ii) administer to the infected biological environment a chlorate amount in combination with an antibiotic amount, the chlorate amount and the antibiotic amount effective to inhibit viability of the bacteria, by actuating the first compartment and the second compartment, when the nitrate concentration is determined to be above 500 uM and
   ii) administer, by actuating the third compartment, to the infected biological environment nitrate for a nitrate contacting time and in a nitrate amount effective to increase expression of a Nar gene in the Nar-containing bacteria, and
   (iv) after the nitrate contacting time, administering to the biological environment chlorate in combination with antibiotics in a chlorate amount and an antibiotic amount effective to inhibit viability of the Nar containing bacteria by actuating the first compartment and the second compartment, the chlorate amount and the nitrate amount in a ratio from 4:1 to 10:1.

In some embodiments, a system to treat an infected biological environment, the infected biological environment infected by a NAR containing bacteria, the system comprising:
(a) i) a sensor configured to measure an oxygen level, ii) a sensor configured to measure a redox potential and iii) a sensor configured to measure a nitrate concentration; the sensors configured in the system to measure the infected biological environment when in use;
(b) a processor in communication with each of the sensors from (a);
(c) i) a first compartment containing an antibiotic payload, ii) a second compartment containing a chlorate payload, and iii) a third compartment containing a nitrate payload;
(d) wherein the compartments of (c) can be actuated to release their payload selectively by the processor;
(e) wherein the processor is configured to:
   i) repeatedly monitor from the sensors of (c) and in one or more target areas of the infected biological environment i) an oxygen level, ii) a redox potential and iii) a nitrate concentration of the infected biological environment,
   ii) upon detection in a target area of an oxygen level above 100 uM, and a redox potential above 200 mV and no nitrate concentration,
      administer to the target area an antibiotic in an antibiotic amount effective to inhibit viability of the bacteria by actuating the first compartment and the second compartment,
   iii) upon detection in a target area of an oxygen level below 100 uM, a redox potential below 200 mV and a nitrate concentration above 500 uM
      administer to the target a chlorate amount in combination with an antibiotic amount, the chlorate amount and the antibiotic amount effective to inhibit viability of the bacteria by actuating the first compartment and the second compartment,
   iv) upon detection in a target area of a nitrate concentration below 500 uM,
      first administer to the infected biological environment, nitrate for a nitrate contacting time and in a nitrate amount effective to increase expression of a Nar gene in the Nar-containing bacteria by actuating the third compartment,
      then, after the nitrate contacting time, administer to the biological environment chlorate in combination with antibiotics in a chlorate amount and an antibiotic amount effective to inhibit viability of the Nar containing bacteria, the chlorate amount and the nitrate amount in a ratio from 4:1 to 10:1 by actuating the first compartment and the second compartment; and
   v) repeat the actions of i) to iv) until receiving communication of a completion of treatment of the infected biological environment.

In some embodiments the system can be provided in the form of a kit of parts. In a kit of parts, the chlorate, antibiotics, nitrate antimicrobials, wound healing agent, and/or other reagents to perform the methods herein described can be included in the kit alone or together with sensors and devices herein described or in the combination with of one or more chlorate, nitrate, antibiotic and/or antimicrobial agent compositions. In particular in kit of parts for the treatment of an individual the chlorate, antibiotics, nitrate and/or other reagents can be comprised together with the antibiotic and/or antimicrobial formulated for administration to the individual as well as additional components identifiable by a skilled person upon reading of the present disclosure.

The methods and systems as well as related devices and compositions herein described, can be performed in vivo and/or in vitro as will be understood by a skilled person.

Further embodiments will be illustrated in connection with the following examples EXAMPLES

Features and embodiments, methods and systems for timed and/or targeted administration of chlorate to treat a bacterial infection and related matrices, compounds, compositions and implants are exemplified in the following examples which are incorporated into and constitute integral part of this specification.

In particular, Examples 1 to 6 that several classes of antibiotics display limited toxicity against hypoxic cultures of P. aeruginosa, where the pathogen exhibits antibiotic tolerance or resistance, exhibit toxicity in hypoxic colture when used in combination with chlorate. Examples 3 to 6 also show that chlorate interacts synergistically with all tested classes of antibiotics to eradicate hypoxic P. aeruginosa populations. Examples 1 to 6 further show, that while neither chlorate or antibiotics alone efficiently kill O₂-limited P. aeruginosa, combined chlorate-antibiotic treatment overcomes both antibiotic tolerance and resistance to kill this pathogen.

Examples 1 to 6 additionally show, that chlorate addition reduced the lethal dose of the antibiotic ceftazidime by >100-fold, highlighting chlorate's capacity to potentiate antibiotic treatment.

Examples 7 to 10 illustrates an exemplary model to identify and select timing of administration of chlorate to a biological environment in accordance with embodiments of the present disclosure. The enclosed Examples 11 and 12 illustrates exemplary oxic and anoxic portions of exemplary biological environment together with biomarkers for the related identification as well as exemplary methods and systems for the related detection. The enclosed Examples 13 to 15 illustrates exemplary features and embodiments of the methods, systems for timed and/or targeted administration of chlorate to treat a bacterial infection in accordance with the disclosure, as understood by a skilled person. Examples 1 to 34 show exemplary embodiments of the methods and systems and related devices matrices and composition herein described which are expected to be effective in accordance with the indications of the present disclosure.

The exemplary features and embodiments of the methods, systems for timed and/or targeted administration of chlorate to treat a bacterial infection illustrated in the following examples will provide guidance for additional embodiments of methods, systems for timed and/or targeted administration of chlorate as well as for related matrices, compounds, compositions and implants according to the description and, together with the summary and description sections of the present disclosure serve to explain the principles and implementations of the present disclosure.

The matrices, compositions, compounds, methods and systems herein described are further illustrated in the following Examples showing exemplary protocols applying methods and systems of the disclosure in connection which are provided by way of illustration and are not intended to be limiting.

The following Materials and methods were used in connection with the experiments discussed in Examples 1 to 6.

Bacterial strains and growth conditions Strains used in this study include WT Pseudomonas aeruginosa UCBPP-PA14 and an isogenic strain with a markerless deletion of the narGHJI genes (referred to as Anar) [32] All strains were grown in Luria Broth (Miller's LB Broth) (Research Products International) and supplemented with 40 mM potassium nitrate (KNO₃; Sigma Aldrich) where specified to stimulate Nar activity. Oxic cultures were incubated at 37° C. with shaking at 250 rpm. Hypoxic cultures were incubated at 37° C. under static conditions. Anoxic cultures were incubated statically at 37° C. in an anaerobic glove box with a 95% N₂ and 5% H₂ atmosphere.

Antibiotic treatment assays_Overnight cultures of P. aeruginosa were grown hypoxically for 24 hours in LB with 40 mM potassium nitrate. Overnight cultures were pelleted and resuspended in LB without nitrate to an OD₅₀₀ of 2. Next, 180 μl of resuspended culture was added to the well of a 96-well plate (Genesee Scientific) along with 20 μl of treatment (total volume per well=200 μl). The 20 μl treatment volume consisted of 20 μl of sterile water for control conditions, 10 μl of sterile water and 10 μl of drug solution for single drug conditions, and 10 μl each of two different drug solutions for drug combination conditions. Cultures were incubated with or without drug(s) for 24 hours at 37° C. under oxic, hypoxic, or anoxic conditions before plating for viable plate counts to determine percent survival. To limit evaporation, cultures exposed to drugs under hypoxic or anoxic conditions were incubated in humidified chambers. Under oxic conditions, evaporation was prevented by sealing the 96-well plate with micropore tape and filling empty wells with 200 μl of sterile water.

The following drug stock solutions (made in water) were added to wells of a 96-well plate in antibiotic treatment assays: 10 μl of 200 mM sodium chlorate (final=10 mM), 10 μl of 625 μg/mL tobramycin (final=31.25 μg/mL), 10 μl of 20 μg/mL ciprofloxacin (final=1 μg/mL), 10 μl of 300 μg/mL colistin (final=15 μg/mL), 10 μl of 200 μg/mL ceftazidime (final=10 μg/mL). Additional experiments were conducted using a range of ceftazidime concentrations that were achieved by adding 10 μl of a 20, 2000, or 20000 μg/mL stock solution to wells of a 96-well plate.

Viable plate counts for quantifying percent survival. Viable plate counts were determined for untreated or drug-treated samples by serially diluting samples in phosphate-buffered saline (PBS). Six 1:10 serial dilutions were made in PBS, and 10 μl of each dilution and of the undiluted culture were plated onto LB agar, allowing for viability quantification across 7 orders of magnitude. LB plates were incubated for 24 hours at 37° C. and then moved to the bench top to incubate for another 24 hours at room temperature to allow for the growth of slow-growing colonies. Colonies were counted to calculate colony forming units (CFU) per mL for each sample. Percent survival was determined by dividing the CFU/mL value of each treated sample by the CFU/mL value of a control sample and multiplying by 100; the control sample value was the average of triplicate CFU/mL values of the culture at t=0 (i.e. after washing and resuspending cultures to OD₅₀₀=2 and just prior to the 24 hour drug incubation).

### Example 1: Chlorate and Antibiotic Efficacy Varies with the Oxygen Level and Chlorate Administration can Overcome Hypoxia-Associated Antibiotic Treatment Failure

Bacterial pathogens regularly encounter oxygen (O₂) limitation at sites of infection in the human body. Many pathogens exhibit tolerance to antibiotics under low O₂ conditions, spotlighting the need for new therapeutics that are effective in hypoxic/anoxic environments. Nitrate respiration is a widespread anaerobic bacterial metabolism that many pathogens employ in O₂-limited host environments, marking it as an excellent drug target. Chlorate is a nitrate analog that hijacks nitrate respiration to kill the opportunistic pathogen, Pseudomonas aeruginosa. Chlorate acts as a prodrug: nitrate reductase (Nar) binds chlorate and reduces it to generate chlorite, the latter of which is a toxic oxidizing agent. Chlorate treatment is most toxic to P. aeruginosa under anoxic conditions, but nontoxic under hypoxic or oxic conditions. We also show that several classes of antibiotics display limited toxicity against hypoxic cultures of P. aeruginosa, where the pathogen exhibits antibiotic tolerance or resistance. Excitingly, chlorate interacts synergistically with all tested classes of antibiotics to eradicate hypoxic P. aeruginosa populations.

Thus, while neither chlorate or antibiotics alone efficiently kill O₂-limited P. aeruginosa, combined chlorate-antibiotic treatment overcomes both antibiotic tolerance and resistance to kill this pathogen. Chlorate addition reduced the lethal dose of the antibiotic ceftazidime by >100-fold, highlighting chlorate's capacity to potentiate antibiotic treatment.

Unlike chlorate, most antibiotics do not synergize with different classes of drugs, with the exception of colistin. Given that combination therapy is a promising strategy for combatting antibiotic treatment failure, future studies should continue exploring chlorate's therapeutic potential, including by examining mechanisms of chlorate-antibiotic synergy.

Bacterial pathogens routinely encounter hypoxic microenvironments within the human body. In healthy adults, oxygen (O₂) concentrations range from 1-10% across different tissues and concentrations can be even lower within the gastrointestinal or urinary tract [181]. [182]. [183]) O₂ tensions drop below physiologically normal levels when rates of O₂ delivery and/or consumption are altered, which happens in a variety of circumstances [184] [185]. [186]) During infection, O₂ concentrations are locally depleted by both host cells and microbes through aerobic respiration, and immune cells rapidly consume O₂ through processes like the respiratory burst [187].[188]

Additionally, within the host environment, pathogens typically grow adhered to one another as multicellular aggregate biofilms [189] [190]. These high-cell density aggregates generate steep O₂ gradients, where biofilm-interior populations experience hypoxia/anoxia due to rapid O₂ consumption by biofilm-exterior populations [191].

Because pathogens frequently inhabit hypoxic microenvironments in the host, there is a need for antimicrobial therapies that are highly effective under hypoxia. However, pathogens can exhibit tolerance to conventional antibiotics under hypoxic/anoxic conditions. [192]. [25].

Many antibiotics are less effective at killing slow- or non-growing bacteria, and O₂ availability is a key determinant of growth rate for many pathogenic facultative anaerobes [194]. The relationship between environmental hypoxia and antibiotic treatment failure is thought to underpin different types of recalcitrant infections. In chronic wounds, which affect ˜2% of the U.S. population [195] tissue hypoxia stems from insufficient blood supply and local O₂ consumption by microbes and overactive immune cells. [186]

Chronic wounds cannot heal so long as there is an active infection, yet antibiotic treatments often fail to resolve wound infections, which can ultimately lead to further complications like limb amputation [196] [197], [198], [199], [200].

The airways of people with cystic fibrosis (CF) are coated with a thick mucus that is largely hypoxic/anoxic due to immune cell activity [187] [201]. Pathogens grow slowly in the CF airway environment [202]. [203]. [204] [205]. which likely contributes to the decades-long persistence of CF lung infections despite aggressive antibiotic regimens [205].

Also biofilms are a hallmark of recalcitrant infections, and pathogens are known to form biofilms in a variety of contexts, including in both chronic wound tissue and CF sputum [206]. [207]. [208] [209]

Biofilms are notoriously tolerant to high antibiotic concentrations because they harbor slow-growing, O₂-limited populations. [191]). [210] which use nitrate respiration as physiological process under hypoxic and anoxic conditions.

During nitrate respiration, nitrate reductase reduces nitrate to nitrite. Although there are several types of nitrate reductases, only the Nar enzyme directly contributes to energy conservation by coupling nitrate reduction to the formation of a proton motive force. [211]

Low but appreciable nitrate concentrations (˜100-400 μM) are available at infection sites. [212] [213]. [214]. as a byproduct of inflammation: reactive oxygen and reactive nitrogen species generated by immune cells react nonenzymatically to form nitrate [215].

There is also strong, often direct evidence that Nar-mediated nitrate respiration supports pathogen survival or growth in the host. Enteric pathogens, including Salmonella enterica and Escherichia coli, respire host-derived nitrate to boost their growth in the inflamed gut. [216][217]. [218]. [219]. In Mycobacterium spp., nitrate respiration supports persistence in different host models, and there is evidence that this nitrate is generated as a byproduct of immune cell activity [220]. [221] [222]. Brucella suis also appears to use nitrate respiration to replicate in macrophages and hypoxic environments [223] [224] and Burkholderia spp. were shown to upregulate nar within the host environment [225]. and when grown as a biofilm. [226]. Additionally that the opportunistic pathogen, Pseudomonas aeruginosa, requires nitrate respiration to cause persistent chronic wound infections [227].

Chlorate (ClO₃⁻) is a nitrate (NO₃⁻) analog that acts as a prodrug: chlorate itself is relatively nontoxic, but Nar can bind and reduce chlorate to generate chlorite (ClO₂⁻), which is a toxic oxidizing agent [228] [229] [230]. [231] [228](FIG. 1A). Because mammals lack Nar, it is unsurprising that chlorate shows low toxicity against mammals [230]. with an estimated lethal oral dose of 20-35 grams for humans [232]. [233]

It was previously shown that chlorate kills O₂-limited, antibiotic-tolerant populations of P. aeruginosa biofilms via Nar activity [32].

In addition to those in vitro studies, it has been recently found that topical chlorate treatment supports the healing of P. aeruginosa-infected chronic wounds using a diabetic mouse model [227].

Experiments reported herein show that chlorate administration can overcome hypoxia-associated antibiotic treatment failure, and interfere with Nitrate a widespread form of anaerobic energy metabolism that supports the growth or survival of pathogens in hypoxic host environments [234]. [235]

Experiments reported herein also show chlorate's remarkable ability to synergize with various classes of antibiotics to eradicate hypoxic, antibiotic recalcitrant populations of P. aeruginosa. In one case, the addition of chlorate substantially reduced the effective dose of an antibiotic by >100-fold.

Experiments reported herein further tested the toxicity of different antibiotic-antibiotic combinations against hypoxic P. aeruginosa, finding that most antibiotics do not exhibit synergistic interactions across multiple drug classes. Our results demonstrate that combined chlorate-antibiotic treatment holds promise for combatting antibiotic treatment failure in hypoxic host environments.

### Example 2 P. aeruginosa Exhibits Antibiotic Recalcitrance Under Hypoxic Conditions

Given the prevalence of hypoxia in host environments, the relationship between O₂ availability and drug efficacy in P. aeruginosa was explored. Prior work from ourselves and others has shown that some antibiotics are less effective at killing pathogens under O2-limited, slow growth conditions [194, 210]. [32].

The drug efficacy against P. aeruginosa cultures under a range of O₂ tensions was investigated. To achieve oxic, hypoxic, or anoxic conditions, overnight P. aeruginosa cultures were resuspended in fresh LB medium at a high cell density (OD₅₀₀=2). Cultures were then treated with or without drug(s) and incubated with vigorous shaking (oxic conditions), statically (hypoxic conditions), or in an anaerobic glove box (anoxic conditions) for 24 hours before plating to determine viable cell counts.

The Nar enzyme is most active under low O₂ conditions, where it reduces chlorate to generate toxic chlorite (FIG. 1A). As such, it was unsurprising that chlorate treatment was nontoxic to oxic P. aeruginosa cultures, but resulted in ˜2.5-log killing under anoxic conditions (FIG. 1B). Interestingly, chlorate showed almost no toxicity against hypoxic P. aeruginosa cultures (0.2-log killing, FIG. 1B), suggesting that either Nar was inactive under these conditions or that the cell was able to defend itself from Nar-generated chlorite stress. We found that the antibiotic, tobramycin, was highly lethal to P. aeruginosa under oxic conditions (>6-log killing) but much less toxic to P. aeruginosa under hypoxic or anoxic conditions (˜1.5-log killing; FIG. 1B). These results are consistent with prior work, demonstrating that O₂ limitation causes P. aeruginosa to grow slowly and adopt an antibiotic-tolerant state [210]. [32]

Testing was next performed to verify whether other antibiotics exhibit O₂-dependent toxicities, similar to tobramycin. The related studies were focused on different classes of anti-pseudomonal antibiotics, using concentrations that approximate those measured in patient samples [236] [237].

As before, P. aeruginosa cultures were incubated with or without antibiotic treatment under oxic or hypoxic conditions for 24 hours before plating cells to determine viability. Both tobramycin and ciprofloxacin were substantially less toxic to P. aeruginosa under hypoxic (˜1.5-log killing) compared to oxic (>6-log killing) conditions.

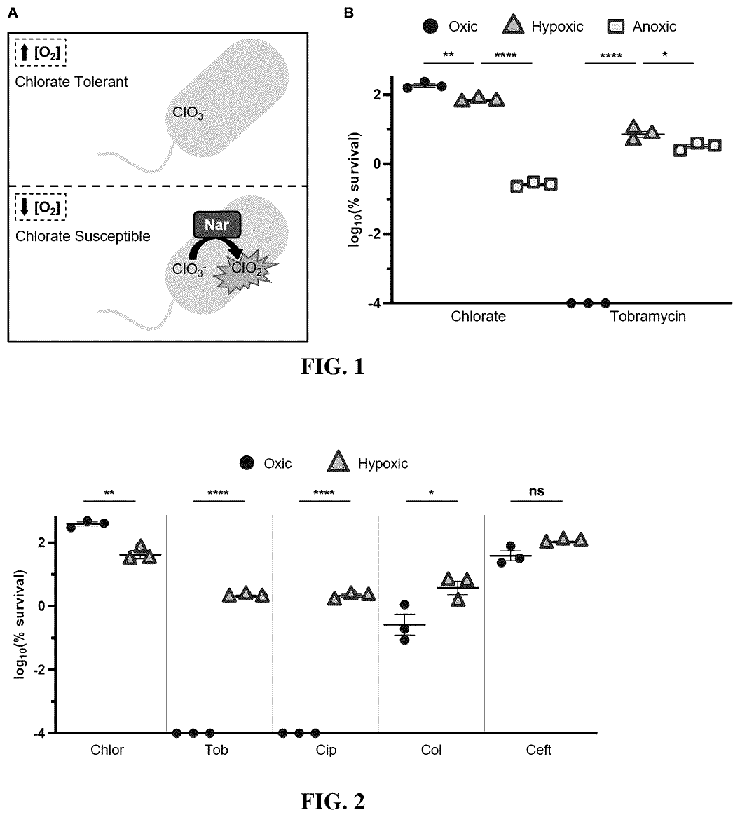

However, colistin was only marginally less effective at killing hypoxic cultures, and P. aeruginosa was completely resistant to ceftazidime under both hypoxic and oxic conditions (FIG. 2). Thus, P. aeruginosa exhibits antibiotic recalcitrance under hypoxic conditions through two mechanisms: hypoxia-induced tolerance (tobramycin, ciprofloxacin) or via O₂-independent genetic resistance (colistin, ceftazidime).

### Example 3 Chlorate Synergizes with Different Classes of Antibiotics to Eliminate Hypoxic P. aeruginosa Populations

Although all tested drug treatments showed modest-to-no toxicity against hypoxic cultures of P. aeruginosa (FIG. 2), it was hypothesized that each drug might impose sufficient stress on the cell such that combined chlorate-antibiotic treatment would interact synergistically to efficiently kill hypoxic P. aeruginosa cultures. Synergy occurs when the efficacy of two drugs in combination is greater than the additive effect of each drug on its own. Indeed, it was found that all chlorate-antibiotic combinations were synergistic (FIG. M3). Despite chlorate-only treatment resulting in little-to-no killing of hypoxic P. aeruginosa cultures, chlorate's addition to each antibiotic treatment increased P. aeruginosa killing by more than 4 orders of magnitude for all tested classes of antibiotic (FIG. M3).

To confirm that chlorate reduction (i.e. chlorite generation) is required for chlorate-antibiotic synergy (as opposed to chlorate itself driving synergy), similar experiments were performed using hypoxic cultures of a P. aeruginosa Anar strain (FIG. 4). As expected, synergy was abolished in the Δnar strain, with antibiotic-only and combined chlorate-antibiotic treatments resulting in the same amount of killing (FIG. 4).

The interaction between chlorate and ceftazidime were further examined, given that P. aeruginosa was resistant to ceftazidime-only treatment (FIG. 2) yet highly susceptible to combined chlorate-ceftazidime treatment (FIG. M3). The initial experiments used ceftazidime at a concentration of 10 μg/mL, but it was found that P. aeruginosa is resistant to much higher concentrations (FIG. 5). By treating hypoxic cultures of P. aeruginosa for 24 hours with increasing ceftazidime concentrations, P. aeruginosa was observed to be resistant to ceftazidime concentrations as a high as 1,000 μg/mL, which is substantially higher than concentrations that can be achieved in the body when this drug is administered to patients[236][237] [238] [239].

Treating hypoxic cultures of P. aeruginosa with combined chlorate-ceftazidime reduced the effective ceftazidime dose by >100-fold, since chlorate-ceftazidime is toxic to P. aeruginosa at a ceftazidime concentration of just 10 μg/mL (3.5-log killing; FIG. 5), which is well below concentrations achieved in patients.

Thus, the data show that chlorate can reduce the effective dose of an antibiotic to which P. aeruginosa is highly resistant.

### Example 4 Chlorate-Antibiotic Synergy is Effective Across a Range of O₂ Availabilities

Given that chlorate-only and some antibiotic-only treatments exhibit O₂-dependent efficacy, it was expected that chlorate-antibiotic synergy might also vary across different O₂ tensions. To test this idea, we treated P. aeruginosa for 24 hours with different drug combinations under oxic, hypoxic, or anoxic conditions.

Chlorate-tobramycin synergy was first tested (FIG. 6). During single drug treatments, these drugs exhibit opposing O₂-dependent toxicities (i.e. chlorate and tobramycin are most effective under anoxic and oxic conditions, respectively). Tobramycin treatment eliminates P. aeruginosa to below our detection limit under oxic conditions, so we cannot determine whether chlorate synergizes with tobramycin under these conditions (FIG. 6A).

However, similar to hypoxic conditions (FIG. 6B), it was found that chlorate-tobramycin treatment also kills anoxic cultures of P. aeruginosa to below our detection limit (FIG. 6C). Since chlorate-only and tobramycin-only treatments both show some toxicity against anoxic cultures of P. aeruginosa, it was less surprising yet promising to find that these drugs maintain synergy under anoxic conditions.

Next, chlorate-ceftazidime synergy was examined under different O₂ tensions. Unlike tobramycin, ceftazidime does not exhibit O₂-dependent efficacy against P. aeruginosa (FIG. 2). Interestingly, chlorate-ceftazidime treatment showed similar levels of synergistic killing across all O₂ tensions (˜3.5- to 4.5-log killing) (FIG. 6). These results further demonstrate the potential for chlorate-antibiotic synergy under anoxic conditions (FIG. 6C).

Unexpectedly, these results show that chlorate treatment induces sufficient cellular stress to synergize with antibiotics even under more oxygenated conditions, when Nar is predicted to be less active. Importantly, we recognize that our use of the term “oxic” to describe the experimental conditions is relative, since even a vigorously shaken high-density culture will experience O₂ limitation; however, we also note that O₂ concentrations are sufficiently different between oxic and hypoxic conditions to drive large changes in antibiotic efficacy (e.g. tobramycin, ciprofloxacin, FIG. 2).

Overall, chlorate's ability to synergize with antibiotics across different O₂ tensions is promising, given that pathogens like P. aeruginosa encounter a range of O₂ concentrations in host environments.

### Example 5 Most Antibiotics do not Synergize with Multiple Classes of Antibiotics

Having observed chlorate's ability to synergize with different classes of antibiotics to overcome antibiotic resistance or tolerance, we next sought to determine whether antibiotics display a similar capacity for synergy.

After treating hypoxic P. aeruginosa cultures for 24 hours with different antibiotic-antibiotic combinations, it was found that most classes do not exhibit widespread synergy (FIG. 7). Like chlorate, colistin synergized with each of the other tested antibiotics (tobramycin, ciprofloxacin, ceftazidime) to reduce P. aeruginosa viability below our detection limit. However, the remaining three antibiotics did not synergize with each other (FIG. 7) Thus, of the five drugs included in this study, two (chlorate, colistin) displayed a capacity for wide-ranging synergistic interactions.

### Example 6 Hijacking Anaerobic Metabolism to Restore Antibiotic Efficacy in Pseudomonas aeruginosa

Many infection sites harbor low O₂ concentrations, which marks anaerobic bacterial metabolisms as promising targets for new drug therapies [234].

Chlorate is toxic to bacteria that respire nitrate via the Nar enzyme, which reduces chlorate to generate highly toxic chlorite (FIG. 1A) [228] [229].

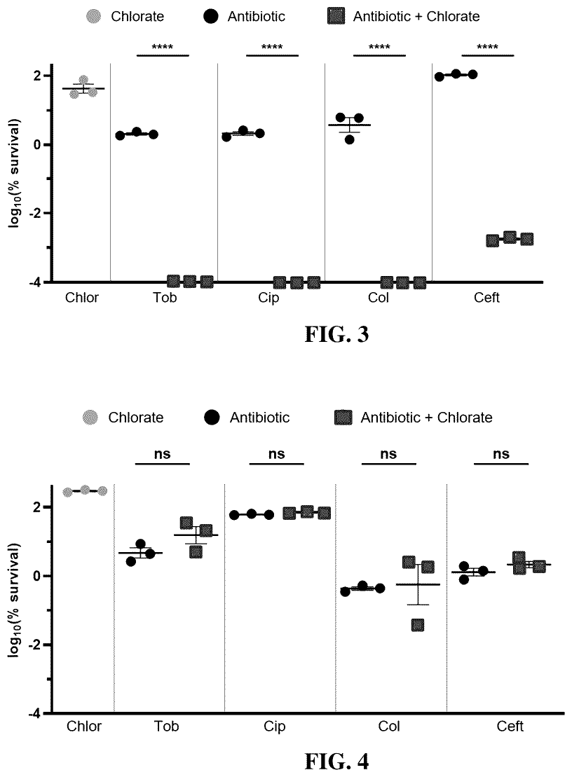

In further support of chlorate's therapeutic potential, we show that chlorate-antibiotic combinations overcome antibiotic-only treatment failure across varying O₂ concentrations (FIG. 6). Chlorate promotes efficient killing of P. aeruginosa by synergizing with different classes of antibiotics: aminoglycosides (tobramycin), fluoroquinolones (ciprofloxacin), beta-lactams (cephalosporins: ceftazidime), and polymyxins (colistin) (FIG. 3). In the case of ceftazidime, chlorate addition reduced the toxic dose by >100-fold (FIG. 5). These findings underscore the potential for chlorate-antibiotic combinations to enhance the efficacy of antibiotic treatments against P. aeruginosa in environments that typically are characterized by antibiotic recalcitrance.

The rise of antibiotic resistance vastly outpaces the discovery rate of new antibiotics [240]. Identifying synergistic drug combinations is considered a key strategy in combatting antibiotic treatment failure [241]. [242].yet numerous challenges limit our ability to leverage this approach. Synergistic drug combinations are typically identified through high-throughput screens, wherein each drug combination is empirically tested for its ability to inhibit bacterial growth [243].

These screens have several drawbacks. First, synergistic interactions are rare, necessitating the use of large screens that are resource intensive. [243]

For example, screening a relatively small drug library of just 1,000 molecules results in about 500,000 pairwise combinations. Second, these screens do not shed light on the underlying biology, so while they might identify synergistic drug combinations, they do little to elucidate mechanisms of drug synergy that could be used recommend other effective drug pairings.

Finally, because drug screens typically use growth inhibition as a readout, they do not work towards combatting antibiotic tolerance. To identify such drugs, screens would need to be conducted under slow- or non-growing conditions and quantify bacterial killing rather than growth inhibition.

Examining mechanisms of drug synergy will enable more effective drug screens and possibly guide rational design of novel, synergistic drug combinations [244]. [245]. [246]

Our current understanding of drug synergy mechanisms is remarkably poor, which represents a troubling knowledge gap in the infectious disease research field; we do not understand why some drug combinations are exceptionally lethal to bacteria, while others are not [243] [245].

Without understanding the underlying mechanisms, we cannot predict drug interactions and must resort to empirically testing each drug combination. The results illustrated herein highlight these challenges. The result show that hypoxic cultures of P. aeruginosa remain viable when treated with high concentrations of ceftazidime or chlorate (FIG. 5). While it might be tempting to conclude that these drugs have no effect on P. aeruginosa, that interpretation is undermined by the observation that combined chlorate-ceftazidime treatment is highly lethal.

More likely, chlorate and ceftazidime each exert stresses on the cell that are not captured by viability measurements. These results highlight the promise of drug synergy, but also underscore its unpredictable nature; chlorate and ceftazidime would have been discarded as useless drugs for killing O₂-limited P. aeruginosa, despite the combination proving quite toxic.

Chlorate is a promising candidate for pursuing future studies to uncover drug synergy mechanisms because it synergizes with several classes of antibiotics to overcome antibiotic resistance and tolerance in P. aeruginosa. In prior work, we showed that chlorate treatment kills P. aeruginosa by causing widespread protein oxidation, being particularly damaging to newly synthesized proteins (69). 69. [247].

Thus, one possible explanation for chlorate's impressive capacity for synergy is that it disrupts the cell's translational response to antibiotic stress. Chlorate toxicity and chlorate-antibiotic synergy is expected in other bacteria, since many pathogenic facultative anaerobes use Nar-mediated nitrate respiration [212, 216, 217]. [218]. [219].[220], [221], [222] [223, 224],[225]. [226].[227]

Although this work was conducted with a limited number of antibiotics, the related findings demonstrate that drugs could be sorted into two groups: drugs that exhibit a broad capacity for synergy (chlorate, colistin) and drugs that do not (tobramycin, ciprofloxacin, ceftazidime) (FIG. 3; FIG. 7). Interestingly, colistin is similar to chlorate in that it is more toxic to P. aeruginosa under O2-limited compared to O₂-replete conditions [248]. [249].

By studying chlorate and colistin, we can identify the cellular stresses that render P. aeruginosa highly susceptible to combination therapy with another antibiotic. If synergy-promoting cellular stresses are determined, more targeted screens can be conducted to identify other drugs that impair specific cellular processes to induce those stresses. Ultimately, defining the stresses that drugs impose individually and in combination will begin to illuminate mechanisms of drug synergy, which will enhance our ability to find or predict powerful new drug combinations in the fight against antibiotic treatment failure.

### Example 7: Measurement of Oxygen Concentration in Cystic Fibrosis Sputum Samples

A total of 48 different sputum samples from 22 different pediatric patients living with Cystic Fibrosis (CF) were profiled for oxygen, and all measurements were performed within 15 min of expectoration.

After collection, sputum samples were immediately transferred to 3-ml cylindrical cavities molded into 20 ml of agar in a truncated 50-ml Falcon tube. The tubes were placed in a heating block to maintain a constant temperature of 37° C. prior to and throughout any measurements.

Two different oxygen microsensors were used: an oxygen microsensor with a tip diameter of 25 μm, and a switchable trace oxygen (STOX) sensor with a tip diameter of 50 μm.

The O₂ microsensor was a Clark-type amperometric electrode with a detection limit of 0.30 μM. This O₂ sensor responds linearly to changes in oxygen concentration; thus, a two-point calibration was performed by immersing the sensor tip in an oxygen-free solution made of sodium hydroxide and sodium ascorbate (both at a final concentration of 0.1 M) to obtain the zero-oxygen reading and in a 100%-air-saturated 0.72% salt solution, which corresponds to 209.3 μM oxygen at the given temperature and salinity level.

The STOX sensor is a specific measuring unit for detecting trace amounts of oxygen with a reported detection limit of 2 nM. The design of the STOX sensor was also based on an amperometric oxygen sensor but was modified by adding a second cathode, which can be switched on and off via a controller unit that is connected between the sensor and the multimeter. This second cathode, or front guard, consumes any traces of oxygen that might enter the electrode, thus enabling reliable measurements of ultralow oxygen concentrations.

In sets of three, sensors were positioned exactly at the air-sputum interface (“depth zero”) by visual inspection using a Leica MZ 9.5 stereomicroscope. Automatic profiling and data acquisition were controlled using SensorTrace Pro 3.1.3 software. Vertical profiles were measured in intervals of 50 or 150 μm through the sputum samples. At each depth, the measuring time was set at 3 s with 2 s between measuring points for all sensors. The time interval between adjacent profiles in the same sample was 60 s. The profiles extended into the agar and began at least 250 μm above the surface of the sputum to determine the boundaries of the sputum sample easily. All samples were profiled, and the sensor was never positioned at a specific spot to log the concentration over time; rather, for data logging of maxima over time, multiple profiles were determined and the maximum concentration from each individual profile was plotted as a function of time since expectoration.

After profile completion, samples were removed from the agar plugs, added to a sterile tube, flash frozen in liquid nitrogen, and stored at −80° C. Time until freezing varied depending on the time course of the profiles recorded and ranged from 30 min to 25 h.

A characteristic oxygen profile was observed using the Clark-type amperometric microelectrode (detection limit of 0.30 μM) for all samples. This profile consisted of three characteristic regions that can be referred to as the “oxic zone” near the top of the air-sputum interface, followed by a steep oxycline below this interface leading through the “hypoxic zone” into the “anoxic zone”. The relative depths of these zones can vary between different bacterial infections, wounds and tissues but are characterized generally by the oxygen concentrations in each zone and their characteristic bacterial respiration mode. In the oxic zone, oxygen concentration is sufficient for normal respiration to occur (for example, at oxygen levels of 200 uM or above); the hypoxic zone has limited oxygen availability and bacterial respiration begins to switch to anaerobic mode (for example, at oxygen levels from 100-10 uM, or from 85-65 uM (7-9% O₂)). Below this, effectively zero O₂ availability in the anoxic zone is found, as confirmed by using the STOX sensor, which can detect ultralow (as low as 2 nM) oxygen concentrations (FIG. 8 Panel A). Six samples were profiled with the STOX sensor, 3 of which were also profiled with the standard O₂ microelectrode. Since negligible O₂ levels were detected in all STOX electrode profiles, all subsequent profiles were profiled with the standard O₂ sensor only.

FIG. 8 Panel B displays the oxycline for 5 of the smaller samples by volume (<1,000 mm³), while FIG. 8 Panel C displays the oxycline for 5 of the samples with larger volumes (>1,000 mm³). The average volumetric fraction of all samples containing detectable oxygen was ˜30%.

Oxygen profiles measured in the same sputum sample at different starting positions and different time intervals, from 6 min to 4 h after collection, demonstrated similar patterns with very little variation over time in oxygen penetration depth. This likely reflects a steady state between oxygen diffusion into the sputum sample and consumption.

### Example 8: Measurement of Electrochemical Redox Potential in Cystic Fibrosis Sputum Samples

The electrochemical oxidation-reduction potential (ORP) of the sputum samples was measured by profiling 28 samples with a redox microelectrode in tandem with the oxygen sensor.

The redox electrode was used together with an Ag/AgCl reference electrode to establish the redox potential, and the values are given relative to the standard hydrogen electrode (SHE) and were determined by measuring the offset of the reference electrode in saturated quinhydrone buffer solutions (pH 4 and pH 7) with known redox potentials. The electrode has a detection limit of 0.10 mV. All calibrations were done at the experimental temperature of 37° C. and at 0.72% salinity. Microsensors were mounted on a motorized micromanipulator in a custom-made probe holder, allowing three sensors to be used simultaneously. The reference electrode for redox measurements was mounted separately in a free-standing ring stand and remained in one place in the sample throughout the measurements that was not controlled by the motorized micromanipulator. All microsensors and related equipment were purchased from Unisense A/S, Arhus, Denmark.

As shown in Figure. 9, the ORP profiles could be separated into two groups: one comprising 17 samples with a positive redox potential range (16 mV to 355 mV), representing an oxidized, albeit anoxic, microenvironment (FIG. 9 Panel A), and another comprising 11 samples displaying a negative redox potential range (−300 mV to −107 mV), indicating a reducing microenvironment (FIG. 9 Panel B).

### Example 9: Modeling Predicts Extensive Sputum Hypoxia and Anoxia In Vivo

A model was prepared to predict sputum oxygen dynamics solely as a function of microbial growth and aerobic respiration. It was assumed that inhaled air is the dominant source of oxygen for mucus. For simplicity, it was assumed microbial cell density is homogenous and host oxygen consumption is neglected; creating a more complex model accounting for host oxygen consumption and reductive metabolite scavenging would lead to steeper oxyclines and lower bacterial densities to account for the oxygen concentrations. Accordingly, this model is conservative and likely overestimates oxygen content in vivo.

Within the airways, oxygen diffuses from the boundary layer at the air-mucus interface into the mucus. Mucus oxygen concentrations can be modeled as a steady-state balance between oxygen diffusion and microbial respiration and as a function of mucus layer thickness, microbial density, and various physical and physiological constraints. The model is formalized as follows:

$$\frac{\partial [ {O}_{2} ]}{\partial t} = {D}_{{O}_{2}} \cdot {∇}^{2} [ {O}_{2} ] - Q \cdot a \qquad ( Eq . 1 )$$

where oxygen [O₂](mol O₂·m⁻³) diffuses with diffusivity DO2 (m²·s⁻¹) depending on the local oxygen concentration gradient and is consumed at rate Q (mol O₂·s⁻¹·g cells⁻¹) by a respiring microbial community with density a (g cells·m⁻³). Assuming O₂ to be the limiting nutrient in the system, the oxygen consumption rate Q can be expressed in terms of a Monod-type growth rate saturation model and a maintenance term:

$$Q = \frac{μ}{{Y}_{{O}_{2}}} + {P}_{{O}_{2}} = \frac{{μ}_{max}}{{Y}_{{O}_{2}}} \cdot \frac{[ {O}_{2} ]}{{k}_{{O}_{2}} + [ {O}_{2} ]} + {P}_{{O}_{2}} \qquad ( Eq . 2 )$$

with a maximum specific growth rate μ max (s⁻¹), yield coefficient YO2 (g cells·mol O₂⁻¹), half-saturation constant kO2 (mol O₂·m⁻³), and the maintenance oxygen requirement PO2 (mol O₂·s⁻¹·g cells⁻¹).

At the steady state

$$( \frac{\partial [ {O}_{2} ]}{\partial t} = 0 ) ,$$

diffusion and respiration are balanced and describe a stable oxygen profile whose shape depends on the parameters discussed and on geometry-specific boundary conditions.

Three relevant geometries to consider in the context of CF lung mucus aggregation are outlined in FIG. 10 (scenarios A, B, and C). By keeping the physical parameters (oxygen diffusivity, mucus thickness) and the physiological parameters (maximal growth rate, oxygen half-saturation) constant but adjusting bacterial densities at realistic values for CF mucus, the shape of mucus in these different contexts can be investigated to show the impact expected for oxygen gradients. Despite the limited (500-μm) depth assumed for these calculations, the model predicts a steep oxycline leading to extensive anoxic zones at higher cell densities, regardless of geometry. By fitting 32 expectorated sputum oxygen microsensor profiles with this spherical mucus aggregate model, average cell densities of 8.3×10⁷ cell/ml for these sputum samples can be calculated. Such fitting is justified because sputum itself is quite hydrated and surrounded by saliva, so the limiting diffusivity is always essentially that of the surrounding water. This suggested that, on average, at these densities, anoxic zones would be expected in clogged airways with mucus layer thickness greater than 1 mm.

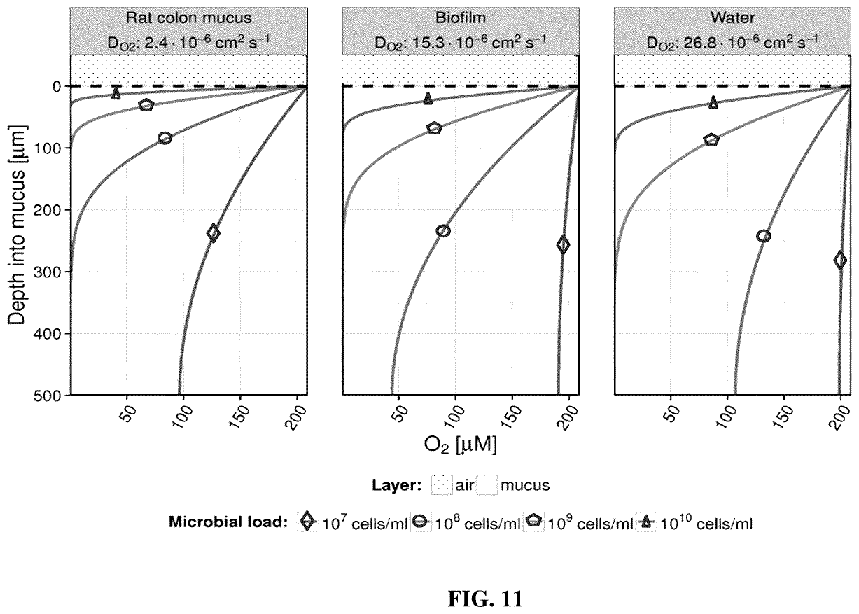

The shape and thickness of the oxycline are also affected by all other physical and physiological parameters. Unlike cell density, however, most other physical and physiological parameters are unlikely to change by orders of magnitude. This reduces their potential effects on oxycline scaling, with oxygen diffusivity likely the second most impactful variable parameter. For comparison to FIG. 10 the potential effect of variable oxygen diffusivities with 3 different literature estimates (for water, biofilm, and rat colon mucus) is shown in FIG. 11. The intermediate biofilm diffusivity value was used for all other modeling results.

### Example 10: Extending this Modeling to 3 Dimensions

This model can be extended to 2 and 3 dimensions by applying appropriate geometric boundaries. For example, considering mucus restrictions in the lung to begin by a thickening of the normal airway surface layer, the two-dimensional (2D) distribution of oxygen in bronchioles to illustrate the spatial distribution. As for the 1D models shown in Figures. 10 and 11 the bacterial density was adjusted from 10⁷ cells/ml to 10¹⁰ cells/ml; in addition, for the 2D models, the mucus layer thickness was adjusted from 50 to 400 μm.

FIG. 19 reveals that airways with a 50-μm-thick layer of mucus would be expected to never become anoxic for the cell densities used in the model. In mucus layers that are 250 μm thick, anoxic conditions would be attainable only with very dense microbial populations (˜10⁹ cells/ml). In 400-μm-thick layers, however, anoxia could be reached at the epithelial cell boundary with population densities as low as 10⁸ cells/ml. A corollary to the oxygen distributions modeled in Figure. 19 Panel A is that oxygen-dependent respiratory growth rates would range from low to zero in many cases (FIG. 19 Panel B).

As before, this simple model neglects oxygen consumption by the host as well as abiotic oxidation by reduced metabolites; thus, its predictions with respect to oxygen abundance are conservative. Accordingly, it is probable that hypoxia and anoxia dynamically occur in vivo and would likely dominate the microenvironments in larger regions of the airways even at relatively low cell densities (e.g., 10⁷ cells/ml). Collectively, these results reinforce the importance of considering mechanisms of adaptation to variable oxygen concentrations within the CF lung.

### Example 11: Detection of mRNA Expression by Hybridization Chain Reaction (HCR) in Pseudomonas aeruginosa Aggregates to Identify Metabolic State

The hybridization chain reaction (HCR) is a technique that provides in situ molecular signal amplification, enabling simultaneous mapping of multiple target RNAs at small spatial scales. HCR is a form of fluorescence in situ hybridization (FISH), where the binding of fluorescent probes to a target RNA of interest results in the binding of additional fluorescent probes, thus amplifying the signal. Probes were designed to visualize the expression of key catabolic genes in Pseudomonas aeruginosa aggregates. Specifically, these were designed to identify the presence of dissimilatory nitrate reductase (narG), terminal oxidase (ccoN1), nitrite reductase (nirS), nitrous oxide reductase (nosZ), and acetate kinase (ackA) genes.

P. aeruginosa strains UCBPP-PA14 (WT) and isogenic ΔnarGHJI, ΔccoN1, ΔackA, ΔnirS, and ΔnosZ were routinely grown in 5 mL lysogeny broth (LB) supplemented with 40 mM KNO₃ with shaking at 37° C. Aerobic cultures were incubated at 37° C. with shaking at 250 rpm unless described otherwise. Anaerobic cultures were incubated in an anaerobic chamber at 37° C. without shaking.

WT and deletion mutant cultures were grown overnight from freezer stocks in LB broth supplemented with 40 mM KNO₃. 1 mL of culture was pelleted, and cells were fixed by resuspending in 1 mL of 4% paraformaldehyde in phosphate-buffered saline (PBS), pH 7.2, and incubating resuspensions at 25° C. for 90 min. After fixing, cells were pelleted, washed with PBS, resuspended in 1 mL of a 1:1 ethanol/PBS solution, and stored at −20° C. until HCR 3.0 experiments were performed.

Prior to performing HCR, cell suspensions were washed in PBS and treated with 1 mg/mL lysozyme (catalog number L6876; Sigma) in 10 mM Tris-HCl (pH 7.6) for 1 h at 37° C. with shaking. After these treatments, cells were washed with PBS and resuspended in 1 mL of a 1:1 ethanol/PBS solution. Cell solutions were suspended in hybridization buffer for 1 h at 37° C. Probe solutions were prepared containing 5 nM appropriate HCR v3.0 initiator odd and even probe pairs in filter-sterilized hybridization buffer. 20 L of probe solutions (Integrated DNA Technologies) was added to each cell solution. The target sequences are included in Table 2. Solutions were hybridized overnight at 37° C. and then washed twice with 200 L probe wash buffer at 37° C. Cells were then resuspended in 40 L amplification buffer and preamplified for 30 min, while fluorescent hairpin probes (Molecular Technologies) were thawed, each denatured in separate tubes in a thermal cycler for 90 s at 95° C., and allowed to cool for at least 30 min at 25° C. in the dark.

Hairpins with Alexa Fluor 488 were used for the rRNA probes, and hairpins with Alexa Fluor 647 were used for the mRNA probes. The B1 initiator/hairpin system from Molecular Technologies was used for rRNA, while B2 was used for nosZ and ccoN1 mRNA, B3 was used for narG mRNA, and B4 was used for nirS and ackA mRNA. Twenty-five microliters of each hairpin were suspended in 20 L of amplification buffer and added to the cell solutions and incubated overnight in the dark at 25° C. The cells were then washed three times in 200 L 5× saline sodium citrate buffer with 0.1% Tween 20 (5×SSCT) before being resuspended in 20 μL 5×SSCT and spotted onto a microscope slide (catalog number 3039-002; Thermo Fisher) and allowed to dry overnight in the dark at 4° C.

Finally, slides were washed with ice-cold ˜0° C. ultrapurified water and then with −20° C. ethanol, and slides were allowed to air dry at 25° C. in the dark. Each dried cell spot was then covered with 2.5 mL ProLong Gold antifade mountant (catalog number P36930; Thermo Fisher), and slides were incubated for 48 h in the dark at 25° C. before being stored and imaged using a Nikon Eclipse Ti2 fluorescence microscope at ×40 magnification with 50% laser power and 1 s exposure for the fluorescent channels. Image analysis was performed using Fiji. Phase images were thresholded to produce a mask and detect cells, the marked cells were thresholded by rRNA signal to eliminate nonbacterial debris from the data set, and then, mean intensity was determined for each labeled cell. Quantitative graphs were generated using Bokeh. Hybridization, probe wash, and amplification buffers were acquired from Molecular Technologies (Caltech, Pasadena, CA).

Aerobic, denitrifying, and fermentative metabolisms are all relevant to bacterial populations in chronic infection environments. The expression levels of cco1, narG, nirS, nosZ, and ackA mRNA molecules are useful to investigate the spatiometabolic patterning of catabolic gene expression within and across P. aeruginosa aggregate biofilm populations. In P. aeruginosa, the primary respiratory oxidase under highly aerobic conditions is cbb₃-1, encoded by cco1. cco1 is constitutively expressed when oxygen is replete but slightly downregulated in the stationary phase. Denitrification is catalyzed by a series of reductases, in particular, nar, nir and nos. Where nitrate is available and oxygen is limited, the nitrate reductase gene, nar, is upregulated. The nitrite reductase gene, nir, is highly upregulated under anoxic conditions and has higher differential expression from oxic to anoxic conditions than nar. The nitrous oxide reductase, nos, is upregulated in response to nitric oxide availability. In contrast, the acetate kinase gene, ackA, is responsible for ATP synthesis during pyruvate- and phenazine-mediated glucose fermentation.

To validate the probes, their specificity was tested using wild-type (WT) and mutant strains in which the target gene had been cleanly deleted. Correspondingly, the narG, nirS, nosZ, ccoN1 and ackA probes showed an average mean intensity per cell or aggregate that was an order of magnitude higher in the WT than the deletion strains, indicating that the probe sets are indeed specific for each target mRNA (FIG. 12).

### Example 12: Detection of mRNA from Metabolic Genes Expressed in Ion Patterns within Agar Block Biofilm Assays (ABBAs)

Using these HCR probes, gene expression across oxygen gradients in aggregate populations grown using the agar block biofilm assay (ABBA) was quantified.

Two sequential overnight cultures of wild-type P. aeruginosa PA14 were grown in the same liquid media as the final ABBAs, shaking aerobically at 37° C., and then diluted to an OD₅₀₀ of 0.001 in molten LB supplemented with nitrate, depending on experimental conditions, and 0.5% noble agar. The agar suspension was transferred to chambered coverglass slides and allowed to cool for 10 min before being transferred to a humidified chamber and incubated at 37° C. for 12 h. After 12 h, blocks were carefully removed from their chambers using a sterilized metal spatula into prechilled 1 mL of 4% paraformaldehyde in PBS with ProtectRNA and incubated for 24 h at 4° C.

Blocks were washed three times for 30 min each in 1 mL PBS, incubated for 1 h in 1 mg/mL lysozyme in 10 mM Tris-HCl at 37° C. to lyse cell walls, and then washed again three times for 30 min each in 1 mL PBS and stored in 1 mL PBS. Blocks were checked on a microscope for proper growth patterning (large aggregates at the top and smaller ones in the bottom for WT in LB plus 40 mM nitrate) prior to continuing. For each condition and each gene target, three biological replicates were performed in addition to one blank (no cell inoculation) per condition.

Oxygen profiles were determined from ABBA samples grown as described above. ABBAs were suspended in a sand bath at 37° C. during oxygen measurement. The oxygen probe was a Clark-type amperometric electrode with a tip diameter of 25 μm, as described above. The probe was connected to a high-sensitivity picoampere amplifier in a multimeter (Unisense, Denmark). The probe was calibrated using an anoxic solution of 0.1 M NaOH and 0.1 M sodium ascorbate at 37° C. and an air-saturated solution of LB medium at 37° C. Depth was measured from the air-agar interface, defined as the last depth before the oxygen values diverged from atmospheric levels and labeled as 0 m. The probe was positioned 75 μm or more away from the air-agar interface in a motorized micromanipulator. Measurements were taken using SensorTrace Pro 3.1.3 software at 25 μm steps for 1,000 m, with 3 measurements at each step and 4-s increments between measurements. The oxygen profile data are averaged from 8 biological replicates and were reported for 500 μm of depth using seaborn.

For each sample, 500 μL of hybridization buffer was mixed with 1 μL even and odd HCR initiator probes (2 μM stock) for 4 nM ultimate concentration. Each block was incubated in the buffer overnight at 37° C. and then washed three times for 2 h in the 1 mL prewarmed wash buffer at 37° C. Each sample was then combined with 250 μL of preheated amplification buffer and 5 μL snap-cooled fluorescent hairpins (3 μM stock) for 60 nM final concentration. Samples were incubated overnight in the dark at room temperature, washed for 6 h in 1 mL 5×SSCT at room temperature in the dark, and then transferred to 500 μL PBS and stored at 4° C. in the dark until imaging by using confocal fluorescence microscopy. Imaging was performed on a Zeiss LSM 800 in the Caltech Bioimaging Facility. Agar blocks were mounted on a glass-bottomed dish with the original air-agar interface oriented toward the glass bottom. Both the Alexa Fluor 488 rRNA and Alexa Fluor 647 mRNA channels were imaged simultaneously, with a laser power of 0.2% and a gain of 650 for the 488 channel and a laser power of 2% and a gain of 750 for the 647 channel.

At least four image fields were imaged per sample using a 10× objective with 2× zoom for a final image field of 319.45 by 319.45 μm. For each image field, a 100-slice z-stack was imaged with an interslice distance of 6.24 m. Images were framed such that the surface, which appears visible as a plane of rRNA signal due to a lawn of cells growing on the agar surface, was within 4 slices of the top of the image. Quantification of fluorescence signal per aggregate was performed using Imaris v9.5.1 (Imaris [RRID SCR_007370]). A batch pipeline was created to segment aggregates.

The 488 signal intensity of each z-plane was normalized, and then, aggregates were segmented based on the thresholder 488 signal, with the threshold chosen manually per imaging date. The normalized signal was only used for the purpose of segmentation, and the mean signal intensity values reported here are based on the raw signal rather than the normalized signal. The mean 488 and 647 mRNA signal intensities, as well as aggregate size, were calculated and exported per aggregate. Depth was defined as the distance from the top of the image, and images were cropped for analysis such that the surface was at the top slice using native Imaris functions. Quantitative graphs were generated by using seaborn and Bokeh. Raw images were displayed by using Fiji.

Exemplary detection gene expression across oxygen gradients in aggregate populations grown using ABBA is illustrated in FIG. 13: showing catabolic Genes with Distinct Patterns Across Intra- and FIG. 14: showing ABBA images Replicate 2: 3D Micrographs of Probe Signal in LB+40 mM Nitrate ABBAs.

In an ABBA, an oxygen gradient develops, and the oxygen minimum migrates upwards toward the air-agar interface over the course of the incubation as cell density increases, and thus, oxygen availability decreases (FIG. 15 Panel A). The oxidase ccoN1 was maximally expressed in the oxic region (0 to 50 μm from the air-agar interface at the top of the ABBA blocks) and steeply decreased in expression deeper into the block along the oxygen gradient, which is the same pattern shown by the 16S rRNA signal (FIG. 15 Panel B). In the larger aggregates closer to the surface, the signal for ccoN1 and 16S rRNA was higher along the aggregate exterior than in the centers, while the signal for the ackA gene was highly expressed in the interior (FIG. 15 Panel B and 15 Panel C). An elevated signal for the denitrification genes was not detected in this region.

In the region below the oxic portion, rRNA, ccoN1, and ackA expression was reduced, and expression of the denitrification genes was elevated, correlating with low oxygen concentrations that decreased with depth across this region (FIG. 15 panels A-C). All three denitrification genes were expressed at their maximal levels in aggregates in this region. The nitrate reductase narG was upregulated under hypoxic conditions in the 40-mM nitrate samples, peaking in average mean intensity per aggregate at a depth of 100 to 150 m from the air-agar interface (FIG. 15 Panel B and 15 Panel C). narG signal was restricted to the interior of aggregates, where oxygen availability is expected to be reduced compared to cells on the aggregate exterior (FIG. 15 Panel B). The nitrite reductase, nirS, was upregulated under hypoxic and anoxic conditions, peaking between 100 and 200 m from the surface. The nitrous oxide reductase nosZ was expressed in cells in the cores of some aggregates in the hypoxic and anoxic regions but was not uniformly expressed by aggregates at any depth (FIG. 15 Panel C). Oxygen levels reached their minimal levels between 150 and 250 m from the air-agar interface (FIG. 15 Panel A).

Oxygen levels were uniformly low below 250 m from the air-agar interface. All genes measured showed reduced expression with depth across this anoxic region, likely due to oxidant limitation, although nirS expression was elevated in the cores of aggregates at all depths. nirS signal was restricted to the interior of aggregates in the hypoxic zone but was expressed across entire aggregates further into the anoxic zone (FIG. 15 Panel B). An appreciable narG signal was not detected in the deepest anoxic region (FIG. 15 Panel C).

These findings confirm that in P. aeruginosa, at a single time point, maximal narG expression occurs closer to the air-agar interface than maximal nirS and nosZ expression, as oxygen limitation and nitrate limitation increase with depth. Aerobic respiration (ccoN1) showed peak expression under oxic conditions, whereas fermentation (ackA) showed peak expression in the anoxic cores of high metabolic activity aggregates near the air-agar interface. Denitrification genes narG, nirS, and nosZ showed peak expression in hypoxic and anoxic regions, although nirS expression remained at peak levels deeper into anoxic environments than other denitrification genes.

### Example 13: In Vitro Drug Treatment Assays

All experiments used Luria-Bertani (LB; Difco) as the growth medium, supplemented with KNO₃ as specified. Aerobic liquid cultures were incubated at 37° C. with shaking at 250 RPM. Anaerobic work was conducted in an anaerobic glove box with a 95% N₂-5% H₂ atmosphere, and anaerobic cultures were incubated at 37° C. For these studies, a P. aeruginosa strain referred to as RPA (Riverside PA) was used that was previously isolated from a chronic wound mouse model. Additionally, these studies used a specially constructed an RPA strain lacking genes for the Nar enzyme (AnarGHJI, hereby referred to as Anar), with the mutants confirmed through PCR fragment size and their inability to grow anaerobically with nitrate.

For in vitro planktonic drug treatment studies, overnight RPA cultures were grown aerobically in LB broth supplemented with 40 mM KNO₃. Overnight cultures were pelleted, pellets were washed once with LB, and then resuspended to OD500=2 in either LB, LB with 10 μg/mL ciprofloxacin, LB with 10 mM sodium chlorate, or LB with 10 μg/mL ciprofloxacin and 10 mM sodium chlorate. Cell resuspensions were added to 96-well plates (150 μL per well), and one 96-well plate was incubated statically (not shaking) at 37° C. at ambient oxygen, while a duplicate 96-well plate was incubated statically at 37° C. under anoxia (anaerobic glove box). Drug treatments were incubated for 24 h, after which cultures were serially diluted for viable cell counts. Viable cell counts were performed by serially diluting samples in phosphate-buffered saline (PBS). Dilutions spanning 7 orders of magnitude were plated on LB agar plates as 10 μL drips. After incubation at 37° C., colonies were counted to quantify colony-forming units (CFU) per milliliter for each culture. Percent survival was calculated for each condition by dividing CFU/mL values from a drug treated sample by the average CFU/mL value from control (no drug) samples and multiplying by 100. All viable cell counts were carried out under oxic conditions.

For in vitro biofilm drug studies, agar block biofilm assays (ABBAs) were used. Briefly, to set up ABBA, overnight RPA cultures were diluted to OD500=0.001 into cooled molten agar (44° C.) composed of LB with 5 mM KNO₃ and 1% agar. Before the agar solidified, 500 μL of the cell-agar suspension was added to 2-mL Eppendorf tubes, after which samples were allowed to solidify. Eppendorf tubes were incubated at 37° C. for 24 h to allow cells to grow as aggregate biofilms suspended within the agar medium. After this growth incubation, ABBA samples were washed twice with 200 μL of PBS to remove cell growth that was not suspended within the agar. ABBA samples were then treated with 100 μL of PBS (control), 100 μL of 60 g/mL ciprofloxacin (final concentration=10 μg/mL), 100 μL of 60 mM sodium chlorate (final concentration=10 mM), or 100 μL of a 60 μg/mL ciprofloxacin plus 60 mM sodium chlorate solution (final concentrations=10 μg/mL ciprofloxacin and 10 mM sodium chlorate). Drug treatments were incubated for 24 h at 37° C. To determine viable cell counts, ABBA samples were first homogenized by adding 300 μL of PBS to each sample and homogenizing on speed 3 with a Bio-Gen PRO200 Homogenizer (PRO Scientific). Viable cell counts and percent survival were calculated for ABBA samples as described above.

It was found that oxic planktonic cultures of RPA are highly susceptible to ciprofloxacin and highly tolerant to chlorate, whereas anoxic planktonic cultures of RPA are highly susceptible to both chlorate and ciprofloxacin (FIG. 15). These results also show that the combined ciprofloxacin and chlorate treatment is more effective in killing planktonic cultures than either drug alone; cell survival is below our detection limit (>6-log killing) for combined treatment under both oxic and anoxic conditions. Similar experiments for RPA aggregate biofilms reveal that RPA biofilms are susceptible to ciprofloxacin but show greater survival than planktonic cells. Combined ciprofloxacin and chlorate treatment also resulted in more killing of bacterial cells in biofilms than either drug alone. Finally, tests of the RPA Anar strain showed that the Anar cultures are highly resistant to chlorate treatment, and the combined ciprofloxacin and chlorate treatment resembles ciprofloxacin-only treatment in the RPA Anar strain. Therefore, chlorate toxicity requires the hypoxically induced Nar enzyme in RPA.

### Example 14: Treatment of RPA Biofilms in db/db−/− Mice Chronic Wounds with Chlorate, Ciprofloxacin, or Both, Leads to Healing of Chronic Wounds

A diabetic mouse model (db/db−/−) of chronic wounds that mimics chronic wounds in humans was used to investigate wound healing. First, it was confirmed that mouse wounds contained RPA biofilms at day 10 post-wounding using the tissue clearing and microbial visualization method, MiPACT-HCR51 (FIG. 17 Panel A). Fluorescence microscopy revealed confluent biofilm growth to thicknesses >50 m at the surface of the wound. Given that the rate of oxygen consumption by P. aeruginosa outpaces its diffusion, it is well established that 10-20 m below the surface of such densely packed biofilms, interior cells experience steep oxygen gradients. RPA growth was also observed deep within the wound tissue (˜1 mm), where direct measurements have shown the environment is hypoxic and/or anoxic.

Wound treatments began at 10 days postwounding when the biofilms were already present, and included 100 μL of either ciprofloxacin alone (200 g/mL, i.e., 20 g total/wound), chlorate alone (10 mM), or combined ciprofloxacin and chlorate treatment. Wounds were treated daily between 10 and 40 days postwounding. Untreated RPA-infected wounds remained opened and contained biofilms until day 40. All treated wounds showed a visual decrease in biofilm formation compared with untreated wounds (FIG. 17 Panel B). By measuring wound area over time, it was determined that the wound area increased during the first 15 days after injury for all mice (both untreated and treated) (FIG. 17 Panel C). However, by day 40 post-injury, untreated wounds maintained their maximum surface area, whereas all treated wounds had significantly decreased in size (FIG. 17 Panel C and 17 Panel D). Therefore, chlorate treatment is as effective as ciprofloxacin at promoting wound healing, and that combined ciprofloxacin and chlorate treatment is as effective as either drug alone.

### Example 15: Treatment with Ciprofloxacin, Chlorate, or Both Promote Healing of Chronic Wounds

The quality of wound healing in treated mice was characterized by performing histological and immunofluorescence analyses on day 40 wounds (FIG. 18 Panel A). Hematoxylin and eosin (HE) staining showed that untreated RPA-infected wounds fail to properly undergo re-epithelialization or granulation tissue formation. Masson's trichrome (MT) staining showed that no collagen formed in the granulation tissue of untreated wounds. By contrast, HE staining of all healed treated wound tissues showed strong re-epithelialization with granulation tissue formation beneath the newly formed epidermis. MT staining showed that collagen fibers were present in the granulation tissue of all treated wounds, suggesting that the quality of the wound tissue is significantly improved when wounds are treated with ciprofloxacin, chlorate, or both (FIG. 18 Panel A).

To distinguish between and visualize the distribution of collagen I and collagen III in the granulation tissue, the wounds were also stained with Picrosirius Red (PSR). PSR staining of untreated wounds showed thick bandings of collagen I (FIG. 18 Panel A, stained red) in the granulation tissue, indicating that the wounds did not close and heal. Healed wounds treated with ciprofloxacin, chlorate, or both showed staining of both collagen I and collagen III (FIG. 18 Panel A, stained red and green, respectively overlap appears as yellow) in the granulation tissue, indicative of healing wounds. Collagen IV immunolabeling was used to show whether basal lamina under the epidermis was present, an important indicator of proper connectivity of the epithelium with the underlying dermis. Collagen IV acts as a physical, chemical, and functional structure between the epidermis and the dermis. Immunofluorescence labeling for collagen IV showed that untreated RPA-infected wounds, which were not re-epithelialized, did not show collagen IV-containing basement membrane even at the margins of the wound (FIG. 18 Panel A). Healed wounds treated with either chlorate, ciprofloxacin, or both formed a basement membrane under the epithelium as is the case in normal healing. Keratin 14/16 is a cytokeratin found in proliferating keratinocytes of the epidermis and is observed in the cytoplasm surrounding the nucleus. Keratin 14/16 was absent in the untreated wounds but present in all treated wounds. The quality of the keratinocytes of the epidermis was observably better in wounds treated with both ciprofloxacin and chlorate (FIG. 18 Panel A).

Blood vessel development during wound healing is another indicator of proper healing. The number of microvessels present in the granulation tissue was quantified by immunolabeling the wound tissue with a-smooth muscle actin (aSMA), a protein that is present in the cells surrounding blood vessels. Untreated RPA-infected wounds showed no aSMA labeling, indicating that these wounds contained few or no blood vessels and confirming that wound healing did not occur properly in these wounds. In contrast, wounds treated with cipro floxacin, chlorate, or both showed microvessels in the granulation tissue (FIG. 18 Panel A). Compared with untreated controls, wounds treated with ciprofloxacin, chlorate, or both, all had significantly higher number of microvessels in the granulation tissue, although there was no statistically significant difference between the treatments (FIG. 18 Panel B).

### Example 16: Detection of Sample Oxygen Concentration Using Electrochemical Methods (Prophetic)

Sample oxygen concentration can be measured using a Clark-type amperometric electrode, the general operation of which is described in en.wikipedia.org/wiki/Clark_electrode. Depending on the sample size and/or sample volume, the electrode can be of macroscopic type (e.g. with tip diameters of 1-10 mm) or can be a microelectrode (e.g. with tip diameter of 10-50 m). For microelectrode measurements the electrode probe can be connected to a high-sensitivity picoampere amplifier in a multimeter (for example, those made by Unisense, Denmark).

The probe can be calibrated using known methods, for example by performing a two-point calibration by immersing the sensor tip in an oxygen-free solution made of sodium hydroxide and sodium ascorbate (both at a final concentration of 0.1 M) to obtain the zero-oxygen reading and in a 100%-air-saturated 0.72% salt solution, which corresponds to 209.3 mM oxygen at the given temperature and salinity level.

A general description of the electrochemical oxygen sensor method is given in Spero M A, Newman D K. 2018. Chlorate specifically targets oxidant starved, antibiotic-tolerant populations of Pseudomonas aeruginosa biofilms. mBio 9:e01400-18. doi.org/10.1128/mBio.01400-18., incorporated herein by reference.

### Example 17: Detection of Sample Redox Potential Using Electrochemical Methods (Prophetic)

Sample redox potential can be measured using a redox electrode together with an appropriate reference electrode, for example the Ag/AgCl (sat. KCl) reference (standard potential +0.197 V vs SHE at 25° C.) or the saturated calomel reference (+0.244 V vs SHE at 25° C.). Depending on the sample size and/or sample volume, the redox electrode can be of macroscopic type (e.g. with tip diameters of 1-10 mm) or can be a microelectrode (e.g. with tip diameter of 10-50 m).

The reference electrode can be a bulk reference electrode or can be of needle-type depending on the size and geometry of the measurement. For microelectrode measurements the electrode probe can be connected to a high-sensitivity picoampere amplifier in a multimeter (for example, those made by Unisense, Denmark). The probe can be calibrated using known methods, for example by performing a two-point calibration by measuring the offset of the reference electrode in saturated quinhydrone buffer solutions (pH 4 and pH 7) with known redox potentials.

A general description of the measurement of sample electrode potential, and of the use of microelectrode arrays, is given in Cowley E, Kopf S, LaRiviere A, et al. Pediatric cystic fibrosis sputum can be chemically dynamic, anoxic, and extremely reduced due to hydrogen sulfide formation. mBio 2015; 6:e00767; doi: 10.1128/mBio.00767-15, incorporated herein by reference.

### Example 18: Detection of Sample Nitrate Concentration Using Electrochemical Methods (Prophetic)

Sample nitrate concentration can be measured using a nitrate-selective electrode. Depending on the sample size and/or sample volume, the electrode can be of macroscopic type (e.g. with tip diameters of 1-10 mm) or can be a microelectrode (e.g. with tip diameter of 10-50 μm). For microelectrode measurements the electrode probe can be connected to a high-sensitivity picoampere amplifier in a multimeter (for example, those made by Unisense, Denmark). The probe can be calibrated using known methods, for example by performing a multi-point calibration by immersing the sensor tip in solutions of KNO₃ in suitable aqueous buffer at different concentrations (e.g., 100 μM, 1 mM, 10 mM KNO₃) and recording the variation in observed electrode potential at those concentrations, anticipating a linear response.

A general description of nitrate-sensitive microelectrodes is given in RUI-GUANG ZHEN, SUSAN J. SMITH, ANTHONY J. MILLER, A Comparison of Nitrate-selective Microelectrodes made with Different Nitrate Sensors and the Measurement of Intracellular Nitrate Activities in Cells of Excised Barley Roots, Journal of Experimental Botany, Volume 43, Issue 2, February 1992, Pages 131-138, doi.org/10.1093/jxb/43.2.131, incorporated herein by reference.

### Example 19: Detection of Sample Nitrate Concentration Using Fluorescence Methods (Prophetic)

Sample nitrate concentration can also be measured using a fluorescence method, for example a FRET sensor such as that described in Yen-Ning Chen et al., In vivo visualization of nitrate dynamics using a genetically encoded fluorescent biosensor.Sci. Adv.8,eabg4915(2022).DOI:10.1126/sciadv.abg4915 and in Chen Y N, Ho C H. Fluorescent Biosensor Imaging of Nitrate in Arabidopsis thaliana. Bio Protoc. 2023 Aug. 20; 13(16):e4743. doi: 10.21769/BioProtoc.4743, both incorporated herein by reference.

### Example 20: Detection of mRNA Expression by Nitrate-Respiring Bacteria in a Test Sample Using Fluorescence Methods (Prophetic)

Wound bacteria can signal their nitrate-respiring activity through the expression of specific genes into the local environment. The corresponding mRNA can be detected in a sample of this environment through the use of a hybridization chain reaction (HCR) procedure with specific fluorescent hairpin probes featuring the appropriate target sequence.

This method is described in detail for narG and other reductases found in P. aeruginosa wild-type PA14 in Livingston etl 2022 [250], incorporated herein by reference.

### Example 21: Detection of mRNA Expression by Nitrate-Respiring Bacteria in a Test Sample Using Electrochemical Methods (Prophetic)

Alternatively, the HCR method outlined in Example 20 can be modified to use specific redox-active hairpins functionalized with the appropriate target sequence, for example hairpins functionalized with methylene blue or with redox-active tyrosines or tryptophan residues. Binding of expressed nar mRNA to these hairpin can be detected by voltametric methods, thus this electrochemical method can be employed to detect the markers of nitrate respiration by nar-containing bacteria. Suitable electrodes can be included as part of a microelectrode array with the oxygen level, redox potential and nitrate concentration sensor electrodes in a suitable device, as described earlier.

### Example 22: Ex-Situ Analysis of Infected Biological Environment, Followed by Treatment (1—Antibiotic Only) (Prophetic)

A small sample of an infected biological environment can be removed by swab, and the sample can be analyzed for oxygen content, redox potential and nitrate concentration using an appropriate electrode setup and electrochemical analyzer (e.g., potentiostat such as those made by Gamry Instruments or Biologic, Inc).

Each measurement can be made using the same instrument and changing out the electrodes between measurement, or can be measured on three separate instruments each appropriately configured, or can be measured simultaneously using three different channels on the same instrument. In the case where an oxygen concentration above 100 μM is detected alongside a redox potential above 200 mV relative SHE and measured to SHE and no nitrate concentration detected, antibiotics are then provided to the infected biological environment in an amount effective to inhibit viability of the bacteria. The antibiotic can be applied as a cream, or a lotion, or in pill or intravenous or intramuscular form.

### Example 23: Ex-Situ Analysis of Infected Biological Environment, Followed by Treatment (2—Antibiotic Plus Chlorate) (Prophetic)

A small sample of an infected biological environment can be removed by swab, and the sample can be analyzed for oxygen content, redox potential and nitrate concentration using an appropriate electrode setup and electrochemical analyzer (e.g., potentiostat such as those made by Gamry Instruments or Biologic, Inc).

Each measurement can be made using the same instrument and changing out the electrodes between measurement, or can be measured on three separate instruments each appropriately configured, or can be measured simultaneously using three different channels on the same instrument.

In the case where an oxygen concentration is below 100 μM is detected alongside a redox potential below 200 mV relative to SHE and a nitrate concentration above 500 μM is detected, both antibiotics and chlorate materials are then provided to the infected biological environment in an amount effective to inhibit viability of the bacteria. The antibiotic can be applied as a cream, or a lotion, or in pill or intravenous or intramuscular form; the chlorate material can be applied as a cream, or a lotion, or in pill or intravenous or intramuscular form.

### Example 24: Ex-Situ Analysis of Infected Biological Environment, Followed by Treatment (3—Nitrate, Followed by Antibiotic Plus Chlorate) (Prophetic)

A small sample of an infected biological environment can be removed by swab, and the sample can be analyzed for oxygen content, redox potential and nitrate concentration using an appropriate electrode setup and electrochemical analyzer (e.g., potentiostat such as those made by Gamry Instruments or Biologic, Inc).

Each measurement can be made using the same instrument and changing out the electrodes between measurement, or can be measured on three separate instruments each appropriately configured, or can be measured simultaneously using three different channels on the same instrument. In the case where an oxygen concentration below 100 μM is detected alongside a redox potential below 200 mV relative to SHE and a nitrate concentration below 500 μM is detected, a nitrate material is administered to the infected biological environment for a nitrate contacting time and in a nitrate amount effective to increase expression of a Nar gene in the Nar-containing bacteria in the infected environment.

Optionally, the expression of the Nar gene after the nitrate contacting time has elapsed can be assessed by the HCR procedure described herein. After this nitrate contacting time, antibiotics, chlorate and nitrate materials are then provided to the infected biological environment in an amount effective to inhibit viability of the bacteria, with the amount of chlorate and nitrate material in a ratio from 4:1 to 10:1.

The antibiotic can be applied as a cream, or a lotion, or in pill or intravenous or intramuscular form; the chlorate material can be applied as a cream, or a lotion, or in pill or intravenous or intramuscular form. Likewise, the nitrate material can be applied as a cream, or a lotion, or in pill or intravenous or intramuscular form.

### Example 25: Ex-Situ Analysis of Infected Biological Environment to Reduce Chronicity of a Wound (1—High Nitrate) (Prophetic)

A small sample of an infected biological environment can be removed by swab, and the sample can be analyzed for nitrate concentration using an appropriate electrode setup and electrochemical analyzer (e.g., potentiostat such as those made by Gamry Instruments or Biologic, Inc).

In the case where a nitrate concentration above 500 μM is detected, both antibiotics and chlorate materials are then provided to the infected biological environment in an amount effective to inhibit viability of the bacteria. The antibiotic can be applied as a cream, or a lotion, or in pill or intravenous or intramuscular form; the chlorate material can be applied as a cream, or a lotion, or in pill or intravenous or intramuscular form.

### Example 26: Ex-Situ Analysis of Infected Biological Environment to Reduce Chronicity of a Wound (2—Low Nitrate) (Prophetic)

A small sample of an infected biological environment can be removed by swab, and the sample can be analyzed for nitrate concentration using an appropriate electrode setup and electrochemical analyzer (e.g., potentiostat such as those made by Gamry Instruments or Biologic, Inc).

In the case where a nitrate concentration between 0 and 500 μM is detected, a nitrate material is administered to the infected biological environment for a nitrate contacting time and in a nitrate amount effective to increase expression of a Nar gene in the Nar-containing bacteria in the infected environment.

Optionally, the expression of the Nar gene after the nitrate contacting time has elapsed can be assessed by the HCR procedure described herein. After this nitrate contacting time, antibiotics, chlorate and nitrate materials are then provided to the infected biological environment in an amount effective to inhibit viability of the bacteria, with the amount of chlorate and nitrate material in a ratio from 4:1 to 10:1.

The antibiotic can be applied as a cream, or a lotion, or in pill or intravenous or intramuscular form; the chlorate material can be applied as a cream, or a lotion, or in pill or intravenous or intramuscular form. Likewise, the nitrate material can be applied as a cream, or a lotion, or in pill or intravenous or intramuscular form.

### Example 27: Ex-Situ Analysis of Infected Biological Environments to Reduce Chronicity of a Wound (3—Fluorescent Nitrate Detection) (Prophetic)

A small sample of an infected biological environment can be removed by swab, and the sample can be analyzed for nitrate concentration using an appropriate fluorescence method with a suitable nitrate-sensitive fluorescent material and fluorometer (e.g., such as those made by Horiba or by Thermo Fisher).

Depending on the detected nitrate concentration (between 0 and 500 μM, and above 500 μM respectively) the correct course of action can be chosen as for the electrochemically-measured samples.

### Example 28: Repeated Ex-Situ Analysis of Infected Biological Environment Followed by Appropriate Treatment (Prophetic)

The examples above can be combined by taking repeated ex-situ analyses of samples of the infected biological area as a function of time over several minutes, or hours, or days, or weeks, or months, or longer depending on the nature of the infection.

Measurement of sample oxygen concentration, redox potential and nitrate concentration by electrochemical methods can be carried out as described, and the appropriate course of action determined from the results, with antibiotics, chlorate and/or nitrate materials being applied to the infected biological area as described, until completion of the treatment of the infected biological environment.

### Example 29: In-Situ Analysis of Infected Biological Environment, Followed by Appropriate Treatment (Prophetic)

An infected biological environment was contacted with a bandage or prosthetic that contained sensing apparatus to configured determine oxygen content, redox potential and nitrate concentration using an appropriate microelectrode array connected to external power source, amplifier and detection instrument (e.g., potentiostat). Measurements of oxygen concentration, redox potential and nitrate concentration were made at defined time points such as every few minutes, every few hours, or hours, or days, or weeks, or months or combinations of these periods. In the case where an oxygen concentration above 100 μM was detected alongside a redox potential above 200 mV relative to SHE and no nitrate concentration detected, antibiotic release was triggered from the bandage or prosthetic to inhibit viability of the bacteria in the infected environment. Alternatively, in the case where an oxygen concentration below 100 μM was detected alongside a redox potential below 200 mV relative to SHE and a nitrate concentration above 500 μM was detected, both antibiotics and chlorate material release was triggered from the bandage or prosthetic to inhibit viability of the bacteria in the infected environment. In a second alternative, in the case where an oxygen concentration below 100 μM was detected alongside a redox potential below 200 mV relative to SHE which can be measured with other electrodes such as Ag/AgCl (sat. KCl) and a nitrate concentration below 500 μM was detected, release of a nitrate material from the bandage or prosthetic was triggered to administer the material to the infected biological environment for a nitrate contacting time and in a nitrate amount effective to increase expression of a Nar gene in the Nar-containing bacteria in the infected environment. After this nitrate contacting time, release of antibiotics, chlorate and nitrate materials from the bandage or prosthetic was triggered to administer each of these materials to the infected biological environment in an amount effective to inhibit viability of the bacteria, with the amount of chlorate and nitrate material in a ratio from 4:1 to 10:1. In situ measurement of oxygen concentration, redox potential and nitrate concentration is then repeated at a defined time point and appropriate treatment is continued until completion of the treatment of the infected biological environment.

### Example 30: Detection of Sample Oxygen Concentration Using Electrochemical Methods (Prophetic)

Sample oxygen concentration can be measured using a Clark-type amperometric electrode, the general operation of which is described in [251]. Depending on the sample size and/or sample volume, the electrode can be of macroscopic type (e.g. with tip diameters of 1-10 mm) or can be a microelectrode (e.g. with tip diameter of 10-50 um). For microelectrode measurements the electrode probe can be connected to a high-sensitivity picoampere amplifier in a multimeter (for example, those made by Unisense, Denmark).

The probe can be calibrated using known methods, for example by performing a two-point calibration by immersing the sensor tip in an oxygen-free solution made of sodium hydroxide and sodium ascorbate (both at a final concentration of 0.1 μM) to obtain the zero-oxygen reading and in a 100%-air-saturated 0.72% salt solution, which corresponds to 209.3 mM oxygen at the given temperature and salinity level.

### Example 131: Detection of Sample Redox Potential Using Electrochemical Methods (Prophetic)

Sample redox potential can be measured using a redox electrode together with an appropriate reference electrode, for example the Ag/AgCl (sat. KCl) reference (standard potential +0.197 V vs SHE at 25° C.) or the saturated calomel reference (+0.244 V vs SHE at 25° C.). Depending on the sample size and/or sample volume, the redox electrode can be of macroscopic type (e.g. with tip diameters of 1-10 mm) or can be a microelectrode (e.g. with tip diameter of 10-50 um).

The reference electrode can be a bulk reference electrode or can be of needle-type depending on the size and geometry of the measurement. For microelectrode measurements the electrode probe can be connected to a high-sensitivity picoampere amplifier in a multimeter (for example, those made by Unisense, Denmark). The probe can be calibrated using known methods, for example by performing a two-point calibration by measuring the offset of the reference electrode in saturated quinhydrone buffer solutions (pH 4 and pH 7) with known redox potentials.

### Example 32: Detection of Sample Nitrate Concentration Using Electrochemical Methods (Prophetic)

Sample nitrate concentration can be measured using a nitrate-selective electrode. Depending on the sample size and/or sample volume, the electrode can be of macroscopic type (e.g. with tip diameters of 1-10 mm) or can be a microelectrode (e.g. with tip diameter of 10-50 um). For microelectrode measurements the electrode probe can be connected to a high-sensitivity picoampere amplifier in a multimeter (for example, those made by Unisense, Denmark). The probe can be calibrated using known methods, for example by performing a multi-point calibration by immersing the sensor tip in solutions of KNO₃ in suitable aqueous buffer at different concentrations (e.g., 100 uM, 1 mM, 10 mM KNO₃) and recording the variation in observed electrode potential at those concentrations, anticipating a linear response.

Nitrate-sensitive electrodes, in general, can be made by combining a reference electrode and a working electrode exposed to nitrate-sensitive molecules such as quaternary ammonium salts (e.g. methyltridodecylammonium nitrate, tridodecylhexylammonium nitrate or tricaprylmethylammonium nitrate) or substituted tris(1,10-phenanthroline) nickel (II) nitrate salts in solution, separated from the test solution by an appropriate nitrate-permeable membrane. Similarly, microelectrodes can be made from their corresponding macroelectrode by embedding into a micropipette of suitable size.

### Example 33: Detection of Sample Nitrate Concentration Using Spectroscopic or Chromatographic Methods (Prophetic)

Sample nitrate concentration can also be measured using a spectroscopic methods. For example, a solid or liquid sample can be analyzed in an infrared spectrometer, using the peak around 835 cm⁻¹ corresponding to an N—O stretch, and can be quantified by comparing the absorbance of this peak with standards of known concentration.

Similarly, Raman spectroscopy can be used, for example by investigating an N—O stretch around 1045 cm⁻¹ that can be quantified by comparing the peak intensity to standards of known concentration.

UV-visible spectroscopy can be used to characterize the concentration of nitrate in solution by direct measurement of UV absorption of the nitrate ion in a test solution (around 200 nm in water) or through selective binding of nitrate to a suitable target molecule, and can be quantified when compared to calibrated known standards in a colorimetric assay or similar process such as by using a commercial assay similar to that sold by Roche (part number 11746081001, Nitrite/Nitrate, Colorimetric Test). Likewise, fluorescence spectroscopy can be used to determine the concentration of nitrate through binding to a suitable selective fluorescent species, and quantified by comparison to calibrated known standards in a colorimetric assay or similar process.

Ion chromatography can also be used to characterize the nitrate concentration of a test sample, for example by determining the anion and cation concentrations in biological samples using a Dionex ICS-2000 ion chromatography system with AS-19 and CS-12A columns, allowing simultaneous measurement of anions and cations during one sample run and quantified using suitable standards.

Samples can be prepared, for example, by centrifuging biological samples at 5,000 rpm for 10 min, filtering the supernatant using e.g. a 0.20-μm-pore-size filter at 10,000 rpm for 20 min. Filtered supernatants can then be diluted in a e.g. 1:10, 1:20, 1:25, 1:50 or 1:100 ratios with Millipore water before analysis.

### Example 34 Detection of mRNA Markers Expressed by Respiring Bacteria in a Test Sample Using Fluorescence Methods (Prophetic)

Wound bacteria can signal their nitrate-respiring activity through the expression of specific genes into the local environment. The corresponding mRNA can be detected in a sample of this environment through the use of a hybridization chain reaction (HCR) procedure with specific fluorescent hairpin probes featuring the appropriate target sequence as described in Examples 11 and 12 herein.

### Example 35: Analysis of mRNA Markers Expressed by Respiring Bacteria in a Test Sample Using Electrochemical Methods (Prophetic)

Alternatively, the HCR method outlined in the previous example is modified to employ electrochemical detection methods. A working electrode surface such as gold, platinum or a transparent conducting oxide can be functionalized, for instance by deposition of biotinylated-gold nanoparticles, which are then further modified by exposure to avidin-functionalized RNA target sequences of interest (e.g., for narG, nirS and/or nosZ as listed in Table 2). HCR of the sample is then performed as described for Examples 11 and 12 and the sample then contacted with the electrode probe; presence of the target RNA is detected through cyclic voltammetry measurement or by a change in the electrochemical impedance spectrum.

### Example 36: Application of Detection Methods Ex Situ

A small sample of an infected biological environment can be removed by swab, or by excision, or by drawing a blood sample, for example, and will therefore be in either solid, gel or liquid form.

After suitable preparation (e.g., dissolution, centrifugation and/or dilution) the sample can be analyzed for oxygen content, redox potential and nitrate concentration by contacting the sample with an appropriate electrode setup connected to an electrochemical analyzer (e.g., potentiostat such as those made by Gamry Instruments or Biologic, Inc). Each measurement can be made using the same instrument by changing out the electrodes between measurement, or can be measured on three separate instruments each appropriately configured, or can be measured simultaneously using three different channels on the same instrument.

Likewise, the sample can be further, or alternatively, analyzed for nitrate concentration using an appropriate spectroscopic method (infra-red, Raman, UV-vis and/or fluorescence) or chromatographic method as described in Example 33.

Furthermore, the sample can be analyzed for genetic markers of bacterial nitrate respiration using the HCR fluorescence method described in Example 34 or the electrochemical method described in Example 35.

Analyses can be combined by taking repeated ex-situ analyses of samples of the infected biological area as a function of time over several minutes, or hours, or days, or weeks, or months, or longer depending on the nature of the infection. Measurement of sample oxygen concentration, redox potential and nitrate concentration by electrochemical methods can be carried out as described, and the appropriate course of action determined from the results as described below, with antibiotics, chlorate and/or nitrate materials being applied to the infected biological area, until completion of the treatment of the infected biological environment.

### Example 37 Application of Detection Methods In Situ

An infected biological environment was contacted with a bandage or prosthetic that contained sensing apparatus to configured determine oxygen content, redox potential and nitrate concentration using an appropriate microelectrode array connected to external power source, amplifier and detection instrument (e.g., potentiostat). Measurements of oxygen concentration, redox potential and nitrate concentration were made at defined time points such as every few minutes, every few hours, or hours, or days, or weeks, or months or combinations of these periods. The appropriate course of action was determined from the results as outlined below, with antibiotics, chlorate and/or nitrate materials being applied to the infected biological area, until completion of the treatment of the infected biological environment.

### Example 838 Analysis Indicates for Antibiotic Treatment Only

Analysis of a wound sample returned the following results:
Oxygen concentration: 200 uM
Redox potential: 220 mV
Nitrate concentration: not detected

The combination of these three data points indicated that the wound bacteria are in the “oxic zone” and are respiring normally. Therefore, antibiotics are provided to the infected biological environment in an amount effective to inhibit viability of the bacteria (for example, 10 ug/mL ciprofloxacin).

### Example 39: Analysis Indicates for Antibiotic and Chlorate Treatment

Analysis of a wound sample returned the following results:
Oxygen concentration: 12 uM
Redox potential: −245 mV
Nitrate concentration: 580 uM

The combination of these three data points indicated that the wound bacteria are in the “anoxic zone” and are respiring through the nitrate pathway. Therefore, both chlorate and antibiotics are provided to the infected biological environment in an amount effective to inhibit viability of the bacteria (for example, 50 mM chlorate in addition to 100 ug/mL ciprofloxacin).

### Example 40: Analysis Indicates for Antibiotic and Chlorate Treatment after Additional Nitrate is Provided for a Nitrate-Contacting Time

Analysis of a wound sample returned the following results:
Oxygen concentration: 15 uM
Redox potential: −145 mV
Nitrate concentration: 35 uM

The combination of these three data points indicated that the wound bacteria are likely in the “anoxic zone” but have not yet fully switched to respiration through the nitrate pathway. Therefore, additional nitrate is provided to the wound for a nitrate contacting time, intended to drive the bacteria into full nitrate respiration to improve their sensitivity to chlorate. The amount of nitrate administered was 10 uM. Measurements were repeated after the nitrate contacting time, and revealed the nitrate concentration in the wound had increased to 450 uM. After this measurement, chlorate, nitrate and antibiotics are provided to the infected biological environment in an amount effective to inhibit viability of the bacteria (13 uM nitrate and 50 mM chlorate in addition to 100 ug/mL ciprofloxacin).

### Example 41: Analysis Indicates for Antibiotic and Chlorate Treatment after Confirmation of Nitrate Respiration Pathway Activation

Analysis of a wound sample returned the following results:
Oxygen concentration: 10 uM
Redox potential: +35 mV
Nitrate concentration: 4 uM

The combination of these three data points indicated that the wound bacteria are in the “anoxic zone” but have not yet fully switched to respiration through the nitrate pathway. Therefore, additional nitrate is provided to the wound for a nitrate contacting time, intended to drive the bacteria into full nitrate respiration to improve their sensitivity to chlorate. The amount of nitrate administered was 20 uM. However, when measurements were repeated after the nitrate contacting time, the nitrate concentration in the wound had not increased. HCR fluorescence detection of the narG gene was carried out, confirming the gene was being expressed strongly, consistent with the majority of the bacteria being in the nitrate respiration mode. Therefore, after this confirmatory measurement, chlorate, nitrate and antibiotics are provided to the infected biological environment in an amount effective to inhibit viability of the bacteria (for example, 1 mM nitrate and 50 mM chlorate in addition to 100 ug/mL ciprofloxacin).

### Example 42: Analysis Indicates the Wound Bacteria are in the “Hypoxic” Zone

Analysis of a wound sample returned the following results:
Oxygen concentration: 95 uM
Redox potential: +185 mV
Nitrate concentration: 0 uM

The combination of these three data points indicated that the wound bacteria are in the “hypoxic zone” experiencing considerable oxic stress yet not fully transitioned to nitrate respiration. Therefore, additional nitrate is provided to the wound for a nitrate contacting time, intended to drive the bacteria into full nitrate respiration to improve their sensitivity to chlorate. The amount of nitrate administered was 1 mM. Measurements were repeated after the nitrate contacting time, and revealed the nitrate concentration in the wound had increased to 450 uM. Therefore, both chlorate and antibiotics are provided to the infected biological environment in an amount effective to inhibit viability of the bacteria (for example, 20 mM chlorate in combination with 10 ug/mL ceftazidime).

### Example 43: Analysis Indicates the Wound Bacteria are in the “Hypoxic” Zone

Analysis of a wound sample returned the following results:
Oxygen concentration: 135 uM
Redox potential: −300 mV
Nitrate concentration: 100 uM

The oxygen concentration measured indicated that the wound bacteria are likely in, or close to, the “hypoxic zone” experiencing considerable oxic stress yet not fully transitioned to nitrate respiration. However, the redox potential measured suggested that the bacteria had indeed switched to nitrate respiration, supported by the measurable nitrate concentration. To resolve this conflict and understand the situation better, HCR fluorescence detection of the narG gene was carried out, indicating the gene was being expressed in a limited manner, consistent with the transition of the bacteria from oxygen to nitrate respiration under hypoxic conditions. Therefore, both chlorate and antibiotics are provided to the infected biological environment in an amount effective to inhibit viability of the bacteria (for example, 25 mM chlorate in combination with 31.25 ug/mL tobramycin).

### Example 44: Detection of Multiple “Zones” in a Single Wound

Analysis of a visually-heterogeneous wound sample returned the following results:
Top of wound: Oxygen concentration: 65 uM
Redox potential: +45 mV
Nitrate concentration: 1 uM
Bottom of wound:
Oxygen concentration: 8 uM
Redox potential: −175 mV
Nitrate concentration: 275 uM

The clear differences between measurements at the top and bottom of the wound indicate two scenarios: a “hypoxic zone” at the top of the wound and an “anoxic zone” at the bottom. Therefore, chlorate and antibiotics are provided to the different infected biological environments in an amount effective to inhibit viability of the bacteria based on this data: for the top section, 25 mM chlorate in combination with 31.25 ug/mL tobramycin; for the bottom section 50 mM chlorate in combination with 500 ug/mL tobramycin).

### Example 45: Exemplary Smart Bandage Configuration

FIGS. 20A and 20B show an example of a smart bandage configured to enact embodiments of the methods described herein. FIG. 20A shows the skin facing side, with an adhesive portion (9805) and a wound-covering portion (9810), the wound-covering portion including circuitry connecting the elements of the smart bandage. On the wound-covering portion (9810), there are an array of capsules that can be opened/emptied upon an electrical signal, the array including antibiotic payloads (9815), chlorate payloads (9820), and nitrate payloads (9835). In some embodiments, other capsules can be included for other releasing other substances used in treatment. There are sensors that are configured to measure O₂ levels (9830A), redox potential levels (9830B), and nitrate levels (9830C). The bandage also includes integrated circuits (9840) that connect to the sensors through vias (9850A, 9850B, 9850C) and to the capsules by vias (not shown), the integrated circuits (9840) can include elements like processing elements for determining when to open various capsules based on sensor readings, battery/capacitive elements, communication elements, and antenna elements (for communications and/or powering). As the sensors determine oxygen, redox, and nitrate levels, the bandage releases appropriate payloads (individually or in combination) according to various methods herein.

### Example 46: Exemplary Prosthetic Device

FIGS. 21A and 21B shows an example of a prosthetic configured to enact embodiments of the methods described herein. In this example, a portion of a knee replacement prosthetic is shown (the tibial component), but other portions or types of prosthetic (bone replacement, hip replacement, organ replacement/augmentation, ports, etc.) can be likewise modified.

There are sensors that are configured to measure O2 levels (9920A), redox potential levels (9920B), and nitrate levels (9920C). There are also capsules configured for controlled (electronic) release of payloads: antibiotics (9910), chlorate (9915), and nitrate (9925). In some embodiments, other capsules can be included for other releasing other substances used in treatment. A processor (9930) reads the sensors and controls the capsule release based on the sensor readings, according to various methods described herein.

An antenna (9935) allows control and/or powering from an external (e.g., outside the body) controller (9940) so that the system can be activated once an infection is determined or suspected. The example shows a single set of capsules and sensors, but a prosthesis could include an array of capsule/sensor combinations (9999) to cover different areas of the surface of the prosthesis (9905), such as shown in FIG. 21B. The sensor/payload groups (9999) can be located anywhere there is concern for an infection developing.

### Example 47: Depth-Based Drug Delivery

FIG. 22 shows an example of depth-based drug delivery to be used with the methods herein, and compatible (optionally) with the delivery devices described herein (e.g. Examples NN). The delivery system (310), such as the bandage shown in FIGS. 20A and 20B or the prosthetic shown in FIGS. 2A and 2B, is placed in contact or close proximity with the infected tissue/wound (305).

A given payload capsule (315), containing e.g. chlorate, antibiotics, and/or nitrates, is set up to release, when activated by an actuator (320), the payload to a particular depth in the tissue (305) by hollow microneedles (321). The actuator (320) is controlled by a microprocessor (325) connected to a sensor (330) that has a probe (331) set to that particular depth. In some embodiments, the devices are configured with probes and microneedles set for multiple depths, allowing for releasing different payloads at different depths based on the sensor readings taken at each depth, for each portion of the region being treated.

### Example 48 Smart Pill

FIG. 23 shows an example of a smart pill set to work with the methods described herein. Sensors (420) in the pill are configured to, using configured probes (405), measure oxygen, nitrate, and/or redox levels around the pill.

A microprocessor (410) determines, based at least on the sensor (420) readings, on when and how to activate an actuator (411) that controls the release of one or more payload regions (425A, 425B, 425C) to release antibiotics, chlorate, and/or nitrate payloads according to the methods herein. An antenna/communications system (415) can also send/receive data to an external system to allow further control of the release of the payloads (spatially, temporally, etc.).

### Example 49: Devices in Use

The devices as described herein (e.g. Examples 31 to 33) and other similar devices configured to carry out the methods described herein can be configured to release payloads of chlorate, antibiotics, nitrate, etc. in a controlled manner, such that the amount of payload being released is controlled (either by sub-dividing the payloads into smaller capsules, or by controlled actuation of the capsules).

The payloads are released in accordance with one or more of the methods described herein, including releasing different payloads at different times (when the readings being measured by the sensors change over time) and/or at different locations and/or depths in the tissue being treated (when the sensors in those areas/depths report different readings).

Materials that can be used in fabricating the devices described herein can include (but are not limited to): polycarbonate, polyurethane, polymethyl methacrylate, polypropylene, polyethylene, polyvinyl chloride, acrylonitrile butadiene styrene, polyethylene terephthalate, stainless steel, titanium, polyoxymethylene, polysulfone (psu), polytetrafluoroethylene, silicones, ceramics, gold, cobalt chrome, aluminum, magnesium, copper, silver, iridium, tantalum, and platinum.

Additional, exemplary embodiments, features, objects, and advantages of the present disclosure will be apparent to a skilled person from the claims and the instant disclosure in its entirety.

In particular, additional exemplary methods tools reagents and devices are illustrated in the Annex A, Annex B and Annex C in particular in Annex C of U.S. Provisional Application 63/519,537 and U.S. Provisional Application 63/670,084 the content of each of which is incorporated into references and constitute a part of this specification, together with the detailed description section serve to explain the principles and implementations of the disclosure. Other features, objects, and advantages will be apparent from the entire description and drawings, and from the claims.

In particular, the examples set forth in the enclosed Annex C of U.S. Provisional Application 63/519,537 and U.S. Provisional Application 63/670,084, are provided to give those of ordinary skill in the art a disclosure and description of how to make and use embodiments of the materials, compositions, systems and methods of the disclosure, and are not intended to limit the scope of what the inventors regard as their disclosure. Those skilled in the art will recognize how to adapt the features of the exemplified methods and systems based on the specific methods, systems for timed and/or targeted administration of chlorate to treat a bacterial infection and related matrices, compounds, compositions and implants according to various embodiments and scope of the claims.

In summary methods and systems described herein and related compositions, matrices and devices, for timed and/or targeted administration of chlorate for treatment and/or prevention of infections of a biological environment and related compositions, devices, matrices and implants. Chlorate administration can be performed alone or in combination with an antibiotic in a location and/or time targeted manner, the concentration and use of the chlorate and/or the antibiotic agents depending on the oxic/hypoxic/anoxic condition of the area being treated.

All patents and publications mentioned in the instant specification inclusive of the Annexes A to C of U.S. Provisional Application 63/519,537 and U.S. Provisional Application 63/670,084 are indicative of the levels of skill of those skilled in the art to which the disclosure pertains.

A person skilled in the art will appreciate the applicability and the necessary modifications to adapt the features described in detail in the present section, to additional agents and related compositions, methods and systems according to embodiments of the present disclosure.

The entire disclosure of each document cited (including patents, patent applications, journal articles, abstracts, laboratory manuals, books, or other disclosures) in the Background, Summary, Detailed Description, and Examples is hereby incorporated herein by reference. All references cited in this disclosure are incorporated by reference to the same extent as if each reference had been incorporated by reference in its entirety individually. However, if any inconsistency arises between a cited reference and the present disclosure, the present disclosure takes precedence.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the disclosure claimed. Thus, it should be understood that although the disclosure has been specifically disclosed by preferred embodiments, exemplary embodiments and optional features, modification and variation of the concepts herein disclosed can be resorted to by those skilled in the art upon the reading of the present disclosure, and that such modifications and variations are considered to be within the scope of this disclosure as defined by the appended claims.

It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains. The measurement in in the micro range, such as micromolar, micrograms, and microliters, are typically indicated using the Greek letter “mu” (μ) or interchangeably with letter u. This symbol is used as a prefix to denote “micro,” which means one-millionth (10\hat{ }-6) of a unit. Therefore, a micromalar is abbreviated as “PM,” or uM a microgram as “μg,” or “uM” and a microliter as “μl” “ul” or “uL” as will be understood by a skilled person.

When a Markush group or other grouping is used herein, all individual members of the group and all combinations and possible sub combinations of the group are intended to be individually included in the disclosure. Every combination of components or materials described or exemplified herein can be used to practice the disclosure, unless otherwise stated. One of ordinary skill in the art will appreciate that methods, device elements, and materials other than those specifically exemplified can be employed in the practice of the disclosure without resort to undue experimentation. All art-known functional equivalents, of any such methods, device elements, and materials are intended to be included in this disclosure. Whenever a range is given in the specification, for example, a temperature range, a frequency range, a time range, or a composition range, all intermediate ranges and all sub-ranges, as well as all individual values included in the ranges given are intended to be included in the disclosure. Any one or more individual members of a range or group disclosed herein can be excluded from a claim of this disclosure. The disclosure illustratively described herein suitably can be practiced in the absence of any element or elements, limitation or limitations which are not specifically disclosed herein.

A number of embodiments of the disclosure have been described. The specific embodiments provided herein are examples of useful embodiments of the disclosure and it will be apparent to one skilled in the art that the disclosure can be carried out using a large number of variations of the devices, device components, methods steps set forth in the present description. As will be obvious to one of skill in the art, methods and devices useful for the present methods can include a large number of optional composition and processing elements and steps.

In particular, it will be understood that various modifications may be made without departing from the spirit and scope of the present disclosure. Accordingly, other embodiments are within the scope of the following claims.

### REFERENCES

1. 2023, W.i.; Available from: webpage en.wikipedia.org/wiki/Infection at the filing date of the disclosure.
2. Corkery, K., Inhalable drugs for systemic therapy. Respiratory care, 2000. 45(7): p. 831-835.
3. Altschul S F, M. T., Schaffer A A, Zhang J, Zhang Z, Miller W, Lipman D J., Gapped BLAST and PSI-BLAST: a new generation of protein database search programs. Nucleic Acids Res., 1997. 25(17): p. 14.
4. Smith T F, W. M., Identification of common molecular subsequences. J Mol Biol, 1981. 147(1): p. 3.
5. W R, P., Searching protein sequence libraries: comparison of the sensitivity and selectivity of the Smith-Waterman and FASTA algorithms. Genomics, 1991. 11(3): p. 16.
6. Pearson W R, L. D., Improved tools for biological sequence comparison. Proc Natl Acad Sci USA, 1988. 85(8): p. 5.
7. Johnson L S, E. S., Portugaly E, Hidden Markov model speed heuristic and iterative HMM search procedure. BMC Bioinformatics, 2010. 11(431): p. 8.
8. Myers, E. W. and W. Miller, Optimal alignments in linear space. Computer applications in the biosciences: CABIOS, 1988. 4(1): p. 11-17.
9. Smith, T. F. and M. S. Waterman, Comparison of biosequences. Advances in applied mathematics, 1981. 2(4): p. 482-489.
10. Needleman, S. B. and C. D. Wunsch, A general method applicable to the search for similarities in the amino acid sequence of two proteins. Journal of molecular biology, 1970. 48(3): p. 443-453.
11. Pearson, W. R. and D. J. Lipman, Improved tools for biological sequence comparison. Proceedings of the National Academy of Sciences, 1988. 85(8): p. 2444-2448.
12. Karlin, S. and S. F. Altschul, Methods for assessing the statistical significance of molecular sequence features by using general scoring schemes. Proceedings of the National Academy of Sciences, 1990. 87(6): p. 2264-2268.
13. Karlin, S. and S. F. Altschul, Applications and statistics for multiple high-scoring segments in molecular sequences. Proceedings of the National Academy of Sciences, 1993. 90(12): p. 5873-5877.
14. Moreno-Vivian, C., et al., Prokaryotic nitrate reduction: molecular properties and functional distinction among bacterial nitrate reductases. J Bacteriol, 1999. 181(21): p. 6573-84.
15. D′Argenio, D. A., et al., Growth phenotypes of Pseudomonas aeruginosa lasR mutants adapted to the airways of cystic fibrosis patients. Mol Microbiol, 2007. 64(2): p. 512-33.
16. Hoffman, L. R., et al., Nutrient availability as a mechanism for selection of antibiotic tolerant Pseudomonas aeruginosa within the CF airway. PLoS Pathog, 2010. 6(1): p. e1000712.
17. Smith, E. E., et al., Genetic adaptation by Pseudomonas aeruginosa to the airways of cysticfibrosis patients. Proc Natl Acad Sci USA, 2006. 103(22): p. 8487-92.
18. Hoffman, L. R., et al., Pseudomonas aeruginosa lasR mutants are associated with cystic fibrosis lung disease progression. J Cyst Fibros, 2009. 8(1): p. 66-70.
19. Heydorn, A., et al., Quantification of biofilm structures by the novel computer program COMSTAT. Microbiology, 2000. 146(10): p. 2395-2407.
20. Ciofu, O. and T. Tolker-Nielsen, Tolerance and resistance of Pseudomonas aeruginosa biofilms to antimicrobial agents—how P. aeruginosa can escape antibiotics. Frontiers in microbiology, 2019. 10: p. 913.
21. Sakuragi, Y. and R. Kolter, Quorum-sensing regulation of the biofilm matrix genes (pel) ofi Pseudomonas aeruginosa. Journal of bacteriology, 2007. 189(14): p. 5383-5386.
22. Wikipedia Biofil. Available from: webpage en.wikipedia.org/wiki/Biofilm at the fling date of the present disclosure.
23. Wisniewski, N. A., et al., Tissue-integrating oxygen sensors: continuous tracking of tissue hypoxia. Oxygen transport to tissue XXXIX, 2017: p. 377-383.
24. Jorth, P., et al., Quantitative visualization of gene expression in mucoid and nonmucoid Pseudomonas aeruginosa aggregates reveals localized peak expression of alginate in the hypoxic zone. MBio, 2019. 10(6): p. 10.1128/mbio. 02622-19.
25. Gupta, S., N. Laskar, and D. E. Kadouri, Evaluating the effect of oxygen concentrations on antibiotic sensitivity, growth, and biofilm formation of human pathogens. Microbiology insights, 2016. 9: p. MBI. S40767.
26. Lubbers, J. and J. Bianchine, Effects of the acute rising dose administration of chlorine dioxide, chlorate and chlorite to normal healthy adult male volunteers. Journal of environmental pathology, toxicology and oncology: official organ of the International Society for Environmental Toxicology and Cancer, 1984. 5(4-5): p. 215-228.
27. Lubbers, J. R., S. Chauhan, and J. R. Bianchine, Controlled clinical evaluations of chlorine dioxide, chlorite and chlorate in man. Fundamental and applied Toxicology, 1981. 1(4): p. 334-338.
28. Lubbers, J. R., S. Chauan, and J. R. Bianchine, Controlled clinical evaluations of chlorine dioxide, chlorite and chlorate in man. Environmental health perspectives, 1982. 46: p. 57-62.
29. Lubbers, J., et al., The effects of chronic administration of chlorine dioxide, chlorite and chlorate to normal healthy adult male volunteers. Journal of environmental pathology, toxicology and oncology: official organ of the International Society for Environmental Toxicology and Cancer, 1984. 5(4-5): p. 229-238.
30. Lubbers, J., et al., The effects of chronic administration of chlorite to glucose-6-phosphate dehydrogenase deficient healthy adult male volunteers. Journal of environmental pathology, toxicology and oncology: official organ of the International Society for Environmental Toxicology and Cancer, 1984. 5(4-5): p. 239-242.
31. Orenstein, D. M., Cystic fibrosis: A guide for patient and family 4th ed. 4th ed. 2011: Lippincott Williams & Wilkins.
32. Spero, M. A. and D. K. Newman, Chlorate specifically targets oxidant-starved, antibiotic-tolerant populations of Pseudomonas aeruginosa biofilms. MBio, 2018. 9(5): p. 10.1128/mbio. 01400-18.
33. Luo, Z., et al., Hypoxia signaling in human health and diseases: implications and prospects for therapeutics. Signal transduction and targeted therapy, 2022. 7(1): p. 218.
34. Della Rocca, Y., et al., Hypoxia: molecular pathophysiological mechanisms in human diseases. Journal of physiology and biochemistry, 2022. 78(4): p. 739-752.
35. The Annual UK Drug Tariff, in The Stationary Office. 1988 (November), 1998 (May), 2007 (February) The UK Drug Tariff: London, UK.
36. Lazarus, G. S., et al., Definitions and guidelines for assessment of wounds and evaluation of healing. Arch Dermatol, 1994. 130(4): p. 489-493.
37. Boateng, J. S., et al., Wound healing dressings and drug delivery systems: a review. Journal of pharmaceutical sciences, 2008. 97(8): p. 2892-2923.
38. Gilliland, E. L., et al., Bacterial colonisation of leg ulcers and its effect on the success rate of skin grafting. Annals of the Royal College of Surgeons of England, 1988. 70(2): p. 105-108.
39. Armstrong, S. and C. Ruckley, Use of a fibrous dressing in exuding leg ulcers. Journal of Wound Care, 1997. 6(7): p. 322-324.
40. Hareendran, A., et al., Measuring the impact of venous leg ulcers on quality of life. Journal of Wound Care, 2005. 14(2): p. 53-57.
41. Naradzay, F. and R. Alson, Burns, thermal. Web M D, 2005.
42. Bolton, L. and L. Van Rijswijk, Wound dressings: meeting clinical and biological needs. Dermatology nursing, 1991. 3(3): p. 146-161.
43. Krasner, D., et al., The ABCs of wound care dressings. wound management, 1993. 66: p. 68-69.
44. Strodtbeck, F., Physiology of wound healing. Newborn Infants Nurs Rev, 2001. 1: p. 43-52.
45. Russell, L., Understanding physiology of wound healing and how dressings help. British journal of nursing, 2000. 9(1): p. 10-21.
46. B A, M., The Skin Wound healing: biochemical and clinical aspects, ed. D.-m.R. Choen I K, Lindblad W,. 1999, Philadelphia, P A: Saunders.
47. G S, S., Molecular regulation of wound healing. Acute and chronic wounds: Nursing management. 2nd edition., ed. B. R A. 1999,. St. Louis, M O: Mosby.
48. Rothe, M. and V. Falanga, Growth factors: their biology and promise in dermatologic diseases and tissue repair. Archives of dermatology, 1989. 125(10): p. 1390-1398.
49. Shakespeare, P., Burn wound healing and skin substitutes. Burns, 2001. 27(5): p. 517-522.
50. D M, C., Wound healing: New understandings. Nurs Pract Forum 1999. 10: p. 74-86.
51. Martin, P., Wound healing—aiming for perfect skin regeneration. Science, 1997. 276(5309): p. 75-81.
52. Percival, N. J., Classification of wounds and their management. Surgery (Oxford), 2002. 20(5): p. 114-117.
53. Harding, K., H. Morris, and G. Patel, Clinical review Healing chronic wounds. Br Med J, 2002. 324: p. 160-163.
54. Moore, K., et al., Prediction and monitoring the therapeutic response of chronic dermal wounds. International wound journal, 2006. 3(2): p. 89-98.
55. Martins-Green, Wound Healing, Tissue Repair, and Regeneration in Diabetes 9780128164136. 2020.
56. Schreml, S., et al., Luminescent dual sensors reveal extracellular pH-gradients and hypoxia on chronic wounds that disrupt epidermal repair. Theranostics, 2014. 4(7): p. 721.
57. Ferreira, M. C., et al., Complex wounds. Clinics, 2006. 61(6): p. 571-578.
58. Chen, W. J., A. A. Rogers, and M. J. Lydon, Characterization of biologic properties of wound fluid collected during early stages of wound healing. Journal of Investigative Dermatology, 1992. 99(5): p. 559-564.
59. Hemilä, H. and R. M. Douglas, Vitamin C and acute respiratory infections. The international journal of tuberculosis and lung disease, 1999. 3(9): p. 756-761.
60. Rojas, A. I. and T. J. Phillips, Patients with chronic leg ulcers show diminished levels of vitamins A and E, carotenes, and zinc. Dermatologic Surgery, 1999. 25(8): p. 601-604.
61. Patel, G. K., The role of nutrition in the management of lower extremity wounds. The international journal of lower extremity wounds, 2005. 4(1): p. 12-22.
62. Pierce, G. F., et al., Transforming growth factor beta reverses the glucocorticoid-induced wound-healing deficit in rats: possible regulation in macrophages by platelet-derived growth factor. Proceedings of the National Academy of Sciences, 1989. 86(7): p. 2229-2233.
63. Beck, L. S., et al., One systemic administration of transforming growth factor-beta 1 reverses age-or glucocorticoid-impaired wound healing. The Journal of clinical investigation, 1993. 92(6): p. 2841-2849.
64. Chedid, M., et al., Glucocorticoids inhibit keratinocyte growth factor production in primary dermalfibroblasts. Endocrinology, 1996. 137(6): p. 2232-2237.
65. Growth factor” at Dorland's Medical Dictionary. 2011; Available from: https://web.archive.org/web/20110607011425/http://www.mercksource.com/pp/us/cns/cns cns_hl_dorlands_split.jsp?pa=/ppdocs/us/common/dorlands/dorland/three/000038554.h
66. Mandla, S., L. Davenport Huyer, and M. Radisic, Multimodal bioactive material approaches for wound healing. APL bioengineering, 2018. 2(2): p. 021503.
67. Komarcević, A., The modern approach to wound treatment. Medicinski pregled, 2000. 53(7-8): p. 363-368.
68. Ten Dijke, P. and K. K. Iwata, Growth factors for wound healing. Bio/Technology, 1989. 7(8): p. 793-798.
69. Mayet, N., et al., A comprehensive review of advanced biopolymeric wound healing systems. Journal of pharmaceutical sciences, 2014. 103(8): p. 2211-2230.
70. Steenfos, H. H., Growth factors and wound healing. Scandinavian journal of plastic and
reconstructive surgery and hand surgery, 1994. 28(2): p. 95-105.
71. Greenhalgh, D. G., The role of growth factors in wound healing. Journal of Trauma and Acute Care Surgery, 1996. 41(1): p. 159-167.
72. Mann, A., et al. Granulocyte-macrophage colony-stimulating factor is essential for normal wound healing. in Journal of Investigative Dermatology Symposium Proceedings. 2006. Elsevier.
73. Lee, A.-R. C., et al., Reversal of silver sulfadiazine-impaired wound healing by epidermal growth factor. Biomaterials, 2005. 26(22): p. 4670-4676.
74. Fang, R. C. and R. D. Galiano, A review of becaplermin gel in the treatment of diabetic neuropathicfoot ulcers. Biologics: targets & therapy, 2008. 2(1): p. 1-12.
75. Miller, M. S., Use of topical recombinant human platelet-derived growth factor-BB (becaplermin) in healing of chronic mixed arteriovenous lower extremity diabetic ulcers. The Journal of foot and ankle surgery, 1999. 38(3): p. 227-231.
76. Pierce, G., et al., Platelet-derived growth factor-BB and transforming growth factor beta 1 selectively modulate glycosaminoglycans, collagen, and myofibroblasts in excisional wounds. The American journal of pathology, 1991. 138(3): p. 629.
77. Greenhalgh, D. G., et al., PDGF and FGF stimulate wound healing in the genetically diabetic mouse. The American journal of pathology, 1990. 136(6): p. 1235-1246.
78. Judith, R., et al., Application of a PDGF-containing novel gel for cutaneous wound healing. Life sciences, 2010. 87(1-2): p. 1-8.
79. Smiell, J. M., et al., Efficacy and safety of becaplermin (recombinant human platelet-derived growth factor-BB) in patients with nonhealing, lower extremity diabetic ulcers: a combined analysis of four randomized studies. Wound Repair and Regeneration, 1999. 7(5): p. 335-346.
80. Gökşen, S., B. Balabanh₁, and Ş. Coşkun-Cevher, Application of platelet derived growth factor-BB and diabetic wound healing: the relationship with oxidative events. Free radical research, 2017. 51(5): p. 498-505.
81. Choi, S. M., et al., Development of stabilized growth factor-loaded hyaluronate-collagen dressing (HCD) matrix for impaired wound healing. Biomaterials research, 2016. 20(9): p. 1-7.
82. Choi, S. M., et al., Effects of structurally stabilized EGF and bFGF on wound healing in type I and type H diabetic mice. Acta biomaterialia, 2018. 66: p. 325-334.
83. Garcia-Orue, I., et al., Novel nanofibrous dressings containing rhEGF and Aloe vera for wound healing applications. International journal of pharmaceutics, 2017. 523(2): p. 556-566.
84. Lin, Y.-J., et al., Stimulation of wound healing by PU/hydrogel composites containing fibroblast growth factor-2. Journal of Materials Chemistry B, 2015. 3(9): p. 1931-1941.
85. Bao, P., et al., The role of vascular endothelial growth factor in wound healing. Journal of Surgical Research, 2009. 153(2): p. 347-358.
86. Lord, M. S., et al., Perlecan and vascular endothelial growth factor-encoding DNA-loaded chitosan scaffolds promote angiogenesis and wound healing. Journal of Controlled Release, 2017. 250: p. 48-61.
87. Elçin, Y. M., V. Dixit, and G. Gitnick, Extensive in vivo angiogenesis following controlled release of human vascular endothelial cell growth factor: implications for tissue engineering and wound healing. Artificial organs, 2001. 25(7): p. 558-565.
88. Long, D. W., et al., Controlled delivery of platelet-derived proteins enhances porcine wound healing. Journal of Controlled Release, 2017. 253: p. 73-81.
89. Gu, D.-l., et al., Adenovirus encoding human platelet-derived growth factor-B delivered in collagen exhibits safety, biodistribution, and immunogenicity profiles favorable for clinical use. Molecular therapy, 2004. 9(5): p. 699-711.
90. Wallace, E., Feeding the wound: nutrition and wound care. British Journal of Nursing, 1994. 3(13): p. 662-667.
91. Linder, M. C., Nutritional biochemistry and metabolism: with clinical applications, 2nd edition. collagen synthesis and bone tissue development. 1991, East Norwalk, C T: Appleton & Lange
92. Flanigan, K. H., Nutritional aspects of wound healing. Advances in wound care: the journal for prevention and healing, 1997. 10(3): p. 48-52.
93. Ehrlich, H. P., H. Tarver, and T. K. Hunt, Effects of vitamin A and glucocorticoids upon inflammation and collagen synthesis. Annals of surgery, 1973. 177(2): p. 222-227.
94. Eaglstein, W. H., et al., Optimal use of an occlusive dressing to enhance healing: Effect of delayed application and early removal on wound healing. Archives of dermatology, 1988. 124(3): p. 392-395.
95. Taylor, T., et al., Ascorbic acid supplementation in the treatment of pressure-sores. The Lancet, 1974. 304(7880): p. 544-546.
96. ter Riet, G., A. G. Kessels, and P. G. Knipschild, Randomized clinical trial of ascorbic acid in the treatment of pressure ulcers. Journal of clinical epidemiology, 1995. 48(12): p. 1453-1460.
97. Fitzmaurice, S., R. K. Sivamani, and R. R. Isseroff, Antioxidant therapies for wound healing: a clinical guide to currently commercially available products. Skin pharmacology and physiology, 2011. 24(3): p. 113-126.
98. da Rocha, R. P., et al., Effects of a vitamin pool (vitamins A, E, and C) on the tissue necrosis process: experimental study on rats. Aesthetic plastic surgery, 2002. 26(3): p. 197-202.
99. Baumann, L. S. and J. S. Md, The effects of topical vitamin E on the cosmetic appearance of scars. Dermatologic Surgery, 1999. 25(4): p. 311-315.
100. H, O., Nutrients in profile. 2nd edition. Bioconcepts. 1995, Queensland: Kelvin Grove.
101. Kirschmann G, K. J., Nutrition almanac, 4th edition. 1996, New York, N Y: McGraw-Hill.
102. Rasik, A. M. and A. Shukla, Antioxidant status in delayed healing type of wounds. International journal of experimental pathology, 2000. 81(4): p. 257-263.
103. Ågren, M. S., Studies on zinc in wound healing. Acta Derm Venereol Suppl 1990. 154: p. 1-36.
104. Kietzmann, M. and M. Braun, Effects of the zinc oxide and cod liver oil containing ointment Zincojecol in an animal model of wound healing. DTW. Deutsche tierarztliche Wochenschrift, 2006. 113(9): p. 331-334.
105. Lansdown, A. B., Metallothioneins: potential therapeutic aids for wound healing in the skin. Wound repair and regeneration, 2002. 10(3): p. 130-132.
106. Yang, H., J. Chai, and Z. Guo, Effect of improved topical agents on healing time of deep second-degree burn wound. Zhongguo xiu fu chong jian wai ke za zhi=Zhongguo xiufu chongjian waike zazhi=Chinese journal of reparative and reconstructive surgery, 2001. 15(3): p. 162-164.
107. Ågren, M. S., et al., A randomized, double-blind, placebo-controlled multicenter trial evaluating topical zinc oxide for acute open wounds following pilonidal disease excision. Wound repair and regeneration, 2006. 14(5): p. 526-535.
108. Marczin, N., et al., Antioxidants in myocardial ischemia-reperfusion injury: therapeutic potential and basic mechanisms. Archives of Biochemistry and Biophysics, 2003. 420(2): p. 222-236.
109. Tenhunen, R., H. S. Marver, and R. Schmid, The enzymatic conversion of heme to bilirubin by microsomal heme oxygenase. Proceedings of the National Academy of Sciences of the United States of America, 1968. 61(2): p. 748-755.
110. Barañano, D. E., et al., Biliverdin reductase: a major physiologic cytoprotectant. Proceedings of the national academy of sciences, 2002. 99(25): p. 16093-16098.
111. Lu, J. and A. Holmgren, The thioredoxin antioxidant system. Free Radical Biology and Medicine, 2014. 66: p. 75-87.
112. Hanschmann, E.-M., et al., Thioredoxins, glutaredoxins, and peroxiredoxins-molecular mechanisms and health significance: from cofactors to antioxidants to redox signaling. Antioxidants & redox signaling, 2013. 19(13): p. 1539-1605.
113. Hofmann, B., H.-J. Hecht, and L. Flohé, Peroxiredoxins. Biological chemistry, 2002. 383(3-4): p. 347-364.
114. Wood, Z. A., et al., Structure, mechanism and regulation of peroxiredoxins. Trends in biochemical sciences, 2003. 28(1): p. 32-40.
115. Peshenko, I. V. and H. Shichi, Oxidation of active center cysteine of bovine 1-Cys peroxiredoxin to the cysteine sulfenic acid form by peroxide and peroxynitrite. Free Radical Biology and Medicine, 2001. 31(3): p. 292-303.
116. Kümin, A., et al., Peroxiredoxin 6 is a potent cytoprotective enzyme in the epidermis. The American journal of pathology, 2006. 169(4): p. 1194-1205.
117. Schäfer, M. and S. Werner, Oxidative stress in normal and impaired wound repair. Pharmacological research, 2008. 58(2): p. 165-171.
118. Yoshida, T., et al., The involvement of thioredoxin and thioredoxin binding protein-2 on cellular proliferation and aging process. ANNALS-NEW YORK ACADEMY OF SCIENCES, 2005. 1055: p. 1-12.
119. Cano Sanchez, M., et al., Targeting oxidative stress and mitochondrial dysfunction in the treatment of impaired wound healing: a systematic review. Antioxidants, 2018. 7(8): p. 98.
120. Vervaart, P. and K. Knight, Oxidative stress and the cell. Clinical Biochemist Reviews, 1996. 17: p. 3-16.
121. Shukla, A., A. M. Rasik, and G. K. Patnaik, Depletion of reduced glutathione, ascorbic acid, vitamin E and antioxidant defence enzymes in a healing cutaneous wound. Free Radical Research, 1997. 26(2): p. 93-101.
122. Adamson, B., et al., Delayed repair: the role of glutathione in a rat incisional wound model. Journal of Surgical Research, 1996. 62(2): p. 159-164.
123. Mudge, B. P., et al., Role of glutathione redox dysfunction in diabetic wounds. Wound Repair and Regeneration, 2002. 10(1): p. 52-58.
124. Musalmah, M., et al., Comparative effects of palm vitamin E and α-tocopherol on healing and wound tissue antioxidant enzyme levels in diabetic rats. Lipids, 2005. 40(6): p. 575-580.
125. Wlaschek, M. and K. Scharffetter-Kochanek, Oxidative stress in chronic venous leg ulcers. Wound Repair and Regeneration, 2005. 13(5): p. 452-461.
126. Loo, A. E. K., et al., Effects of hydrogen peroxide on wound healing in mice in relation to oxidative damage. PloS one, 2012. 7(11): p. e49215.
127. Taylor, R. and T. James, The role of oxidative stress in the development and persistence of pressure ulcers, in Pressure ulcer research. 2005, Springer. p. 205-232.
128. Wiseman, H., Vitamin D is a membrane antioxidant Ability to inhibit iron-dependent lipid peroxidation in liposomes compared to cholesterol, ergosterol and tamoxifen and relevance to anticancer action. FEBS letters, 1993. 326(1-3): p. 285-288.
129. Grandjean, E. M., et al., Efficacy of oral long-term N-acetylcysteine in chronic bronchopulmonary disease: a meta-analysis of published double-blind, placebo-controlled clinical trials. Clinical therapeutics, 2000. 22(2): p. 209-221.
130. Petersen Shay, K., et al., Is α-lipoic acid a scavenger of reactive oxygen species in vivo?Evidence for its initiation of stress signaling pathways that promote endogenous antioxidant capacity. IUBMB life, 2008. 60(6): p. 362-367.
131. Kundukad, B., et al., Mechanistic action of weak acid drugs on biofilms. Scientific reports, 2017. 7(1): p. 1-12.
132. Marchese, A., et al., Effect of fosfomycin alone and in combination with N-acetylcysteine on E. coli biofilms. International journal of antimicrobial agents, 2003. 22: p. S95-S100.
133. Gomes, F., et al., Farnesol in combination with N-acetylcysteine against Staphylococcus epidermidis planktonic and biofilm cells. Brazilian Journal of Microbiology, 2012. 43(1): p. 235-242.
134. Khalaf, A., et al., Antibacterial, anti-biofilm activity of some non-steroidal anti-inflammatory drugs and N-acetyl cysteine against some biofilm producing uropathogens. American Journal of Epidemiology, 2015. 3(1): p. 1-9.
135. Cotgreave, I., P. Moldéus, and I. Schuppe, The metabolism of N-acetylcysteine by human endothelial cells. Biochemical pharmacology, 1991. 42(1): p. 13-16.
136. Hart, A., et al., Sensory neuroprotection, mitochondrial preservation, and therapeutic potential of N-acetyl-cysteine after nerve injury. Neuroscience, 2004. 125(1): p. 91-101.
137. Raftos, J. E., et al., Kinetics of uptake and deacetylation of N-acetylcysteine by human erythrocytes. The international journal of biochemistry & cell biology, 2007. 39(9): p. 1698-1706.
138. Bernard, G., et al., Effect of N-acetylcysteine on the pulmonary response to endotoxin in the awake sheep and upon in vitro granulocyte function. The Journal of clinical investigation, 1984. 73(6): p. 1772-1784.
139. Aihara, M., et al., Effects of N-acetylcysteine and ambroxol on the production of IL-12 and IL-10 in human alveolar macrophages. Respiration, 2000. 67(6): p. 662-671.
140. Paterson, R. L., H. F. Galley, and N. R. Webster, The effect of N-acetylcysteine on nuclear factor-K B activation, interleukin-6, interleukin-8, and intercellular adhesion molecule-1 expression in patients with sepsis. Critical care medicine, 2003. 31(11): p. 2574-2578.
141. Yang, C., et al., Rapamycin and mTOR inhibitors probably have therapeutic effects for post-operative cognitive dysfunction. Medical hypotheses, 2013. 81(3): p. 487-488.
142. Tsai, M.-L., et al., Topical N-acetylcysteine accelerates wound healing in vitro and in vivo via the PKC/Stat3 pathway. International journal of molecular sciences, 2014. 15(5): p. 7563-7578.
143. Sen, C. K. and S. Roy, Redox signals in wound healing. Biochimica et Biophysica Acta (BBA)—General Subjects, 2008. 1780(11): p. 1348-1361.
144. Ram, M., et al., Antioxidant potential of bilirubin-accelerated wound healing in streptozotocin-induced diabetic rats. Naunyn-Schmiedeberg's archives of pharmacology, 2014. 387(10): p. 955-961.
145. Ram, M., et al., Bilirubin modulated cytokines, growth factors and angiogenesis to improve cutaneous wound healing process in diabetic rats. International immunopharmacology, 2016. 30: p. 137-149.
146. Chigurupati, S., et al., A synthetic uric acid analog accelerates cutaneous wound healing in mice. PLoS One, 2010. 5(4): p. e10044.
147. Nery, R. A., et al., Uric acid and tissue repair. ABCD. Arquivos Brasileiros de Cirurgia Digestiva (São Paulo), 2015. 28(4): p. 290-292.
148. Jagetia, G. C. and G. Rajanikant, Role of curcumin, a naturally occurring phenolic compound of turmeric in accelerating the repair of excision wound, in mice whole-body exposed to various doses of γ-radiation. Journal of Surgical Research, 2004. 120(1): p. 127-138.
149. Panchatcharam, M., et al., Curcumin improves wound healing by modulating collagen and decreasing reactive oxygen species. Molecular and cellular biochemistry, 2006. 290(1): p. 87-96.
150. Mohanty, C. and S. K. Sahoo, Curcumin and its topical formulations for wound healing applications. Drug Discovery Today, 2017. 22(10): p. 1582-1592.
151. Hussain, Z., et al., Exploring recent developments to improve antioxidant, anti-inflammatory and antimicrobial efficacy of curcumin: A review of new trends and future perspectives. Materials science and engineering: C, 2017. 77: p. 1316-1326.
152. Calabrese, V., et al., Curcumin and the cellular stress response in free radical-related diseases. Molecular nutrition & food research, 2008. 52(9): p. 1062-1073.
153. Alvarez-Suarez, J. M., et al., The composition and biological activity of honey: a focus on Manuka honey. Foods, 2014. 3(3): p. 420-432.
154. Khan, R. U., S. Naz, and A. M. Abudabos, Towards a better understanding of the therapeutic applications and corresponding mechanisms of action of honey. Environmental Science and Pollution Research, 2017. 24(36): p. 27755-27766.
155. Lindberg, T., et al., A systematic review and meta-analysis of dressings used for wound healing: the efficiency of honey compared to silver on burns. Contemporary nurse, 2015. 51(2-3): p. 121-134.
156. Mukai, K., et al., Effectiveness of Changing the Application of Japanese Honey to a Hydrocolloid Dressing in Between the Inflammatory and Proliferative Phases on Cutaneous Wound Healing in Male Mice. Wounds: a compendium of clinical research and practice, 2016. 29(1): p. 1-9.
157. Niaz, K., et al., Health benefits of manuka honey as an essential constituent for tissue regeneration. Current drug metabolism, 2017. 18(10): p. 881-892.
158. Jull, A. B., et al., Honey as a topical treatment for wounds. Cochrane Database of Systematic Reviews, 2015(3): p. Art. No.: CD005083.
159. Long, M., et al., An essential role of NRF2 in diabetic wound healing. Diabetes, 2016. 65(3): p. 780-793.
160. Zheng, H., et al., Therapeutic potential of Nrf2 activators in streptozotocin-induced diabetic nephropathy. Diabetes, 2011. 60(11): p. 3055-3066.
161. Suzuki, T. and M. Yamamoto, Stress-sensing mechanisms and the physiological roles of the Keap1-Nrf2 system during cellular stress. Journal of Biological Chemistry, 2017. 292(41): p. 16817-16824.
162. Uruno, A., Y. Yagishita, and M. Yamamoto, The Keap1-Nrf2 system and diabetes mellitus. Archives of biochemistry and biophysics, 2015. 566: p. 76-84.
163. Jiang, T., et al., Nrf2 suppresses lupus nephritis through inhibition of oxidative injury and the NF-κB-mediated inflammatory response. Kidney international, 2014. 85(2): p. 333-343.
164. Chu, H.-q., et al., Aminoglycoside ototoxicity in three murine strains and effects on NKCC1 of stria vascularis. Chinese medical journal, 2006. 119(12): p. 980-985.
165. Patrick, B. N., M. P. Rivey, and D. R. Allington, Acute renal failure associated with vancomycin-and tobramycin-laden cement in total hip arthroplasty. Annals of Pharmacotherapy, 2006. 40(11): p. 2037-2042.
166. Misra, A. and J. Nanchahal, Use of gauze soaked in povidone iodine for dressing acute open wounds. Plastic and reconstructive surgery, 2003. 111(6): p. 2105-2106.
167. Motta, G. J., C. T. Milne, and L. Q. Corbett, Impact of antimicrobial gauze on bacterial colonies in wounds that require packing. Ostomy/wound management, 2004. 50(8): p. 48-62.
168. Fukunaga, A., et al., Our method of povidone-iodine ointment and gauze dressings reduced catheter-related infection in serious cases. Dermatology, 2006. 212(Suppl. 1): p. 47-52.
169. Dealey, C., Role of hydrocolloids in wound management. British Journal of Nursing, 1993. 2(7): p. 358-365.
170. Hudspith, J. and S. Rayatt, First aid and treatment of minor burns. Bmj, 2004. 328(7454): p. 1487-1489.
171. Liao, Z., et al., Multi-center clinical study of the effect of silver nitrate ointment on the partial-thickness burn wounds. Zhonghua shao shang za zhi=Zhonghua shaoshang zazhi=Chinese journal of burns, 2006. 22(5): p. 359-361.
172. Queen, D., et al., A dressing history. International wound journal, 2004. 1(1): p. 59-77.
173. Falabella, A. F., Debridement and wound bed preparation. Dermatologic therapy, 2006. 19(6): p. 317-325.
174. G M, E., Wound dressings. Pharmaceutics: The science of dosage form design. 3rd edition, ed. A. M E. 2007, U K: Churchill Livingstone.
175. Van Rijswijk, L., Ingredient-based wound dressing classification: a paradigm that is passe and in need of replacement. Journal of wound care, 2006. 15(1): p. 11-14.
176. Purner S K, B. M., Collagen based dressings—a review. Burns, 2000. 26(1): p. 54-62.
177. Saghazadeh, S., et al., Drug delivery systems and materials for wound healing applications. Advanced drug delivery reviews, 2018. 127: p. 138-166.
178. Wawrzyńska, E. and D. Kubies, Alginate matrices for protein delivery—a short review. Physiological research, 2018. 67 (Suppl. 2): p. S319-S334.
179. Murray, R. Z., et al., Development and use of biomaterials as wound healing therapies. Burns & trauma, 2019. 7(7:2).
180. Dhall, S., et al., Generating and reversing chronic wounds in diabetic mice by manipulating wound redox parameters. Journal of diabetes research, 2014. 2014: p. 18.
181. Shannon, M. B., et al., Bladder urinary oxygen tension is correlated with urinary microbiota composition. International Urogynecology Journal, 2019. 30(8): p. 1261-1267.
182. Ortiz-Prado, E., et al., Partial pressure of oxygen in the human body: a general review. American journal of blood research, 2019. 9(1): p. 1.
183. Carreau, A., et al., Why is the partial oxygen pressure of human tissues a crucial parameter? Small molecules and hypoxia. Journal of cellular and molecular medicine, 2011. 15(6): p. 1239-1253.
184. Aanaes, K., et al., Decreased mucosal oxygen tension in the maxillary sinuses in patients with cystic fibrosis. Journal of Cystic Fibrosis, 2011. 10(2): p. 114-120.
185. Schaffer, K. and C. T. Taylor, The impact of hypoxia on bacterial infection. The FEBS journal, 2015. 282(12): p. 2260-2266.
186. Schreml, S., et al., Oxygen in acute and chronic wound healing. British Journal of Dermatology, 2010. 163(2): p. 257-268.
187. Kolpen, M., et al., Polymorphonuclear leucocytes consume oxygen in sputum from chronic Pseudomonas aeruginosa pneumonia in cystic fibrosis. Thorax, 2010. 65(1): p. 57-62.
188. Light, D. R., et al., Characteristics of the cofactor requirements for the superoxide-generating NADPH oxidase of human polymorphonuclear leukocytes. Biochemistry, 1981. 20(6): p. 1468-1476.
189. Wu, H., et al., Strategies for combating bacterial biofilm infections. International journal of oral science, 2015. 7(1): p. 1-7.
190. Wolcott, R. D. and G. D. Ehrlich, Biofilms and chronic infections. Jama, 2008. 299(22): p. 2682-2684.
191. Stewart, P. S. and M. J. Franklin, Physiological heterogeneity in biofilms. Nature Reviews Microbiology, 2008. 6(3): p. 199-210.
192. Hamad, M. A., et al., Adaptation and antibiotic tolerance of anaerobic Burkholderia pseudomallei. Antimicrobial agents and chemotherapy, 2011. 55(7): p. 3313-3323.
193. Hess, D. J., et al., Anoxia inhibits biofilm development and modulates antibiotic activity. Journal of Surgical Research, 2013. 184(1): p. 488-494.
194. Brauner, A., et al., Distinguishing between resistance, tolerance and persistence to antibiotic treatment. Nature Reviews Microbiology, 2016. 14(5): p. 320-330.
195. Sen, C. K., et al., Human skin wounds: a major and snowballing threat to public health and the economy. Wound repair and regeneration, 2009. 17(6): p. 763-771.
196. Lipsky, B. A. and U. Stoutenburgh, Daptomycin for treating infected diabetic foot ulcers: evidence from a randomized, controlled trial comparing daptomycin with vancomycin or semi-synthetic penicillins for complicated skin and skin-structure infections. Journal of antimicrobial chemotherapy, 2005. 55(2): p. 240-245.
197. Han, G. and R. Ceilley, Chronic wound healing: a review of current management and treatments. Advances in therapy, 2017. 34: p. 599-610.
198. Siami, G., et al., Clinafloxacin versus piperacillin-tazobactam in treatment of patients with severe skin and soft tissue infections. Antimicrobial agents and chemotherapy, 2001. 45(2): p. 525-531.
199. Lipsky, B. A., et al., Treating foot infections in diabetic patients: a randomized, multicenter, open-label trial of linezolid versus ampicillin-sulbactam/amoxicillin-clavulanate. Clinical Infectious Diseases, 2004. 38(1): p. 17-24.
200. Schaper, N., et al., Efficacy and safety of IV/PO moxifloxacin and IV piperacillin/tazobactam followed by PO amoxicillin/clavulanic acid in the treatment of diabetic foot infections: results of the RELIEF study. Infection, 2013. 41: p. 175-186.
201. Cowley, E. S., et al., Pediatric cystic fibrosis sputum can be chemically dynamic, anoxic, and extremely reduced due to hydrogen sulfide formation. MBio, 2015. 6(4): p. 10.1128/mbio. 00767-15.
202. Kopf, S. H., et al., Trace incorporation of heavy water reveals slow and heterogeneous pathogen growth rates in cystic fibrosis sputum. Proceedings of the National Academy of Sciences, 2016. 113(2): p. E110-E116.
203. Kragh, K. N., et al., Polymorphonuclear leukocytes restrict growth of Pseudomonas aeruginosa in the lungs of cystic fibrosis patients. Infection and Immunity, 2014. 82(11): p. 4477-4486.
204. Valentini, T. D., et al., Bioorthogonal non-canonical amino acid tagging reveals translationally active subpopulations of the cystic fibrosis lung microbiota. Nature communications, 2020. 11(1): p. 2287.
205. Chmiel, J. F. and P. B. Davis, State of the art: why do the lungs of patients with cystic fibrosis become infected and why can't they clear the infection? Respiratory research, 2003. 4: p. 1-12.
206. Perry, E. K. and M.-W. Tan, Bacterial biofilms in the human body: Prevalence and impacts on health and disease. Frontiers in Cellular and Infection Microbiology, 2023. 13: p. 1237164.
207. James, G. A., et al., Biofilms in chronic wounds. Wound Repair and regeneration, 2008. 16(1): p. 37-44.
208. DePas, W. H., et al., Exposing the three-dimensional biogeography and metabolic states of pathogens in cystic fibrosis sputum via hydrogel embedding, clearing, and rRNA labeling. MBio, 2016. 7(5): p. 10.1128/mbio. 00796-16.
209. Bjarnsholt, T., et al., The in vivo biofilm. Trends in microbiology, 2013. 21(9): p. 466-474.
210. Borriello, G., et al., Oxygen limitation contributes to antibiotic tolerance of Pseudomonas aeruginosa in biofilms. Antimicrobial agents and chemotherapy, 2004. 48(7): p. 2659-2664.
211. González, P., et al., Bacterial nitrate reductases: molecular and biological aspects of nitrate reduction. Journal of inorganic biochemistry, 2006. 100(5-6): p. 1015-1023.
212. Winter, S. E., et al., Host-derived nitrate boosts growth of E. coli in the inflamed gut. science, 2013. 339(6120): p. 708-711.
213. Bernatchez, S. F., et al., Nitric oxide levels in wound fluid may reflect the healing trajectory. Wound Repair and Regeneration, 2013. 21(3): p. 410-417.
214. Palmer, K. L., L. M. Aye, and M. Whiteley, Nutritional cues control Pseudomonas aeruginosa multicellular behavior in cystic fibrosis sputum. Journal of bacteriology, 2007. 189(22): p. 8079-8087.
215. Radi, R., Oxygen radicals, nitric oxide, and peroxynitrite: Redox pathways in molecular medicine. Proceedings of the National Academy of Sciences, 2018. 115(23): p. 5839-5848.
216. Lopez, C. A., et al., Phage-mediated acquisition of a type III secreted effector protein boosts growth of salmonella by nitrate respiration. MBio, 2012. 3(3): p. 10.1128/mbio. 00143-12.
217. Spees, A. M., et al., Streptomycin-induced inflammation enhances Escherichia coli gut colonization through nitrate respiration. MBio, 2013. 4(4): p. 10.1128/mbio. 00430-13.
218. Jones, S. A., et al., Anaerobic respiration of Escherichia coli in the mouse intestine. Infection and immunity, 2011. 79(10): p. 4218-4226.
219. Rivera-Chávez, F., et al., Energy taxis toward host-derived nitrate supports a Salmonella pathogenicity island 1-independent mechanism of invasion. MBio, 2016. 7(4): p. 10.1128/mbio. 00960-16.
220. Williams, M. J., et al., bis-Molybdopterin guanine dinucleotide is required for persistence of Mycobacterium tuberculosis in guinea pigs. Infection and Immunity, 2015. 83(2): p. 544-550.
221. Jung, J.-Y., et al., The intracellular environment of human macrophages that produce nitric oxide promotes growth of mycobacteria. Infection and immunity, 2013. 81(9): p. 3198-3209.
222. Fritz, C., et al., Dependence of Mycobacterium bovis BCG on anaerobic nitrate reductase for persistence is tissue specific. Infection and immunity, 2002. 70(1): p. 286-291.
223. Abdou, E., et al., RegA, the regulator of the two-component system RegB/RegA of Brucella suis, is a controller of both oxidative respiration and denitrification required for chronic infection in mice. Infection and Immunity, 2013. 81(6): p. 2053-2061.
224. Köhler, S., et al., The analysis of the intramacrophagic virulome of Brucella suis deciphers the environment encountered by the pathogen inside the macrophage host cell. Proceedings of the National Academy of Sciences, 2002. 99(24): p. 15711-15716.
225. Kim, H. S., et al., Bacterial genome adaptation to niches: divergence of the potential virulence genes in three Burkholderia species of different survival strategies. BMC genomics, 2005. 6: p. 1-13.
226. Chin, C.-Y., et al., Global transcriptional analysis of Burkholderia pseudomallei high and low biofilm producers reveals insights into biofilm production and virulence. BMC genomics, 2015. 16: p. 1-15.
227. Kim, J. H., et al., Targeting anaerobic respiration in Pseudomonas aeruginosa with chlorate improves healing of chronic wounds. Advances in Wound Care, 2024. 13(2): p. 53-69.
228. Hochstein, L. I. and G. A. Tomlinson, The enzymes associated with denitrification. 1988.
229. Puig, J., et al., Genetic mapping of the chl C gene of the nitrate reductase A system in Escherichia coli K12. Biochemical and biophysical research communications, 1969. 35(5): p. 659-662.
230. Smith, D., et al., Invited review: Efficacy, metabolism, and toxic responses to chlorate salts in food and laboratory animals. Journal of animal science, 2012. 90(11): p. 4098-4117.
231. Canada, H., Guidelines for Canadian drinking water quality: Guideline technical document—Chlorite and chlorate.. 2008.
232. Jackson, R., W. Elder, and H. McDonnell, Sodium-chlorate poisoning complicated by acute renal failure. The Lancet, 1961. 278(7217): p. 1381-1383.
233. Helliwell, M. and J. Nunn, Mortality in sodium chlorate poisoning. British Medical Journal, 1979. 1(6171): p. 1119.
234. Cook, G. M., et al., Energetics of pathogenic bacteria and opportunities for drug development. Advances in microbial physiology, 2014. 65: p. 1-62.
235. Vázquez-Torres, A. and A. J. Bäumler, Nitrate, nitrite and nitric oxide reductases: from the last universal common ancestor to modern bacterial pathogens. Current opinion in microbiology, 2016. 29: p. 1-8.
236. Moriarty, T., et al., Sputum antibiotic concentrations: implications for treatment of cystic fibrosis lung infection. Pediatric pulmonology, 2007. 42(11): p. 1008-1017.
237. Gallagher, T., et al., Liquid chromatography mass spectrometry detection of antibiotic agents in sputum from persons with cystic fibrosis. Antimicrobial Agents and Chemotherapy, 2021. 65(2): p. 10.1128/aac. 00927-20.
238. Aubert, G., et al., Prospective determination of serum ceftazidime concentrations in intensive care units. Therapeutic drug monitoring, 2010. 32(4): p. 517-519.
239. Buijk, S., et al., Pharmacokinetics of ceftazidime in serum and peritoneal exudate during continuous versus intermittent administration to patients with severe intra-abdominal infections. Journal of Antimicrobial Chemotherapy, 2002. 49(1): p. 121-128.
240. Iskandar, K., et al., Antibiotic discovery and resistance: the chase and the race. Antibiotics, 2022. 11(2): p. 182.
241. Murugaiyan, J., et al., Progress in alternative strategies to combat antimicrobial resistance: Focus on antibiotics. Antibiotics, 2022. 11(2): p. 200.
242. Coates, A. R., et al., Antibiotic combination therapy against resistant bacterial infections: synergy, rejuvenation and resistance reduction. Expert review of Anti-infective therapy, 2020. 18(1): p. 5-15.
243. Bollenbach, T., Antimicrobial interactions: mechanisms and implications for drug discovery and resistance evolution. Current opinion in microbiology, 2015. 27: p. 1-9.
244. Brown, J. C., et al., Unraveling the biology of a fungal meningitis pathogen using chemical genetics. Cell, 2014. 159(5): p. 1168-1187.
245. Sullivan, G. J., et al., How antibiotics work together: molecular mechanisms behind combination therapy. Current opinion in microbiology, 2020. 57: p. 31-40.
246. Wambaugh, M. A., et al., High-throughput identification and rational design of synergistic small-molecule pairs for combating and bypassing antibiotic resistance. PLoS Biology, 2017. 15(6): p. e2001644.
247. Spero, M. A., et al., Mechanisms of chlorate toxicity and resistance in Pseudomonas aeruginosa. Molecular microbiology, 2022. 118(4): p. 321-335.
248. Pompilio, A., et al., In vitro activity of colistin against biofilm by Pseudomonas aeruginosa is significantly improved under “cystic fibrosis—like” physicochemical conditions. Diagnostic microbiology and infectious disease, 2015. 82(4): p. 318-325.
249. Kolpen, M., et al., Increased bactericidal activity of colistin on Pseudomonas aeruginosa biofilms in anaerobic conditions. FEMS Pathogens and Disease, 2016. 74(1): p. ftv086.
250. Livingston, J., et al., Visualization of mRNA expression in Pseudomonas aeruginosa aggregates reveals spatial patterns of fermentative and denitrifying metabolism. Applied and Environmental Microbiology, 2022. 88(11): p. e00439-22.
251. electrodes, W.C., 2024.

1. A method for timed and/or targeted chlorate administration of a biological environment or a region thereof, the method comprising
administering an antibiotic to the biological environment or region thereof when the biological environment or region thereof is in an oxic condition, the administering performed at an oxic antibiotic effective amount to inhibit viability of Nar-containing bacteria in an oxic environment,
administering chlorate in combination with an antibiotic to the biological environment or region thereof, when the biological environment or region thereof in a hypoxic condition, the administering performed at a hypoxic chlorate effective amount and a hypoxic antibiotic effective amount to inhibit viability of Nar-containing bacteria in a hypoxic environment; and
administering chlorate to the biological environment or the region thereof, the biological environment or region thereof in an anoxic condition, the administering performed at an anoxic chlorate effective amount to inhibit viability of Nar-containing bacteria in an anoxic environment
the administering chlorate to the biological environment or region thereof in anaerobic conditions, performed optionally in combination with an antibiotic in an antibiotic effective among in anoxic environment.

2. The method of claim 1, wherein the antibiotic effective amount in a hypoxic environment and/or the antibiotic effective amount in an anoxic environment is lower than the antibiotic amount in an oxic environment.

3. The method of claim 2, wherein the antibiotic effective amount in a hypoxic environment and/or the antibiotic effective amount in an anoxic environment is from ¼ to 1/100 of the minimum inhibitory concentration of the antibiotic.

4. The method of claim 1, wherein the method further comprises detecting in the biological environment or target region thereof or a sample thereof at least one of oxygen level, nitrate concentration and redox potential to the detect an oxygenation status of the biological environment or target region thereof, to determine whether the biological environment or target region thereof are in an oxic condition, a hypoxic condition or an anoxic condition.

5. The method of claim 4, wherein biological environment or target region thereof is in an oxic condition, when a detected oxygen level is above a threshold detected oxygen level of 200 uM, a nitrate concentration is below 100 uM and/or a redox potential is above a threshold detected redox potential above 300 mV.

6. the method of claim 4, wherein biological environment or target region thereof is in a hypoxic condition, when a detected oxygen level is from 20 uM to 200 uM a redox potential below 300 mV and a detectable concentration of nitrate above 100 uM.

7. The method of claim 4, wherein biological environment or target region thereof is in an anoxic condition, when a detected oxygen level is lower than 20 uM, a detected nitrate concentration above 100 uM more preferably above 500 uM, and/or a detected redox potential below 200 mV.

8. The method of claim 4, wherein the detecting is performed on the biological environment or target region thereof or a sample thereof at a plurality of times to monitor the change in oxygenation status of the at least one target region of the biological environment.

9. The method of claim 1, wherein the biological environment or target region thereof are in hypoxic condition and/or in anoxic condition at a depth of 50-100 um from a surface exposed to oxygen.

10. The method of claim 1, wherein the biological environment or target region thereof are in hypoxic condition and/or in anoxic condition at depth greater than ˜10-20 um from a surface exposed to oxygen.

11. The method of claim 1, wherein the biological environment or target region thereof are in hypoxic condition and/or in anoxic condition at depth within ˜5 μm from a surface exposed to oxygen.

12. The method of claim 1, wherein the biological environment or target region thereof are in hypoxic condition and/or in anoxic condition comprises a biofilm.

13. The method of claim 12, wherein the biofilm has of a diameter >20 um.

14. The method of claim 1, wherein at least one of
administering chlorate under hypoxic condition is performed at a hypoxic chlorate administration time,
administering chlorate under anoxic condition is performed at an anoxic chlorate administration time,
administering antibiotic under oxic condition is performed an oxic antibiotic administration time and
administering antibiotic under hypoxic condition is performed at a hypoxic antibiotic administration time and
wherein at least one of the hypoxic chlorate administration time, the anoxic chlorate administration time, oxic antibiotic administration time and hypoxic antibiotic administration time is selected based on oxygen levels in the biological environment estimated through modeling.

15. The method of claim 1, at least one of
administering chlorate under hypoxic condition is performed at a hypoxic chlorate administration time
administering chlorate under anoxic condition is performed at an anoxic chlorate administration time,
administering antibiotic under oxic condition is performed an oxic antibiotic administration time and
administering antibiotic under hypoxic condition is performed at a hypoxic antibiotic administration time and
wherein at least one of the hypoxic chlorate administration time, the anoxic chlorate administration time, oxic antibiotic administration time and hypoxic antibiotic administration time is selected based on oxygen levels in the biological environment detected by one or more oxygen sensors.

16. The method of claim 1, at least one of
administering chlorate under hypoxic condition is performed at a hypoxic chlorate administration time
administering chlorate under anoxic condition is performed at an anoxic chlorate administration time,
administering antibiotic under oxic condition is performed an oxic antibiotic administration time and
administering antibiotic under hypoxic condition is performed at a hypoxic antibiotic administration time and
wherein at least one of the hypoxic chlorate administration time, the anoxic chlorate administration time, oxic antibiotic administration time and hypoxic antibiotic administration time is selected based on detection in the biological environment of one or more biomarkers of anaerobic respiration of the Nar-containing bacteria.

17. The method of claim 14, wherein at least one of hypoxic chlorate administration time and the anoxic chlorate administration time is selected from 1 day to 4 months from onset of an infection by the Nar-containing bacteria in the biological environment or the region thereof.

18. The method of claim 14, wherein at least one of hypoxic chlorate administration time and the anoxic chlorate administration time is selected from than 1 day to 30 days from onset of an infection by the Nar-containing bacteria in the biological environment or the target region thereof.

19. The method of claim 14, wherein at least one of hypoxic chlorate administration time and the anoxic chlorate administration time is selected from 10 days to 15 days from the onset of an infection by the NAR-containing bacteria.

20. The method of claim 4, wherein the detecting is performed by
(a) detecting at least one of i) an oxygen level, ii) a redox potential, and iii) a nitrate concentration of the infected biological environment,
and wherein the
(b) administering to the biological environment or region thereof an antibiotic in the oxic antibiotic amount effective to inhibit viability of the bacteria, is performed when at least one of an oxygen level above a threshold level of 200 uM, preferably 150 uM or more, or preferably of 100 uM, a redox potential, above a threshold redox potential of 300 mV, preferably of 250 mV or more, or preferably 200 mV, and preferably no nitrate concentration is detected,
(c2a) administering a chlorate and an antibiotic to the biological environment or region thereof, the chlorate and the antibiotic administered in a hypoxic chlorate effective amount and a hypoxic antibiotic effective amount effective to inhibit viability of the bacteria, is performed when at least one of an oxygen level is below a threshold level of 200 uM, preferably 150 uM or more, or preferably of 100 uM and above a threshold level of 20 uM, preferably in combination with a redox potential below a threshold redox potential of 300 mV, preferably of 250 mV or more preferably 200 mV and a nitrate concentration above a threshold level of 500 uM is detected, and
(c2b) the administering a chlorate to the biological environment or region thereof, the chlorate and the antibiotic administered in a anoxic chlorate effective amount to inhibit viability of the bacteria, is performed when at least one of an oxygen level is below the detected threshold of 20 uM, preferably when a redox potential below the detected threshold of 300 uM, more preferably 200 mV and/or a nitrate concentration above the detected threshold, preferably 500 uM is detected, the administering optionally performed in combination with administering an anoxic antibiotic effective amount.

21. The method of claim 1, wherein the method comprises
administering chlorate in combination with an antibiotic to the biological environment or region thereof, when the biological environment or region thereof in a hypoxic condition and/or
and the method further comprises
(d1) administering nitrate to the biological environment or region thereof in a hypoxic condition for a nitrate contacting time and in a nitrate amount effective to increase expression of a Nar gene in the Nar-containing bacteria, and
(d2) after the nitrate contacting time, administering to the biological environment or region thereof the chlorate in combination with antibiotics in the hypoxic chlorate effective amount and the hypoxic antibiotic effective amount, the chlorate effective amount and the nitrate effective amount in a ratio from 4:1 to 10:1.

22. The method of claim 21, wherein the nitrate effective amount is from 0.1 mM to 50 mM, and the amount selected to stimulate expression of Nar without preventing processing of chlorate.

23. The method of claim 21, wherein the nitrate effective amount is an amount from 0.1 mM to 50 mM selected to have a chlorate:nitrate concentration ratio of at least 10:1 in the biological environment or region thereof.

24.-27. (canceled)

28. The method of claim 1, wherein
the administering an antibiotic to the biological environment or region thereof when the biological environment or region thereof is in an oxic condition,
the administering chlorate in combination with an antibiotic to the biological environment or region thereof, when the biological environment or region thereof in a hypoxic condition and
administering chlorate to the biological environment or the region thereof, the biological environment or region thereof in an anoxic condition,
are performed to treat and/or prevent an infection of the biological environment or region thereof by the Nar containing bacteria.

29.-88. (canceled)

89. The method of claim 1, wherein administering an antibiotic and/or administering chlorate is performed from a medical implant configured for
releasing from the medical implant to the oxic environment the oxic antibiotic effective amount,
releasing from the medical implant to the hypoxic environment the hypoxic chlorate effective amount and the hypoxic antibiotic effective, and
releasing from the medical implant to the anoxic environment the anoxic chlorate effective amount.

90. The method of claim 89, wherein the medical implant is further configured for
detecting at least one of oxygen level, nitrate concentration and redox potential to the detect an oxygenation status of the biological environment or target region thereof, and wherein at least one of
releasing from the medical implant to the oxic environment the oxic antibiotic effective amount to the one or more oxic portions,
releasing from the medical implant to the hypoxic environment the hypoxic chlorate effective amount and the hypoxic antibiotic effective amount, and
releasing from the medical implant to the anoxic environment, the anoxic chlorate effective amount, is performed following detecting the one or more anoxic portions by the medical implant
is performed following detecting the one or more oxic portions by the medical implant.

91. The method of claim 89, wherein the medical implant is further configured for releasing nitrate to the hypoxic environment for a nitrate contacting time and in a nitrate amount effective to increase expression of a Nar gene in the Nar-containing bacteria, and
wherein the method further comprises
releasing from the medical implant to the hypoxic environment the nitrate effective amount for the nitrate contacting time and
after the nitrate contacting time, releasing from the medical implant to the hypoxic environment the chlorate effective amount in combination with antibiotics in the hypoxic chlorate effective amount and the hypoxic antibiotic effective amount,
the chlorate effective amount and the nitrate effective amount in a ratio from 4:1 to 10:1.

92. The method of claim 1, wherein administering an antibiotic and/or administering chlorate is performed from a bandage configured for
releasing from the bandage to the oxic environment the oxic antibiotic effective amount,
releasing from the bandage to the hypoxic environment the hypoxic chlorate effective amount and the hypoxic antibiotic effective, and
releasing from the bandage to the anoxic environment the anoxic chlorate effective amount.

93. The method of claim 92, wherein the bandage is further configured for
detecting at least one of oxygen level, nitrate concentration and redox potential to the detect an oxygenation status of the biological environment or target region thereof, and wherein at least one of
releasing from the bandage to the oxic environment the oxic antibiotic effective amount to the one or more oxic portions,
releasing from the bandage to the hypoxic environment the hypoxic chlorate effective amount and the hypoxic antibiotic effective amount, and
releasing from the bandage to the anoxic environment the anoxic chlorate effective amount to the one or more anoxic portions,
is performed following detecting the one or more anoxic portions by the bandage.

94. The method of claim 92, wherein the bandage is further configured for releasing nitrate to the hypoxic environment for a nitrate contacting time and in a nitrate amount effective to increase expression of a Nar gene in the Nar-containing bacteria, and
wherein the method further comprises
releasing from the bandage to the hypoxic environment the nitrate effective amount for the nitrate contacting time, and
after the nitrate contacting time, releasing from the bandage to the hypoxic environment the chlorate effective amount in combination with antibiotics in the hypoxic chlorate effective amount and the hypoxic antibiotic effective amount, the chlorate effective amount and the nitrate effective amount in a ratio from 4:1 to 10:1.


SEQUENCE LISTING (95 sequences)

SEQ ID NO: 1 - protein, 1261 aa, Pseudomonas aeruginosa
MSHLLDRLQFFKKKQGEFADGHGETSNESRAWEGAYRQRWQHDKIVRSTHGVNCTGSCSWKIYVKNGLITWETQQTDYPRTRPDLPNHEPRGCPRGASYSWYIYSANRLKYPKVRKPLLKLWREARAQHGDPVNAWASIVEDAAKAKSYKSQRGLGGFVRSSWDEVTEIIAAANVYTAKTYGPDRVIGFSPIPAMSMVSYAAGARYLSLIGGVCLSFYDWYCDLPPASPQIWGEQTDVPESADWYNSSYIIAWGSNVPQTRTPDAHFFTEVRYKGTKTVSITPDYSEVAKLTDLWLNPKQGTDAALGMAFGHVILKEFHLDRPSAYFVDYCRQYTDMPMLVLLEEHAGGAFKPTRYLRAADLADNLGQDNNPEWKTIAYDERSGGLVSPTGAIGYRWGESGKWNIAELDGRSGDQTRLQLSLLDGPEHACEVAFPYFAGQEHPHFKGVANDEVLLRRVPFREIVAADGKRLRVATVYDLQMANYSIDRGLGGDNVATSYEDADTPYTPAWQERITGVPAARATQVAREFADSADKTRGKAMVIIGAAMNHWYHMDMNYRAVINMLMMCGCIGQSGGGWAHYVGQEKLRPQTGWAPLAFGLDWSRPPRQMNGTSFFYLHSSQWRHEKLSMHEVLSPLADASRFAEHALDYNIQAERLGWLPSAPQLNRNPLRIAAEAEAAGLPVADYVVRELKSGGLRFASESPDDPQNFPRNMFIWRSNLLGSSGKGHEYMLKYLLGAKNGVMNDDLGKAGGPRPTEVDWVDDGAEGKLDLVTTLDFRMSSTCMYSDIVLPTATWYEKDDLNTSDMHPFIHPLSAATDPAWEAKSDWEIYKAIAKKFSAVAEGHLGVEQDLVTVPLLHDTPTELAQPFGGDGHDWKKGECEPMPGRNLPTLHLVERDYPNVYRKFTSLGPLLDKLGNGGKGIGWNTEKEVKLVGDLNHRVVESGVSQGRPRIDSAIDAAEVVLALAPETNGQVAVKAWEALSKITGREHAHLALPKEDEKIRFRDIQVQPRKIISSPTWSGLEDEHVSYNAGYTNVHELIPWRTITGRQQFYQDHPWMQAFGEGFVSYRPPVNTRTTEKLLNRKPNGNPEITLNWITPHQKWGIHSTYSDNLLMLTLSRGGPIIWLSEHDAAKAGIVDNDWVEVFNANGAATCRAVVSQRVKDGMVMMYHAQERIVNVPGSETTGTRGGHHNSVTRVVLKPTHMIGGYAQQAWGFNYYGTVGCNRDEFVVVRKMSKVDWLDEPRHGGLGGDALPQPLPQDI

SEQ ID NO: 2 - DNA, 3786 bp, Pseudomonas aeruginosa
  source 1..3786: mol_type = genomic DNA; note = Sequence of narG gene
atgagtcacctgctcgaccgcctgcagttcttcaagaagaagcagggcgaattcgccgatggccacggcgagaccagcaacgagagccgcgcctgggaaggtgcctaccggcagcgctggcagcacgacaagatcgtgcgctccacccacggggtgaactgcaccggctcctgctcctggaagatctacgtgaagaacggcctgatcacctgggaaacccagcagaccgactacccgcgcacccgtccggacctgcccaaccacgagccgcgcggctgcccgcgcggggccagctattcctggtacatctacagcgccaaccgcctgaagtacccgaaggtgcgcaagccgttgctcaagctctggcgcgaggcgcgggcgcagcacggcgacccggtgaacgcctgggccagcatcgtcgaggacgccgccaaggcgaagagctacaagagccagcgcggcctgggcggcttcgtccgttccagctgggacgaggtcaccgagatcatcgccgcggccaacgtctacaccgccaagacctacggtccggaccgggtgatcggcttctcgccgatcccggccatgtcgatggtcagctacgccgccggcgcccgctacctgtcgctgatcggcggggtctgcctgagcttctacgactggtactgcgacctgccgccggccagcccgcagatctggggcgagcagaccgacgtgccggagtcggccgactggtacaactccagctacatcatcgcctggggctccaacgtgccgcagacgcggaccccggacgcgcacttcttcaccgaggtgcgctacaagggcaccaagaccgtctccatcaccccggactattccgaggtggccaagctcaccgacctctggctcaaccccaagcagggcaccgacgccgcgctgggcatggccttcggtcacgtgatcctgaaggaattccacctcgaccggccgagcgcctacttcgtcgactactgccgccagtacaccgacatgccgatgctggtgttgctggaagaacacgccggcggcgcgttcaagccgacccgctacctgcgcgccgccgacctggcggacaacctcggccaggacaacaaccccgagtggaagaccatcgcctacgacgagcgcagcggcgggctggtctcgcccaccggcgccatcggctatcgctggggcgagtcaggcaagtggaacatcgccgagctggacggcaggagcggtgaccagacgcgcctgcaactgtcgctgctcgatggcccggaacatgcctgcgaggtggccttcccgtatttcgccgggcaggagcacccgcacttcaagggcgtcgccaacgacgaggtactgctgcgccgggtgccgttccgcgagatcgtcgcggcggacggcaagcgcctgcgggtggccaccgtctacgacctgcagatggccaactacagcatcgaccgcggcctgggcggcgacaacgtggcgacctcctacgaggacgccgacacgccctataccccggcctggcaggagcgcatcaccggcgttccggcggcgcgcgcgacgcaggtcgcccgcgagttcgccgacagcgccgacaagacccgcggcaaggcgatggtgatcatcggcgcggcgatgaaccactggtaccacatggacatgaactaccgcgcggtcatcaacatgctgatgatgtgcggctgcatcggccagagcggcggcggctgggcgcactatgtcggccaggagaagctgcgcccgcagaccggctgggcgccgctggccttcggcctggactggagccggccgccgcggcagatgaacggcaccagcttcttctacctgcacagctcgcaatggcgccacgagaagctgtcgatgcacgaggtgctgtcgccgctggccgacgccagccgcttcgccgaacacgccctggactacaacatccaggccgaacgcctcggctggctgccgtcggcgccgcaactgaaccgcaacccgctgcgcatcgccgccgaggccgaggccgccggcctgccggtcgccgactacgtggtgcgcgaactgaagagcggcggcctgcgcttcgccagcgaatcgccggacgatccgcagaacttcccgcgcaacatgttcatctggcgctccaacctgctgggctcctccggcaagggccacgagtacatgctcaagtacctgctcggggcgaagaacggggtgatgaacgatgacctcggcaaggccggcggtccgcgtcccaccgaggtcgactgggttgacgacggtgccgagggcaagctcgacctggtcaccaccctggacttccgcatgtcctccacctgcatgtactcggacatcgtcctgccgaccgctacctggtacgagaaggacgacctcaacacctccgacatgcaccccttcatccatccgctgtcggcggccaccgatccggcctgggaagccaagagcgactgggagatctacaaggccatcgccaagaagttctccgccgtcgccgaaggccacctcggcgtggagcaggacctggtcacggtgccgctgctgcacgacacccccaccgagctggcgcagccgttcggcggcgacggccatgactggaagaagggcgagtgcgagccgatgccgggacgcaacctgccgacgctgcacctggtcgagcgcgactacccgaacgtctaccgcaagttcacctcgctcggtccgctgctggacaagctgggcaacggcggcaagggcatcggctggaacaccgagaaggaagtgaagctggtcggcgacctcaaccatcgcgtcgtcgagagcggcgtcagccagggccgcccgcgcatcgacagcgccatcgacgccgctgaggtggtcctcgccctggctccggaaaccaacggccaggtcgcggtcaaggcctgggaagcgctgtcgaagatcaccggccgcgagcatgcccacctggcgctgcccaaggaagacgagaagatccgcttccgcgacatccaggtgcagccgcgcaagatcatctccagcccgacctggtccggcctcgaggacgagcacgtcagctacaacgccggctacaccaacgtccacgagctgatcccgtggcgcaccatcaccggtcgccagcagttctaccaggaccacccgtggatgcaggcgttcggcgaaggcttcgtcagctaccggccgccggtcaacacccggaccaccgagaaactgttgaacaggaagcccaacggcaacccggagatcaccctgaactggatcaccccgcaccagaaatggggcatccactccacctacagcgacaacctgctgatgctcaccctgtcgcgcggcggtccgatcatctggctcagcgagcacgacgcggccaaggccgggatcgtcgataacgactgggtcgaggtgttcaacgccaacggcgcggcgacctgccgcgcggtggtcagccagcgggtcaaggacggcatggtgatgatgtaccacgcccaggaacgcatcgtgaacgtacccggcagcgagaccaccggcacccgtggcggccaccacaactcggtgacccgcgtggtgctcaagccgacccacatgatcggcggctacgcccagcaggcctggggcttcaactactacggcacggtcggctgcaaccgcgacgagttcgtcgtggtgcgcaagatgagcaaggtcgactggctggacgaaccccgccacggcggactcggcggcgacgccctgccgcaaccgctgccccaggacatttga

SEQ ID NO: 3 - DNA, 1542 bp, Pseudomonas aeruginosa
  source 1..1542: mol_type = genomic DNA; note = Sequence of narH gene
atgaaaattcgttcgcaagtcggcatggtgctgaacctcgacaagtgcattggttgccacacctgctcgatcacctgcaagaacgtctggaccagccgcgaaggcatggagtacgcctggttcaacaacgtcgagaccaagcccgggatcggctacccgaaggaatgggaaaaccaggagaagtggaagggcggctgggtgcgcgcggcggacggttcgatccgcccgcgcatcggcggcaagttccgcgtgctggcgaacatcttcgccaacccggacctgcccgagatcgacgactactacgaaccgttcgacttcgattaccagcacctgcataccgcgcccaaggccgagcaccagccggtggcgcgcccgcgctcgctggtctccgggcagcgcatggagaagatcgagtggggcccgaactgggaggagatcctcggcaccgagttcgccaagcggcgcaaggacaagaacttcgaccaggtccaggcggacatctacggtgagtacgagaacaccttcatgatgtacctgccgcgcctctgcgagcactgcctgaacccggcgtgcgtggcgtcctgcccgagcggggcgatctacaagcgcgaggaggacggcatcgtcctgatcgaccaggacaagtgccgcggctggcggatgtgcatctccggctgcccgtacaagaagatctacttcaactggaagagcggcaagtccgagaagtgcatcttctgctacccgcgcatcgaggccggccagcccactgtctgctcggagacctgcgtcgggcgcatccgctacctcggcgtgctgctctacgacgccgaccgcatccacgaagtggccagttgcgagaacgagcgcgagctgtacgagaagcaactggagatcttcctcgatccgttcgacccggcggtgatcgcccaggcgcgcaaggacggggtggccgacagcgtcatcgaggcggcgcagaagtcgccggtgtacaagctggcgatggactggaagctggccctgccgctgcacccggaataccgcacgctgccgatggtctggtacgtgccaccgctgtcgccgatccagaacgccgccgccgaggggcacatcggcagcgacggggtgatcccggacgtggagtcgctgcgcatccccgtgcagtacctggccaacctgctcaccgccggcgacaccgcgccggtgctgctggcgctcaagcgcctgctggcgatgcgcgcctacaagcgcgccgagcacgtcgaaggccgccaggacctggaggtgctggccaaggtcgggttgagcgtggagcaggtggaggagatgtaccgctacctggccatcgccaactacgaggatcgcttcgtgatccccagcgcgcaccgcgaggaagcgctttccgatgccttcgccgagcgttccggctgcggcttcagcttcggcaacggctgttccggcggcagcaactccgccgtcaacctgttcggcggcaagccgaccaaccgccgcgacgtgatccaggtcgtgcagatccaggagtga

SEQ ID NO: 4 - DNA, 741 bp, Pseudomonas aeruginosa
  source 1..741: mol_type = genomic DNA; note = Sequence of narJ gene
atgaacgatcacagccaactgttccgcctgctcgccctgctgctcgactatccacgcgccgagctgcgcgaggagagcctcggcctgcatgcgctgatccgcacctgcgaactgccggaagcgctgcgcgacggcctcgcggcgctgctcaacgagctctgccagggcgacctgctggacgtccaggcgcgctacgacggtctcttcgagcgcggccgctcggtctcgctgctgctcttcgagcacgtccacggcgagagccgcgaccgtggccaggcgatggtcgacctgctcgaccgctataccggggccggcctgcagatcgacgtaccggagctgccggactacctgccgctgtacctcgaatacctgtcgctgctgccgttcgcggcggccagcgaagggctcgccgaagtggcgcacatcctcggcctgctggcgctgcgcctggaggaacgcggcagcgcctacgcggcgattttcgaggcgttgctggaactcggcggcgagcgcccggacctcggcgcgttgcgtcgcgaccaggcccaggaacagcgcgacgacagcctggaggccatcgaccgggcctgggaggaaaccccggtgagcttcaccgaccctgccggcggttgcccgtcgagcagcggccgccgtccgacggcgtccaccgaacaaccattgcaatgggtcgcccagccggtaccgcagatgcagtaccgcgcggcccgcgaaggagtctga

SEQ ID NO: 5 - DNA, 684 bp, Pseudomonas aeruginosa
  source 1..684: mol_type = genomic DNA; note = Sequence of narI gene
atgtcgaccaatcttctgttcttcgggatctatccctatgtcgcgctgctgatctgcctggtcggcagctgggcgcgcttcgacctctcgcagtacacctggaaggccggttccagccagatgctcagcaagaagggcatgcgggtatacagcaacctgttccacgtcggcgtgctgttcatcctcgccggccacttcgtcggcctgctgaccccggcctcggtctaccaccacctgatcagcaccgagaacaagcaactgctggcgatggtctccggcggcttcttcggcgtgctctgcttcatcggcctgagcggactgatcctgcgccgcctgaccgacgcgcgggtgcgcgccaccggcaacgcctctgacctgatgatcctgctggtgctctacgcccagctgatcctcggcctctccaccatcgtcgcctcgacccatcacatggatggctcggtgatggtgatgctcgccgactgggcccaggccatcgtcaccctgcgtccgctggcggcggccgaagccatcgcgccggtgggcctggtctacaagctgcacgtcggcctgggcctgaccctgttcgtgctgttccccttcacccgcctggtgcacatcgtcagcgcgccggtgtggtacctgggccggcgctaccagatcgtgcgtcagaaacgtcctgcctga

SEQ ID NO: 6 - DNA, 50 bp, Pseudomonas aeruginosa
  source 1..50: mol_type = genomic DNA; note = target sequence nosZ 1
gagcgacgacacgaaaagcccccacgaagaaacccacggcctgaaccgcc

SEQ ID NO: 7 - DNA, 50 bp, Pseudomonas aeruginosa
  source 1..50: mol_type = genomic DNA; note = target sequence nosZ 2
gcgcagcaaggccgaggtcgcccccggcgaactggatgagtactacgggt

SEQ ID NO: 8 - DNA, 50 bp, Pseudomonas aeruginosa
  source 1..50: mol_type = genomic DNA; note = target sequence nosZ 3
ggagcggcggacattccggcgaagtacgcgtgctcggcgtgccgtcgatg

SEQ ID NO: 9 - DNA, 50 bp, Pseudomonas aeruginosa
  source 1..50: mol_type = genomic DNA; note = target sequence nosZ 4
gagctgatgcgcataccggtgttcaacgtcgactcggccaccggctgggg

SEQ ID NO: 10 - DNA, 50 bp, Pseudomonas aeruginosa
  source 1..50: mol_type = genomic DNA; note = target sequence nosZ 5
gaccaacgagagcaagcgggtcctcggcgacagcgcgcgcttcctcaacg

SEQ ID NO: 11 - DNA, 50 bp, Pseudomonas aeruginosa
  source 1..50: mol_type = genomic DNA; note = target sequence nosZ 6
actgccaccatccgcacatctcgatgaccgacggcaagtacgacggcaag

SEQ ID NO: 12 - DNA, 50 bp, Pseudomonas aeruginosa
  source 1..50: mol_type = genomic DNA; note = target sequence nosZ 7
catgaaatgcgaccgcatcgtcaccattcccaacgtccaggcgatccacg

SEQ ID NO: 13 - DNA, 50 bp, Pseudomonas aeruginosa
  source 1..50: mol_type = genomic DNA; note = target sequence nosZ 8
tgcgcctgcaaaaggtgccgcatacccgctacgtgttctgcaacgccgag

SEQ ID NO: 14 - DNA, 50 bp, Pseudomonas aeruginosa
  source 1..50: mol_type = genomic DNA; note = target sequence nosZ 9
atcatcccccatcccaacgacggctcgaccttcgacctgtccggcgacaa

SEQ ID NO: 15 - DNA, 50 bp, Pseudomonas aeruginosa
  source 1..50: mol_type = genomic DNA; note = target sequence nosZ 10
cttcaccctgtacaacgccatcgacgccgagaccatggaagtggcctggc

SEQ ID NO: 16 - DNA, 50 bp, Pseudomonas aeruginosa
  source 1..50: mol_type = genomic DNA; note = target sequence nosZ 11
tgatcgtcgacggcaacctcgacaacaccgacatggactacagcggcagg

SEQ ID NO: 17 - DNA, 50 bp, Pseudomonas aeruginosa
  source 1..50: mol_type = genomic DNA; note = target sequence nosZ 12
gccgcctccacctgctacaactcggagaaggccgtcgacctcggcggcat

SEQ ID NO: 18 - DNA, 50 bp, Pseudomonas aeruginosa
  source 1..50: mol_type = genomic DNA; note = target sequence nosZ 13
agatcaaggcgaagcgcttcgtcaccctcggcgactcgaaggtgccggtg

SEQ ID NO: 19 - DNA, 50 bp, Pseudomonas aeruginosa
  source 1..50: mol_type = genomic DNA; note = target sequence nosZ 14
gacggccggcgcaaggacggcaaggacagcccggtgacccgctacatccc

SEQ ID NO: 20 - DNA, 50 bp, Pseudomonas aeruginosa
  source 1..50: mol_type = genomic DNA; note = target sequence nosZ 15
accgaagaacccccacgggctgaacacctcgccggacggcaagtacttca

SEQ ID NO: 21 - DNA, 50 bp, Pseudomonas aeruginosa
  source 1..50: mol_type = genomic DNA; note = target sequence nosZ 16
ccaacggcaagctctcgccgacctgcaccatgatcgccatcgagcgcctc

SEQ ID NO: 22 - DNA, 50 bp, Pseudomonas aeruginosa
  source 1..50: mol_type = genomic DNA; note = target sequence nosZ 17
gacctgttcgccggcaagctggccgacccgcgcgacgtggtggtgggcga

SEQ ID NO: 23 - DNA, 50 bp, Pseudomonas aeruginosa
  source 1..50: mol_type = genomic DNA; note = target sequence nosZ 18
ggaactgggcctcggcccgctgcacaccactttcgatggccgaggcaacg

SEQ ID NO: 24 - DNA, 50 bp, Pseudomonas aeruginosa
  source 1..50: mol_type = genomic DNA; note = target sequence nosZ 19
ataccacgctgttcatcgacagccagttggtgaagtggaacctggccgac

SEQ ID NO: 25 - DNA, 50 bp, Pseudomonas aeruginosa
  source 1..50: mol_type = genomic DNA; note = target sequence nosZ 20
tgccacgccctgcacatggaaatgtgcgggcggatgctggtggaaaaggc

SEQ ID NO: 26 - DNA, 50 bp, Pseudomonas aeruginosa
  source 1..50: mol_type = genomic DNA; note = target sequence ccoN1 1
tcgccgttatgacggtggtctggggggtcattggaatgggtctcggtgtc

SEQ ID NO: 27 - DNA, 50 bp, Pseudomonas aeruginosa
  source 1..50: mol_type = genomic DNA; note = target sequence ccoN1 2
tcggtgggtgcgccctcttcgccacctcgtactacgtggtgcaacgtacc

SEQ ID NO: 28 - DNA, 50 bp, Pseudomonas aeruginosa
  source 1..50: mol_type = genomic DNA; note = target sequence ccoN1 3
ctgatttcagacacgctggcggccttcaccttctggggttggcaggccgt

SEQ ID NO: 29 - DNA, 50 bp, Pseudomonas aeruginosa
  source 1..50: mol_type = genomic DNA; note = target sequence ccoN1 4
tcgtgggcgccgtgctgaccctgccgcagggtttcaccacctccaaggaa

SEQ ID NO: 30 - DNA, 50 bp, Pseudomonas aeruginosa
  source 1..50: mol_type = genomic DNA; note = target sequence ccoN1 5
gccgaactggaatggccgctggccatcctcctggcgatcgtctggatcac

SEQ ID NO: 31 - DNA, 50 bp, Pseudomonas aeruginosa
  source 1..50: mol_type = genomic DNA; note = target sequence ccoN1 6
gccttcatcctggtgacggcgatgctgcacatcgtcaaccacatgtcgct

SEQ ID NO: 32 - DNA, 50 bp, Pseudomonas aeruginosa
  source 1..50: mol_type = genomic DNA; note = target sequence ccoN1 7
actggtcgcaagccacccgggcttcatcgtgcgcatgatcggcggtggtt

SEQ ID NO: 33 - DNA, 50 bp, Pseudomonas aeruginosa
  source 1..50: mol_type = genomic DNA; note = target sequence ackA 1
gccctcacgcaacatactggtgatcaactgcggcagttcgtcgatcaagt

SEQ ID NO: 34 - DNA, 50 bp, Pseudomonas aeruginosa
  source 1..50: mol_type = genomic DNA; note = target sequence ackA 2
ccctggtcaacgaggcccactccctgtttcccctgcacggcctcgccgag

SEQ ID NO: 35 - DNA, 50 bp, Pseudomonas aeruginosa
  source 1..50: mol_type = genomic DNA; note = target sequence ackA 3
ctgggcagccgcgatgcggtgctgcgctggaagcgcggcggcgacagcga

SEQ ID NO: 36 - DNA, 50 bp, Pseudomonas aeruginosa
  source 1..50: mol_type = genomic DNA; note = target sequence ackA 4
cctgatgattcccaacgccgaccaccgcgccgccctcgcccagttgctgc

SEQ ID NO: 37 - DNA, 50 bp, Pseudomonas aeruginosa
  source 1..50: mol_type = genomic DNA; note = target sequence ackA 5
tggtgcagaacgccgcgggcggcaagctccacggcatcggccaccgggtg

SEQ ID NO: 38 - DNA, 50 bp, Pseudomonas aeruginosa
  source 1..50: mol_type = genomic DNA; note = target sequence ackA 6
catggcggcgagctgttcacccatgccacgcgcatcgacgaccgggtggt

SEQ ID NO: 39 - DNA, 50 bp, Pseudomonas aeruginosa
  source 1..50: mol_type = genomic DNA; note = target sequence ackA 7
ggcgatccgggccaccgcgccgctggcgccgctgcacaacccggccaacc

SEQ ID NO: 40 - DNA, 50 bp, Pseudomonas aeruginosa
  source 1..50: mol_type = genomic DNA; note = target sequence ackA 8
aaggcatcgaggcagcgatgacgctgtttcccaagctgccccacgtcgcc

SEQ ID NO: 41 - DNA, 50 bp, Pseudomonas aeruginosa
  source 1..50: mol_type = genomic DNA; note = target sequence ackA 9
ttcgacaccgccttccaccagagcctgccggagcacgcctaccgctacgc

SEQ ID NO: 42 - DNA, 50 bp, Pseudomonas aeruginosa
  source 1..50: mol_type = genomic DNA; note = target sequence ackA 10
agcatggcgtgcgccgctacggcttccacggcaccagccaccgctacgtc

SEQ ID NO: 43 - DNA, 50 bp, Pseudomonas aeruginosa
  source 1..50: mol_type = genomic DNA; note = target sequence ackA 11
ggcgacagcagttggctcagcgcccacctcggcaacggcagctcgacctg

SEQ ID NO: 44 - DNA, 50 bp, Pseudomonas aeruginosa
  source 1..50: mol_type = genomic DNA; note = target sequence ackA 12
catcgtcaacggccagagcctcgacaccagcatgggcctgaccccgctgg

SEQ ID NO: 45 - DNA, 50 bp, Pseudomonas aeruginosa
  source 1..50: mol_type = genomic DNA; note = target sequence ackA 13
gcctggtaatgggcacccgcagcggcgacgtcgaccccaacctgcacagc

SEQ ID NO: 46 - DNA, 50 bp, Pseudomonas aeruginosa
  source 1..50: mol_type = genomic DNA; note = target sequence ackA 14
ctggcgcggaccctgggctggagcctggagcgcatcgactcgatgctgaa

SEQ ID NO: 47 - DNA, 50 bp, Pseudomonas aeruginosa
  source 1..50: mol_type = genomic DNA; note = target sequence ackA 15
cgaaagcggcctgctcggcctctccgacctgtccaacgacatgcgcaccc

SEQ ID NO: 48 - DNA, 50 bp, Pseudomonas aeruginosa
  source 1..50: mol_type = genomic DNA; note = target sequence ackA 16
agcaggagcgcgagcagggccaccccggcgcggccctggcgatcgaggtg

SEQ ID NO: 49 - DNA, 50 bp, Pseudomonas aeruginosa
  source 1..50: mol_type = genomic DNA; note = target sequence ackA 17
tgctaccgcctggccaagtccctggcggcgatgagctgcgccctgccgca

SEQ ID NO: 50 - DNA, 50 bp, Pseudomonas aeruginosa
  source 1..50: mol_type = genomic DNA; note = target sequence ackA 18
ggacggggtgatcttcaccggtggcatcggcgagaactcgccgctggtgc

SEQ ID NO: 51 - DNA, 50 bp, Pseudomonas aeruginosa
  source 1..50: mol_type = genomic DNA; note = target sequence ackA 19
ccaagaccgccgcccacctgcggctgttcgacctgcgcctcgaccaggag

SEQ ID NO: 52 - DNA, 50 bp, Pseudomonas aeruginosa
  source 1..50: mol_type = genomic DNA; note = target sequence ackA 20
aacgcccgctgcgtgcgcggcgtcgccgggccgatccaggccgcgggaca

SEQ ID NO: 53 - DNA, 50 bp, Pseudomonas aeruginosa
  source 1..50: mol_type = genomic DNA; note = target sequence ackA 21
gcgggtactggtgatcccgaccaacgaagagcggcagatcgccctcgaca

SEQ ID NO: 54 - DNA, 50 bp, Pseudomonas aeruginosa
  source 1..50: mol_type = genomic DNA; note = target sequence nirS 1
gccatttggcaagccactggtgggcaccttgctcgcctcgctgacgctgc

SEQ ID NO: 55 - DNA, 50 bp, Pseudomonas aeruginosa
  source 1..50: mol_type = genomic DNA; note = target sequence nirS 2
gcctggccaccgctcacgccaaggacgacatgaaagccgccgagcaatac

SEQ ID NO: 56 - DNA, 50 bp, Pseudomonas aeruginosa
  source 1..50: mol_type = genomic DNA; note = target sequence nirS 3
ggtgccgcttccgccgtcgatcccgctcacgtggtgcgcaccaacggtgc

SEQ ID NO: 57 - DNA, 50 bp, Pseudomonas aeruginosa
  source 1..50: mol_type = genomic DNA; note = target sequence nirS 4
cgacatgagtgaaagcgagttcaacgaggccaagcagatctacttccaac

SEQ ID NO: 58 - DNA, 50 bp, Pseudomonas aeruginosa
  source 1..50: mol_type = genomic DNA; note = target sequence nirS 5
ccggacatcacccagcaacgcggccagcaatacctggaagcgctgatcac

SEQ ID NO: 59 - DNA, 50 bp, Pseudomonas aeruginosa
  source 1..50: mol_type = genomic DNA; note = target sequence nirS 6
cggcaccccgctgggcatgccgaactggggcagctccggcgagctgagca

SEQ ID NO: 60 - DNA, 50 bp, Pseudomonas aeruginosa
  source 1..50: mol_type = genomic DNA; note = target sequence nirS 7
aacagatcaccctgatggccaagtacatccagcacaccccgccgcaaccg

SEQ ID NO: 61 - DNA, 50 bp, Pseudomonas aeruginosa
  source 1..50: mol_type = genomic DNA; note = target sequence nirS 8
gagtggggcatgccggagatgcgcgaatcgtggaaggtgctggtgaagcc

SEQ ID NO: 62 - DNA, 50 bp, Pseudomonas aeruginosa
  source 1..50: mol_type = genomic DNA; note = target sequence nirS 9
ggaccggccgaagaaacagctcaacgacctcgacctgcccaacctgttct

SEQ ID NO: 63 - DNA, 50 bp, Pseudomonas aeruginosa
  source 1..50: mol_type = genomic DNA; note = target sequence nirS 10
tgaccctgcgcgacgccgggcagatcgccctggtcgacggcgacagcaag

SEQ ID NO: 64 - DNA, 50 bp, Pseudomonas aeruginosa
  source 1..50: mol_type = genomic DNA; note = target sequence nirS 11
atcgtcaaggtcatcgataccggctatgccgtgcatatctcgcggatgtc

SEQ ID NO: 65 - DNA, 50 bp, Pseudomonas aeruginosa
  source 1..50: mol_type = genomic DNA; note = target sequence nirS 12
ttccggccgctacctgctggtgatcggccgcgacgcgcggatcgacatga

SEQ ID NO: 66 - DNA, 50 bp, Pseudomonas aeruginosa
  source 1..50: mol_type = genomic DNA; note = target sequence nirS 13
acctgtgggccaaggagccgaccaaggtcgccgagatcaagatcggcatc

SEQ ID NO: 67 - DNA, 50 bp, Pseudomonas aeruginosa
  source 1..50: mol_type = genomic DNA; note = target sequence nirS 14
gcgcgctcggtggaaagctccaagttcaagggctacgaggaccgctacac

SEQ ID NO: 68 - DNA, 50 bp, Pseudomonas aeruginosa
  source 1..50: mol_type = genomic DNA; note = target sequence nirS 15
cgccggcgcctactggccgccgcagttcgcgatcatggacggcgagaccc

SEQ ID NO: 69 - DNA, 50 bp, Pseudomonas aeruginosa
  source 1..50: mol_type = genomic DNA; note = target sequence nirS 16
aaccgaagcagatcgtctccacccgcggcatgaccgtagacacccagacc

SEQ ID NO: 70 - DNA, 50 bp, Pseudomonas aeruginosa
  source 1..50: mol_type = genomic DNA; note = target sequence nirS 17
cacccggaaccgcgcgtggcggcgatcatcgcctcccacgagcaccccga

SEQ ID NO: 71 - DNA, 50 bp, Pseudomonas aeruginosa
  source 1..50: mol_type = genomic DNA; note = target sequence nirS 18
catcgtcaacgtgaaggagaccggcaaggtcctgctggtcaactacaagg

SEQ ID NO: 72 - DNA, 50 bp, Pseudomonas aeruginosa
  source 1..50: mol_type = genomic DNA; note = target sequence nirS 19
tcgacaacctcaccgtcaccagcatcggtgcggcgccgttcctccacgac

SEQ ID NO: 73 - DNA, 50 bp, Pseudomonas aeruginosa
  source 1..50: mol_type = genomic DNA; note = target sequence nirS 20
ggctgggacagcagccaccgctacttcatgaccgccgccaacaactccaa

SEQ ID NO: 74 - DNA, 50 bp, Pseudomonas aeruginosa
  source 1..50: mol_type = genomic DNA; note = target sequence nirS 21
ggttgccgtgatcgactccaaggaccgtcgcctgtcggccctggtcgacg

SEQ ID NO: 75 - DNA, 50 bp, Pseudomonas aeruginosa
  source 1..50: mol_type = genomic DNA; note = target sequence nirS 22
gcaagaccccgcacccggggcgtggcgccaacttcgtgcatcccaagtac

SEQ ID NO: 76 - DNA, 52 bp, Pseudomonas aeruginosa
  source 1..52: mol_type = genomic DNA; note = target sequence narG 1
atgagtcacctgctcgaccgcctgcagttcttcaagaagaagcagggcgaat

SEQ ID NO: 77 - DNA, 52 bp, Pseudomonas aeruginosa
  source 1..52: mol_type = genomic DNA; note = target sequence narG 2
cggcagcgctggcagcacgacaagatcgtgcgctccacccacggggtgaact

SEQ ID NO: 78 - DNA, 52 bp, Pseudomonas aeruginosa
  source 1..52: mol_type = genomic DNA; note = target sequence narG 3
accggctcctgctcctggaagatctacgtgaagaacggcctgatcacctggg

SEQ ID NO: 79 - DNA, 52 bp, Pseudomonas aeruginosa
  source 1..52: mol_type = genomic DNA; note = target sequence narG 4
ctgaagtacccgaaggtgcgcaagccgttgctcaagctctggcgcgaggcgc

SEQ ID NO: 80 - DNA, 52 bp, Pseudomonas aeruginosa
  source 1..52: mol_type = genomic DNA; note = target sequence narG 5
tgggacgaggtcaccgagatcatcgccgcggccaacgtctacaccgccaaga

SEQ ID NO: 81 - DNA, 52 bp, Pseudomonas aeruginosa
  source 1..52: mol_type = genomic DNA; note = target sequence narG 6
gtgcgctacaagggcaccaagaccgtctccatcaccccggactattccgagg

SEQ ID NO: 82 - DNA, 52 bp, Pseudomonas aeruginosa
  source 1..52: mol_type = genomic DNA; note = target sequence narG 7
ggcatggccttcggtcacgtgatcctgaaggaattccacctcgaccggccga

SEQ ID NO: 83 - DNA, 52 bp, Pseudomonas aeruginosa
  source 1..52: mol_type = genomic DNA; note = target sequence narG 8
gcctacttcgtcgactactgccgccagtacaccgacatgccgatgctggtgt

SEQ ID NO: 84 - DNA, 52 bp, Pseudomonas aeruginosa
  source 1..52: mol_type = genomic DNA; note = target sequence narG 9
cagacgcgcctgcaactgtcgctgctcgatggcccggaacatgcctgcgagg

SEQ ID NO: 85 - DNA, 52 bp, Pseudomonas aeruginosa
  source 1..52: mol_type = genomic DNA; note = target sequence narG 10
gccttcccgtatttcgccgggcaggagcacccgcacttcaagggcgtcgcca

SEQ ID NO: 86 - DNA, 52 bp, Pseudomonas aeruginosa
  source 1..52: mol_type = genomic DNA; note = target sequence narG 11
atggtgatcatcggcgcggcgatgaaccactggtaccacatggacatgaact

SEQ ID NO: 87 - DNA, 52 bp, Pseudomonas aeruginosa
  source 1..52: mol_type = genomic DNA; note = target sequence narG 12
agcttcttctacctgcacagctcgcaatggcgccacgagaagctgtcgatgc

SEQ ID NO: 88 - DNA, 52 bp, Pseudomonas aeruginosa
  source 1..52: mol_type = genomic DNA; note = target sequence narG 13
ccggacgatccgcagaacttcccgcgcaacatgttcatctggcgctccaacc

SEQ ID NO: 89 - DNA, 52 bp, Pseudomonas aeruginosa
  source 1..52: mol_type = genomic DNA; note = target sequence narG 14
gaggtcgactgggttgacgacggtgccgagggcaagctcgacctggtcacca

SEQ ID NO: 90 - DNA, 52 bp, Pseudomonas aeruginosa
  source 1..52: mol_type = genomic DNA; note = target sequence narG 15
ctggacttccgcatgtcctccacctgcatgtactcggacatcgtcctgccga

SEQ ID NO: 91 - DNA, 52 bp, Pseudomonas aeruginosa
  source 1..52: mol_type = genomic DNA; note = target sequence narG 16
gctacctggtacgagaaggacgacctcaacacctccgacatgcaccccttca

SEQ ID NO: 92 - DNA, 52 bp, Pseudomonas aeruginosa
  source 1..52: mol_type = genomic DNA; note = target sequence narG 17
gtctaccgcaagttcacctcgctcggtccgctgctggacaagctgggcaacg

SEQ ID NO: 93 - DNA, 52 bp, Pseudomonas aeruginosa
  source 1..52: mol_type = genomic DNA; note = target sequence narG 18
ggcaagggcatcggctggaacaccgagaaggaagtgaagctggtcggcgacc

SEQ ID NO: 94 - DNA, 52 bp, Pseudomonas aeruginosa
  source 1..52: mol_type = genomic DNA; note = target sequence narG 19
gtcgcggtcaaggcctgggaagcgctgtcgaagatcaccggccgcgagcatg

SEQ ID NO: 95 - DNA, 52 bp, Pseudomonas aeruginosa
  source 1..52: mol_type = genomic DNA; note = target sequence narG 20
cacctggcgctgcccaaggaagacgagaagatccgcttccgcgacatccagg